SEQUENCE LISTING (47 sequences)

SEQ ID NO: 1 - protein, 5 aa, Artificial Sequence
  Description of Artificial Sequence Synthetic peptide
RRXXX

SEQ ID NO: 2 - protein, 7 aa, Artificial Sequence
  Description of Artificial Sequence Synthetic peptide
LRRASLG

SEQ ID NO: 3 - protein, 6 aa, Artificial Sequence
  Description of Artificial Sequence Synthetic peptide
RXXXFF

SEQ ID NO: 4 - protein, 7 aa, Artificial Sequence
  Description of Artificial Sequence Synthetic peptide
RQGSFRA

SEQ ID NO: 5 - protein, 4 aa, Artificial Sequence
  Description of Artificial Sequence Synthetic peptide
XPXX

SEQ ID NO: 6 - protein, 4 aa, Artificial Sequence
  Description of Artificial Sequence Synthetic peptide
PXXP

SEQ ID NO: 7 - protein, 7 aa, Artificial Sequence
  Description of Artificial Sequence Synthetic peptide
RRIPLSP

SEQ ID NO: 8 - protein, 9 aa, Artificial Sequence
  Description of Artificial Sequence Synthetic peptide
KKKKRFSFK

SEQ ID NO: 9 - protein, 8 aa, Artificial Sequence
  Description of Artificial Sequence Synthetic peptide
XRXXSXRX

SEQ ID NO: 10 - protein, 9 aa, Artificial Sequence
  Description of Artificial Sequence Synthetic peptide
LRRLSDSNF

SEQ ID NO: 11 - protein, 10 aa, Artificial Sequence
  Description of Artificial Sequence Synthetic peptide
KKLNRTLTVA

SEQ ID NO: 12 - protein, 7 aa, Artificial Sequence
  Description of Artificial Sequence Synthetic peptide
EEIYXXF

SEQ ID NO: 13 - protein, 8 aa, Artificial Sequence
  Description of Artificial Sequence Synthetic peptide
EEIYGEFR

SEQ ID NO: 14 - protein, 6 aa, Artificial Sequence
  Description of Artificial Sequence Synthetic peptide
EIYEXX

SEQ ID NO: 15 - protein, 6 aa, Artificial Sequence
  Description of Artificial Sequence Synthetic peptide
IYMFFF

SEQ ID NO: 16 - protein, 4 aa, Artificial Sequence
  Description of Artificial Sequence Synthetic peptide
YMMM

SEQ ID NO: 17 - protein, 5 aa, Artificial Sequence
  Description of Artificial Sequence Synthetic peptide
EEEYF

SEQ ID NO: 18 - protein, 12 aa, Artificial Sequence
  Description of Artificial Sequence Synthetic peptide
RIGEGTYGVVRR

SEQ ID NO: 19 - protein, 7 aa, Artificial Sequence
  Description of Artificial Sequence Synthetic peptide
RPRTSSF

SEQ ID NO: 20 - protein, 7 aa, Artificial Sequence
  Description of Artificial Sequence Synthetic peptide
PRTPGGR

SEQ ID NO: 21 - protein, 8 aa, Artificial Sequence
  Description of Artificial Sequence Synthetic peptide
RLNRTLSV

SEQ ID NO: 22 - protein, 8 aa, Artificial Sequence
  Description of Artificial Sequence Synthetic peptide
DRRLSSLR

SEQ ID NO: 23 - protein, 12 aa, Artificial Sequence
  Description of Artificial Sequence Synthetic peptide
EAIYAAPFARRR

SEQ ID NO: 24 - protein, 15 aa, Artificial Sequence
  Description of Artificial Sequence Synthetic peptide
KVEKIGEGTYGVVYK

SEQ ID NO: 25 - protein, 8 aa, Artificial Sequence
  Description of Artificial Sequence Synthetic peptide
EEEIYGEF

SEQ ID NO: 26 - protein, 12 aa, Artificial Sequence
  Description of Artificial Sequence Synthetic peptide
RHSSPHQSEDEE

SEQ ID NO: 27 - protein, 18 aa, Artificial Sequence
  Description of Artificial Sequence Synthetic peptide
RRKDLHDDEEDEAMSITA

SEQ ID NO: 28 - protein, 4 aa, Artificial Sequence
  Description of Artificial Sequence Synthetic peptide
SXXX

SEQ ID NO: 29 - protein, 4 aa, Artificial Sequence
  Description of Artificial Sequence Synthetic peptide
SXXX

SEQ ID NO: 30 - protein, 10 aa, Artificial Sequence
  Description of Artificial Sequence Synthetic peptide
RRRDDDSDDD

SEQ ID NO: 31 - protein, 17 aa, Artificial Sequence
  Description of Artificial Sequence Synthetic peptide
KGPWLEEEEEAYGWLDF

SEQ ID NO: 32 - protein, 7 aa, Artificial Sequence
  Description of Artificial Sequence Synthetic peptide
EEIYGEF

SEQ ID NO: 33 - protein, 8 aa, Artificial Sequence
  Description of Artificial Sequence Synthetic peptide
RREIYGRF

SEQ ID NO: 34 - protein, 7 aa, Artificial Sequence
  Description of Artificial Sequence Synthetic peptide
RRIYGRF

SEQ ID NO: 35 - protein, 10 aa, Artificial Sequence
  Description of Artificial Sequence Synthetic peptide
RRIPLSPLSP

SEQ ID NO: 36 - protein, 7 aa, Artificial Sequence
  Description of Artificial Sequence Synthetic peptide
PXSPXSP

SEQ ID NO: 37 - protein, 6 aa, Artificial Sequence
  Description of Artificial Sequence Synthetic peptide
PFHLVI

SEQ ID NO: 38 - protein, 10 aa, Artificial Sequence
  Description of Artificial Sequence Synthetic peptide
SLRRRRFSKG

SEQ ID NO: 39 - protein, 8 aa, Artificial Sequence
  Description of Artificial Sequence Synthetic peptide
LRRRRFSK

SEQ ID NO: 40 - protein, 7 aa, Artificial Sequence
  Description of Artificial Sequence Synthetic peptide
LRRASLG

SEQ ID NO: 41 - protein, 15 aa, Artificial Sequence
  Description of Artificial Sequence Synthetic peptide
LSPSLSRHSSHQRRR

SEQ ID NO: 42 - protein, 5 aa, Artificial Sequence
  Description of Artificial Sequence Synthetic peptide
SRHSS

SEQ ID NO: 43 - protein, 5 aa, Artificial Sequence
  Description of Artificial Sequence Synthetic peptide
SPSLS

SEQ ID NO: 44 - protein, 12 aa, Artificial Sequence
  Description of Artificial Sequence Synthetic peptide
RGRPRTSSFAEG

SEQ ID NO: 45 - protein, 7 aa, Artificial Sequence
  Description of Artificial Sequence Synthetic peptide
RREGSFR

SEQ ID NO: 46 - protein, 15 aa, Artificial Sequence
  Description of Artificial Sequence Synthetic peptide
KSPSKSRHSSHQRRR

SEQ ID NO: 47 - protein, 6 aa, Artificial Sequence
  Description of Artificial Sequence Synthetic peptide
RRESFR

(19) United States
(12) Patent Application Publication
(10) Pub. No.: US 2010/0112619 A1
(43) Pub. Date: May 6, 2010

(54) LIGAND-CONTAINING MICELLES AND USES THEREOF

(76) Inventors: Ronald J. Graham, Carlsbad, CA (US); Linda G. Lee, Palo Alto, CA (US); Hongye Sun, San Mateo, CA (US)

Correspondence Address: LIFE TECHNOLOGIES CORPORATION;C/O INTELLEVATE, P.O. BOX 52050, MINNEAPOLIS, MN, 55402, US

(21) Appl. No.: 12/469,597

(22) Filed: May 20, 2009

Related U.S. Application Data
(63) Continuation of application No. 10/997,066, filed on Nov. 24, 2004, now abandoned.
(60) Provisional application No. 60/525,492, filed on Nov. 26, 2003.
(60) Provisional application No. 60/628,509, filed on Nov. 15, 2004.

(51) Int. Cl. C12Q 1/34; C09K 11/06

(52) U.S. Cl. 435/18; 252/301.16

(57) ABSTRACT

Ligand-containing micelles and various compositions, kits and methods for their preparation and use are provided.

### 1. CROSS-REFERENCES TO RELATED APPLICATIONS

This application claims benefit under 35 U.S.C. §119(e) to application Ser. No. 60/525,492, entitled “Ligand Containing Micelles and Uses Thereof,” filed Nov. 26, 2003, and to application Ser. No. ______, entitled “Ligand Containing Micelles and Uses Thereof,” filed Nov. 15, 2004; the disclosures of which are incorporated herein by reference in their entirety.

### 2. FIELD

The present disclosure relates to compositions and methods for detecting and/or characterizing binding interactions.

### 3. INTRODUCTION

Binding interactions between molecules such as ligands and receptors mediate numerous biological processes. For example, many disease pathways are effected by the binding of a ligand to a receptor, which can either “turn on” or “turn off” a cascade of events that leads to manifestation of the disease. The ability to identify ligands for newly identified receptors, or to identify compounds that inhibit binding interactions between ligands and receptors is extremely desirable. For example, compounds that act as inhibitors of ligand-receptor interactions, or compounds that can disrupt or inhibit protein-protein interactions might have clinical or other significances. The ability to detect, identify, characterize, and screen for binding interactions and/or compounds capable of inhibiting or disrupting binding interactions is therefore desirable. More generally, there is a need for new detection methodologies.

### 4. SUMMARY

In one aspect, provided herein are ligand-containing micelles useful for, among other things, detecting or evaluating binding interactions between ligands and other molecules. The micelles comprise a labeling system that permits the micelles to be selectively “turned on” by treatment with specified agents. The micelles can exist in a variety of different forms, ranging from non-lamellar “detergent-like” micelles which do not enclose or encapsulate solvent, to lamellar vesicle-like micelles which do enclose or encapsulate solvent (e.g., aqueous solvent), such as, for example, liposomes. The lamellar vesicle-like micelles may be unilamellar or multilamellar, and may vary in size, ranging from small to large. In some embodiments, such micelles comprise small unilamellar vesicles or liposomes (“SUVs”), small multilamellar vesicles or liposomes (SMVs”), large unilamellar vesicles or liposomes (“LUVs”) and/or large multilamellar vesicles or liposomes (“LMVs”). A collection of micelles may all be of the same type or, alternatively, may comprise mixtures of two or more of the various different micellar forms. Vesicle-like micelles may be unfilled, or all or a subset of them may encapsulate or enclose an agent, such as a fluorescent molecule, a quencher molecule or a mixture thereof.

The ligand-containing micelles generally comprise a ligand and an amphipathic signal molecule capable of generating or providing a detectable fluorescent signal under specified conditions. The amphipathic signal molecule comprises one or more hydrophobic moieties, one or more fluorescent moieties, an optional modification moiety, and/or an optional charge balance moiety.

The hydrophobic moiety(ies) are selected such that, taken together, they are capable of integrating the signal compound into the micelle. In some embodiments, each hydrophobic moiety comprises a saturated or unsaturated hydrocarbon comprising from 6 to 30 carbon atoms. When a signal molecule comprises more than one hydrophobic moiety, the hydrophobic moieties may be the same, some of them may be the same and others different, or they may all differ from one another. In some embodiments, the signal molecule comprises two hydrophobic moieties, each of which comprises a hydrocarbon chain corresponding in structure to a hydrocarbon chain or “tail” of a naturally occurring lipid or phospholipid.

In some embodiments, the hydrophobic moiety(ies) facilitate an increase in the fluorescence of the fluorescent moiety following modification of the signal molecule such that the intensity of the fluorescence following modification is greater than would be obtained with the same signal molecule lacking the hydrophobic moiety(ies).

The fluorescent moiety may be any fluorescent entity that is operative in accordance with the various compositions and methods described herein. In some embodiments, the fluorescent moiety comprises at least one fluorescein dye. In some embodiments, the fluorescent moiety comprises at least one rhodamine dye. In some embodiments, the fluorescent moiety comprises two or more fluorescent dyes that can act cooperatively with one another, such as by, for example, fluorescence resonance energy transfer (“FRET”).

In some embodiments, the fluorescence of the fluorescent moiety is quenched when the signal molecule is integrated into the micelle. This quenching may be accomplished by a variety of different mechanisms. In some embodiments, the signal molecule comprises a fluorescent moiety that is capable of “self-quenching” when in close proximity to another fluorescent moiety of the same type. In such embodiments, the micelle may comprises signal molecules in an amount or concentration high enough to bring the fluorescent moieties of different signal molecules in sufficiently close proximity to one another such that the fluorescence of their fluorescent moieties is quenched.

In some embodiments, quenching can be achieved with the aid of a quenching moiety. The quenching moiety can be any moiety capable of quenching the fluorescence of the fluorescent moiety of a signal molecule when it is in close proximity thereto, such as, for example, by orbital overlap (formation of a ground state dark complex), collisional quenching, FRET, or another mechanism or combination of mechanisms. The quenching moiety can itself be fluorescent, or it can be non-fluorescent. In some embodiments, the quenching moiety comprises a fluorescent dye that has an absorbance spectrum that sufficiently overlaps the emissions spectrum of the fluorescent moiety of the signal molecule such that it quenches the fluorescence of the fluorescent moiety when in close proximity thereto. In such embodiments, selecting a quenching moiety that fluoresces at a wavelength resolvable from that of the fluorescent moiety can provide an internal signal standard to which the fluorescence signal can be referenced and also permits the micelles to be “tracked” by the fluorescence of the quenching moiety.

The quenching moiety can be included in the signal molecule, or it can be included in a distinct quenching molecule that has properties that permit it to integrate into the micelle to quench the fluorescence of the fluorescent moieties of the signal molecules, for example. In some embodiments, a quenching molecule comprises at least one hydrophobic moiety, such as one of the hydrophobic moiety(ies) described above, and a quenching moiety. The quenching molecule can optionally comprise a modification moiety, as will be described in more detail below. When the quenching molecule comprises an optional modification moiety, such that selective modification of the quenching molecule leads to unquenching of the fluorescent moieties of the signal molecules.

In embodiments in which the quenching moiety is included in the signal molecule, treatment with a modification agent results in releasing the quenching and fluorescent moieties from close proximity, typically by cleavage of the signal molecule, thereby unquenching the fluorescence of the fluorescence moiety.

Regardless of the mechanism by which the quenching effect is achieved, modification of the modification moiety of a signal molecule and/or a quenching molecule by a selected modification agent leads to unquenching of the fluorescence signal, thereby producing a detectable change in fluorescence. The mechanism by which the modification leads to unquenching is not critical, and can be selected by the user, depending, in part, on the particular application. For example, modification may involve a change in the overall net charge of the signal molecule (or quenching molecule if it comprises a modification moiety), for example by phosphorylation of a residue with a kinase enzyme or by dephosphorylation of a residue with a phosphatase enzyme. As another specific example, the modification may involve cleavage of the signal molecule, and/or quenching molecule, such as by a cleaving enzyme. Non-limiting examples of cleaving enzymes that could be used are lipases, phospholipases, proteases and nucleases.

The chemical structure of the modification moiety will depend (in part) upon the particular modification agent. The modification moiety may comprise all or a part of one or more of the other moieties or features comprising the signal or quenching molecule, depending upon the requirements of the modification agent.

In some embodiments, the modification moiety comprises an enzyme recognition moiety that is recognized and modified by an enzyme. In other embodiments, the modification moiety comprises an enzyme recognition moiety that comprise one, two, or more recognition sequences for a specified modification agent. When the enzyme recognition moiety comprises two or more enzyme recognition sequences, the enzyme recognition sequences may be the same, some of them may be the same and others different, or they may all differ from another.

In some embodiments, the modification moiety comprises a cleaving enzyme recognition moiety that is recognized and cleaved by a cleaving enzyme. In some embodiments, the cleaving enzyme recognition moiety comprises an oligonucleotide or oligonucleotide analog having a primary sequence that is recognized and cleaved by a nuclease, such as a ribonuclease or a deoxyribonuclease. In some embodiments, the cleaving enzyme recognition moiety comprises a peptide or peptide analog that is recognized and cleaved by a protease.

In still another specific exemplary embodiment, the cleaving enzyme recognition moiety comprises a structure that is recognized and cleaved by a phospholipase. The phospholipase recognition moiety may comprise features that facilitate binding specificity, affinity and/or rate of cleavage. The phospholipase recognition moiety can be designed to be recognized and cleaved by a particular phospholipase or group of phospholipases. In some embodiments, the phospholipase recognition moiety is recognized and cleaved by one or more of the following: a phospholipase C (“PLC”), a phospholipase A (“PLA”), such as a phospholipase A1 (“PLA1”) or a phospholipase A2 (“PLA2”) a phospholipase D (“PLD”), or a phospholipase B (“PLB”).

In some embodiments, the modification moiety comprises at least one protein kinase recognition moiety that comprises one or more unphosphorylated residues that are capable of being phosphorylated by a protein kinase, such as, for example, one or more tyrosine, serine and/or threonine residues (or phosphorylatable analogs thereof). The protein kinase recognition moiety may also comprise additional residues that facilitate binding specificity, affinity and/or rate of phosphorylation of the particular protein kinase. The protein kinase recognition moiety may be designed to be recognized and modified by a particular protein kinase or group of protein kinases. In a specific embodiment, the protein kinase recognition moiety is recognized and phosphorylated by a protein kinase C.

In some embodiments, the modification moiety comprises at least one phosphatase recognition moiety that comprises one or more phosphorylated residues that are capable of being dephosphorylated by a phosphatase, such as one or more phosphorylated tyrosine, serine and/or threonine residues (or dephosphorylatable phosphorylated analogs thereof). The phosphatase recognition moiety may also comprise additional residues that facilitate specificity, affinity and/or rate of dephosphorylation of the particular phosphatase. The phosphatase recognition moiety may be designed to be recognized and dephosphorylated by a particular phosphatase or group of phosphatases.

In some embodiments, the modification moiety comprises a substrate, i.e., a trigger moiety, that when acted on by a specified agent, i.e., a trigger agent, is capable of generating an intermediate compound that spontaneously rearranges resulting in fragmentation of the signal molecule. In some embodiments, fragmentation results in the release of the fluorescent moiety from the signal molecule. In other embodiments, fragmentation results in the release of the hydrophobic moiety from the signal molecule. Regardless of whether the fluorescent moiety or the hydrophobic moiety is released, the fluorescent signal produced by the fluorescent moiety is increased, indicating the presence of the molecule of interest in the sample.

The chemical structure of the trigger moiety will depend, in part, upon the particular trigger agent. In some embodiments, the trigger moiety comprises a cleavage site that is recognized and cleaved by a cleaving enzyme. For example, the cleaving enzyme can be a lipase, an esterase, a phosphatase, a glycosidase, a carboxypeptidase or a catalytic antibody. In some embodiments, the trigger moiety comprises an oligonucleotide or oligonucleotide analog having a sequence that is recognized and cleaved by a nuclease, such as a ribonuclease or a deoxyribonuclease. In some embodiments, the trigger moiety comprises a peptide or peptide analog that is recognized and cleaved by a protease.

In addition to having a cleavage site for a cleaving enzyme, the trigger moiety may comprise additional linkages that facilitate the attachment of the cleavage site to the signal molecule. In these embodiments, the additional linkages are capable of undergoing spontaneous rearrangement such that fragmentation of the substrate compound results.

In other embodiments, reduction of an aromatic nitro or azide compound can be used as a bioreductive trigger agent to generate a r electron-donor species, e.g. —NH—, that is capable of initiating a spontaneous rearrangement reaction, resulting in fragmentation of the signal molecule.

In other embodiments, the trigger moiety is also the linker moiety. In these embodiments, cleavage of the trigger moiety results directly in the release of the hydrophobic moiety or the fluorescent moiety. For example, if the linker moiety is a substrate for β-lactamase, cleavage of the linker moiety by β-lactamase initiates a fragmentation reaction that results in the release of either the hydrophobic moiety or the fluorescent moiety.

In some embodiments, micelle formation can be promoted or encouraged by the inclusion of a charge balance moiety. The charge balance moiety acts to balance the overall charge of the composition. For example, if the signal molecule comprises one or more charged chemical groups, the presence of these groups can interfere with micelle formation and/or destabilize the micelle, thereby promoting the release of the signal molecule from the micelle in the absence of the specified enzyme. Stabilization of the micelle can be promoted by including a charge-balance moiety designed to counter the charge of the signal molecule via inclusion of chemical groups that have the opposite charge of the chemical groups comprising the signal molecule, such that the overall charge of the micelle is approximately neutral.

The charge-balance moiety can be designed to have a net negative or net positive charge by including an appropriate number of negatively and positively charged groups in the charge-balance moiety. For example, to establish a net positive charge (i.e., net charge ⁺2), the charge-balance moiety can be designed to contain positively charged groups, or a greater number of positively charged groups than negatively charged groups. To establish a net negative charge (i.e., net charge ⁻2), the charge-balance moiety can be designed to contain negatively charged groups, or a greater number of negatively charged groups than positively charged groups.

The charge balance moiety can be included in the signal molecule, or it can be included in a distinct charge balance molecule that has properties that permit it to integrate into the micelle. In some embodiments, a charge balance molecule comprises at least one hydrophobic moiety, such as one of the hydrophobic moiety(ies) described above, and a charge balance moiety. The charge balance molecule can optionally comprises a fluorescent moiety, as will be described in more detail below.

In some embodiments, the charge balance moiety also comprises a modification moiety capable of being modified by a modification agent. For example, the modification agent can be a cleaving agent, such as a lipase, a phospholipase, a protease or a nuclease. The use of modification agents that do not cleave the signal and charge balance molecules may result in the formation of new aggregates or micelles comprising the modified signal and charge balance molecules, the fluorescence of which could remain quenched. In some embodiments, the modification moiety of the signal molecule and the modification moiety of the charge balance molecule are cleaved by different cleaving enzymes.

In some embodiments, the charge balance molecule comprises a modification moiety and the signal molecule either does not comprise the optional modification moiety or comprises a modification moiety that is modified by a different modification agent than the modification moiety of the charge balance molecule.

The hydrophobic moiety(ies), fluorescent moiety and optional modification, charge balance, and/or quenching moiety(ies) of the signal molecule can be connected in any way that permits them to perform their respective functions. The connectivities may depend, in part, upon the identity of the modification agent that will be used to modify the optional modification moiety and/or whether any quenching moieties are included in the signal molecule. In some embodiments, the hydrophobic moiety(ies) and fluorescent moiety are linked to each other through a modification moiety. In some embodiments, the hydrophobic moiety(ies) and the modification moiety are linked to each other through a fluorescent moiety. In some embodiments, the hydrophobic moiety(ies), fluorescent moiety and modification moiety are linked to one another by a multivalent linker. Multivalent linkers can be any molecule having two, three, four, or more attachment sites suitable for attaching other molecules and moieties thereto, or that can be appropriately activated to attach other molecules and moieties thereto.

The ligand-containing micelle also comprises a ligand. The ligand can comprise any molecule of interest (or portion or fragment thereof) that can be associated with, or conjugated to, the micelle and for which a binding partner is known or desired. For example, the ligand may be a small organic molecule, a drug, a hapten, a vitamin, a peptide, a protein, a toxin, a hormone, an enzyme, a substrate, a transition state analog, a protein, a protein receptor, an antigen, a receptor ligand, a cytokine, a growth factor, an antibody, a mono- or polysaccharide or a nucleic acid, including, for example, an oligo- or polynucleotide, an mRNA, a cDNA or a gene. In some embodiments, the ligand comprises one member of a pair of specific binding molecules, such as, for example, one member of a receptor-ligand pair. In some embodiments, the ligand comprises a molecule whose ability to bind another molecule is sought to be determined. As another specific example, the ligand can comprise a small organic molecule, such as a drug lead or candidate, whose ability to bind a protein, receptor or other molecule of interest is sought to be determined.

The ligand may be associated with, or conjugated to, the micelle by virtually any suitable means. In some embodiments, the ligand is included as part of an amphipathic ligand molecule that aids integration of the ligand into the micelle. Such ligand molecules generally comprise the ligand and one or more hydrophobic moieties, such as, for example, one or more of the hydrophobic moieties described above, and may optionally comprise additional features, such as, for example, a modification moiety, a fluorescent moiety a charge balance moiety, and/or a quenching moiety, as previously described. For example, the amphipathic ligand molecule can comprise a ligand covalently attached to a fatty acid or a phospholipid, optionally via a linker, which helps integrate the ligand into the micelle. In some embodiments, the ligand is “embedded” in the micelle without the aid of exogenous hydrophobic moiety(ies). For example, the ligand may be an integral membrane protein that resides within a layer of a uni- or multilamellar vesicle-like micelle. In some embodiments, the ligand is aqueously soluble and is stably associated with the micelle via non-covalent interactions, such as, for example, by biotin-streptavidin interactions.

Also provided are methods that utilize ligand-containing micelles such as discussed above. In some embodiments, a method is provided for detecting a binding activity of a ligand-binding molecule in a sample that comprises the steps of:

(a) contacting the sample with a micelle comprising a ligand and a signal molecule comprising at least one hydrophobic moiety, a fluorescent moiety and an optional modification moiety under conditions effective to permit binding between the ligand and the ligand-binding molecule (if present in the sample), wherein the fluorescence of the fluorescent moiety of the signal molecule is quenched within the micelle;

(b) removing unbound micelles from the sample;

(c) subjecting the sample to conditions effective to unquench the fluorescence of the fluorescent moiety of the signal molecule; and

(d) detecting a fluorescence signal, where an increase in the fluorescence signal indicates the presence of a binding activity of a ligand-binding molecule in the sample.

In some embodiments, the ligand-binding molecule is immobilized on, or attached to, a substrate, such as a solid support or a solid surface.

In some embodiments of such methods, the signal molecule comprises the optional modification moiety and step (c) is carried out by contacting the sample with a modification agent under conditions effective to permit the modification agent to modify the modification moiety of the signal molecule, thereby yielding an increase in the fluorescence in the sample.

In some embodiments of such methods, the micelle further comprises a charge balance moiety capable of promoting micelle formation by balancing the overall charge of the composition in the absence of a modification agent. In such embodiments, contacting the sample with a modification agent under conditions effective to permit modification of the modification moiety of the signal molecule can result in the formation of one or more additional charged groups, such that the charge balance moiety is no longer capable of balancing the overall charge of the micelle. Such modification leads to unquenching of the fluorescence signal of the fluorescent moiety, thereby increasing the fluorescence signal.

In some embodiments of such methods, the micelle further comprises a quenching molecule comprising a quenching moiety capable of quenching the fluorescence of the fluorescent moiety of the signal molecule when in close proximity thereto, at least one hydrophobic moiety capable of integrating the quenching molecule into the micelle and an optional modification moiety, which can be the same as or different from the optional modification moiety of the signal molecule. In such embodiments, step (c) may be carried out in a variety of ways. In some embodiments, the signal molecule comprises the optional modification moiety and step (c) can be carried out by contacting the sample with a modification agent under conditions effective to permit modification of the modification moiety of the signal molecule. Such modification leads to unquenching of the fluorescence signal of the fluorescent moiety, thereby increasing the fluorescence signal.

In some embodiments, the quenching molecule also comprises a modification moiety capable of being modified by a modification agent. For example, the modification agent can be a cleaving agent, such as a lipase, a phospholipase, a protease or a nuclease. The use of modification agents that do not cleave the signal and quenching molecules may result in the formation of new aggregates or micelles comprising the modified signal and quenching molecules, the fluorescence of which could remain quenched. In some embodiments, the modification moiety of the signal molecule and the modification moiety of the quenching molecule are cleaved by different cleaving enzymes.

In some embodiments, the quenching molecule comprises a modification moiety and the signal molecule either does not comprise the optional modification moiety or comprises a modification moiety that is modified by a different modification agent than the modification moiety of the quenching molecule. In some embodiments, step (c) can be carried out by contacting the sample with a modification agent that modifies the modification moiety of the quenching molecule, resulting in unquenching of the fluorescent moiety of the signal molecule, thereby increasing the fluorescence signal. As will be appreciated by skilled artisans, modification of the quenching molecule following binding according to this variation yields a fluorescent micelle, making this variation ideally suited to applications in which the binding partner or putative binding partner for the ligand is immobilized or attached to a solid support or surface. An increase in fluorescence of the support following modification of the quenching molecule indicates the presence of a binding partner for the ligand on the solid support or surface.

In some embodiment of methods herein, the signal molecule comprises the optional modification moiety and further comprises a quenching moiety that quenches the fluorescence of its fluorescent moiety. Use of a modification moiety that can be cleaved by a modification agent at step (c) releases the quenching and fluorescent moieties from one another, thereby unquenching the fluorescence signal of the fluorescent moiety.

The micelles and methods described herein may be used in a variety of different contexts. As a specific example, the micelles and methods may be used to characterize binding interactions between a ligand and a ligand-binding molecule. Such characterization can comprise, but is not limited to, measuring or determining the dissociation constant (Kd) of the binding interaction under specified conditions or a variety of different conditions. As another specific example, the micelles and methods may be used to detect, screen for, quantitate and/or identify ligand-binding molecules in a sample. For example, the micelles can be contacted with a plurality of different candidate molecules to identify those molecules that bind the ligand.

Such assays can be carried out in a “single plex” mode in which each candidate compound of the plurality is contacted individually with the micelle, or in a “multiplex” mode in which all or a subset of the candidate compounds are contacted simultaneously with the micelle. For example, in embodiments in which modification of the micelle releases the fluorescent moiety into the assay medium, the candidate compounds can be attached to individual solid supports (one or a plurality of candidate compounds per support) and the supports dispensed into the wells of a multiwell tray or plate, one or a plurality of supports per well. Alternatively, the compounds could be attached directly to the walls or bottoms of the wells. An increase in the fluorescence signal in a well indicates that at least one candidate compound in the well bound the ligand on the micelle.

As another example, in embodiments in which modification of the micelle yields a fluorescent micelle, the candidate compounds can be attached to individual solid supports (one or a plurality of candidate compounds per support) and contacted in a batch-wise fashion with the ligand-containing micelle. Following modification, those supports that fluoresce can be retrieved, for example by handpicking or with the aid of an automated sorter, such as, for example, a FACS machine, and the identities of their immobilized candidate compounds determined. Alternatively, the immobilized candidate compounds could be arranged in an ordered array in which their structures are identifiable by their relative and/or absolute positions or locations within the array (for example by dispensing the individual solid supports described above into the wells of a multiwell tray or plate or by attaching the candidate compounds to a single solid support or surface at specified locations, such as specified xy coordinates). An increase in fluorescence at a particular location within the array following modification not only indicates the presence of a binding partner for the ligand at that particular location, but also its structure. In some embodiments of such multiplexed assays, the complexity of the assay can be increased by using a plurality of different ligand-containing micelles. In some embodiments, each micelle comprises a fluorescent moiety having a fluorescence spectrum or signal that is resolvable from the fluorescence spectra or signals of the fluorescent moieties on the other micelles such that the identities of their ligands can be correlated with a specified fluorescence signal or “color.”

As another example, the micelles and methods can be used to screen for and/or identify a ligand that binds a molecule of interest. For example, a plurality of micelles may be prepared, each of which comprises a different putative ligand (or ligand candidate) and contacted with the molecule of interest to identify those putative ligands that bind the molecule. Such screening assays may be carried out in a “single-plex” mode in which each micelle of the plurality is contacted individually with the molecule of interest, or in a “multiplex” mode in which all or a subset of the micelles are contacted simultaneously with the molecule of interest. In some embodiments of such multiplexed assays, each micelle can comprise a fluorescent moiety having a fluorescence spectrum or signal that is resolvable from the fluorescence spectra or signals of the fluorescent moieties of the signal molecules of the other micelles such that the identities of their putative ligands can be correlated with a specified fluorescence signal or “color.”

As another example, the micelles and methods can be used to screen for, identify and/or characterize inhibitors and/or modulators of the binding interaction between a ligand and another molecule, as discussed further herein.

In another aspect, the present disclosure provides signal molecules, quenching molecules, ligand-containing micelles and kits containing the signal molecules, quenching molecules and/or ligand-containing micelles, as discussed further herein.

These and other features of the compositions and methods described herein will become more apparent from the detailed description below.

### 5. BRIEF DESCRIPTION OF THE DRAWINGS

The skilled artisan will understand that the drawings, described below, are for illustration purposes only. The drawings are not intended to limit the scope of the present teaching in any way. In the drawings, similar elements are referenced with like numbers.

FIGS. 1A-1C illustrate exemplary embodiments of glycerophospholipid signal molecules;

FIGS. 2A-2C illustrate the cleavage products generated by treating the glycerophospholipid signal molecules 100, 200 and 300 of FIGS. 1A-1C, respectively, with various different modification agents;

FIGS. 3A-3B illustrate exemplary schemes for synthesizing exemplary glycerophospholipid signal molecules 60 and 57, respectively;

FIGS. 4A-D illustrate the release of a dye moiety or a hydrophobic moiety following fragmentation of the substrate compound;

FIG. 5A illustrates an exemplary embodiment of a substrate compound in which the trigger moiety also serves as the linker moiety;

FIG. 5B illustrates an exemplary embodiment of a substrate compound comprising an aromatic linker moiety that fragments via 1,6-elimination reaction and the resulting fragmentation products;

FIGS. 5C-5F illustrate exemplary embodiments of substrate compounds comprising linker moieties that fragment via a trimethyl lock lactonization reaction and the resulting fragmentation products;

FIGS. 5G-5H illustrate exemplary embodiments of substrate compounds comprising linker moieties that fragment via a ring closure mechanism and the resulting fragmentation products;

FIGS. 6A-6D illustrate exemplary methods of synthesizing substrate compounds that comprise a linker moiety that fragments via a 1,6-elimination reaction;

FIGS. 7A-7B illustrates another exemplary method of synthesizing a substrate compound that comprises a linker moiety that fragments via a 1,4- and a 1,6-elimination reaction;

FIGS. 8A-8B illustrates an exemplary method of synthesizing a substrate compound that comprises a linker moiety that fragments via a bis 1,4-elimination reaction;

FIGS. 9A-9E illustrate other exemplary methods of synthesizing a substrate compound that comprises a linker moiety that fragments via a 1,6-elimination reaction;

FIGS. 10A-10B illustrate an exemplary method of synthesizing a substrate compound that comprises a linker moiety that fragments via a ring closure mechanism;

FIGS. 11A-11Q illustrate exemplary embodiments of dye-peptide signal molecules;

FIGS. 12A-12N illustrate other exemplary embodiments of dye-peptide signal molecules;

FIG. 13 provides a cartoon illustrating in situ attachment of a ligand to a preformed micelle to yield an embodiment of a ligand-containing micelle;

FIGS. 14A-14C illustrate exemplary embodiments of glycerophospholipid ligand molecules;

FIG. 15A illustrates an exemplary embodiment of dual role ligand/signal molecule;

FIG. 15B illustrates an exemplary embodiment of a glycerophospholipid dual role ligand/signal molecule;

FIG. 15C illustrates the cleavage products generated by treating the ligand/signal molecule 700 of FIG. 15B with phospholipases A1, A2, C and D;

FIG. 15D illustrates an exemplary embodiment of a glycerophospholipid dual role ligand/signal molecule 750 and the cleavage products generated by treatment with phospholipases A1 and A2;

FIG. 15E illustrates an exemplary embodiment of a glycerophospholipid dual role ligand/signal molecule 720 that comprises a quenching moiety and the cleavage products generated by treatment with phospholipases A1 and A2;

FIG. 15F illustrates exemplary embodiments of trivalent linker synthons that can be used to provide a trivalent linker;

FIG. 15G illustrates an exemplary method of synthesizing the dual role ligand/signal molecule 700 of FIG. 11B;

FIGS. 16A-16B illustrate exemplary embodiments of quenching molecules that can comprise a ligand-containing micelle;

FIG. 17A-17B illustrate exemplary embodiments of micelle formation in the presence of a charge balance moiety;

FIGS. 18A-18F illustrate exemplary embodiments of binding assay schemes utilizing exemplary embodiments of ligand-containing micelles;

FIG. 19A shows the addition of varying concentrations (0, 5, 10, 20, 50 μM) of a charge-balance molecule, C₁₆RROOORRIYGRF quenching the fluorescence of a substrate molecule, C₁₆K(Dye2)OOOEETYGEF (10 μM) in 25 mM Tris (pH 7.6);

FIG. 19B shows the rate of reaction of 5 nM tyrosine kinase (Lyn) against the substrate molecule C₁₆K(Dye2)OOOEEIYGEF (2 μM), charge-balance molecule C₁₆RROOORRIYGRF (2 μM), with 0 and 100 μM ATP;

FIG. 20A shows the rate of reaction for a kinase substrate i.e., C₁₂OOK(Dye 2)RRIPLSPOOK(C₁₂)NH₂ (2 μM) comprising two hydrophobic moieties for protein kinase p38βII (14 nM) for several concentrations of ATP (0, 5, 10, 20, 50, 100, 200, and 500 μM);

FIG. 20B shows the rate of reaction for a kinase substrate, i.e. C₁₆OOOK(Dye 2)RRIPLSPNH₂ (4 μM) comprising one hydrophobic moiety for protein kinase p38βII (14 nM) for several concentrations of ATP (0, 5, 10, 20, 50, 100, 200, and 500 μM);

FIG. 21A shows the rate of reaction for a kinase substrate, i.e., C₁₁OOK(Dye2)RRIPLSPLSPOOK(C₁₁)—NH₂ (8 μM) for 10 and 100 μM ATP; and,

FIG. 21B shows the rate of reaction for a kinase substrate, i.e., C₁₁OOK(Dye2)RRIPLSPOOK(C₁₁)—NH₂ (8 μM) for 10 and 100 μM ATP.

### 6. DETAILED DESCRIPTION

It is to be understood that both the foregoing general description and the following detailed description are exemplary and explanatory only and are not restrictive of the compositions and methods described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting.

### 6.1 DEFINITIONS

As used herein, the following terms and phrases are intended to have the following meanings:

“Detect” and “detection” have their standard meaning, and are intended to encompass detection, measurement, and/or characterization of a selected molecule or molecular activity.

As a specific example, binding activity or interactions may be “detected” in the course of detecting the presence of, screening for, and/or characterizing binding partners, modulators or inhibitors of a binding molecule.

“Lipid” has its standard meaning and is intended to refer to a hydrophobic or an amphipathic organic molecule, such as a steroid, a fat, a fatty acid, a phospholipid or a water-insoluble vitamin.

“Fatty Acid” has its standard meaning and is intended to refer to a long-chain hydrocarbon carboxylic acid in which the hydrocarbon chain is saturated, mono-unsaturated or polyunsaturated. The hydrocarbon chain may be linear, branched or cyclic, or may comprise a combination of these features, and may be unsubstituted or substituted. Fatty acids typically have the structural formula R—C(O)OH, where R is a substituted or unsubstituted, saturated, mono-unsaturated or polyunsaturated hydrocarbon comprising from 6 to 30 carbon atoms which has a structure that is linear, branched, cyclic or a combination thereof.

“Phospholipid” has its standard meaning and is intended to comprise compounds which comprise two fatty acid moieties, a backbone moiety, a phosphate moiety, and an organic moiety. Specific examples of phospholipids include glycerophospholipids and sphingolipids. Specifically included within the definition of “phospholipid” are glycerophospholipids having the following structure:

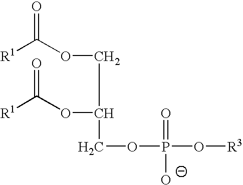

wherein:

R¹ is a saturated, mono-unsaturated or polyunsaturated hydrocarbon having from 6 to 30 carbon atoms;

R² is a saturated, mono-unsaturated or polyunsaturated hydrocarbon having from 6 to 30 carbon atoms; and

R³ is —CH₂CH₂—N⁺ (CH₃)₃ (cholinyl), —CH₂CH₂NH₂ (ethanolamin-2-yl), inositolyl, —CH₂CH(NH₃⁺)C(O)OH (serinyl) or —CH₂CH(NH₂)—CH(OH)—CH═CH—(CH₂)₁₂CH₃ (sphingosinyl).

“Micelle” has its standard meaning and is intended to refer to an aggregate formed by amphipathic molecules in water or an aqueous solvent such that their polar ends or portions are in contact with the water or aqueous solvent and their nonpolar ends or portions are in the interior of the aggregate. A micelle can take any shape or form, including but not limited to, a non-lamellar “detergent-like” aggregate that does not enclose a portion of the water or aqueous solvent, or a unilamellar or multilamellar “vesicle-like” aggregate that encloses a portion of the water or aqueous solvent, such as, for example, a liposome. Specifically included within the definition of “micelle” are small unilamellar vesicles or liposomes (“SUVs”), small multilamellar vesicles or liposomes (“SMVs”), large unilamellar vesicles or liposomes (“LUVs”) and large multilamellar vesicles or liposomes (“LMVs”)

“Quench” has its standard meaning and is intended to refer to a measurable reduction in the fluorescence intensity of a fluorescent group or moiety as measured at a specified wavelength, regardless of the mechanism by which the reduction is achieved. As specific examples, the quenching may be due to molecular collision, energy transfer such as FRET, a change in the fluorescence spectrum (color) of the fluorescent group or moiety or any other mechanism (or combination of mechanisms). The amount of the reduction is not critical and may vary over a broad range. The only requirement is that the reduction be measurable by the detection system being used. Thus, a fluorescence signal is “quenched” if its intensity at a specified wavelength is reduced by any measurable amount. A fluorescence signal is “substantially quenched” if its intensity at a specified wavelength is reduced by at least 50%, for example by 50%, 60%, 70%, 75%, 80%, 85%, 90%, 95%, 96%, 97%, 98%, 99% or even 100%.

Polypeptide sequences are provided with an orientation (left to right) of the N terminus to C terminus, with amino acid residues represented by the standard 3-letter or 1-letter codes (e.g., Stryer, L., Biochemistry, 2^{nd} Ed., W.H. Freeman and Co., San Francisco, Calif., page 16 (1981)).

### 6.2 Exemplary Embodiments

Provided herein are compositions, methods and kits that utilize ligand-containing-micelles. In some embodiments, ligand-containing micelles comprise as one component an amphipathic signal molecule which comprises one or more fluorescent moieties, one or more hydrophobic moieties, and a modification moiety that comprises one or more modification sites that can be modified by a specified agent. The fluorescent moiety(ies), the hydrophobic moiety(ies) and the modification moiety are connected in any way that permits them to perform their respective functions. The fluorescence signal of the fluorescent moiety is quenched when the signal molecule is integrated into the micelle. Modification of the modification moiety by the specified agent reduces or eliminates the quenching effect, thereby producing a detectable increase in fluorescence. Suitable types of modifications include, but are not limited to, cleavage, or addition, deletion or substitution of chemical group(s).

In some embodiments, modification promotes the dissociation of the fluorescent moiety from the micelle, thereby reducing or eliminating the quenching effect caused by the interactions between the fluorescent moiety and the micelle. The dissociation may be caused by cleavage of the signal molecule. The dissociation may also be caused by adding or deleting chemical groups to the signal molecule, such as phosphate groups, which can destabilize the signal molecule in the micelle, promoting its release therefrom.

In another embodiment, the signal molecule further comprises a charge-balance moiety that acts to balance the overall charge of the composition. For example, if the signal molecule comprises one or more charged chemical groups, the presence of these groups can interfere with micelle formation and/or destabilize the signal molecule in the micelle, resulting in a detectable fluorescence in the absence of the specified modification agent. Micelle formation can be promoted or encouraged by including a charge-balance moiety designed to counter the charge of the signal molecule via the inclusion of chemical groups that have the opposite charge of the chemical groups comprising the signal molecule, such that the overall charge of the micelle is approximately neutral. Thus, by including the charge-balance moiety, micelles can be formed in the presence of destabilizing chemical groups.

In another embodiment, the signal molecule further comprises a quenching moiety that quenches the fluorescence of the fluorescent moiety. The quenching moiety can be positioned so that it is able to intramolecularly quench the fluorescence of the fluorescent moiety on the signal molecule which includes it, or, alternatively, the quenching moiety may be positioned so that intramolecular quenching does not occur. In either embodiment, the quenching moiety may intermolecularly quench the fluorescence of a fluorescent moiety on another signal molecule in the micelle which is in close proximity thereto. Modification of the modification moiety of the signal molecule by a specified agent “deactivates” the quenching effect by relieving the close proximity of the quenching and fluorescent moieties, thereby generating a measurable increase in fluorescence signals.

In some embodiments, the ligand-containing micelles comprise a signal molecule as one component and a charge-balance molecule as another component. The signal molecule comprises at least one hydrophobic moiety capable of integrating the signal molecule into the micelle and a modification moiety that can be modified by a specified agent. The charge-balance molecule comprises at least one hydrophobic moiety capable of integrating the charge balance molecule into the micelle and a charge-balance moiety that acts to balance the overall charge of the composition. One or both of the signal and/or charge-balance molecules further comprises a fluorescent moiety. When both the signal and charge balance molecules comprise a modification moiety, they can be modifiable by the same modification agent, or by different agents. The various moieties of the signal and charge-balance molecules are connected in any way that permits them to perform their respective functions. Modification of the modification moiety by the specified agent reduces or eliminates the quenching effect, by relieving their close proximity, thereby producing a detectable increase in fluorescence. Suitable types of modifications comprise those described above.

In some embodiments, the ligand-containing micelles comprise a signal molecule as one component and a quenching molecule as another component. The signal molecule comprises at least one hydrophobic moiety capable of integrating the signal molecule into the micelle and a fluorescent moiety. The quenching molecule comprises at least one hydrophobic moiety capable of integrating the quenching molecule into the micelle and a quenching moiety capable of quenching the fluorescence of the fluorescent moiety of the signal molecule when in close proximity thereto. One or both of the signal and quenching molecules also comprises a modification moiety that can be modified by a specified agent. When both the signal and quenching molecules comprise a modification moiety, they can be modifiable by the same modification agent, or by different agents. The various moieties of the signal and quenching molecules are connected in any way that permits them to perform their respective functions. Modification of the modification moiety(ies) by the specified agent(s) reduces or eliminates the quenching effect, by relieving their close proximity, thereby producing a detectable increase in fluorescence. Suitable types of modifications comprise those described above.

The ligand-containing micelles described herein can be used as selectively activatable dyes to detect and/or evaluate interactions between the ligand and other molecules. The micelles may also be used to identify molecules that can modulate the interactions between the ligand and its binding partner. The ligand may comprise any molecule of interest. For instance, the ligand can comprise a small organic molecule, a drug, a hapten, a vitamin, a receptor, a toxin, a hormone, an enzyme, a substrate, a transition state analog, a protein, an antigen, a receptor ligand, a cytokine, a growth factor, an antibody, a peptide, a protein, a mono- or polysaccharide, a nucleic acid, a gene, or any derivative or fragment thereof.

6.2.1 The Signal Molecule

The signal molecules comprising the ligand-containing micelles typically comprise one, two, or more hydrophobic moieties capable of anchoring or integrating the signal molecule into the micelle. The exact numbers, lengths, sizes and/or composition of the hydrophobic moieties can be selectively varied. In embodiments employing two or more hydrophobic moieties, each hydrophobic moiety can be the same, or some or all of the hydrophobic moieties may differ.

In some embodiments, the hydrophobic moiety comprises a substituted or unsubstituted hydrocarbon of sufficient hydrophobic character (e.g., length and/or size) to cause the signal molecule to become integrated or incorporated into a micelle when the signal molecule is placed in an aqueous environment at a concentration above a micelle-forming threshold, such as at or above its critical micelle concentration (CMC). In another embodiment, the hydrophobic moiety comprises a substituted or unsubstituted hydrocarbon comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The hydrocarbon may be linear, branched, cyclic, or any combination thereof. Exemplary linear hydrocarbon groups comprise C6, C7, C8, C9, C10, C11, C12, C13, C14, C15, C16, C17, C18, C19, C20, C22, C24, and C26 alkyl chains.

In some embodiments, the hydrophobic moiety is fully saturated. In some embodiments, the hydrophobic moiety can comprise one or more carbon-carbon double bonds which may be, independently of one another, in the cis or trans configuration, and/or one or more carbon-carbon triple bonds. In some cases, the hydrophobic moiety may have one or more cycloalkyl groups, or one or more aryl rings or arylalkyl groups, such as one or two phenyl rings.

In some embodiments, the hydrophobic moiety is a nonaromatic moiety that does not have a cyclic aromatic pi electron system. In some embodiments, if the hydrophobic moiety contains one or more unsaturated carbon-carbon bonds, those carbon-carbon bonds are not conjugated. In another embodiment, the structure of the hydrophobic moiety is incapable of interacting with the fluorescent moiety, by a FRET or stacking interaction, to quench fluorescence of the fluorescent moiety. Also encompassed herein are embodiments that involve a combination of any two or more of the foregoing embodiments. Optimization testing can be done by making several signal compounds having different hydrophobic moieties.

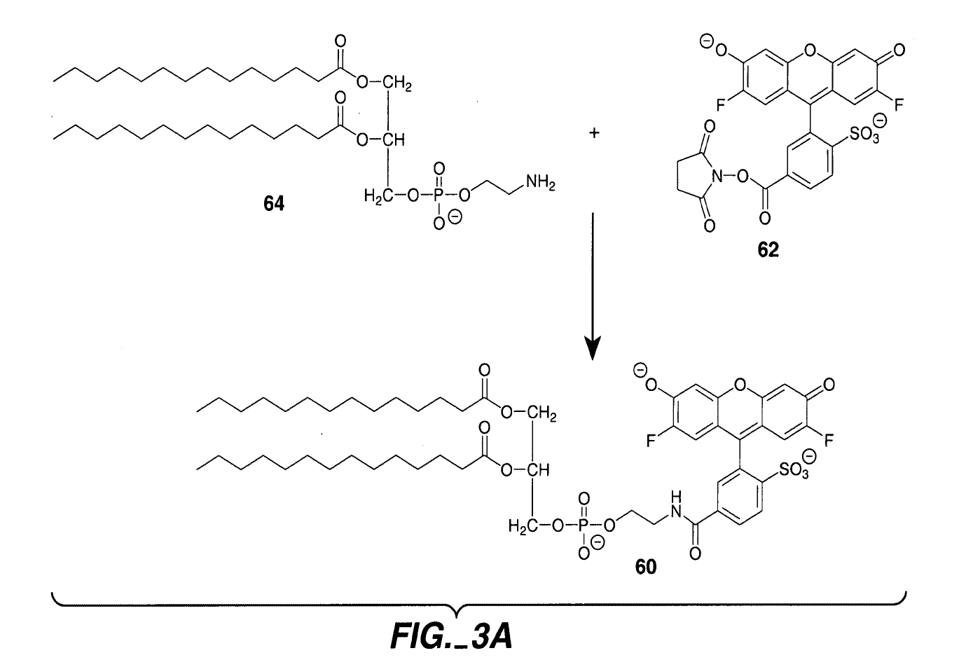

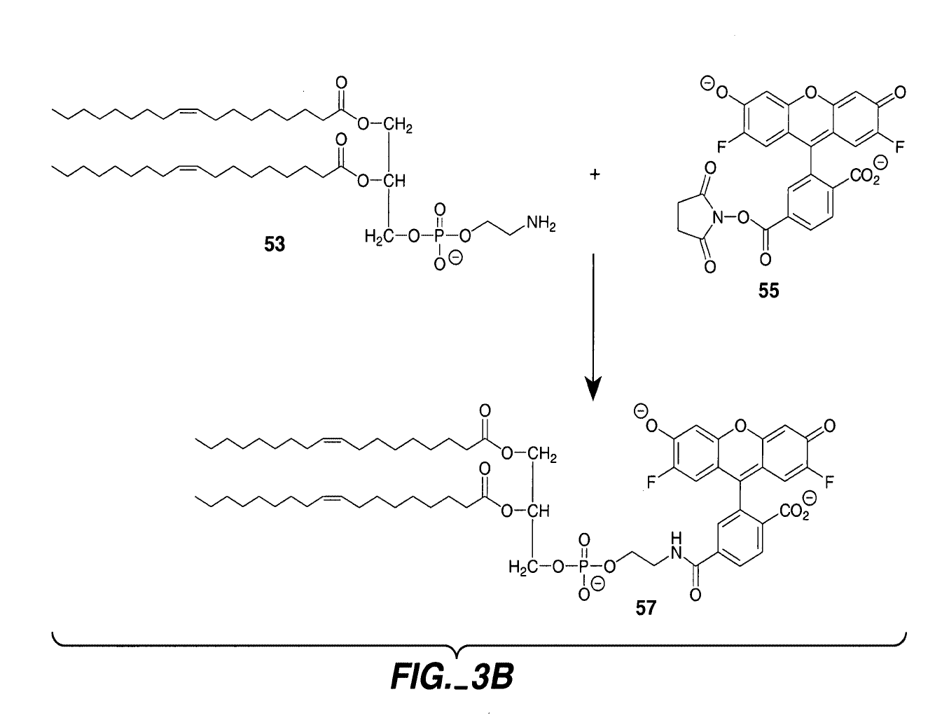

In some embodiments, the hydrophobic moieties comprise amino acids or amino acid analogs that have hydrophobic side chains. The amino acids or analogs are chosen to provide sufficient hydrophobicity to integrate the molecule(s) of the composition into a micelle under the assay conditions used to detect the enzymes. Exemplary hydrophobic amino acids include alanine, glycine, valine, leucine, isoleucine, proline, phenylalanine, methionine, tryptophan, and cysteine as described in Alberts, B., et al., Molecular Biology of the Cell, 4^{th} Ed., Garland Science, New York, N.Y., FIG. 3.3 (2002)). Exemplary amino acid analogs include norvaline, aminobutyric acid, cyclohexylalanine, butylglycine, phenylglycine, and N-methylvaline (see “Amino Acids and Amino Acid Analogs” section 2002-2003 Novabiochem catalog).

The hydrophobicity of a hydrophobic moiety can be calculated by assigning each amino acid a hydrophobicity value and then averaging the values along the hydrophobic moiety. Hydrophobicity values for the common amino acids are shown Table 1.

TABLE 1
Hydrophobicity of Amino Acids

| | Monera et al.¹ | Hopp-Woods² | Kyte-Doolittle³ |
|---|---|---|---|
| Amino Acid | Hydrophobicity at | Hydrophobicity | Hydrophobicity |
| (IUPAC) | pH 7 | scale | scale |
| Alanine (A) | 41 | −0.5 | −1.8 |
| Cysteine (C) | 49 | −1.0 | −2.5 |
| Aspartic acid (D) | −55 | 3.0 | 3.5 |
| Glutamic acid (E) | −31 | 3.0 | 3.5 |
| Phenylalanine (F) | 100 | −2.5 | −2.8 |
| Glycine (G) | 0 | 0.0 | 0.4 |
| Histidine (H) | 8 | −0.5 | 3.2 |
| Isoleucine (I) | 99 | −1.8 | −4.5 |
| Lysine (K) | −23 | 3.0 | 3.9 |
| Leucine (L) | 97 | −1.8 | −3.8 |
| Methionine (M) | 74 | −1.3 | −1.9 |
| Asparagine (N) | −28 | 0.2 | 3.5 |
| Proline (P) | −46 (pH 2) | 0.0 | 1.6 |
| Glutamine (Q) | −10 | 0.2 | 3.5 |
| Arginine (R) | −14 | 3.0 | 4.5 |
| Serine (S) | −5 | 0.3 | 0.8 |
| Threonine (T) | 13 | −0.4 | 0.7 |
| Valine (V) | 76 | −1.5 | −4.2 |
| Tryptophan (W) | 97 | −3.4 | 0.9 |
| Tyrosine (Y) | 63 | −2.3 | 1.3 |

¹Monera et al. J. Protein Sci 1: 219-329 (1995) (The values were normalized so that the most hydrophobic residue (phenylalanine) is given a value of 100 relative to glycine, which is considered neutral (0 value)).
²Hoop TP and Woods KR: Prediction of protein antigenic determinants from amino acid sequences. Proc Natl Acad Sci USA 78: 3824, 1981.
³Kyte J and Doolittle RF: A simple method for displaying the hydropathic character of a protein. J Mol Biol 157: 105, 1982.

The exact number of amino acids and/or amino acid analogs can be selectively varied as long as the hydrophobic moiety comprises sufficient hydrophobic character (e.g., length and/or size) to cause the various molecules described herein to become integrated or incorporated into a micelle when the molecules are placed in an aqueous environment at a concentration at or above its CMC. Thus, in some embodiments, the hydrophobic moiety comprises the same amino acid and/or amino acid analog. In other embodiments, the hydrophobic moiety comprises a mixture of different amino acids and/or amino acid analogs. In yet other embodiments, the hydrophobic moiety comprises a mixture of amino acids and/or amino acid analogs and hydrocarbons.

For embodiments of signal molecules in which the hydrophobic moiety is linked to the fluorescent moiety, it will be understood that the hydrophobic moiety is distinct from the fluorescent moiety because the hydrophobic moiety does not comprise any of the atoms in the fluorescent moiety that are part of the aromatic or conjugated pi-electron system that produces the fluorescent signal. Thus, if a hydrophobic moiety is connected to the C4 position of a xanthene ring (e.g., the C4′ position of a fluorescein or rhodamine dye), the hydrophobic moiety does not comprise any of the aromatic ring atoms of the xanthene ring.

As will be described in more detail below, in some embodiments the signal molecule is an analog or a derivative of a glycerophospholipid. In such embodiments, the signal molecule typically comprises two hydrophobic moieties linked to the C1 and C2 carbons of a glycerolyl group via ester linkages (or other linkages). The two hydrophobic moieties may be the same or they may differ from another. In a specific embodiment, each hydrophobic moiety is selected to correspond to the hydrocarbon chain or “tail” of a naturally occurring fatty acid. In another specific embodiment, the hydrophobic moieties are selected to correspond to the hydrocarbon chains or tails of a naturally occurring phospholipid. Non-limiting examples of hydrocarbon chains or tails of commonly occurring fatty acids are provided in Table 2, below:

TABLE 2

| Length:Number of Unsaturations | Common Name |
|---|---|
| 14:0 | myristic acid |
| 16:0 | palmitic acid |
| 18:0 | stearic acid |
| 18:1 cisΔ⁹ | oleic acid |
| 18:2 cisΔ^{9,12} | linoleic acid |
| 18:3 cisΔ^{9,12,15} | linonenic acid |
| 20:4 cisΔ^{5,8,11,14} | arachidonic acid |
| 20:5 cisΔ^{5,8,11,14,17} | eicosapentaenoic acid |
| | (an omega-3 fatty acid) |

The signal molecule further comprises a fluorescent moiety which can be selectively “turned on” when the signal molecule and/or micelle is modified as described herein. The fluorescent moiety may comprise any entity that provides a fluorescent signal and that can be used in accordance with the methods and principles described herein. The fluorescence of the fluorescent moiety is quenched when the signal molecule is incorporated into the micelle. Modification of the signal molecule (and/or other molecules comprising the micelle as will be described in more detail below) can remove the quenching effect, thereby producing an increase in fluorescence.

Quenching of the fluorescent moiety within the micelle can be achieved in a variety of different ways. In some embodiments, the quenching effect may be achieved or caused by “self-quenching.” Self-quenching can occur when the signal molecules comprising a micelle are present in the micelle at a concentration or molar ratio high enough to bring their fluorescent moieties in close enough proximity to one another such that their fluorescence signals are quenched. Removal of the fluorescent moieties from the micelle reduces or abolishes the “self-quenching,” producing an increase in their fluorescence signals. As used herein, a fluorescent moiety is “released” or “removed” from a micelle if any molecule or molecular fragment that contains the fluorescent moiety is released or removed from the micelle. The fluorescent moiety is preferably soluble under conditions of the assay so as to facilitate removal of the released fluorescent moiety from the micelle into the assay medium.

The quenching effect may also be achieved or caused by other moieties in the signal molecule (or in other “quenching molecules”) comprising the micelle. These moieties are referred to as “quenching moieties,” regardless of the mechanism by which the quenching is achieved. Such quenching moieties and quenching molecules are described in more detail, below. By modifying the quenching moieties to reduce or eliminate their quenching effects, or by removing the fluorescent moiety from proximity of the quenching moieties, the fluorescence of the fluorescent moiety can be substantially restored. As appreciated by those skilled in the art, any mechanism that is capable of causing quenching or changes in fluorescence properties may be used in the micelles and methods described herein.

The degree of quenching achieved within the micelle is not critical for success, provided that it is measurable by the detection system being used. As will be appreciated, higher degrees of quenching are desirable, because the greater the quenching effect, the lower the background fluorescence prior to removal of the quenching effect. In theory, a quenching effect of 100%, which corresponds to complete removal of a measurable fluorescence signal, would be ideal. In practice, any measurable amount will suffice. The molar percentage of signal molecule and optional quenching molecule in a micelle necessary to provide a desired degree of quenching in the micelle may vary depending upon, among other factors, the choice of the fluorescent moiety. The amount of any signal molecule (or mixture of signal molecules) and optional quenching molecule (or mixture of optional quenching molecules) to comprise in a ligand-containing micelle in order to obtain a sufficient degree of quenching can be determined empirically.

Typically, the fluorescent moiety of the signal molecule comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

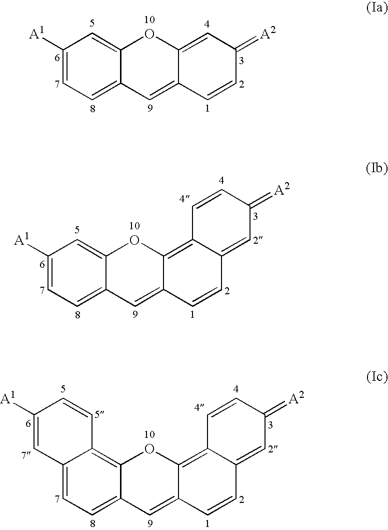

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In some embodiments, typical substituents comprise, but are not limited to, —X, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

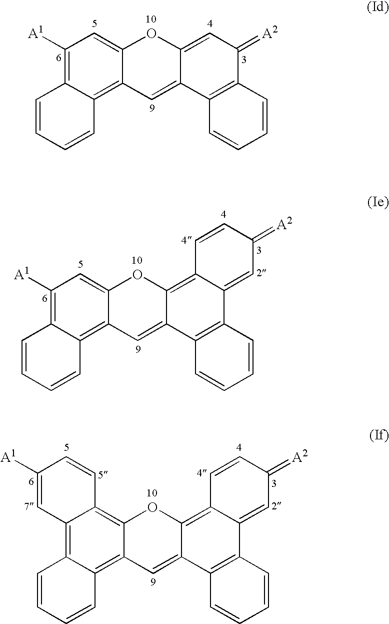

The benzo or aryleno rings may be substituted with a variety of different substituent group, at one or more positions, such as with the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens, are illustrated below:

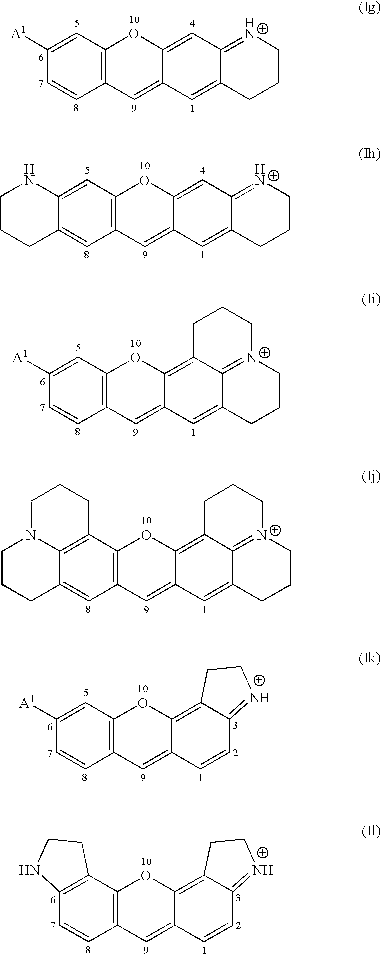

The parent ring may also comprise a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring comprises benzo or aryleno substitutes fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures:

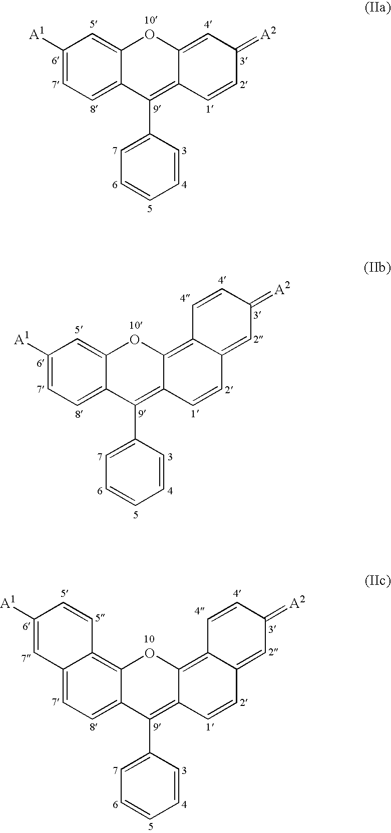

The carbons at positions 3, 4, 5, 6 and 7 may be substituted with a variety of different substituent groups, such as the substituent groups previously described for carbons C1-C8. In a specific embodiment, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In some embodiments, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In some embodiments, the fluorescein moiety comprises a 4,7-dichloro-orthocarboxyfluorescein dye.

In some embodiments, the fluorescent moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent moiety can comprise a network of dyes that operate cooperatively with one another such as, for example by FRET or another mechanism, to provide large Stoke's shifts. Such dye networks typically comprise a fluorescence donor moiety and a fluorescence acceptor moiety, and may comprise additional moieties that act as both fluorescence acceptors and donors. The fluorescence donor and acceptor moieties can comprise any of the previously described dyes, provided that dyes are selected that can act cooperatively with one another. In a specific embodiment, the fluorescent moiety comprises a fluorescence donor moiety which comprises a fluorescein dye and a fluorescence acceptor moiety which comprises a fluorescein or rhodamine dye. Non-limiting examples of suitable dye pairs or networks are described in U.S. Pat. Nos. 6,399,392, 6,232,075, 5,863,727, and 5,800,996.

In many embodiments, the signal molecule also comprises a modification moiety that can be modified by a specified modification agent. Any type of modification may be used, provided that the modification is capable of producing a detectable change (e.g., an increase) in fluorescence. Preferably, the specified agent is substantially active at the interface between the micelle and the assay medium. Selection of a particular modification scheme, and hence modification moiety, may depend, in part, on the structure of the signal molecule, as well as on other factors.

In some embodiments, the modification is based upon cleavage of the signal molecule. In these embodiments, the modification moiety comprises a cleavage site that is cleavable by a chemical reagent or cleaving enzyme. As a specific example, the modification moiety can comprise a cleavage site that is cleavable by a lipase, a phospholipase, a protease, a nuclease or a glycosidase enzyme. The modification moiety may further comprise additional residues and/or features that facilitate the specificity, affinity and/or kinetics of the cleaving enzyme. Depending upon the requirements of the particular cleaving enzyme, such cleaving enzyme “recognition moieties” can comprise the cleavage site or, alternatively, the cleavage site may be external to the recognition moiety. For example, certain endonucleases cleave at positions that are upstream or downstream of the region of the nucleic acid molecule bound by the endonuclease.

The chemical composition of the modification moiety will depend upon, among other factors, the requirements of the cleaving enzyme. For example, if the cleaving enzyme is a protease, the modification moiety can comprise a peptide (or analog thereof) recognized and cleaved by the particular protease. If the cleaving enzyme is a nuclease, the modification moiety can comprise an oligonucleotide (or analog thereof) recognized and cleaved by a particular nuclease. If the cleaving enzyme is a phospholipase, the modification moiety can comprise a diacylglycerolphosphate group recognized and cleaved by a particular phospholipase.

Sequences and structures recognized and cleaved by the various different types of cleaving enzymes are well-known. Any of these sequences and structures comprise the modification moiety. Although the cleavage can be sequence specific, in some embodiments it can be non-specific. For example, the cleavage can be achieved through the use of a non-sequence specific nuclease, such as, for example, an RNase.

Structures recognized and cleaved by lipases such as phospholipases are also well-known. Specific examples of glycerophospholipid signal molecules comprising modification moieties cleavable by phospholipases are described in more detail, below.

Cleavage of the modification moiety of the signal molecule by the corresponding cleaving enzyme can release the fluorescent moiety from the micelle, reducing or eliminating its quenching and producing a measurable increase in fluorescence.

In other embodiments, the modification can be based upon addition, deletion, or substitution of chemical moieties to the signal molecule. These modifications can destabilize the signal molecule in the micelle, thereby promoting its release from the micelle. The release of the signal molecule increases the fluorescence of its fluorescent moiety.

As a specific example, in some embodiments, the modification can be based upon a change in the net charge of the signal molecule, such as by phosphorylation of one or more unphosphorylated residues by a kinase enzyme or dephosphorylation of one or more phosphorylated residues by a phosphatase enzyme. Specific examples of signal molecules comprising modification moieties modifiable by protein kinase and phosphatase enzymes are described in more detail, below.

6.2.2 Glycerophospholipid Signal Molecules

In some embodiments, the signal molecule is an analog or derivative of a glycerophospholipid that has a fluorescent moiety attached thereto, either directly or through an optional linker. The fluorescent moiety can be attached to any portion of the glycerophospholipid. For example, the fluorescent moiety can be attached to the polar “head group” of the glycerophospholipid, or it can be attached to one of the fatty acid “tails” of the glycerophospholipid. In some embodiments the fluorescent moiety can replace the polar head group of the glycerophospholipid and be attached to the phosphate moiety, either directly or through a linker. In some embodiments, the fluorescent moiety can replace one or both of the fatty acid chains of the glycerophospholipid. In these latter embodiments, fluorescent moieties having sufficient hydrophobic character to integrate the resultant glycerophospholipid signal molecule into a micelle should be selected.

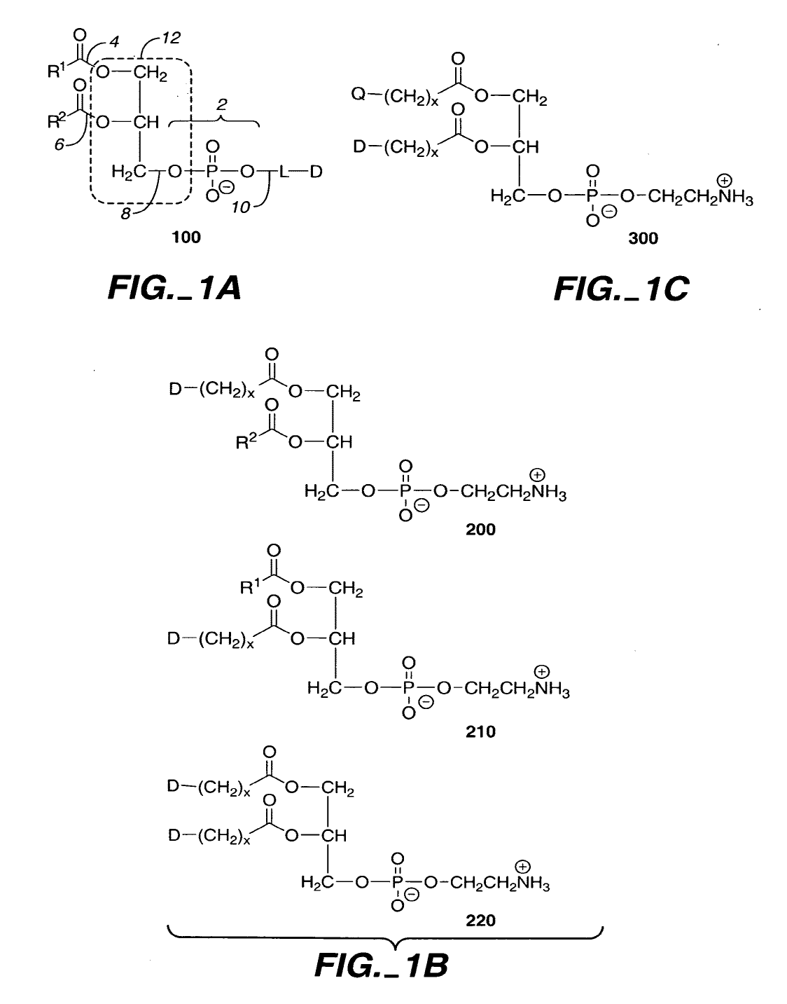

FIG. 1A illustrates an exemplary embodiment of a glycerophospholipid signal molecule 100 that can be used as described herein. Glycerophospholipid signal molecule 100 generally comprises two hydrophobic moieties (represented by R¹ and R²), a phosphate moiety 2 and a fluorescent moiety (represented by “D”). The fluorescent moiety is attached to the phosphate moiety, either directly or by way of an optional linker “L.” The molecule also comprises four modification moieties, each of which comprises a modification site that can be cleaved by PLA1, PLA2, PLC or PLD. The cleavage sites for PLA1, PLA2, PLC and PLD are shown at 4, 6, 8 and 10, respectively. Signal molecule 100 also comprises a glyceroyl “backbone” (highlighted by dashed enclosure 12). The two hydrophobic moieties R¹ and R² and the glyceroyl backbone comprise a part of the various modification moieties. Phosphate moiety 2 may also comprise a part of one or more of the modification moieties.

The hydrophobic moieties R¹ and R² are capable of integrating glycerophospholipid signal molecule 100 into a micelle, such as, for example, into a liposome. Although illustrated in FIG. 1A as ester linkages, the hydrophobic moieties may be attached to the remainder of the molecule via virtually any type of linkage, provided that the resultant glycerophospholipid is cleavable by a specified phospholipase. As illustrated in FIG. 1A, phospholipases A1 and A2 cleave a glycerophospholipid signal molecule 100 at the ester linkages 4 and 6, respectively, which connect hydrophobic moieties R¹ and R² to the remainder of the molecule. Thus, in embodiments in which phospholipase A1 and/or A2 is used to modify signal molecule 100, ester linkages such as those illustrated in FIG. 1A may be preferred. Glycerophospholipid signal molecules having alternative linkages at one or both of these positions 4 and 6, such as thioester, amide, sulfonamide, carbamate or other linkages, may also be employed.

Unlike phospholipases A1 and A2, phospholipases C and D cleave glycerophospholipid signal molecule 100 at phosphate bonds 8 and 10, respectively. In embodiments where phospholipase C or D is used as the modifying agent, the hydrophobic moieties R¹ and R² can be attached to the remainder of the molecule via virtually any type of linkage, provided that the resultant signal molecule 100 can be cleaved by the desired phospholipase.

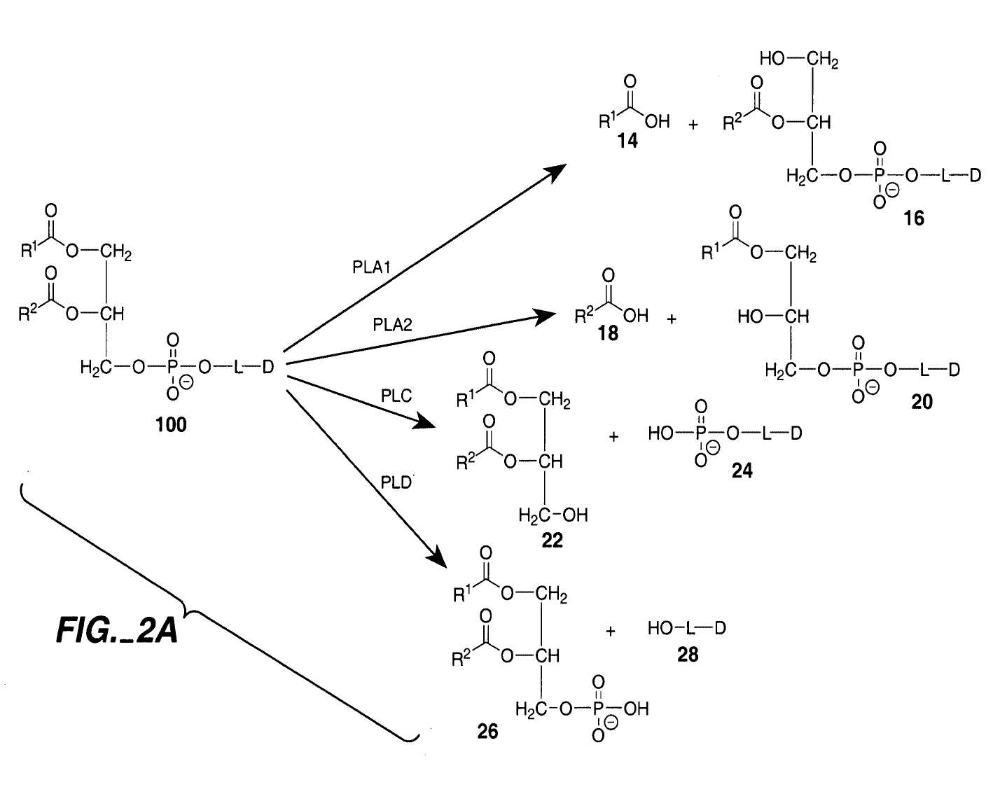

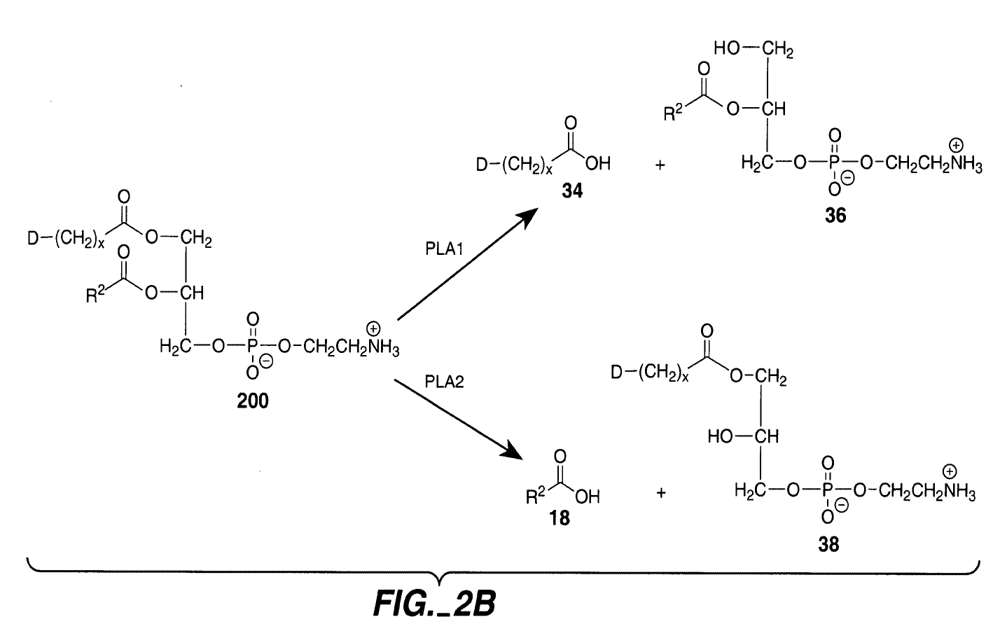

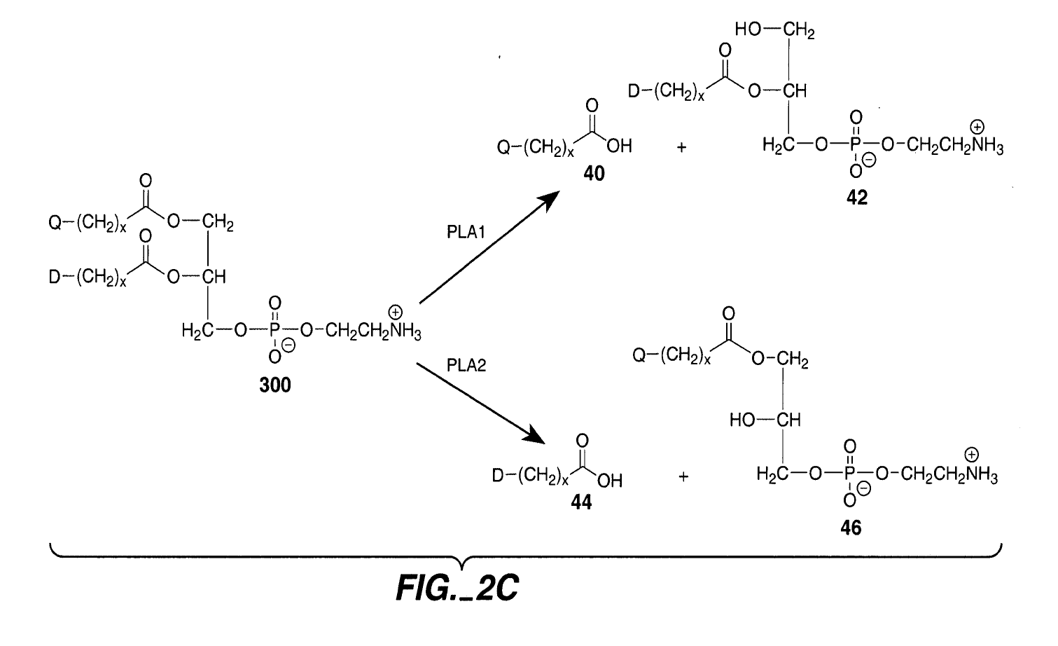

The cleavage products of signal molecule 100 that are generated by treatment with phospholipases A1, A2, C and D are illustrated in FIG. 2A. When integrated into a micelle, cleavage of signal molecule 100 by PLC releases fluorescent moiety “D” into the aqueous environment in the form of phosphorylated fragment 24. Similarly, cleavage by PLD releases fluorescent moiety “D” into the aqueous environment in the form of fragment 28. Once released from the micelle, the fluorescence of the fluorescent moieties of fragments 24 and 28 becomes unquenched, leading to an increase in observed fluorescence. While not intending to be bound by any particular theory of operation, it is believed that, owing to their amphipathic character, lipid fragments 22 and 26 can remain integrated in the micelle, although the micelles and assays work as described herein regardless of whether fragments 22 and 26 remain in the micelle.

Cleavage of signal molecule 100 by PLA1 or PLA2 yields lysophospholipid derivatives 16 and 20, respectively, and fatty acids 14 and 18, respectively. While not intending to be bound by any theory of operation, it is believed that lysophospholipids 16 and 20 dissociate from the micelle into the aqueous environment, which unquenches the fluorescence of their fluorescent moieties and results in an increase in observed fluorescence. The dissociation may lead to the collapse of the liposome altogether.

In signal molecule 100, hydrophobic moieties R¹ and R² can be any of the previously-described substituted or unsubstituted hydrocarbon groups. In a specific embodiment, each of R¹ and R² is a saturated or unsaturated hydrocarbon comprising from 6 to 30 carbon atoms. In still another specific embodiment, each of R¹ and R² is a saturated or unsaturated C6, C7, C8, C9, C10, C11, C12, C13, C14, C15, C16, C17, C18, C19, C20, C22, C24 or C26 alkyl. In another specific embodiment, hydrophobic moieties R¹ and R² correspond in structure to the hydrocarbon tails of naturally occurring fatty acids or phospholipids, such as, for example, the hydrophobic tails of the fatty acids provided in Table 1, supra.

Fluorescent moiety D can be any of the fluorescent moieties described above. In FIG. 1A, the fluorescent moiety D is attached to the remainder of the glycerophospholipid via an optional linker “L.” The chemical composition of linker “L” is not critical. Any type of linker that permits the resultant signal molecule to function as described herein can be used.

The linker “L” can be selected to have specified properties. For example, the linker can be hydrophobic in character, hydrophilic in character, long or short, rigid, semirigid or flexible, depending upon the particular application. The linker can be optionally substituted with one or more substituents or one or more linking groups for the attachment of additional substituents, which may be the same or different, thereby providing a “polyvalent” linking moiety capable of conjugating or linking additional molecules or substances to the signal molecule. In certain embodiments, however, linker “L” does not comprise such additional substituents or linking groups.

A wide variety of linkers “L” comprised of stable bonds are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, linker “L” can include single, double, triple or aromatic carbon-carbon bonds, nitrogen-nitrogen bonds, carbon-nitrogen bonds, carbon-oxygen bonds, carbon-sulfur bonds and combinations of such bonds, and may therefore include functionalities such as carbonyls, ethers, thioethers, carboxamides, sulfonamides, ureas, urethanes, hydrazines, etc. In some embodiments, linker “L” has from 1-20 non-hydrogen atoms selected from the group consisting of C, N, O, and S and is composed of any combination of ether, thioether, amine, ester, carboxamide, sulfonamides, hydrazide, aromatic and heteroaromatic groups.

Choosing a linker “L” having properties suitable for a particular application is within the capabilities of those having skill in the art. For example, where a rigid linker is desired, “L” may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible linker is desired, “L” may comprise a flexible polypeptide such as polyglycine or a flexible saturated alkanyldiyl or heteroalkanyldiyl. Hydrophilic linkers may comprise, for example, polyalcohols or polyethers such as polyalkyleneglycols. Hydrophobic linkers may comprise, for example, alkyldiyls or aryldiyls.

In some embodiments, linker “L” is a peptide bond. Skilled artisans will appreciate that while using peptide bonds may be convenient, the various moieties comprising the substrates can be linked to one another via any linkage that is stable to the conditions under which the substrates will be used.

In some embodiments, the linker “L” comprises atoms and linkages contributed by the polar head group of the glycerophospholipid and/or atoms and linkages used to space the fluorescent moiety “D” from the remainder of the molecule. In a specific embodiment, the linker “L” comprises atoms and linkages formed when a glycerophospholipid having a polar head group including a reactive functional group R^{x} (or precursor thereof that can be activated to be reactive under specified conditions) is covalently coupled to a fluorescent moiety including a “complementary” functional group capable of reacting with R^{x} (or a precursor thereof that can be activated to be reactive with R^{x}), as illustrated in Scheme (I), below:

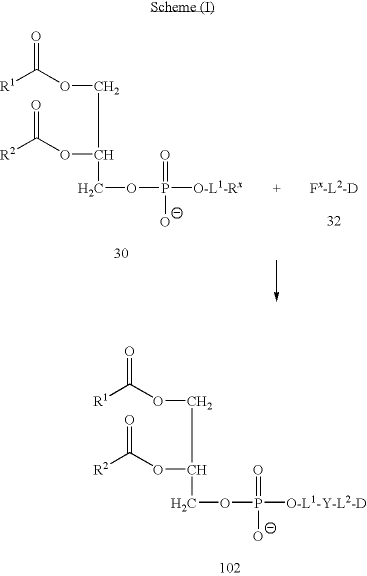

In Scheme (I), R¹, R² and “D” are as defined for FIG. 1A, and R^{x} and F^{x} comprise any complementary reactive groups capable of forming covalent linkages with one another. Pairs of complementary groups capable of forming covalent linkages are well known. In some embodiments, one of R^{x} or F^{x} comprises a nucleophilic group and the other one of R^{x} or F^{x} comprises an electrophilic group. “Complementary” nucleophilic and electrophilic groups (or precursors thereof that can be suitable activated) useful for effecting linkages stable to biological and other assay conditions are well known. Examples of suitable complementary nucleophilic and electrophilic groups, as well as the resultant linkages formed therefrom (represented by “Y” in Scheme (I)), are provided in Table 3.

TABLE 3

| Electrophilic Group | Nucleophilic Group | Resultant Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| Alkyl halides | amines/anilines | alkyl amines |
| Alkyl halides | carboxylic acids | esters |
| Alkyl halides | thiols | thioethers |
| Alkyl halides | alcohols/phenols | ethers |
| Alkyl sulfonates | thiols | thioethers |
| Alkyl sulfonates | carboxylic acids | esters |
| Alkyl sulfonates | alcohols/phenols | esters |
| anhydrides | alcohols/phenols | esters |
| anhydrides | amines/anilines | caroboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | Thiols | thioethers |
| phosphoramidites | Alcohols | phosphate esters |
| silyl halides | Alcohols | silyl ethers |
| sulfonate esters | amines/anilines | alkyl amines |
| sulfonate esters | Thiols | thioethers |
| sulfonate esters | carboxylic acids | esters |
| sulfonate esters | Alcohols | esters |
| sulfonyl halides | amines/anilines | sulfonamides |
| sulfonyl halides | phenols/alcohols | sulfonate esters |
| Diazonium salt | aryl | azo |

*Activated esters, as understood in the art, generally have the formula —C(O)Z, where Z is, a good leaving group (e.g., oxysuccinimidyl, oxysulfosuccinimidyl, 1-oxybenzotriazolyl, etc.).
**Acyl azides can rearrange to isocyanates.

In Scheme (I), moieties “L¹” and “L²” represent optional linkers that space functionalities R^{x} and F^{x} from the remainder of their respective molecules. As can be seen from Scheme (I), the moiety -L¹-Y-L²- of compound 102 corresponds to, and is a specific embodiment of, linker “L” of FIG. 1A. Accordingly, the linkers “L¹” and “L²” of Scheme (I) are similar in concept and composition to linker “L” of FIG. 1A, and can comprise any of the various different types of atoms and groups discussed above in connection with linker “L.”

In some embodiments, the —S—R^{x} portion of glycerophospholipid 30 corresponds to the polar head group of a naturally occurring glycerophospholipid. As a specific example, —S—R^{x} can be selected from —CH₂CH₂NH₃⁺ (ethanolamin-2-yl), —CH₂CH₂N⁺(CH₃)₂ (cholinyl) and —CH₂C(NH₃⁺)C(O)O⁻ (serinyl). The identity of —S—R^{x} can be selected based upon the phospholipase that will be used to cleave the resultant signal molecule 102.

Glycerophospholipid signal molecule 102 can be prepared using conventional synthetic methods, as exemplified by Scheme (I), supra. Phospholipid starting materials, such as phospholipids corresponding in structure to compound 30 of Scheme (I), can be prepared using conventional synthetic methods, extracted from natural sources (e.g., from egg yolk, brain or plant sources) or purchased commercially (e.g., from Sigma-Aldrich and/or Avanti Polar Lipids). The synthesis of phospholipids is described in PHOSPHOLIPIDS HANDBOOK (G. Cevc, ed., Marcel Dekker (1993)), BIOCONJUGATE TECHNIQUES (G. Hermanson, Academic Press (1996)), and Subramanian et al., ARKIVOC VII:116-125 (2002). As a specific example, glycerophospholipid 30 can be prepared from the reaction of a 3-substituted phosphoglycerol compound with selected fatty acid anhydrides. Examples of suitable phosphoglycerol compounds comprise glycero-3-phosphoethanolamine and glycerol-3-phosphoserine, either of which can be obtained commercially (e.g. from Sigma-Aldrich). Fatty acid anhydrides can be prepared from fatty acids, which in turn can be synthesized by conventional methods, extracted from natural sources, or purchased commercially.

Non-limiting examples of commercially available phospholipids corresponding in structure to compound 30 of Scheme (I) that can be used to prepare glycerophospholipid signal molecule 102 according to Scheme (I) are provided in Table 4, below.

TABLE 4

| | | Avanti Catalog |
|---|---|---|
| Product Acyl Composition | M.W. | Number |
| Phosphatidylethanolamine 16:0 | 691.97 | 850705 |
| Phosphatidylethanolamine 18:1 | 744.05 | 850725 |
| N-Caproylamine-PE 16:0 | 805.13 | 870125 |
| N-Caproylamine-PE 18:1 | 857.21 | 870122 |
| N-Dodecanylamin-PE 16:0 | 889.29 | 870140 |
| N-Dodecanylamin-PE 18:1 | 941.37 | 870142 |
| Phosphatidylthio-ethanol 16:0 | 731.00 | 870160 |
| N-MCC-PE 16:0 | 928.24 | 780200 |
| N-MCC-PE 18:1 | 980.32 | 780201 |
| N-MPB-PE 16:0 | 955.20 | 870013 |
| N-MPB-PE 18:1 | 1,007.27 | 870012 |
| N-PDP-PE 16:0 | 911.22 | 870205 |
| N-PDP-PE 18:1 | 963.30 | 870202 |
| N-Succinyl-PE 16:0 | 814.03 | 870225 |
| N-Succinyl-PE 18:1 | 866.10 | 870222 |
| N-Glutaryl-PE 16:0 | 828.05 | 870245 |
| N-Glutaryl-PE 18:1 | 880.13 | 870242 |
| N-Dodecanyl-PE 16:0 | 926.24 | 870265 |
| N-Dodecanyl-PE 18:1 | 978.32 | 870262 |
| N-Biotinyl-PE 16:0 | 940.25 | 870285 |
| N-Biotinyl-PE 18:1 | 992.32 | 870282 |
| N-Biotinyl Cap-PE 16:0 | 1,053.40 | 870277 |
| N-Biotinyl Cap-PE 18:1 | 1,105.48 | 870273 |
| Phosphatidyl (Ethylene Glycol)16:0 | 714.94 | 870305 |
| Phosphatidyl (Ethylene Glycol)18:1 | 767.01 | 870302 |

In Table 4, N-MCC-PE 16:0 refers to 1,2-Dipalmitoyl-sn-glycero-3-phosphoethanolamine-N-[4-(p-maleimidomethyl)cyclohexane-carboxamide]; 16:0 MPB PE refers to 1,2-Dipalmitoyl-sn-Glycero-3-Phosphoethanolamine-N-[4-(p-maleimidophenyl)butyramide] (sodium salt); and 16:0 PDP PE refers to 1,2-Dipalmitoyl-sn-Glycero-3-Phosphoethanolamine-N-[3-(2-pyridyldithio)propionate] (sodium salt).

Fluorescent dyes corresponding in structure to compound 32 of Scheme (I) that can be used to prepare glycerophospholipid signal molecule 102, can be prepared synthetically using conventional methods or purchased commercially (e.g. Sigma-Aldrich and/or Molecular Probes). Non-limiting examples of methods that can be used to synthesize suitably reactive fluorescein and/or rhodamine dyes can be found in the various patents and publications discussed above in connection with the fluorescent moiety. Non-limiting examples of suitably reactive fluorescent dyes that are commercially available from Molecular Probes (Eugene, Oreg.) are provided in Table 5, below:

TABLE 5

| Catalog | |
|---|---|
| Number | Product Name |
| C-20050 | 5-carboxyfluorescein-bis-(5-carboxymethoxy-2-nitrobenzyl) |
| | ether, -alanine-carboxamide, succinimidyl ester (CMNB-caged |
| | carboxyfluorescein, SE) |
| C-2210 | 5-carboxyfluorescein, succinimidyl ester (5-FAM, SE) |
| C-1311 | 5-(and-6)-carboxyfluorescein, succinimidyl ester (5(6)-FAM, |
| | SE) |
| D-16 | 5-(4,6-dichlorotriazinyl) aminofluorescein (5-DTAF) |
| F-6106 | 6-(fluorescein-5-carboxamido)hexanoic acid, succinimidyl |
| | ester (5-SFX) |
| F-2182 | 6-(fluorescein-5-(and-6)-carboxamido) hexanoic acid, |
| | succinimidyl ester (5(6)-SFX) |
| F-6129 | 6-(fluorescein-5-(and-6)-carboxamido) hexanoic acid, |
| | succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

The syntheses of two exemplary glycerophospholipid signal molecules 102 according to Scheme (I) are illustrated in FIGS. 3A and 3B, and discussed in more detail in the Examples Section.

Glycerophospholipid signal molecule 102 can also be obtained commercially or synthesized under contract with commercial vendors. Non-limiting examples of commercially available glycerophospholipid signal molecules 100 include 1-Hexanoyl-2-[6-[(7-nitro-2-1,3-benzoxadiazol-4-yl)amino]hexanoyl]-sn-Glycero-3-Phosphocholine (catalog no. 810112, Avanti); 1,2-Dimyristoyl-sn-Glycero-3-Phosphoethanolamine-N-(Lissamine Rhodamine B Sulfonyl) (Ammonium Salt) (catalog no. 810157, Avanti); 1,2-Dioleoyl-sn-Glycero-3-Phosphoethanolamine-N-(5-dimethylamino-1-naphthalenesulfonyl) (catalog no. 790628, Avanti); 1,2-Dioleoyl-sn-Glycero-3-Phosphoethanolamine-N-(1-pyrenesulfonyl) (catalog no. 790627, Avanti); and 1,2-Dioleoyl-sn-Glycero-3-Phosphoethanolamine-N-(Carboxyfluorescein) (catalog no. 790547, Avanti). Other examples include Oregon Green® 488 1,2-dihexadecanoyl-sn-glycero-3-phosphoethanolamine (Oregon Green® 488 DHPE) (catalog no. 0-12650, Molecular Probes) and 1,2-dioleoyl-sn-glycero-3-phosphoethanolamine-N-(carboxyfluorescein) (catalog no. 790547, Avanti).

Referring again to FIG. 1A, as an alternative to covalent linkage, the fluorescent moiety of signal molecule 100 could be attached to the glycerophospholipid head group by the use of pairs of specific binding molecules, as is known in the art (such as listed in U.S. Pat. No. 6,399,392). Examples of specific binding pairs include biotin/avidin (or streptavidin), carbohydrate/lectin, DNA/cDNA, IgG/proteinA, antigen/antibody and ion/chelator.

While in FIG. 1A the fluorescent moiety is illustrated as being attached to the phosphate moiety or polar head group of the glycerophospholipid, skilled artisans will appreciate that the fluorescent moiety can be associated with various positions of the glycerophospholipid structure. For example, the fluorescent moiety can be linked to one of the two hydrophobic tail groups, such as at their terminal position(s). A specific example of this type of glycerophospholipid signal molecule is illustrated in FIG. 1B. In FIG. 1B, R² represents a hydrophobic moiety and “D” represents a fluorescent moiety, as previously described for FIG. 1A. In exemplary signal molecule 200, the fluorescent moiety “D” is linked to the remainder of the molecule via a saturated hydrophobic alkylene moiety —(CH₂)ₓ—, where x is an integer, typically ranging from 0 to 30. Although the illustrated polar head group is an ethanolamin-2-yl group, other polar headgroups could be used, as could a different hydrophobic moiety. Moreover, while the fluorescent moiety in signal molecule 200 is attached to the hydrophobic moiety on the C1 position of the glycerolyl backbone, it could also be attached to the hydrophobic moiety on the C2 carbon (signal molecule 210). Alternatively, fluorescent moieties could be attached to the hydrophobic moieties at both of the C1 and C2 carbons (signal molecule 220). If a fluorescent moiety having sufficient hydrophobic character is selected, it can be attached directly to the C1 and/or C2 hydroxyl (in this case x is 0). In this embodiment, the fluorescent moiety can have the dual role of acting as the fluorescent moiety and the hydrophobic moiety.

As illustrated in FIG. 2B, cleavage of glycerophospholipid signal molecule 200 by phospholipase A1 cleaves the fluorescent moiety from the remainder of the molecule in the form of fatty acid derivative 34. Also generated is lysophospholipid 36. Cleavage by phospholipase A2 yields fatty acid 18 and lysophospholipid derivative 38. In either case, the fragment containing the fluorescent moiety can leave the micelle, thereby unquenching the fluorescence of the fluorescent moiety, leading to an increase in the fluorescence signal. Cleavage of glycerophospholipid signal molecule 210 with PLA1 yields fatty acid 14 and lysopholipid derivative 42 (see FIG. 2C); cleavage with PLA2 yields lysopholipid 20 and fatty acid derivative 34. Similarly, cleavage of glycerophospholipid signal molecule 220 with PLA1 yields fatty acid derivative 34 and lysophospholipid derivative 42; cleavage with PLA2 yields fatty acid derivative 34 and lysophospholipid derivative 42. Like the cleavage products of signal molecule 200, the cleavage products of signal molecules 210 and 220 can leave the micelle, causing an increase in fluorescence.

Glycerophospholipid signal molecules having a fluorescent moiety associated with or replacing one or both of the hydrophobic tails can be synthesized using routine methods, or can be obtained commercially. Non-limiting examples of glycerophospholipid signal molecules of this type that are commercially available from Molecular Probes (Eugene, Oreg.) include 2-decanoyl-1-(O-(11-(4,4-difluoro-5,7-dimethyl-4-bora-3a,4a-diaza-s-indacene-3-propionyl)amino)undecyl)-sn-glycero-3-phosphocholine (cat #D-3771), 2-(4,4-difluoro-5,7-dimethyl-4-bora-3a,4a-diaza-s-indacene-3-dodecanoyl)-1-hexadecanoyl-sn-glycero-3-phosphocholine (β-BODIPY® FL C₁₂—HPC) (cat #D-3792), 2-(4,4-difluoro-5-methyl-4-bora-3a,4a-diaza-s-indacene-3-dodecanoyl)-1-hexadecanoyl-sn-glycero-3-phosphocholine (β-BODIPY® 500/510 C₁₂—HPC) (cat #D-3793), 2-(4,4-difluoro-5-octyl-4-bora-3a,4a-diaza-s-indacene-3-pentanoyl)-1-hexadecanoyl-sn-glycero-3-phosphocholine (β-C₈-BODIPY® 500/510 C₅—HPC) (cat #D-3795), and 2-(4,4-difluoro-5-octyl-4-bora-3a,4a-diaza-s-indacene-3-pentanoyl)-1-hexadecanoyl-sn-glycero-3-phosphocholine (β-C₈-BODIPY® 500/510 C₅—HPC) (cat #D-3803). See HANDBOOK OF FLUORESCENT PROBES AND RESEARCH PRODUCTS (9^{th} edition, Molecular Probes, Inc.), which is incorporated herein by reference in its entirety.

In some embodiments, a quenching moiety can be included in the glycerophospholipid signal molecule. The quenching moiety can act to enhance the quenching effect of self-quenched fluorescent moieties, or it can provide the sole means of the quenching effect. The relative locations of the fluorescent and the quenching moieties is not critical. In some embodiments, the quenching moiety is positioned such that it intramolecularly quenches the fluorescence of the fluorescent moiety in the same molecule. In another embodiment, the quenching moiety is positioned such that it intermolecularly quenches the fluorescence of a fluorescent moiety of another signal molecule in the micelle.

The quenching moiety can comprise any moiety capable of quenching the fluorescence of a fluorescent moiety. Compounds capable of quenching the fluorescence of the various different types of fluorescent dyes discussed above, such as xanthene, fluorescein, rhodamine, cyanine, pthalocyanine and squaraine dyes, are well-known. Such quenching compounds can be non-fluorescent (also referred to as “dark quenchers” or “black hole quenchers”, such as from Epoch Biosciences or Biosearch) or, alternatively, they may themselves be fluorescent. Examples of suitable non-fluorescent dark quenchers that can comprise the quenching moiety comprise, but are not limited to, Dabcyl, the various non-fluorescent quenchers described in U.S. Pat. No. 6,080,868 (Lee et al.) and the various non-fluorescent quenchers described in WO 03/019145 (Ewing et al.). Examples of suitable fluorescent quenchers comprise, but are not limited to, the various fluorescent dyes described above. In some embodiments in which the quenching moiety comprises a fluorescent dye, the fluorescence of the quenching moiety can be used as a secondary label, for example, as an internal standard to which the signal fluorescence can be referenced, or to “track” the micelles.

The ability of a quenching moiety to quench the fluorescence of a particular fluorescent moiety may depend upon a variety of different factors, such as the mechanism(s) of action by which the quenching occurs. The mechanism of the quenching is not critical to success, and may occur, for example, by orbital overlap, by collision, by FRET, by another mechanisms or combination of mechanisms. The selection of a quenching moiety suitable for a particular application can be readily determined empirically. As a specific example, the dark quencher Dabcyl and the fluorescent quencher TAMRA have been shown to effectively quench the fluorescence of a variety of different fluorophores. In a specific embodiment, a quenching moiety can be selected based upon its spectral overlap properties with the fluorescent moiety. For example, a quenching moiety can be selected that has an absorbance spectrum that sufficiently overlaps the emission spectrum of the fluorescent moiety such that the quenching moiety quenches the fluorescence of the fluorescent moiety when in close proximity thereto.

In some embodiments, the quenching moiety can be linked to the fluorescent moiety of the same signal molecule via a cleavable linker. Both the fluorescent and the quenching moieties can be located in the polar head group. The linker may contain a labile functionality, such as an ester or disulfide, that is capable of being cleaved by intermolecular hydrolysis or nucleophilic attack. The linker may also be cleaved by intramolecular mechanisms such as by cyclization. An example of cyclization is a thiophosphorylated serine, threonine or tyrosine group intramolecularly reacting with an ester to form a cyclic thioester bond. Another embodiment of intramolecular cyclization is the reaction of a thiophosphorylated serine, threonine or tyrosine with a disulfide cleavable linker to form a thiophosphate disulfide bond. In addition, the linker may contain a polypeptide, polynucleotide or polysaccharide segment that is cleavable by an appropriate enzyme, such as a protease, nuclease or glycosidase. Cleavage of the linker separates the quenching moiety from the fluorescent moiety, thereby producing an increase in fluorescence.

In another embodiment, the fluorescent moiety can be attached to one of the two hydrophobic moieties, and the quenching moiety can be attached to the other. A specific embodiment of this type of glycerophospholipid signal molecule 300 is illustrated in FIG. 1C. In FIG. 1C, “Q” represents the quenching moiety and “D” represents the fluorescent moiety. Each of these moieties is attached to a saturated alkylene hydrophobic moiety represented by —(CH₂)ₓ—, where each x is an integer ranging from 0 to 30. Although signal molecule 300 is illustrated as having a specific polar head group and hydrophobic moieties, other polar head groups and/or hydrophobic moieties could be used. The lengths and properties of the hydrophobic moieties can be selected such that the quencher moiety “Q” quenches the fluorescence of fluorescent moiety “D.”

Cleavage of signal molecule 300 by phospholipase A1 or A2 (illustrated in FIG. 2C), releases quencher moiety “Q” and fluorescent moiety “D” from their close proximity, resulting in an increase in fluorescence.

While the exemplary signal molecules of FIGS. 1A-1C, as well as certain other exemplary signal molecules, have been described with reference to phospholipids, other lipids, such as sphingolipids, lysophospholipids, tri-, di- or monoacylglycerols, could also be used. Sphingolipids and triacylglycerols including fluorescent moieties are well known in the art, and some of them can be purchased from commercial sources. See, for example, Section 13.3 in Handbook of Fluorescent Probes and Research Products, supra. Like phospholipids, these lipids can form micelles. Fluorescence of such lipid signal molecules can be quenched in the micelles. By treating these lipid signal molecules with suitable agents, such as sphingomyelinases and triacylglycerol lipases (e.g. pancreatic lipase), the fluorescent moieties in these lipid signal molecules can be released from the micelle, thereby producing an increase in fluorescence.

Signal molecules including non-naturally occurring analogs of phospholipids that are resistant to lysis by certain phospholipases can also be used. In some embodiments of such signal molecules, the phosphate group is replaced by a phosphonate or phosphinate group (as described in U.S. Pat. No. 4,888,288). In another embodiment, one or both ester linkages attaching the hydrophobic moieties to the glycerol backbone can be replaced with an ether linkage, thus rendering the signal molecule resistant to cleavage by PLA1 or PLA2 cleavage.

6.2.3 Dye-Peptide Signal Molecules

In some embodiments, the signal molecule is a dye-peptide conjugate which comprises one or more fluorescent moieties, one or more peptide moieties, and one or more hydrophobic moieties. The hydrophobic moiety(ies) can integrate the dye-peptide conjugate into a micelle. The fluorescent signal of the fluorescent moiety(ies) is quenched when the conjugate is integrated in the micelle. The peptide moiety comprises a modification site which is recognizable by an enzyme of interest. Modification of the site by the enzyme results in reduction or elimination of the quenching effect, thereby producing a detectable fluorescence increase. Any of the above-described hydrophobic and fluorescent moieties can be used to construct dye-peptide signal molecules.

A variety of different dye-peptide conjugates suitable for use as signal molecules in the micelles described herein are taught in U.S. Patent Publication No. 2004/0146959, the disclosure of which is incorporated herein by reference.

In some embodiments, the peptide moiety comprises a protein kinase recognition moiety which comprises at least one unphosphorylated residue capable of being phosphorylated by a protein kinase. Phosphorylation changes the charge(s) on the peptide moiety, and therefore destabilizes the dye-peptide conjugate in the micelle and promotes the release of the conjugate from the complex. The release of the dye-peptide conjugate abolishes the quenching effect caused by the interactions between the complex and the fluorescent moiety, thereby producing a measurable increase in fluorescence signals.

The protein kinase recognition moiety generally comprises a recognition sequence for a protein kinase that includes at least one amino acid side chain containing a group that is capable of being phosphorylated by a protein kinase. In some embodiments, the phosphorylatable group is a hydroxyl group. Usually, the hydroxyl group is provided as part of a side chain in a tyrosine, serine, or threonine residue, although any other natural or non-natural amino acid side chain or other entity containing a phosphorylatable hydroxyl group can be used. The phosphorylatable group can also be a nitrogen atom, such as the nitrogen atom in the epsilon amino group of lysine, an imidazole nitrogen atom of histidine, or a guanidinium nitrogen atom of arginine. The phosphorylatable group can also be a carboxyl group in an asparate or glutamate residue.

The protein kinase recognition moiety may further comprise a segment, typically a polypeptide segment, that contains one or more subunits or residues (in addition to the phosphorylatable residue) that impart identifying features to the recognition moiety to make it compatible with the substrate specificity of the protein kinase(s) to be used to modify the signal molecule.

A wide variety of protein kinases have been characterized over the past several decades, and numerous classes have been identified (see, e.g., S. K. Hanks et al., SCIENCE 241:42-52 (1988); R. E. Kemp and R. B. Pearson, TRENDS BIOCHEM. SCI. 15:342-346 (1990); S. S. Taylor et al., ANN. REV. CELL BIOL. 8:429-462 (1992); Z. Songyang et al., CURRENT BIOLOGY 4:973-982 (1994); and CHEM. REV. 101:2209-2600 “Protein Phosphorylation and Signaling” (2001)). Exemplary classes of protein kinases comprise cAMP-dependent protein kinases (also called the protein kinase A family, A-proteins, or PKA), cGMP-dependent protein kinases, protein kinase C enzymes (PKC, including calcium dependent PKC activated by diacylglycerol), Ca²⁺/calmodulin-dependent protein kinase I or II, protein tyrosine kinases (e.g., PDGF receptor, EGF receptor, and Src), mitogen activated protein (MAP) kinases (e.g., ERK1, KSS1, and MAP kinase type I), cyclin-dependent kinases (e.g., Cdk2 and Cdc2), glycogen synthase kinases (GSK), and receptor serine kinases (e.g., TGF-β) Exemplary consensus sequences for various protein kinases are shown in Table 6 below. These various consensus sequences can be used to design particular protein kinase recognition moieties having derived specificities for particular kinase and/or kinase families.

Protein kinase recognition moieties having desired specificities for particular kinases and/or kinase families can also be designed, for example, using the methods and/or exemplary sequences described in Brinkworth et al., PROC. NATL. ACAD. SCI. USA 100(1):74-79 (2003).

TABLE 6

| | | |
|---|---|---|
| | | Consensus Sequence^{a}/ |
| Symbol | Description | Enzyme Substrates |

| | | | |
|---|---|---|---|
| PKA | cAMP-dependent | -R-R-X-S/T-Z- | (SEQ ID NO: 1) |
| | | -L-R-R-A-S-L-G- | (SEQ ID NO: 2) |
| PhK | phosphorylase kinase | -R-X-X-S/T-F-F- | (SEQ ID NO: 3) |
| | | -R-Q-G-S-F-R-A- | (SEQ ID NO: 4) |
| cdk2 | cyclin-dependent kinase-2 | -S/T-P-X-R/K | (SEQ ID NO: 5) |
| ERK2 | extracellular-regulated kinase- | -P-X-S/T-P | (SEQ ID NO: 6) |
| | 2 | -R-R-I-P-L-S-P | (SEQ ID NO: 7) |
| PKC | protein kinase C | K-K-K-K-R-F-S-F-K^{b} | (SEQ ID NO: 8) |
| | | X-R-X-X-S-X-R-X | (SEQ ID NO: 9) |
| CaMKI | Ca²⁺/calmodulin-dependent | L-R-R-L-S-D-S-N-F^{c} | (SEQ ID NO: 10) |
| | protein kinase I | | |
| CaMKII | Ca²⁺/calmodulin-dependent | K-K-L-N-R-T-L-T-V-A^{d} | (SEQ ID NO: 11) |
| | protein kinase II | | |
| c-Src | cellular form of Rous sarcoma | -E-E-I-Y-E/G-X-F | (SEQ ID NO: 12) |
| | virus transforming agent | -E-E-I-Y-G-E-F-R | (SEQ ID NO: 13) |
| v-Fps | transforming agent of | -E-I-Y-E-X-I/V | (SEQ ID NO: 14) |
| | Fujinami sarcoma virus | | |
| Csk | C-terminal Src kinase | -I-Y-M-F-F-F | (SEQ ID NO: 15) |
| InRK | Insulin receptor kinase | -Y-M-M-M | (SEQ ID NO: 16) |
| EGFR | EGF receptor | -E-E-E-Y-F | (SEQ ID NO: 17) |
| SRC | Src kinase | -R-I-G-E-G-T-Y-G-V-V-R-R | (SEQ ID NO: 18) |
| Akt | RAC-beta serine/threonine- | -R-P-R-T-S-S-F- | (SEQ ID NO: 19) |
| | protein kinase | | |
| Erk1 | Extracellular signal-regulated | -P-R-T-P-G-G-R- | (SEQ ID NO: 20) |
| | kinase 1 (MAP kinase 1, | | |
| | MAPK 1) | | |
| MAPKAP | MAP kinase-activated protein | -R-L-N-R-T-L-S-V | (SEQ ID NO: 21) |
| K2 | kinase2 | | |
| NEK2 | Serine/threonine-protein | -D-R-R-L-S-S-L-R | (SEQ ID NO: 22) |
| | kinase Nek2 | | |
| Ab 1 | tyrosine kinase | -E-A-I-Y-A-A-P-F-A-R-R-R | (SEQ ID NO: 23) |
| YES | Proto-oncogene tyrosine- | E-E-I-Y-G-E-F-R | (SEQ ID NO: 13) |
| | protein kinase YES | | |
| LCK | Proto-oncogene tyrosine- | E-E-I-Y-G-E-F-R | (SEQ ID NO: 13) |
| | protein kinase LCK | | |
| SRC | Proto-oncogene tyrosine- | K-V-E-K-I-G-E-G-T-Y-G-V-V- | (SEQ ID NO: 24) |
| | protein kinase Src | Y-K | |
| LYN | Tyrosine-protein kinase LYN | E-E-E-I-Y-G-E-F | (SEQ ID NO: 25) |
| BTK | Tyrosine-protein kinase BTK | E-E-I-Y-G-E-F-R- | (SEQ ID NO: 13) |
| GSK3 | Glycogen synthase kinase-3 | R-H-S-S-P-H-Q-(Sp)-E-D-E-E | (SEQ ID NO: 26) |
| CHI | Casein kinase I | R-R-K-D-L-H-D-D-E-E-D-E-A- | (SEQ ID NO: 27) |
| | | M-S-I-T-A | |
| CKII | Casein kinase II | -(Sp)-X-X-S/T- | (SEQ ID NO: 28) |
| | | S-X-X-E/D | (SEQ ID NO: 29) |
| | | R-R-R-D-D-D-S-D-D-D | (SEQ ID NO: 30) |
| TK | Tyrosine kinase | K-G-P-W-L-E-E-E-E-E-A-Y-G- | (SEQ ID NO: 31) |
| | | W-L-D-F | |

^{a}see, for example, B. E. Kemp and R. B. Pearson, TRENDS BIOCHEM. SCI. 15:342-346 (1990); Z. Songyang et al., CURRENT BIOLOGY 4:973-982 (1994); J. A. Adams, CHEM REV. 101:2272 (2001) and references cited therein; X means any amino acid residue, “/” indicates alternate residues, and Z is a hydrophobic amino acid, such as valine, leucine or isoleucine; p indicates a P0₄²⁻group
^{b}Graff et al., J. BIOL. CHEM. 266:14390-14398 (1991)
^{c}Lee et al., Proc. NATL. ACAD. SCI. 91:6413-6417 (1994)
^{d}Stokoe et al., BIOCHEM. 296:843-849 (1993)

Typically, the protein kinase recognition moiety comprises a sequence of L-amino acid residues. However, any of a variety of amino acid with different backbone or side chain structures can also be used, such as: D-amino acid polypeptides, alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines. A more detailed backbone list includes N-substituted amide (CONR replaces CONH linkages), esters (CO₂), keto-methylene (COCH₂), reduced or methyleneamino (CH₂NH), thioamide (CSNH), phosphinate (PO₂RCH₂), phosphonamidate and phosphonamidate ester (PO₂RNH), retropeptide (NHCO), transalkene (CR═CH), fluoroalkene (CF═CH), dimethylene (CH₂CH₂), thioether (CH₂S), hydroxyethylene (CH(OH)CH₂), methyleneoxy (CH₂O), tetrazole (CN₄), retrothioamide (NHCS), retroreduced (NHCH₂), sulfonamido (SO₂NH), methylenesulfonamido (CHRSO₂NH), retrosulfonamide (NHSO₂), and backbones with malonate and/or gem-diaminoalkyl subunits, for example, as reviewed by M. D. Fletcher et al. CHEM. REV. 98:763 (1998) and the references cited therein. Peptoid backbones (N-substituted glycines) can also be used (e.g., H. Kessler, ANGEW. CHEM. INT. ED. ENGL. 32:543 (1993); R. N. Zuckermann, CHEMTRACTS-MACROMOL. CHEM. 4:80 (1993); and Simon et al., PROC. NATI. ACAD. SCI. 89:9367 (1992)).

In some embodiments, the protein kinase recognition moiety includes all of the residues comprising the recognition sequence for a given protein kinase. The total number of residues comprising the recognition sequence can be defined as N, wherein N is an integer from 1 to 10. In some embodiments, N is an integer from 1 to 15. In other embodiments, N is an integer from 1 to 20. As a specific example of these embodiments, the consensus recognition sequence for PICA is -R-R-X-S/T-Z, thus, N=5. Repetition of the recognition sequence, two, three, or four, or more times can be used to provide a protein kinase recognition moiety with two, three, four or more unphosphorylated residues.

In other embodiments, the protein kinase recognition moiety comprises overlapping recognition sequences. In these embodiments, one or more residues from a recognition sequence are shared between two recognition sequences. As a specific example of these embodiments, the consensus recognition sequence for p38βII is P-X-S-P. A recognition moiety with overlapping consensus sequences can be created by sharing a -P- residue between two recognition sequences, e.g., P-X-S-P-X-S-P.

In other embodiments, the protein kinase recognition moiety can comprise a subset of the residues comprising the recognition sequence. In these embodiments, one or more residues are omitted from the recognition motif. A subset is defined herein as comprising N-u amino acid residues, wherein, as defined above, N represents the total number of amino acid residues comprising the recognition sequence, and u represents the number of amino acid residues omitted from the recognition sequence. In some embodiments, u is an integer from 1 to 9. In other embodiments, u is an integer from 1 to 14. In still other embodiments, u is an integer from 1 to 19. For example, if the total number of amino acids in the recognition motif is 4, subsets comprising 3, 2, or 1 amino acid residue(s) can be made. If the total number of amino acids in the recognition motif is 5, subsets comprising 4, 3, 2, or 1 amino acid residue(s) can be made. If the total number of amino acids in the recognition motif is 6, subsets comprising 5, 3, 2, or 1 amino acid residue(s) can be made. If the total number of amino acids in the recognition motif is 7, subsets comprising 6, 5, 4, 3, 2, or 1 amino acids residue(s) can be made. If the recognition motif comprises 8 amino acids, subsets comprising 7, 6, 5, 4, 3, 2, or 1 amino acid residue(s) can be made. If the total number of amino acids in the recognition motif is 9, subsets comprising 8, 7, 6, 5, 4, 3, 2, or 1 amino acids residue(s) can be made. If the recognition motif comprises 10 amino acids, subsets comprising 9, 8, 7, 6, 5, 4, 3, 2, or 1 amino acids residue(s) can be made. Typically, subsets comprising N-1 or N-2 amino acid residues are made.

The number of residues to include in the recognition sequence, in part, will depend, on the specificity of the protein kinase. For example, some protein kinases, such as p38βII, require all of the residues comprising the recognition sequence to be present for phosphorylation activity to occur. Other protein kinases, such as PKC, can phosphorylate a recognition sequence, in which one or more residues are omitted from the recognition sequence. In other embodiments, recognition sequences comprising a phosphorylated residue are designed for use with protein kinases, such as GSK3, that require a phosphorylated residue in order to phosphorylate one or more unphophosphorylated residue.

Various combinations of the foregoing embodiments can be used in the compositions and methods described herein. For example, kinase substrate compounds comprising recognition moieties that include recognition sequences comprising N residues for a given protein kinase can be selected. In other embodiments, kinase substrate compounds comprising recognition moieties, in which one recognition sequence comprises N residues and the other recognition sequence comprises N-u residues can be selected. Thus, substrate compounds comprising recognition moieties with any combination of N and N-u recognition sequences can be used, provided there is a detectable increase in fluorescence when the protein kinase is present. Moreover, the recognition moieties can be for the same protein kinase, or they may be for different protein kinases.

The distance between unphosphorylated residues depends, in part, on the location of the unphosphorylated residue(s) in each of the selected recognition sequences, and, in part, on the way in which the selected recognition sequences are connected. Unphosphorylated residues capable of being phosphorylated by a protein kinase can be adjacent, or they can be separated by one, two, three, or more residues that are not phosphorylated by a protein kinase. For example, a substrate compound in which the unphosphorylated residues are separated by three residues can be formed by connecting two recognition sequences, each comprising the recognition sequence -S-X-X-X- to each other to form a recognition moiety having the composition -S-X-X-X-S-X-X-X-. In another example, a substrate compound, in which the unphosphorylated residues are separated by two residues can be formed by sharing an amino acid residue between two recognition sequences, e.g., the -P- in the recognition sequence -P-X-S-P- can be shared to form the recognition moiety P-X-S-P-X-S-P-. Thus, any combination of N and N-u recognition sequences, in which the unphosphorylated residues are adjacent, or are separated by one or more residues, can be used in the kinase substrate compounds provided that an increase in fluorescence is observed in the presence of the protein kinase(s).

The protein kinase recognition sequences can be connected in any way that permits them to perform their respective function. In some embodiments, the protein kinase recognition sequences can be directly connected to each other. In other embodiments, the protein kinase recognition sequences can be indirectly connected to each other via one or more linkage groups. In yet other embodiments, the protein kinase recognition moieties are indirectly linked to each other through the fluorescent moiety or the hydrophobic moiety. Examples of protein kinase recognition moieties comprising two recognition sequences are described in more detail below.

The dye-peptide signal molecule may be designed to have a particular net charge in the phosphorylated state. For instance, the unphosphorylated molecule can have a net charge of zero (a net neutral charge), or about zero, when measured at pH 7-8. Phosphorylation of the signal molecule yields a modified signal molecule having a net charge of −2. The modified signal molecule dissociates from the micelle, producing an increase in fluorescence of its fluorescent moiety.

Net charges other than zero may also be selected. The net charge of a dye-peptide signal molecule can be established by including an appropriate number of negatively and positively charged groups in the peptide moiety. For example, to establish a net neutral charge (net charge=zero), the molecule can be designed to contain an equal number of positively and negatively charged groups. Lysine and arginine contain side chains that carry a single positive charge at physiological pH (pH=6 to 8). Aspartate and glutamate contain carboxyl side chains having a single negative charge. A phosphoserine residue carries two negative charges on a phosphate group. The imidazole side chain of histidine has a pK of about 7, so it carries a full positive charge at a pH of about 6 or less. Cysteine has a pK of about 8, so it carries a full negative charge at a pH of about 9 or higher. In addition, the fluorescent moiety may also contain charged groups that should be considered to obtain a particular desired net charge for a dye-peptide signal molecule.

In some embodiments, the peptide moiety comprises a phosphatase recognition moiety containing at least one recognition sequence comprising one or more phosphorylated residues that are capable of being dephosphorylated (hydrolyzed) by a phosphatase. As discussed above for protein kinase recognition moieties, in some embodiments, the phosphatase recognition moiety comprises two or more recognition sequences.

The dye-peptide signal molecule can be designed to have a neutral or near-neutral net charge in the phosphorylated state. Dephosphorylation creates a modified signal molecule having a change in net charge of +2, which dissociates from the micelle, producing an increase in fluorescence of its fluorescent moiety. In some cases, the dye-peptide signal molecule may have positive or negative charges in the phosphorylated state.

A wide variety of protein phosphatases have been identified (e.g., see P. Cohen, ANN. REV. BIOCHEM. 58:453-508 (1989); MOLECULAR BIOLOGY OF THE CELL, 3rd edition Alberts et al., eds., Garland Publishing, NY (1994); and CHEM. REV. 101:2209-2600, “Protein Phosphorylation and Signaling” (2001)). Serine/threonine protein phosphatases represent a large class of enzymes that reverse the action of protein kinases, such as PKAs. The serine/threonine protein phosphatases have been divided among four groups designated I, IIA, IIB, and IIC. Protein tyrosine kinases are also an important class of phosphatase. Histidine, lysine, arginine, and asparate phosphatases are also known (e.g., P. J. Kennelly, Chem. Rev. 101:2304-2305 (2001) and references cited therein). In some cases, phosphatases are highly specific for only one or a few proteins, but in other cases, phosphatases are relatively non-specific and can act on a large range of protein targets. Examples of peptide sequences that can be dephosphorylated by phosphatases are described in P. J. Kennelly, supra.

The peptide moiety can be designed to be reactive with a particular phosphatase or a group of phosphatases. The unphosphorylated residue in the phosphatase recognition sequence may be any group that is capable of being dephosphorylated by a phosphatase. In some embodiments, the residue is a phosphotyrosine residue. In some embodiments, the residue is a phosphoserine residue. In some embodiments, the residue is a phosphothreonine residue.

The phosphatase recognition moiety may further comprises a segment, typically a polypeptide segment, that contains one or more subunits or residues (in addition to the dephosphorylatable residues) that impact identifying features to the recognition site to make it compatible with the substrate specificity of the protein phosphatase(s) to be used to modify the signal molecule.

The protein kinase or phosphatase recognition moiety may comprise a polypeptide segment containing the group or residue that is to be phosphorylated or dephosphorylated. In some embodiments, such a polypeptide segment has a polypeptide length equal to or less than 30 amino acid residues, 25 residues, 20 residues, 15 residues, 10 residues, or 5 residues. In another embodiment, the polypeptide segment has a polypeptide length in a range of 3 to 30 residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In yet another embodiment, the polypeptide segment contains at least 3, 4, 5, 6 or 7 amino acid residues.

In some embodiments, a sulfatase substrate moiety for detecting or characterizing on or more sulfatases in a sample is provided. A wide variety of sulfatases have been identified. In some cases, sulfatases are highly specific for only one or a few substrates, but in other cases, sulfatases are relatively non-specific and can act on a large range of substrates including, but not limited to, proteins, glycosaminoglycans, sulfolipids, and steroid sulfates. Exemplary sulfatases and sulfatase substrates are shown in Table 7, below. These substrates can be used to design sulfatase recognition moieties having desired specificities for particular sulfatases and/or sulfatase families.

TABLE 7

| Sulfatase Description | EC | |
|---|---|---|
| (Alternative Name(s)) | number | Substrate(s) |
| Arylsulfatase | 3.1.6.1 | phenol sulfate |
| (Sulfatase; Aryl-sulphate, | | |
| sulphohydrolase) | | |
| Steryl-sulfatase | 3.1.6.2 | 3-beta-hydroxyandrost-5-en-17- |
| (Steroid sulfatase; Steryl- | | one 3-sulfate and related steryl |
| sulfate sulfohydrolase; | | sulfates |
| Arylsulfatase C) | | |
| Glucosulfatase | 3.1.6.3 | D-glucose 6-sulfate and other |
| | | sulfates of mono- and |
| | | disaccharides and on adenosine |
| | | 5′-sulfate |
| N-acetylgalactosamine-6- | 3.1.6.4 | 6-sulfate groups of the N- |
| sulfatase | | acetyl-D-galactosamine; 6- |
| (Chondroitinsulfatase, | | sulfate units of chondroitin |
| Chondroitinase, Galactose-6- | | sulfate and of the D-galactose |
| sulfate sulfatase) | | 6-sulfate units of keratan |
| | | sulfate. |
| Choline-sulfatase | 3.1.6.6 | Choline sulfate |
| Cellulose-polysulfatase | 3.1.6.7 | 2- and 3-sulfate groups of the |
| | | polysulfates of cellulose and |
| | | charonin |
| Cerebroside-sulfatase | 3.1.6.8 | A cerebroside 3-sulfate; |
| (Arylsulfatase A) | | galactose 3-sulfate residues in a |
| | | number of lipids; ascorbate 2- |
| | | sulfate; phenol sulfates |
| Chondro-4-sulfatase | 3.1.6.9 | 4-deoxy-beta-D-gluc-4- |
| | | enuronosyl-(1,4)-N-acetyl-D- |
| | | galactosamine 4-sulfate |
| Chondro-6-sulfatase | 3.1.6.10 | 4-deoxy-beta-D-gluc-4- |
| | | enuronosyl-(1,4)-N-acetyl-D- |
| | | galactosamine 6-sulfate; N- |
| | | acetyl-D-galactosamine 4,6- |
| | | disulfate |
| Disulfoglucosamine-6- | 3.1.6.11 | N,6-O-disulfo-D-glucosamine |
| sulfatase | | |
| (N-sulfoglucosamine-6- | | |
| sulfatase) | | |
| N-acetylgalactosamine-4- | 3.1.6.12 | 4-sulfate groups of the N- |
| sulfatase | | acetyl-D-galactosamine; 4- |
| (Arylsulfatase B; | | sulfate units of chondroitin |
| Chondroitinsulfatase; | | sulfate; dermatan sulfate; N- |
| Chondroitinase) | | acetylglucosamine 4-sulfate |
| Iduronate-2-sulfatase | 3.1.6.13 | 2-sulfate groups of the L- |
| (Chondroitinsulfatase) | | iduronate; 2-sulfate units of |
| | | dermatan sulfate; heparan |
| | | sulfate and heparin. |
| N-acetylglucosamine-6- | 3.1.6.14 | 6-sulfate group of the N-acetyl- |
| sulfatase | | D-glucosamine 6-sulfate; |
| (Glucosamine-6-sulfatase; | | heparan sulfate; keratan sulfate. |
| Chondroitinsulfatase) | | |
| N-sulfoglucosamine-3- | 3.1.6.15 | 3-sulfate groups of the N-sulfo- |
| sulfatase | | D-glucosamine 3-O-sulfate |
| (Chondroitinsulfatase) | | residues of heparin; N-acetyl- |
| | | D-glucosamine 3-O-sulfate |
| Monomethyl-sulfatase | 3.1.6.16 | Monomethyl sulfate |
| D-lactate-2-sulfatase | 3.1.6.17 | (S)-2-O-sulfolactate |
| Glucuronate-2-sulfatase | 3.1.6.18 | 2-sulfate groups of the 2-O- |
| (Chondro-2-sulfatase) | | sulfo-D-glucuronate residues of |
| | | chondroitin sulfate, heparin and |
| | | heparitin sulfate. |

The sulfatase substrate moiety can be designed to be reactive with a particular sulfatase or a group of sulfatases, or it can be designed to determine substrate specificity and other catalytic features, such as determining a value for kcat or Km. The sulphate ester in the sulfatase recognition moiety can be any group that is capable of being desulfated by a sulfatase.

In addition to having one or more sulphate esters capable of being desulfated, the sulfatase substrate moiety can include additional groups, for example amino acid residues (or analogs thereof) that facilitate binding specificity, affinity, and/or rate of desulfated by the sulfatase.

In other embodiments the peptide moiety can be designed to be reactive with a particular peptidase or group of peptidases. A peptidase is any member of a subclass of enzymes of the hydrolase class that catalyze the hydrolysis of peptide bonds. Generally, peptidases are divided into exopeptidases that act only near a terminus of a polypeptide chain and endopeptidases that act internally in polypeptide chains. The peptidase to be detected can be any peptidase known in the art. Also, the peptidase can be a peptidase candidate, and the methods used to confirm and/or characterize the peptidase activity of the candidate.

A wide variety of peptidases have been identified. Generally, peptidases are classified according to their catalytic mechanisms: 1) serine peptidases (such as such as chymotrypsin and trypsin); 2) cysteine peptidases (such as papain); 3) aspartic peptidases (such as pepsin); and, 4) metallo peptidases (such as thermolysin).

In some cases, peptidases are highly specific for only one or a few proteins, but in other cases, peptidases are relatively non-specific and can act on a large range of protein targets. Accordingly, compositions can be designed to detect particular peptidases by suitable selection of the peptidase substrate moiety. Exemplary peptidases and preferential cleavage sites, as indicated by “-|-” are shown in Table 8, below. These various cleavage sites can be used to design peptidase substrate moieties having desired specificities for particular peptidases and/or peptidase families.

TABLE 8

| Peptidase | EC number | Preferential cleavage |
|---|---|---|
| Chymotrypsin. | 3.4.21.1 | Tyr-\|-Xaa, Trp-\|-Xaa, Phe-\|-Xaa, |
| | | Leu-\|-Xaa |
| Trypsin | 3.4.21.4 | Arg-\|-Xaa, Lys-\|-Xaa. |
| Thrombin | 3.4.21.5 | Arg-\|-Gly |
| Renin | 3.4.23.15 | Pro-Phe-His-Leu-\|-Val-Ile |

Xaa - denotes any amino acid

The peptidase substrate moiety can be designed to be reactive with a particular peptidase or a group of peptidases, or it can be designed to determine substrate specificity and other catalytic features, such as determining a value for kcat or Km.

In addition to having one or more peptide bonds capable of being hydrolyzed, the peptidase substrate moiety can include additional amino acid residues (or analogs thereof) that facilitate binding specificity, affinity, and/or rate of hydrolysis by the peptidase.

In some embodiments, a trigger moiety, is used in the signal molecules described herein. Any means of activating the trigger moiety may be used, provided that the means used to activate the trigger moiety is capable of producing a detectable change (e.g., an increase) in fluorescence. Selection of a particular means of activation, and hence trigger moiety, may depend, in part, on the particular fragmentation reaction, as well as on other factors.

In some embodiments, activation is based upon cleavage of the trigger moiety. In these embodiments, the trigger moiety comprises a cleavage site that is cleavable by a chemical reagent or cleaving enzyme. As a specific example, the trigger moiety can comprise a cleavage site that is cleavable by a lipase, an esterase, a phosphatase, a glycosidase, a protease, a nuclease or a catalytic antibody. The trigger moiety can further comprise additional residues and/or features that facilitate the specificity, affinity and/or kinetics of the cleaving enzyme. Depending upon the requirements of the particular cleaving enzyme, such cleaving enzyme “recognition moieties” can comprise the cleavage site or, alternatively, the cleavage site may be external to the recognition moiety. For example, certain endonucleases cleave at positions that are upstream or downstream of the region of the nucleic acid molecule bound by the endonuclease.

The chemical composition of the trigger moiety will depend upon, among other factors, the requirements of the cleaving enzyme. For example, if the cleaving enzyme is a protease, the trigger moiety can comprise a peptide (or analog thereof) recognized and cleaved by the particular protease. If the cleaving enzyme is a nuclease, the trigger moiety can comprise an oligonucleotide (or analog thereof) recognized and cleaved by a particular nuclease. If the cleaving enzyme is glycosidase, the trigger moiety can comprise a carbohydrate recognized and cleaved by a particular glycosidase.

Sequences and structures recognized and cleaved by the various different types of cleaving enzymes are well known. Any of these sequences and structures can comprise the trigger moiety. Although the cleavage can be sequence specific, in some embodiments it can be non-specific. For example, the cleavage can be achieved through the use of a non-sequence specific nuclease, such as, for example, an RNase.

Structures recognized and cleaved by glycosidases are also well known (see, e.g., Florent, et al., J. MED. CHEM. 41:3572-3581 (1998), Ghosh, et al., TETRAHEDRON LETTERS 41:4871-4874 (2000), Michel, et al., ATTA-UR-RAHMAN (ED) 21:157-180 (2000), and Leu, et al., J. MED. CHEM. 42:3623-3628 (1999)). Specific examples of substrate compounds comprising trigger moieties cleavable by glycosidases are described in more detail below.

Structures recognized and cleaved by lipases and esterases are also well known (see, e.g., Ohwada, et al., BIOORG. MED. CHEM. LETT. 12:2775-2780 (2002), Sauerbrei, et al., ANGEW. CHEM. INT. ED. 37:1143-1146 (1998), Greenwald, et al., J. MED. CHEM. LETT. 43:475-487 (2000), Dillon, et al., BIOORG. MED. CHEM. LETT. 14:1653-1656 (1996), and Greenwald, et al., J. MED. CHEM. 47:726-734 (2004)). Specific examples of substrate compounds comprising trigger moieties cleavable by lipases and esterases are described in more detail below. In embodiments utilizing lipases as the specified trigger agent, it will be understood that the hydrophobic moiety does not comprise any cleavage sites for the lipase trigger agent.

Structures recognized and cleaved by proteases/proteolytic enzymes are also well known (see, e.g., Niculescu-Duvaz, et al., J. MED. CHEM. 41:5297-5309 (1998), Niculescu-Duvaz, et al., J. MED. CHEM. 42:2485-2489 (1999), Greenwald, et al., J. MED. CHEM. 42:3657-3667 (1999), de Groot, et al., BIOORG. MED. CHEM. LETT. 12:2371-2376 (2002), Dubowchik, et al., BIOCONJUGATE CHEM. 13:855-869 (2002), and de Groot, et al., J. ORG. CHEM. 66:8815-8830 (2001)). Specific examples of substrate compounds comprising trigger moieties cleavable by protease plasmin, trypsin, and carboxypeptidase G2 are described in more detail below.

Structures recognized and cleaved by catalytic antibodies are also well known (see, e.g, Gopin, et al., ANGEW. CHEM. INT. ED. 42:327-332 (2003), Dinaut, et al., CHEM. COMMUN. 1386-1387 (2001)). Specific examples of substrate compounds comprising trigger moieties cleavable by catalytic enzymes are described in more detail below.

In some embodiments, cleavage of the trigger moiety by a trigger agent can initiate fragmentation of the substrate compound directly without the formation of an intermediate compound. For example, cleavage of the trigger moiety by a glycosidase can result in the direct formation of a π electron-donor moiety that initiates a spontaneous reaction resulting in the fragmentation of the substrate compound.

In other embodiments, cleavage of the trigger moiety by the specified trigger agent can initiate fragmentation of the substrate compound indirectly via formation of an intermediate compound. In these embodiments, the intermediate compound generates a π electron-donor moiety that initiates a spontaneous reaction resulting in fragmentation of the substrate compound. For example, the trigger moiety can comprise an aromatic nitro or azide group that can be reduced, thereby generating a π electron-donor moiety that is capable of initiating fragmentation of the substrate compound and release of the hydrophobic moiety or the fluorescent moiety.

Fragmentation of the substrate compound following cleavage of the trigger moiety by the corresponding cleaving enzyme can release the fluorescent moiety from the micelle, reducing or eliminating quenching and producing a measurable increase in fluorescence.

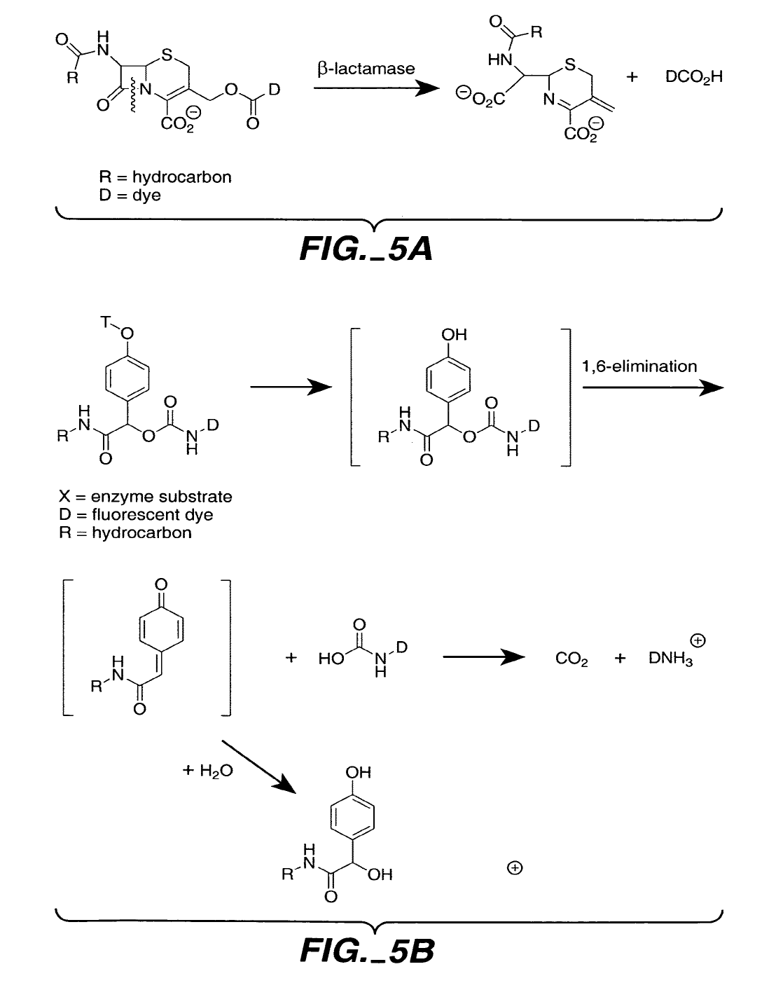

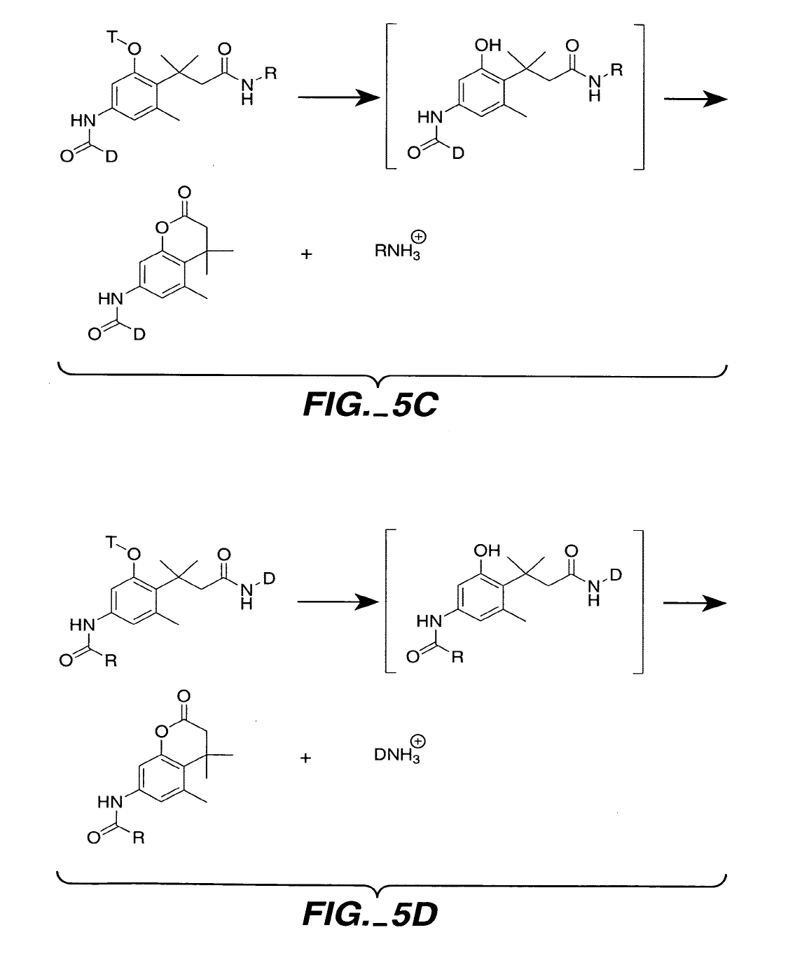

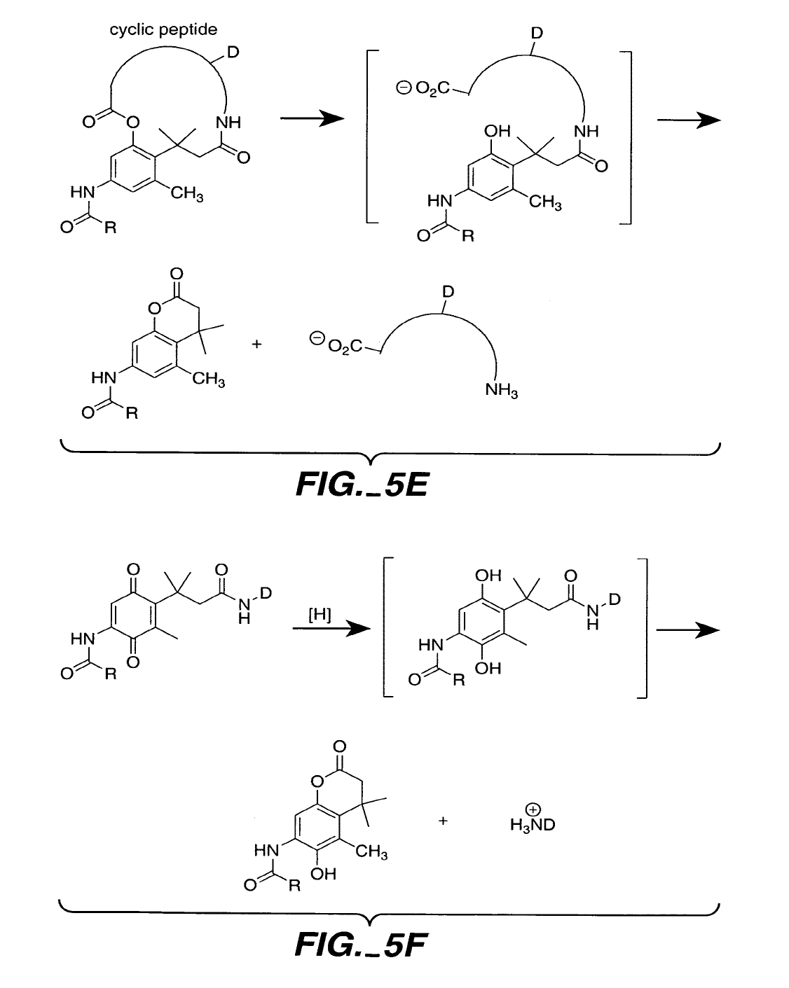

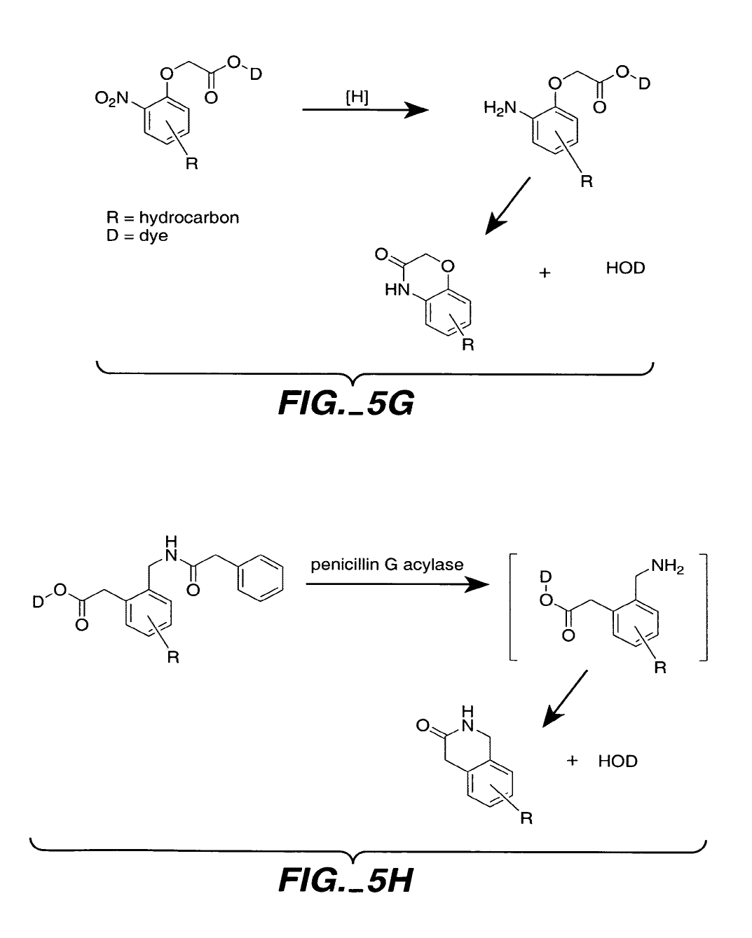

In other embodiments, the trigger moiety also serves as the linker moiety. In these embodiments, cleavage of the trigger moiety by a specified trigger agent also results in fragmentation of the substrate compound and release of the hydrophobic moiety, or the fluorescent moiety. FIG. 5A illustrates an exemplary embodiment of a substrate compound in which the linker moiety serves as the trigger moiety.

In other embodiments, formation of a π electron-donor moiety utilizes the reduction of chemical groups, such as aromatic nitro or azide moieties, connected to the linker moiety. Reduction of the chemical group generates a π electron-donor moiety that can initiate a spontaneous rearrangement reaction, resulting in the fragmentation of the linker, thereby promoting the release of the fluorescent moiety from the micelle. The release of the fluorescent moiety from the micelle produces a measurable increase in the fluorescence of the fluorescent moiety.

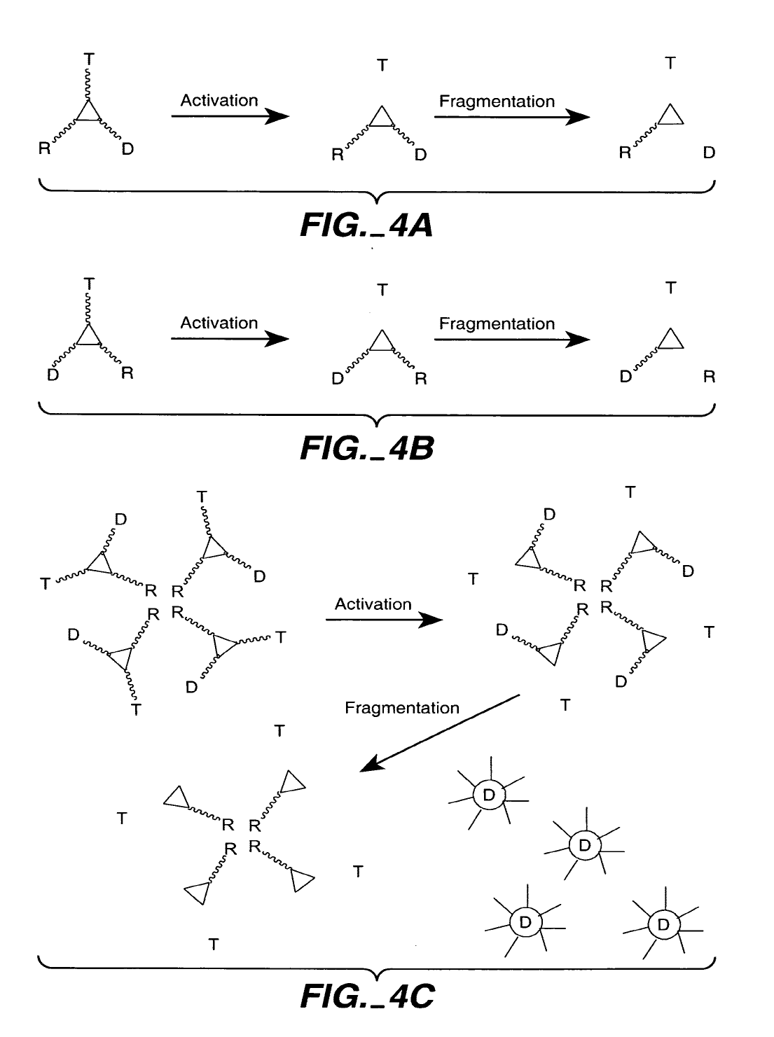

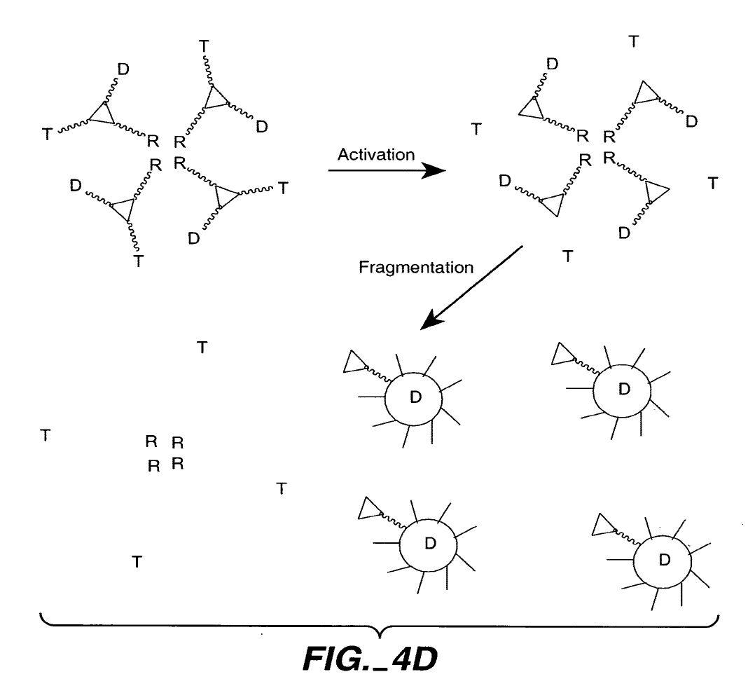

FIGS. 4A and 4B illustrate exemplary embodiments of a substrate compound comprising a trigger moiety T, a fluorescent moiety D, and a hydrophobic moiety, R, each of which, are independently of the other, attached to the backbone of a linker moiety. As illustrated in FIGS. 4A and 4B, the backbone of the linker moiety comprises three sites for the attachment of other molecules. Generally, the attachment site for the trigger moiety includes the r electron-donor moiety. The other two sites can be used for the attachment of optional linkage groups that can be used interchangeably for the attachment of the fluorescent moiety and the hydrophobic moiety. As will be appreciated by a person of skill in the art, the linker moiety illustrated in FIGS. 4A and 4B is merely exemplary, and linker moieties with two, three or more sites for the attachment of T, R, D, and optional substituent groups can be used in the compositions and methods described herein.

As illustrated in FIGS. 4A and 4B, fluorescent moiety D comprises a fluorescent dye. However, any reporter moiety that is operative in accordance with the various compositions and methods described herein can be used in place of D to detect the presence and/or quantity of a molecule of interest.

As illustrated in FIGS. 4A and 4B, R can comprise any of the hydrophobic groups described above. For example, R can comprise saturated or unsaturated alkyl chains, which may be same or different. In other embodiments, R can comprise a phospholipid comprising at least two hydrophobic moieties, e.g., R¹ and R², as described above.

As illustrated in FIGS. 4A and 4B, T can comprise any of the trigger moieties outlined above, which when activated by a specified trigger agent are capable of initiating a spontaneous rearrangement reaction that promotes fragmentation of the substrate compound and release of the fluorescent moiety or the hydrophobic moiety. For example, T can comprise a cleavage site that is recognized and cleaved by a cleaving enzyme, such as a lipase, an esterase, a phosphatase, a glycosidase, a carboxypeptidase or a catalytic antibody. Alternatively, T can comprise an aromatic nitro or azide group that can be reduced, thereby generating a π electron-donor group that is capable of initiating fragmentation of the substrate compound and release of the hydrophobic moiety or the fluorescent moiety.

In the exemplary embodiments illustrated in FIG. 4A or 4B, fluorescent moiety D or hydrophobic moiety R is released from the backbone of the linker moiety via a spontaneous rearrangement reaction. Spontaneous rearrangement reactions capable of fragmenting the substrate compound and releasing D or R include 1,4-, bis 1,4-, 1,6-, mono 1,8-, and bis 1,8-elimination reactions, and ring closure mechanisms, such as trimethyl lock lactonization reactions and intramolecular cyclization reactions.

In the exemplary embodiment illustrated in FIG. 4A, release of fluorescent moiety D is initiated by activation of T by a specified trigger agent. In some embodiments, T comprises a cleavage site for a cleaving enzyme. Activation is initiated when the cleaving enzyme recognizes and cleaves T at the cleavage site, thereby generating a π electron-donor moiety that is capable of initiating a spontaneous rearrangement reaction that results in the cleavage of T from the backbone of the linker moiety. Subsequent rearrangement(s) result in the fragmentation of the linker and release of D.

In other embodiments, T comprises a reactive nitro or azide group. In these embodiments, a π electron-donor moiety is generated when the nitro or azide group is reduced. Reduction of the nitro or azide group generates a π electron-donor moiety, e.g., —NH—, that is capable of initiating a spontaneous rearrangement reaction that results in the cleavage of T from the backbone of the linker. Subsequent rearrangement(s) result in the fragmentation of the linker and release of D.

In the exemplary embodiment illustrated in FIG. 4B, hydrophobic moiety R is released from the backbone of the linker as described above. In this embodiment, D remains attached to the backbone of the linker.

As illustrated in FIG. 4C, if the fluorescent moiety is released by the fragmentation reaction, the “free” fluorescent moiety fluoresces brightly since it remains relatively free from other fluorescent substrate molecules in the solution.

As illustrated in FIG. 4D, if the hydrophobic moiety is released by the fragmentation reaction, it remains associated with the micelle, while the backbone of the linker comprising the fluorescent moiety is released from the micelle. As illustrated in FIG. 4D, the “free” fluorescent moiety fluoresces brightly since it remains relatively free from other fluorescent substrate molecules in the solution.

In some embodiments, the substrate compound comprises a linker moiety that fragments via an elimination reaction. Various elimination reactions, such as 1,4-, 1,6- and 1,8-elimination reactions have been used in the design of prodrugs and can be easily adapted for use in the compositions and methods described herein. See, e.g., WO 02/083180, Gopin, et al., ANGEW. CHEM. INT. ED. 42:327-332 (2003), Niculescu-Duvaz, et al., J. MED. CHEM. 41:5297-5309 (1998), Florent, et al., J. MED. CHEM. 41:3572-3581 (1998), Niculescu-Duvaz, et al., J. MED. CHEM. 42:2485-2489 (1999), Greenwald, et al., J. MED. CHEM. 42:3657-3667 (1999), de Groot, et al., BIOORG. MED. CHEM. LETT. 12:2371-2376 (2002), Ghosh, et al., TETRAHEDRON LETTERS 41:4871-4874 (2000), Dubowchik, et al., BIOCONJUGATE CHEM. 13:855-869 (2002), Michel, et al., ATTA-UR-RAHMAN (ED) 21:157-180 (2000), Dinaut, et al., CHEM. COMMUN. 1386-1387 (2001), Ohwada, et al., BIOORG. MED. CHEM. LETT. 12:2775-2780 (2002), de Groot, et al., J. ORG. CHEM. 66:8815-8830 (2001), Leu, et al., J. MED. CHEM. 42:3623-3628 (1999), Sauerbrei, et al., ANGEW. CHEM. INT. ED. 37:1143-1146 (1998), Veinberg et al., BIOORG. MED. CHEM. LETT. 14:1007-1010 (2004), Greenwald, et al., BIOCONJUGATE CHEM. 14:395-403 (2003), and Lee et al., ANGEW. CHEM. INT. ED. 43:1675-1678 (2004).

FIG. 5B illustrates an exemplary embodiment of a substrate compound in which the substrate compound fragments via a 1,6-elimination reaction. In the embodiment illustrated in FIG. 5B, the substrate compound generally comprises a trigger moiety (represented by T), a fluorescent moiety (represented by D), a hydrophobic moiety (represented by R), and a linker moiety comprising a benzyl backbone. In the embodiment illustrated in FIG. 5B, the π electron-donor moiety attached to the carbon atom at position C1 of the benzyl backbone can comprise a reactive —O— group as shown, or a reactive —NH— or —S— group. In the embodiment illustrated in FIG. 5B, trigger moiety T is connected directly to the reactive —O— group. In other embodiments, T can be indirectly connected to the reactive —O— group via an additional linkage L, such as those described above. In the embodiment illustrated in FIGS. 5B, D and R are both attached to the benzyl linker at the C4 carbon via a CH group. In the embodiment illustrated in FIG. 5A, D is attached via a L² linkage, e.g., —O—C(O)—NH, and R is attached via a stable L¹ linkage, e.g., —C(O)—NH.

The addition of a specified trigger agent to the substrate compound illustrated in FIG. 5B initiates a 1,6-elimination reaction by removing T and generating a reactive hydroxy group at the C1 carbon of the benzyl backbone. The hydroxy group so generated spontaneously promotes the 1,6-elimination reaction resulting in the release of the HOCONHD moiety. Further rearrangement results in the release of CO₂ and DNH₃⁺. In the embodiment illustrated in FIG. 5B, R remains attached to the backbone of the benzyl linker moiety.

Exemplary benzyl linker structures that can be used for 1,4- and 1,6-elimination reactions are shown below in Table 9.

TABLE 9

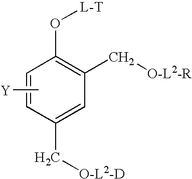

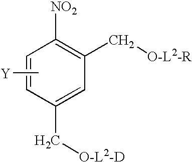

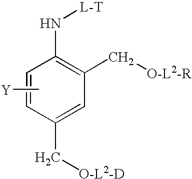

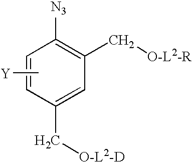

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any site not used for the attachment of the fluorescent moiety or the hydrophobic moiety. For example if the fluorescent moiety is attached to the benzyl linker at the C4 carbon and the hydrophobic moiety is attached to the benzyl linker at the C2 position, then Y can be attached at the C3, C4 and/or C5 carbon atoms.

Exemplary embodiments of benzyl linker structures that can be used in 1,6-elimination reactions are illustrated below in Table 10.

TABLE 10

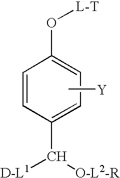

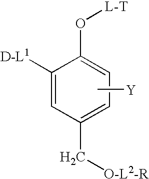

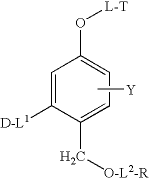

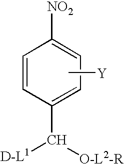

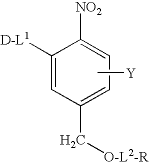

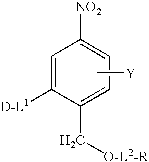

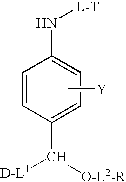

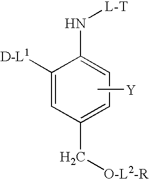

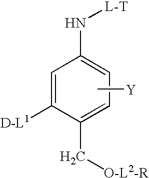

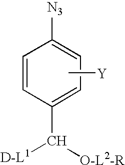

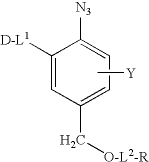

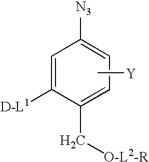

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the hydrophobic moiety attached to the leaving group, similar structures can be designed in which the fluorescent moiety is attached to L².

Y represents one or more optional substituent groups as described above, that can be attached at any attachment site that is not used for the attachment of the fluorescent moiety or the hydrophobic moiety. For example, if both the hydrophobic moiety and the fluorescent moiety are attached to the C4 carbon atom, then Y can be attached at the C2, C3 and/or C5 carbon atoms.

Exemplary embodiments of benzyl linker structures that can be used in 1,4-elimination reactions are illustrated below in Table 11.

TABLE 11

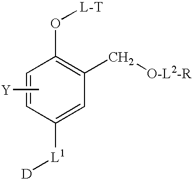

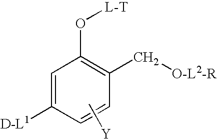

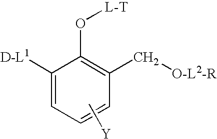

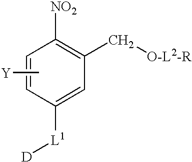

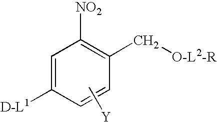

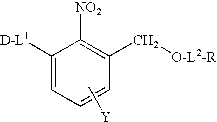

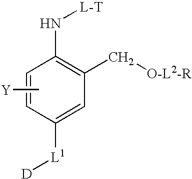

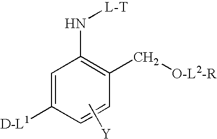

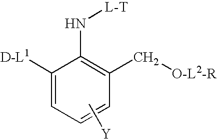

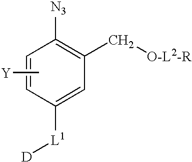

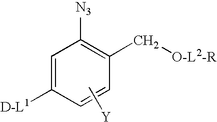

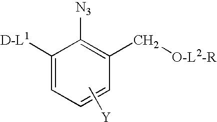

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the hydrophobic moiety attached to the leaving group, similar structures can be designed in which the fluorescent moiety is attached to L².

Y represents one or more optional substituent groups as described above, that can be attached at any attachment site that is not used for the attachment of the fluorescent moiety or the hydrophobic moiety. For example, if the hydrophobic moiety is attached at the C2 carbon atom and the fluorescent moiety is attached to the C5 carbon atom, then Y can be attached at the C3 and/or C4 carbon atoms.

In other embodiments, benzyl linkers for bis 1,4-elimination reactions can be used in the compositions and methods described herein. Exemplary benzyl linker structures for bis 1,4-elimination reactions are shown in Table 12.

TABLE 12

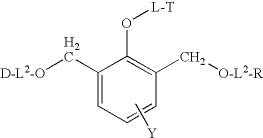

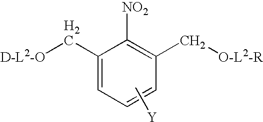

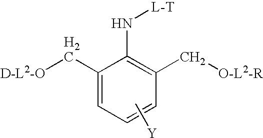

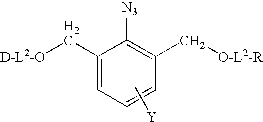

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any attachment site that is not used for the attachment of the fluorescent moiety or the hydrophobic moiety. For example, if the hydrophobic moiety is attached at the C2 carbon atom and the fluorescent moiety is attached to the C6 carbon atom, then Y can be attached at the C3, C4 and/or C5 carbon atoms.

Exemplary embodiments of benzyl linker structures that can be used in 1,8-elimination reactions are illustrated below in Table 13.

TABLE 13

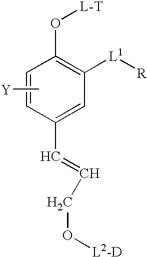

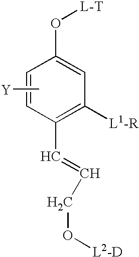

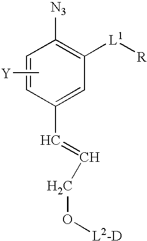

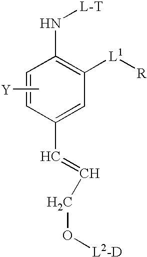

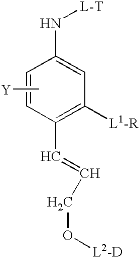

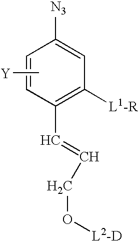

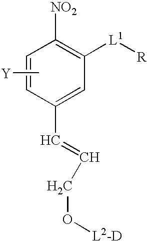

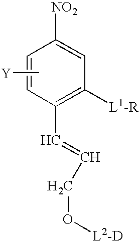

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the fluorescent moiety attached to the leaving group, similar structures can be designed in which the hydrophobic moiety is attached to L². Y represents one or more optional substituent groups as described above, that can be attached at any attachment site that is not used for the attachment of the fluorescent moiety or the hydrophobic moiety. For example, if the hydrophobic moiety is attached to the C3 carbon atom and the fluorescent moiety is attached to the C4 carbon atom, then Y can be attached to the C2, C5 and/or C6 carbon atoms.

In other embodiments, benzyl linkers for bis 1,8-elimination reactions can be used in the compositions and methods described herein. Exemplary benzyl linker structures for bis 1,8-elimination reactions are shown in Table 14.

TABLE 14

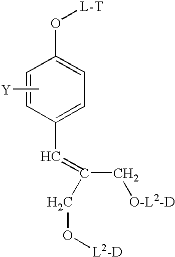

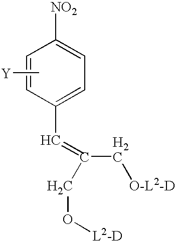

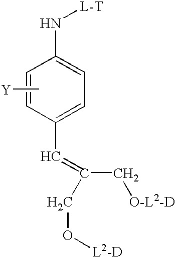

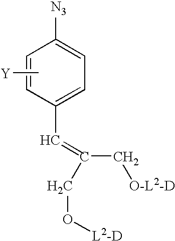

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any attachment site that is not used for the attachment of the fluorescent moiety or the hydrophobic moiety. For example, if the hydrophobic moiety and the fluorescent moiety are attached to the C4 carbon atom, then Y can be attached to the C2, C3, C5, and/or C6 carbon atoms.

Skilled artisans will appreciate that while the substrate compounds illustrated in Tables 9-14 are not exemplified with specific trigger moieties, functional groups, hydrophobic moieties, or fluorescent moieties any one of the various moieties described herein can be used with the generalized linker structures illustrated in Tables 9-14. Moreover, virtually any type of chemical linkage(s) that is stable to the assay conditions and that permit the various moieties to perform their respective functions could be used. Additionally, the various illustrated features can be readily “mixed and matched” to provide other specific embodiments of exemplary substrate compounds.

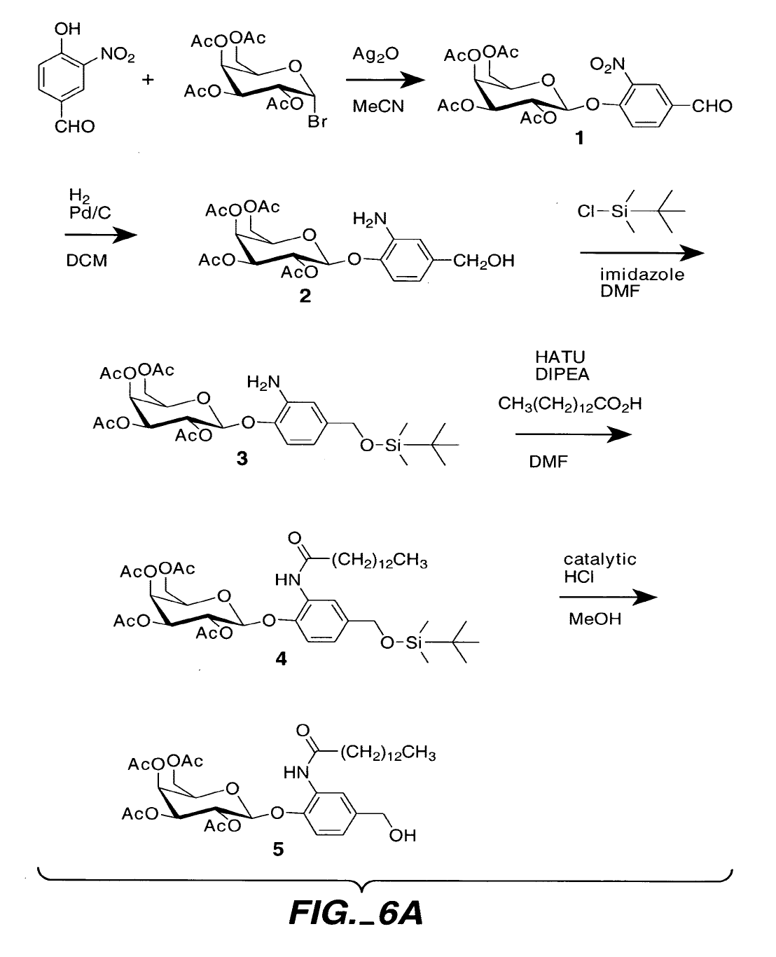

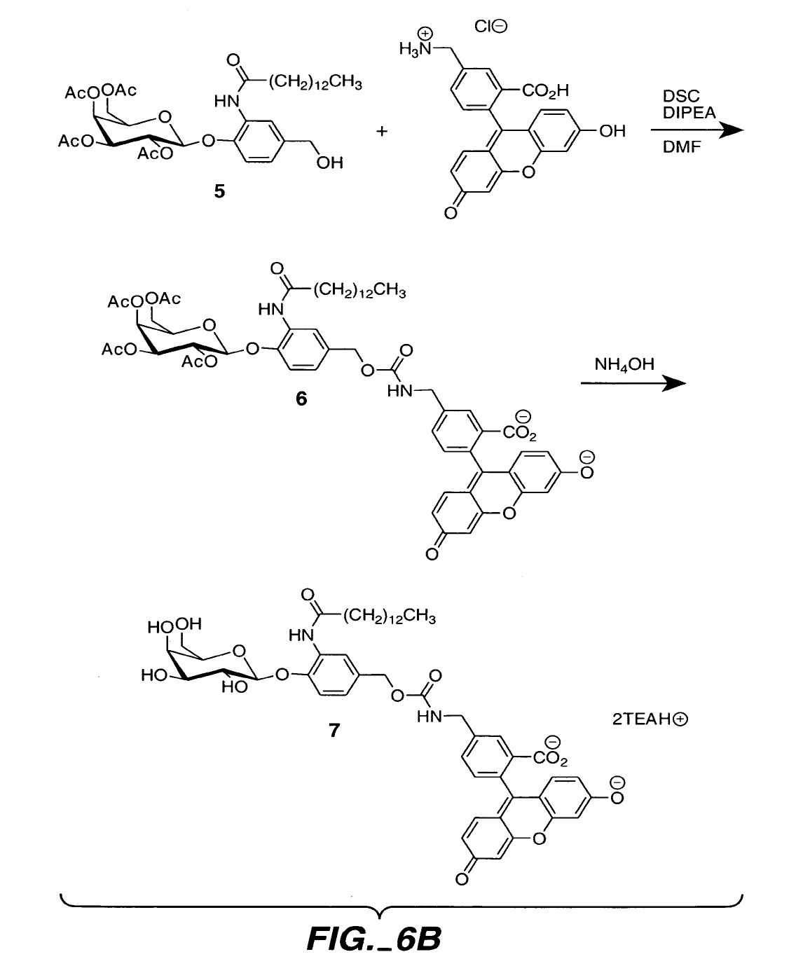

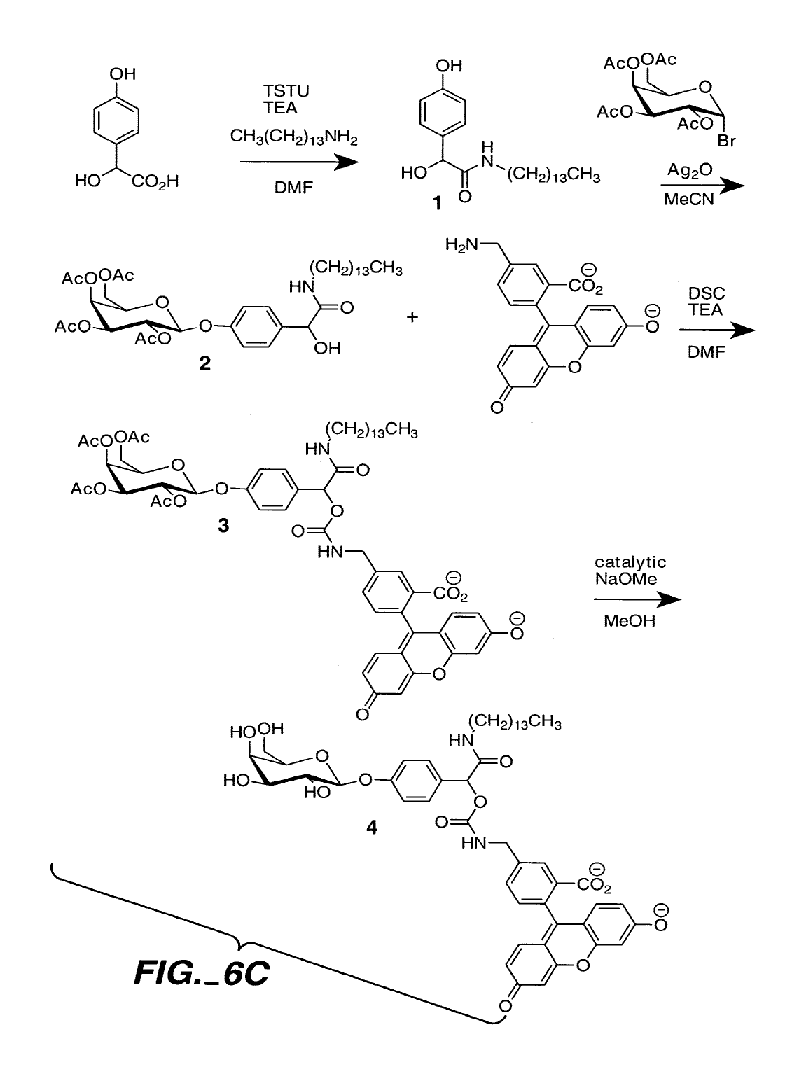

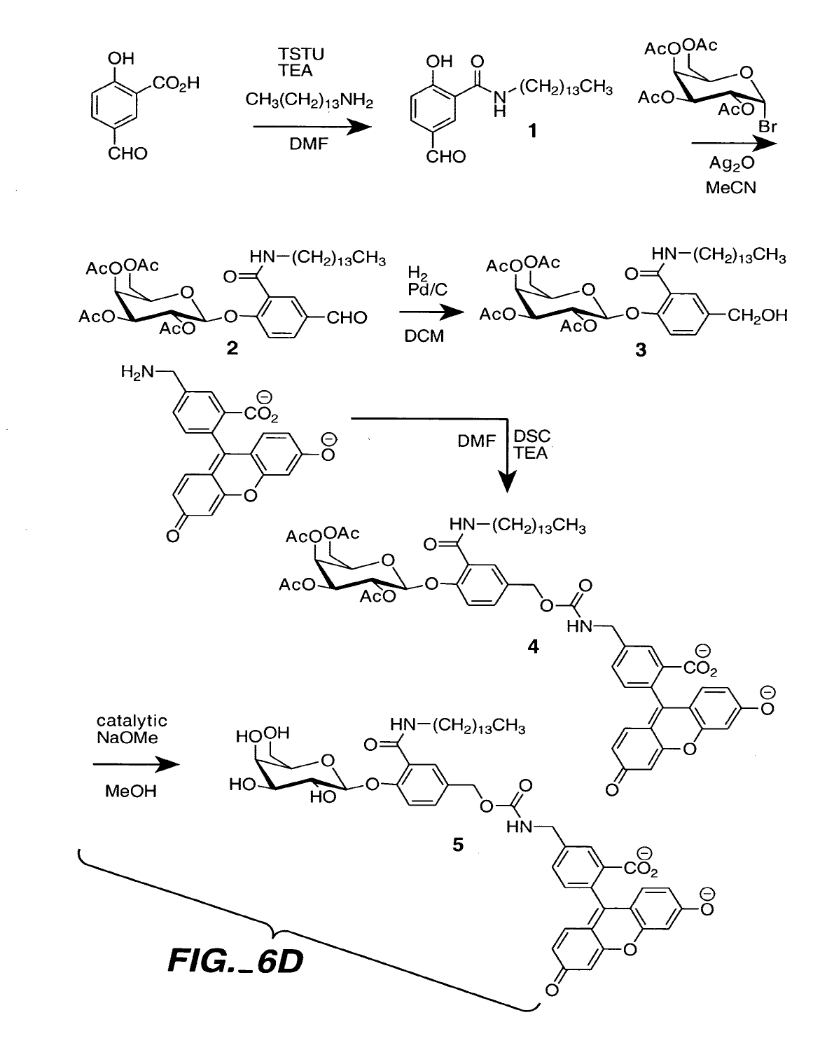

Substrate compounds comprising benzyl linkers capable of undergoing a 1-4- or a 1-6 elimination reaction can be synthesized according to the scheme illustrated in FIGS. 6A-6B and described in Example 7.5

In some embodiments, the substrate compound comprises a linker moiety that fragments via a ring closure mechanism. Exemplary ring closure mechanisms include trimethyl lock lactonization reactions (see, e.g., Greenwald, et al., J. MED. CHEM. LETT. 43:475-487 (2000), Cheruvallath, et al., BIOORG. MED. CHEM. LETT.:281-284 (2003), Zhu, et al., BIOORG. MED. CHEM. LETT. 10:1121-1124 (2000), Dillon, et al., BIOORG. MED. CHEM. LETT. 14:1653-1656 (1996), Ueda, et al., BIOORG. MED. CHEM. LETT. 8:1761-1766 (1993)) and intramolecular cyclization reactions using safety catch linkers (see, e.g., Greenwald, et al., J. MED. CHEM. 47:726-734 (2004).

Exemplary substrate compounds capable of fragmenting by a trimethyl lock lactonization reaction have the structure shown below:

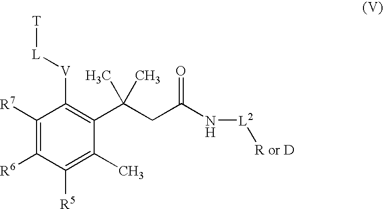

In the embodiment illustrated in Structure V, the backbone of the linker moiety is a phenyl group comprising two, three or more sites that can be used to attach the trigger moiety, hydrophobic moiety and fluorescent moiety to the backbone of the linker moiety. Although the backbone of the linker moiety is illustrated as a phenyl, the linker backbone need not be limited to carbon and hydrogen atoms. For example, the linker backbone could include heteroaryl compounds comprising carbon-nitrogen bonds, nitrogen-nitrogen bonds, carbon-oxygen bond, carbon-sulfur bonds and combinations thereof.

As illustrated in Structure V, R⁵, R⁶, and R⁷ can comprise an optional substituent group “Y”, L¹-R or L¹-D. L, L¹, and L² represent linkage groups as described above. The selection of the various combinations of substituents, will depend in part, on whether the hydrophobic moiety or fluorescent moiety is attached to L². For example, if the fluorescent moiety is attached to L², then any one R⁵, R⁶, and R⁷ can comprise L¹-D and, if desired, optional Y groups, provided that they are connected in a way that permits them to perform their respective functions and in a manner that does not interfere with the fragmentation of the substrate compound and release of the fluorescent moiety. Similarly, if the hydrophobic moiety is attached to L², then any one R⁵, R⁶, and R⁷ can comprise L¹-D and, if desired, optional Y groups, provided that they are connected in a way that permits them to perform their respective functions and in a manner that does not interfere with the fragmentation of the substrate compound and release of the hydrophobic moiety.

A wide variety of optional Y substituents that are suitable for use with linker moieties that fragment via a ring closure method are known in the art, and include by way of example and not limitation —H—, —CH₃—, and —(CH₂)ₙCO₂H—.

The trigger moiety (represented by T) is attached to the C1 carbon of the phenyl linker backbone via a reactive —O—. In other embodiments, the trigger moiety can be attached to the C1 carbon via a reactive —NH— group. In addition, an optional linkage L can be used to link T to the reactive —O— or —NH— moiety, or to facilitate the specificity, affinity and/or kinetics of the specified trigger agent. Examples of suitable trigger moieties and corresponding trigger agents are provided in Table 15 below.

TABLE 15

| Trigger Moiety | Trigger Agent |
|---|---|
| PO₃H⁻ | Phosphatase |
| 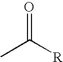 | Lipase |
| 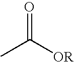 | Esterase |
| 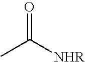 | Protease |

As will be appreciated by a person skilled in the art, the illustrated trigger moieties and trigger agents provided in Table 15 are merely exemplary trigger moieties and trigger agents. Any trigger moiety comprising a cleavage site suitable for cleavage by a cleavage enzyme and that can be appropriately cleaved to provide a reactive —O— or —NH— group could be used to provide a trigger moiety. In some embodiments, an optional linkage can be used to modulate the activity of the trigger agent. For example, a cleavage site comprising a carbohydrate moiety capable of being cleaved and an optional linkage could be used as the trigger moiety and the corresponding glycosidase used as the specified trigger agent.

In the exemplary substrate compound illustrated in Structure V, a linkage group, i.e., —CH(CH₃)₂CH₂CO—Z capable of undergoing a cyclization reaction is attached to the carbon atom at position C2 of the phenyl backbone. This linkage group serves as point of attachment for a leaving group Z to which can be attached the fluorescent moiety or the hydrophobic moiety. Suitable Z moieties include —NH— and —O.

Additional linkages groups can be used for the attachment of the hydrophobic moiety or fluorescent moiety to carbon atoms at positions C3, C4, C5 or C6. Suitable linkage groups include those discussed above for embodiments in which the linker moiety fragments by an elimination reaction.

In the exemplary substrate compound illustrated in FIG. 5C, the hydrophobic moiety (represented by R) is attached to a linkage group that is capable of cyclizing following activation of the trigger moiety by a specified trigger agent. Cyclization of the illustrated linkage group results in the release of the R from the backbone of the linker moiety. As illustrated in FIG. 5D, the fluorescent moiety (represented by D) is attached to a linkage that participates in the cyclization reaction. Thus, in the embodiment illustrated in FIG. 5D, D is released from the backbone of the linker moiety.

An exemplary substrate compound fragmented via a trimethyl lock lactonization reaction is illustrated in FIG. 5E. In the exemplary substrate illustrated in FIG. 5E, T comprises a cleavage site for an esterase, Z comprises a cyclic peptide leaving group to which D is connected, Y comprises a methyl group attached to carbon atom C3, and the hydrophobic moiety is attached to C4 via a —CONH— linkage group. Cleavage of T by an esterase initiates the trimethyl lock lactonization reaction, thereby releasing D.

In the exemplary substrate compound embodiment illustrated in FIG. 5F, fragmentation via a trimethyl lock lactonization reaction is activated under reducing conditions that convert the nitro group to a reactive —NH— group. The reactive —NH— group then initiates a lactonization reaction that results in the release of D.

Substrate compounds capable of fragmenting by a ring closure mechanism utilizing a safety catch linker have the structure shown below:

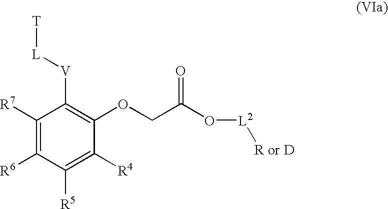

In the embodiment illustrated in Structure VIa, the backbone of the linker moiety is a phenyl group comprising two, three or more sites that can be used to attach the trigger moiety, hydrophobic moiety and fluorescent moiety to the backbone of the linker. Although the backbone of the linker moiety is illustrated as a phenyl, the backbone of the linker moiety need not be limited to carbon and hydrogen atoms. For example, the backbone of the linker could include heteroaryl compounds comprising carbon-nitrogen bonds, nitrogen-nitrogen bonds, carbon-oxygen bond, carbon-sulfur bonds and combinations thereof.

In the exemplary embodiment illustrated in Structure VIa, the trigger moiety (represented by T) is attached to the carbon atom at position C1 of the phenyl backbone. As described above, T comprises a π electron-donor moiety (i.e. V) to which is attached, directly or indirectly via an optional linkage L, a cleavage site for a cleaving enzyme. In other embodiments, e.g., Structure VIb, T can comprise an aromatic nitro or azide group that can be reduced to generate a π electron-donor moiety.

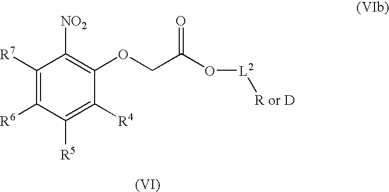

As illustrated in Structure Via or VIb, R⁴, R⁵R⁶ and R⁷ can comprise the hydrophobic moiety, the fluorescent moiety and one or more optional substituent groups (not shown). The location of the fluorescent moiety or the hydrophobic moiety, will depend in part, on whether the hydrophobic moiety or fluorescent moiety is attached to the L² linkage group. For example, if the fluorescent moiety is attached to the L² linkage group, then any one of R⁴, R⁵, R⁶ and R⁷ can comprise L¹-R and, if desired, optional Y groups, provided that L¹-R and Y are connected in a way that permits them to perform their respective functions and in a manner that does not interfere with the fragmentation of the substrate compound and release of the fluorescent moiety. Similarly, if the hydrophobic moiety is attached to the L² linkage group, then any one of R⁴, R⁵, R⁶ and R⁷ can comprise L¹-D and, if desired, optional Y groups, provided that L¹-D and Y are connected in a way that permits them to perform their respective functions and in a manner that does not interfere with the fragmentation of the substrate compound and release of the hydrophobic moiety.

In the exemplary substrate compound illustrated in FIG. 5G, fragmentation via a ring closure reaction using a “safety catch linker” is activated by a reductive environment that converts the nitro group to a reactive —NH— group. In the exemplary embodiment illustrated in FIG. 6G, the electronic cascade reaction initiates cleavage of the ester moiety, ring closure, and release of D.

In the exemplary substrate compound illustrated in FIG. 5H, fragmentation via a ring closure reaction using a “safety catch linker” is activated by a cleaving enzyme, i.e. pencillin G acylase. Cleavage by pencillin G acylase generates a reactive —NH₂— group that initiates a ring closure reaction that results in the release of D.

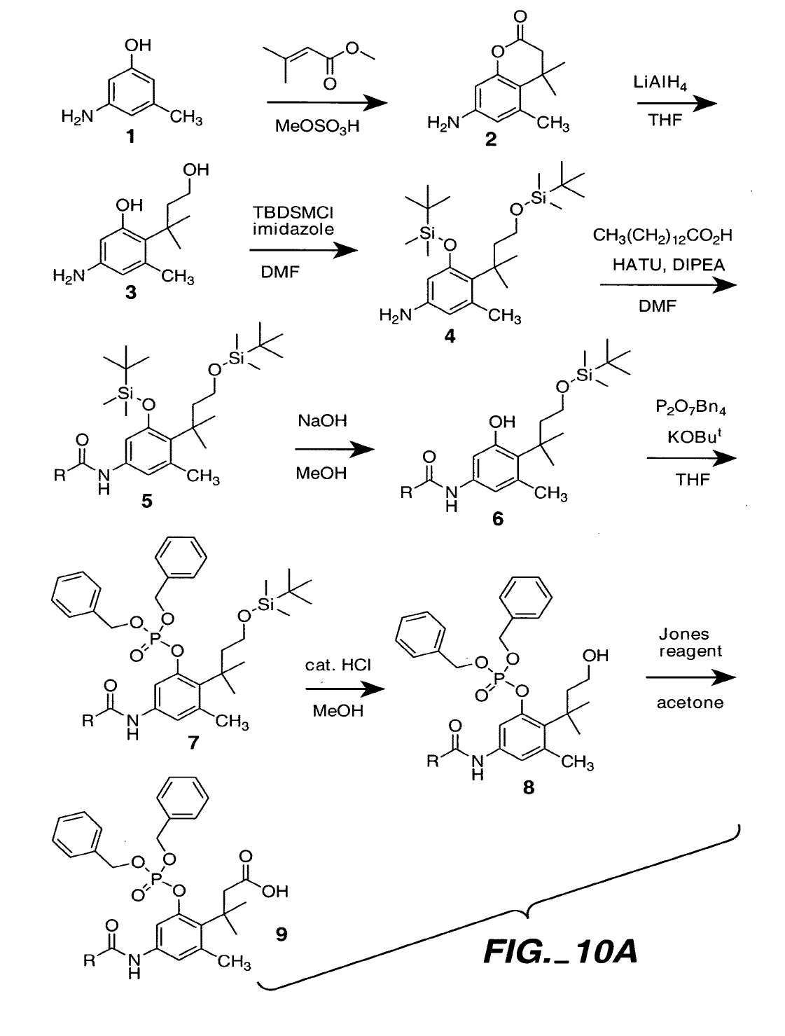

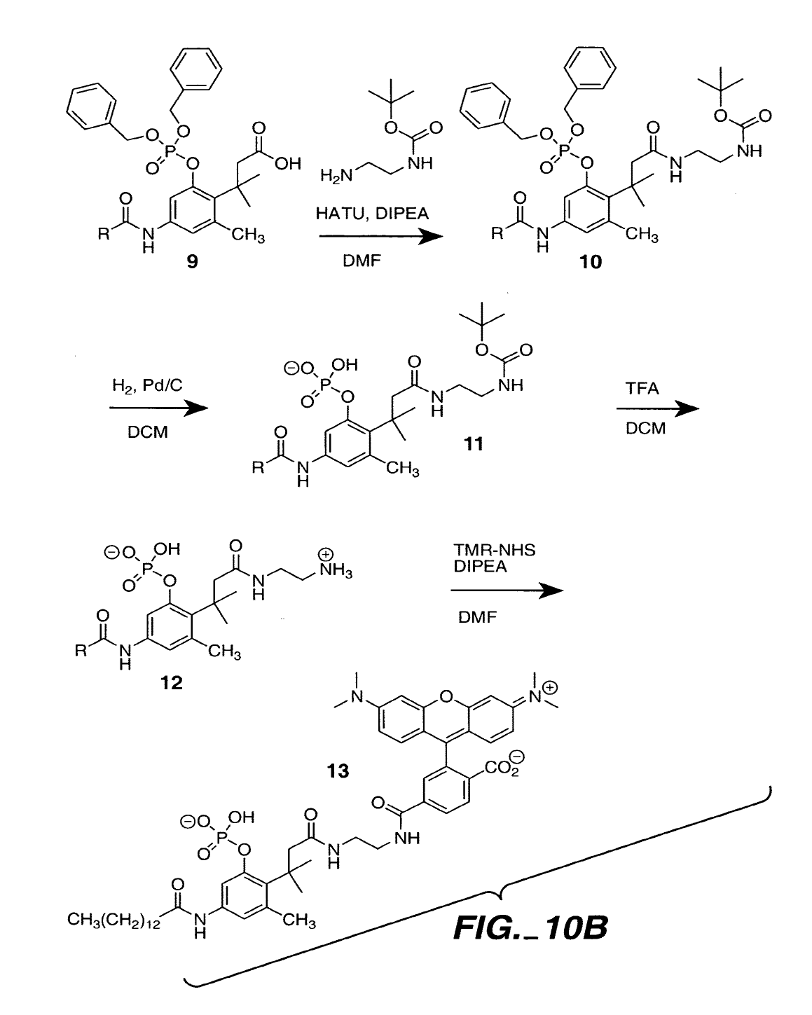

A synthetic scheme for the synthesis of a substrate compound capable of undergoing a ring closure elimination reaction, i.e. a trimethyl lock lactonization reaction, is illustrated in FIGS. 10A-10B and described in Example 7.13.

Skilled artisans will appreciate that any one of the hydrophobic moieties, fluorescent moieties and trigger moieties described herein can be used with the various substrate compounds illustrated in FIGS. 5C-5H. Additionally, the various illustrated features can be readily “mixed and matched” to provide other specific embodiments of exemplary substrate compounds.

The linker moiety comprises attachment sites for the attachment of the fluorescent moiety, hydrophobic moiety, trigger moiety, and one or more optional substituent groups. One of the attachment sites comprises a π electron-donor moiety that can be used for the attachment of the trigger moiety. The trigger moiety can be attached directly to the π electron-donor moiety, or indirectly to the π electron-donor moiety via one or more optional linkages. For example, the trigger moiety can be attached to the backbone of the linker directly via a π electron-donor moiety, such as —O—, —S, or —NH—, or it can be attached indirectly to the backbone of the linker moiety via an optional linkage L, such as a —COO⁻—.

Other attachment sites comprise linkages for the attachment of the fluorescent moiety and the hydrophobic moiety. The fluorescent moiety and hydrophobic moiety can be attached to the same attachment site or to different attachment sites. Linkages useful for attaching the fluorescent moiety and the hydrophobic moiety include linkages having the general formula L¹ and L², wherein L¹ represents a linkage that is stable under the conditions of the assay, such that the linkage does not dissociate from the backbone of the linker moiety following the fragmentation reaction. L² represents a linkage comprising a leaving group. Examples of linkages suitable for use in the compositions and methods are described above.

In some embodiments, substrate compounds capable of fragmenting by an elimination reaction have the structure shown below:

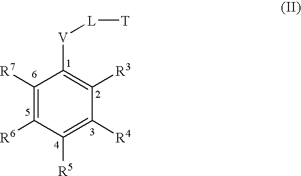

In structure II, “V” represents a π electron donor moiety, “L” represents an optional linkage group, “T” represents a trigger moiety, R3, R4, R5, R6, and R7 each independently comprise attachment sites for the attachment of the fluorescent moiety, the hydrophobic moiety and one or more optional substituent groups, “Y”.

In the exemplary substrate compound illustrated in Structure II, “V” can be O, NH, or S. “L” is an optional linkage group that can be used to attach the trigger moiety “T” to the backbone of the aromatic linker, such as those described below and in Table 16. Typically L is used to module the activity of the trigger agent. For example, if the activity of the trigger agent is susceptible to steric hindrance, an optional linkage can be used to “distance” the trigger moiety from the sterically crowded linker moiety. Alternatively, if the trigger agent is too reactive, an optional linkage can be used to increase the steric hindrance. Linkages suitable for modulating the enzyme activity are known to those of skill in the art, and include —COO⁻—.

Suitable trigger moieties include those that are cleaved by an enzyme or can be reduced under reducing conditions. Typically, the compositions use trigger moieties that are cleaved by an enzyme. Examples of suitable “T” cleavage sites, cleaving enzymes, and optional linkage groups are provided in Table 16.

TABLE 16

| | Cleavage Site with | |
|---|---|---|
| Cleavage Site | Optional Linkage group | Cleaving Enzyme |
| 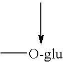 | 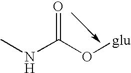 | β-glucuronidase |
| 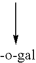 | 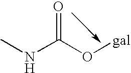 | β-galactosidase |
| 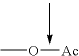 | | lipase/esterase |
| 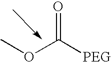 | 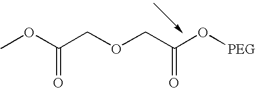 | lipase/esterase |
| 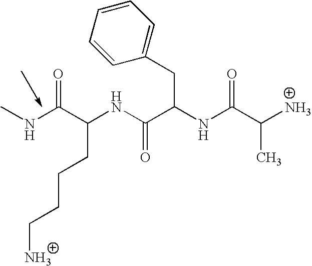 | | protease/plasmin |
| 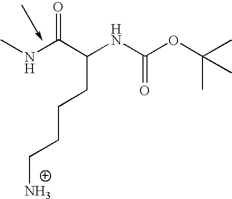 | | trypsin |
| 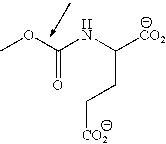 | | carboxypeptidase G2 |
| 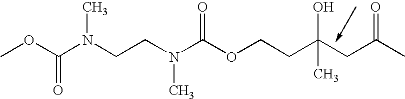 | | catalytic antibody |
| 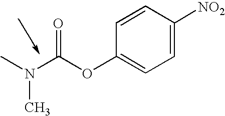 | | catalytic antibody |

Glu and gal represent carbohydrates glucuronide and galactose, respectively. Cleavage sites are indicated by arrows.

The illustrated cleavage sites, cleavage sites with optional linkages and cleaving enzymes are merely exemplary trigger moieties and trigger agents. Any trigger moiety comprising a cleavage site suitable for cleavage by a cleavage enzyme that can be appropriately cleaved, leaving behind the π electron donor moiety could be used to provide an appropriate cleavage site. For example, a cleavage site comprising a phosphate group capable of being cleaved by a phosphatase could be used as trigger moiety and the corresponding phosphatase used as the specified trigger agent (see, e.g., Zhu, et al., BIOORG. MED. CHEM. LETT. 10:1121-1124 (2000), and Ueda, et al., BIOORG. MED. CHEM. LETT. 8:1761-1766 (1993)).

In other embodiments, T can comprise an aromatic nitro or azide group directly attached to the carbon atom at position C1 of the exemplary linker moieties illustrated in Structure II. Similar linker moieties are described in Damen, et al., for the delivery of prodrugs (Damen, et al., BIOORG. MED. CHEM. 10:71-77). Exemplary substrate compounds comprising an aromatic nitro or azide group are shown below:

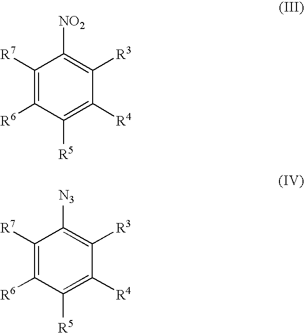

In the illustrated structures II-IV, R³, R⁴, R⁵, R⁶, and R⁷ are each independently the sites of attachment for the fluorescent moiety, the hydrophobic moiety and one or more optional substituent groups. In structures II, III, and IV, R³, R⁴, R⁵, R⁶, and R⁷ can be independently selected from:

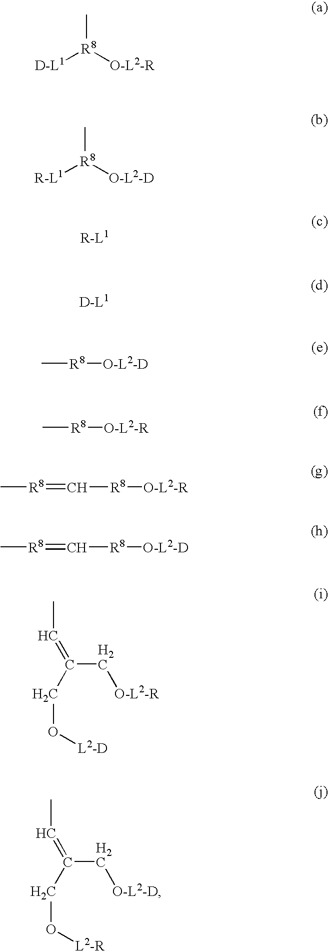

as well as from hydrogen, alkyl, aryl, cycloalkyl, heterocycloalkyl, heteroaryl, alkoxy, hydroxy, thiohydroxy, thioalkoxy, aryloxy, thioaryloxy, amino, nitro, halo, trihalomethyl, cyano, C-amido, N-amido, imidazolyl, alkylpiperazinyl, morpholino, tetrazole, carboxy, carboxylate, sulfoxy, sulfonate, sulfonyl, sulfixy, sulfinate, sulfinyl, phosphonooxy, or phosphate, or alternatively, at least two of R³, R⁴, R⁵, R⁶, and R⁷ can be connected to one another to form an aromatic or aliphatic cyclic structure;
wherein:
D is a fluorescent dye moiety as described herein;
R is a hydrophobic moiety as described herein;
R⁸ can be selected from the group consisting of CH, CR, CHR, and CR₂;
L¹ represents a stable linkage, including but not limited to an amide linkage, an N-O-linkage, and a —N═N— linkage
L² represents a linkage comprising a leaving group Z, and can be selected from the structures shown below:

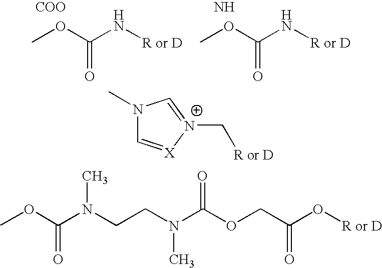

The fragmentable linker moieties illustrated in Structures II-IV comprising a benzyl backbone are merely exemplary linkers. Any molecule which is capable of fragmenting, and which comprises two or more “sites” suitable for attaching other molecule and moieties thereto, or that can be appropriately functionalized to attach other molecules and moieties thereto could be used to provide a divalent or higher order linker moiety. Although the “backbone” of the fragmentable linker moiety depicted in Structures II-IV is illustrated as an aryl compound comprising carbon and hydrogen atoms, the linker backbone need not be limited to carbon and hydrogen atoms. Thus, a linker backbone suitable for use in the compositions and methods described herein can include single, double, triple or aromatic carbon-carbon bonds, carbon-nitrogen bonds, nitrogen-nitrogen bonds, carbon-oxygen bonds, carbon-sulfur bonds and combinations thereof, and therefore can include substituents such as carbonyls, ethers, thioethers, carboxamides, sulfonamides, ureas, urethanes, hydrazines, etc. Moreover, the backbone of the linker moiety can comprise a mono or polycyclic aryl or an arylalkyl moiety.

In the exemplary substrate compounds of Structure II-IV, one or more optional “Y” substituents can be attached to R³, R⁴, R⁵, R⁶, and R⁷. The substituents may all be the same, or some or all of them may be different. Examples of suitable Y substituents groups include, but are not limited, —NO₂—, —CH₃—, —OCH₃—, —OR—, —Cl—, —F—, —NH₂—, —CO₂H—, and CH₂ CO₂NH₂—.

Any hydrophobic moiety described herein can be used to construct a dye-peptide signal molecule. The hydrophobic moiety is preferably chosen to facilitate an increase in fluorescence upon modification of the peptide moiety, such that the amplitude of the increase is greater than would be obtained with the same molecule lacking the hydrophobic moiety. Specific exemplary embodiments comprise any of the specific embodiments described previously.

Likewise, any fluorescent moiety described herein can be used to construct a dye-peptide signal molecule.

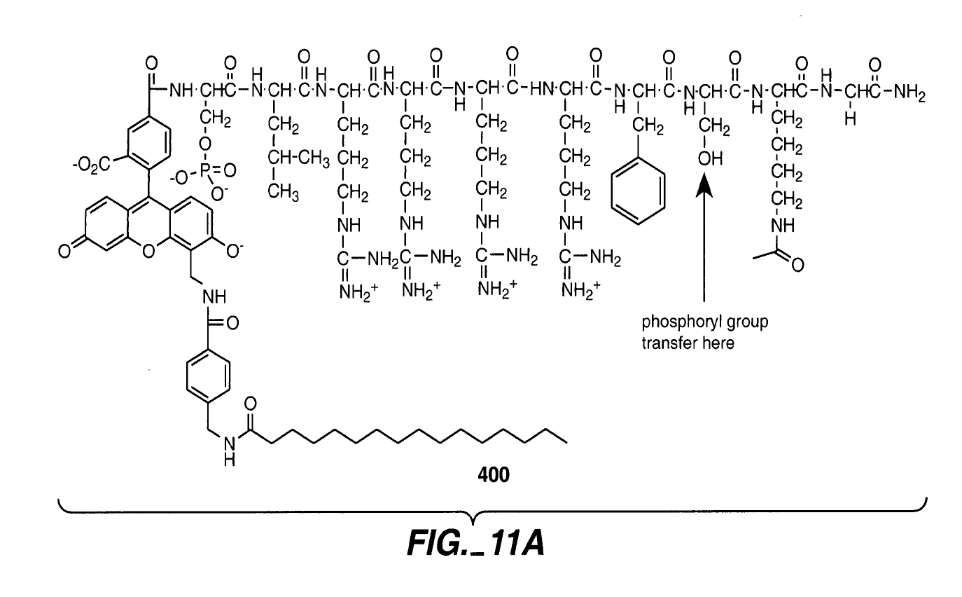

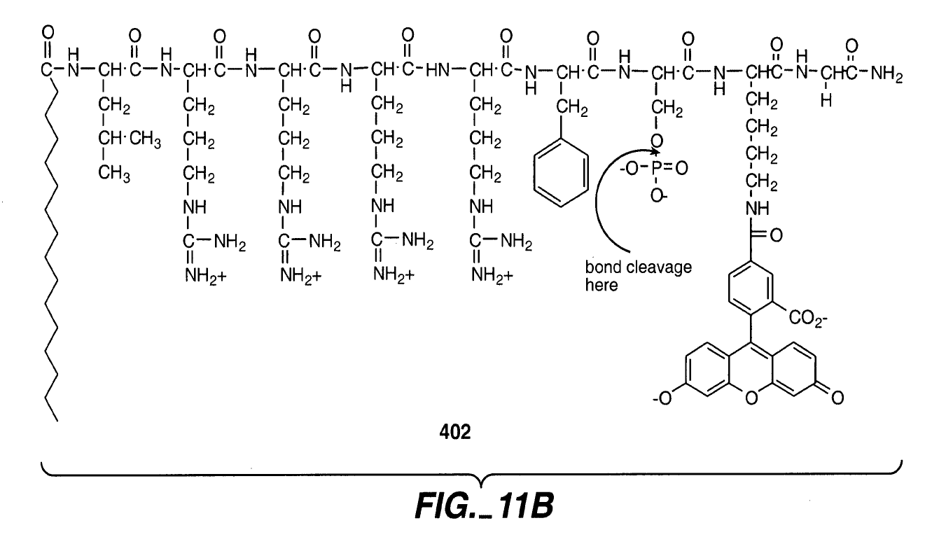

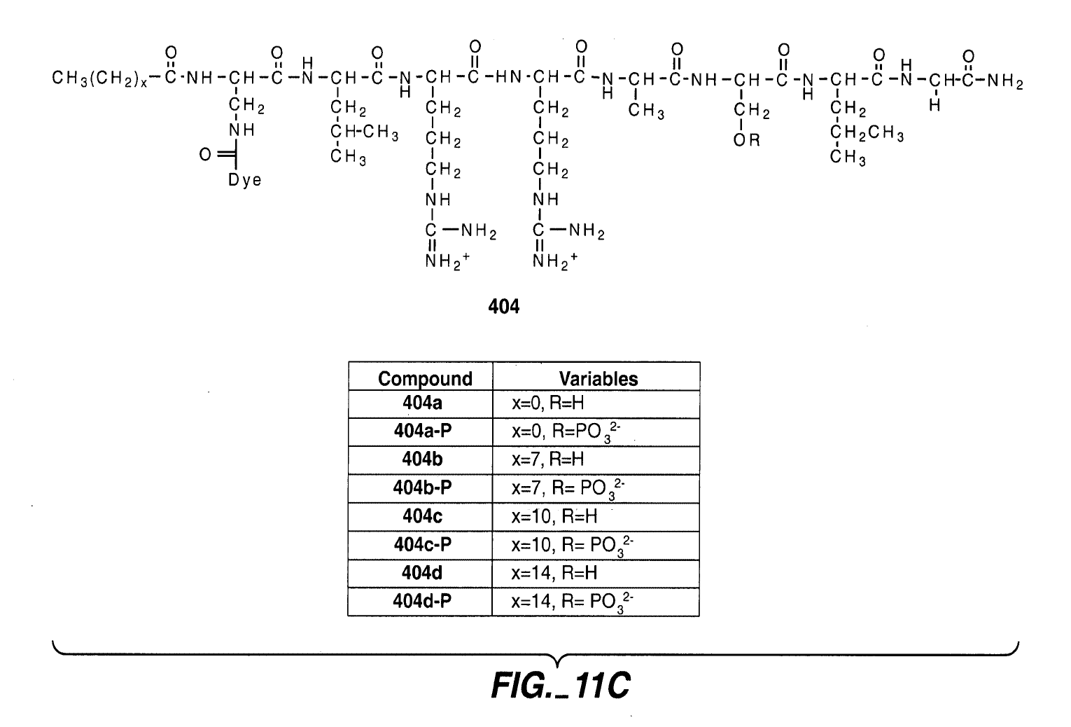

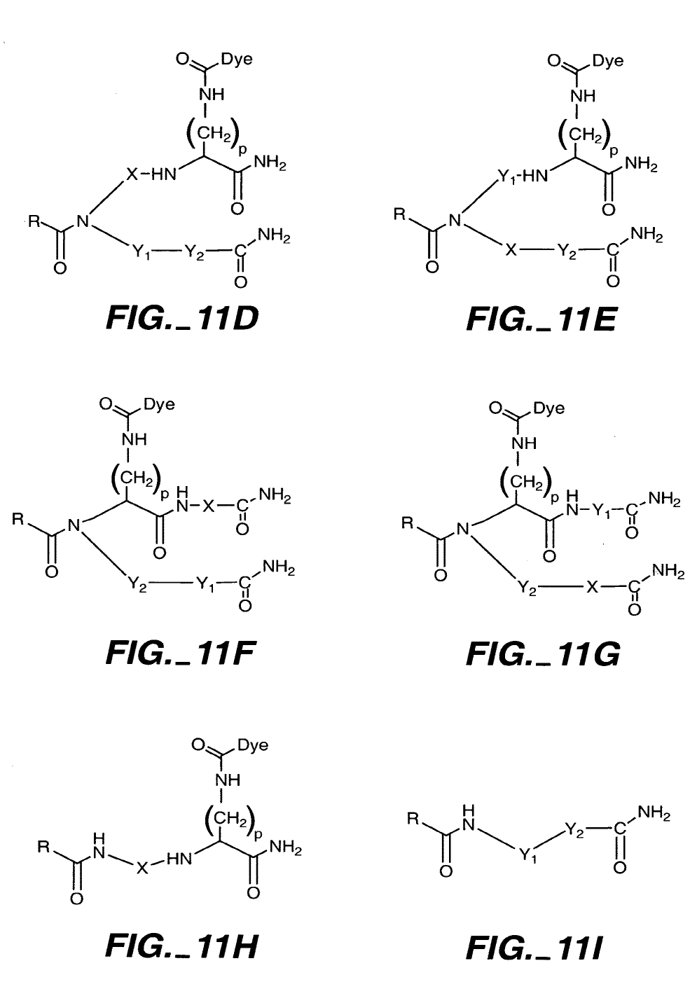

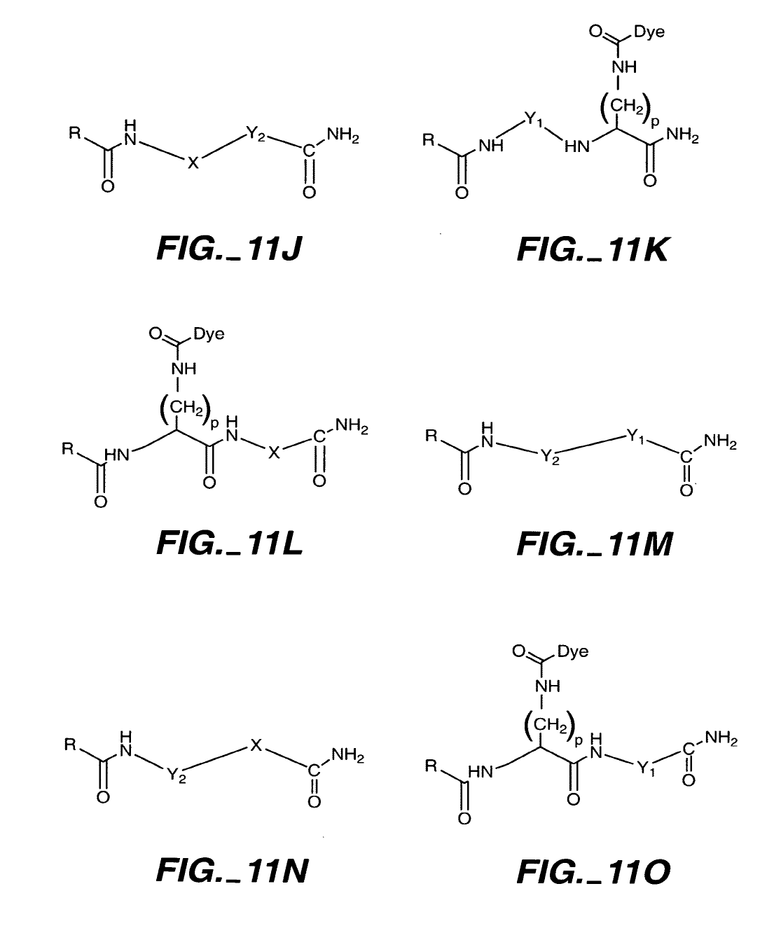

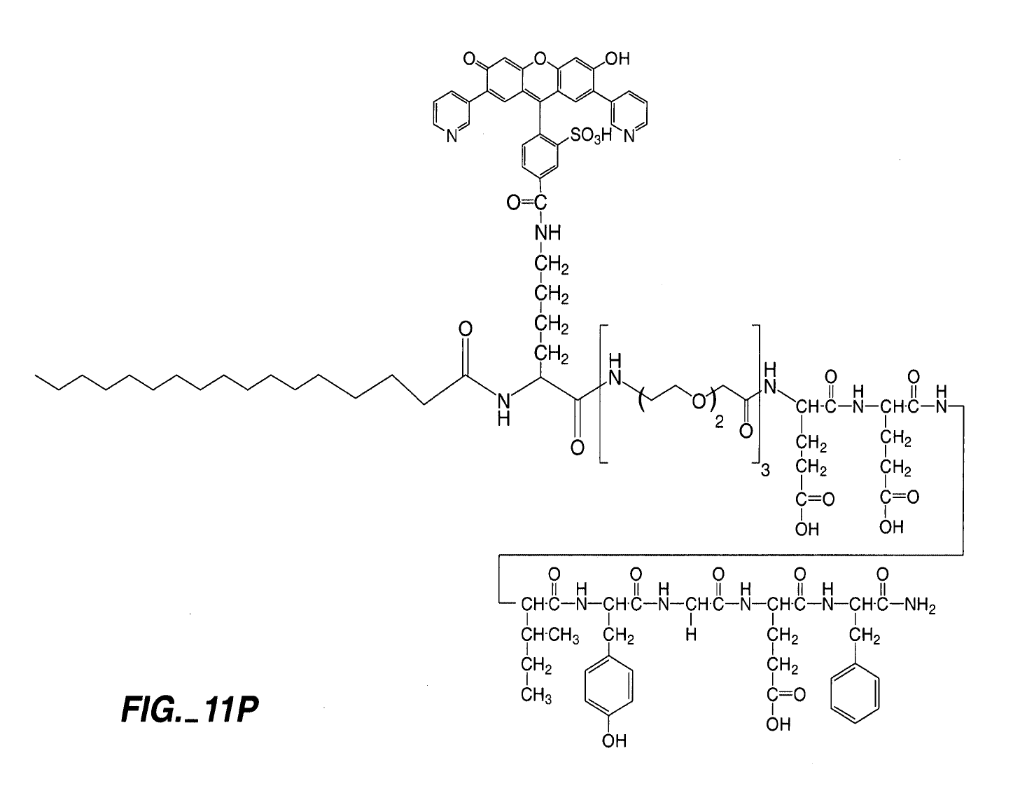

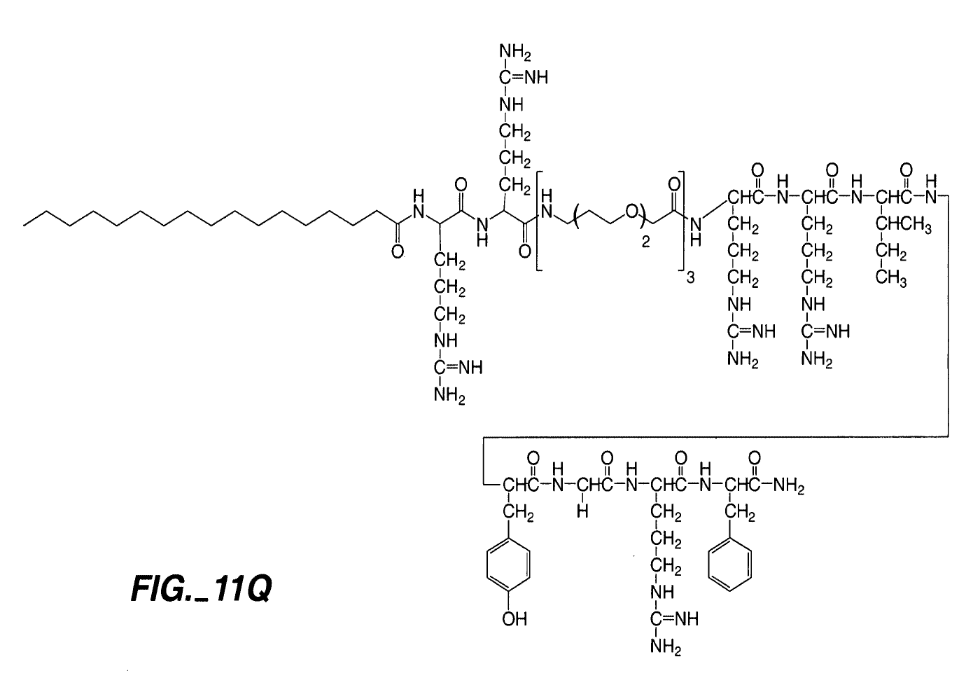

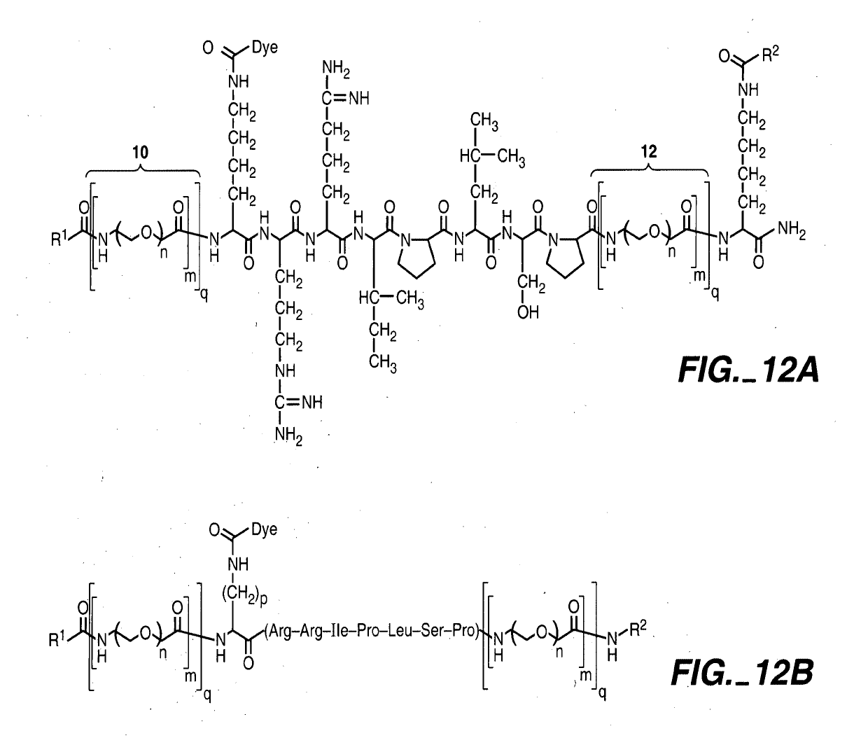

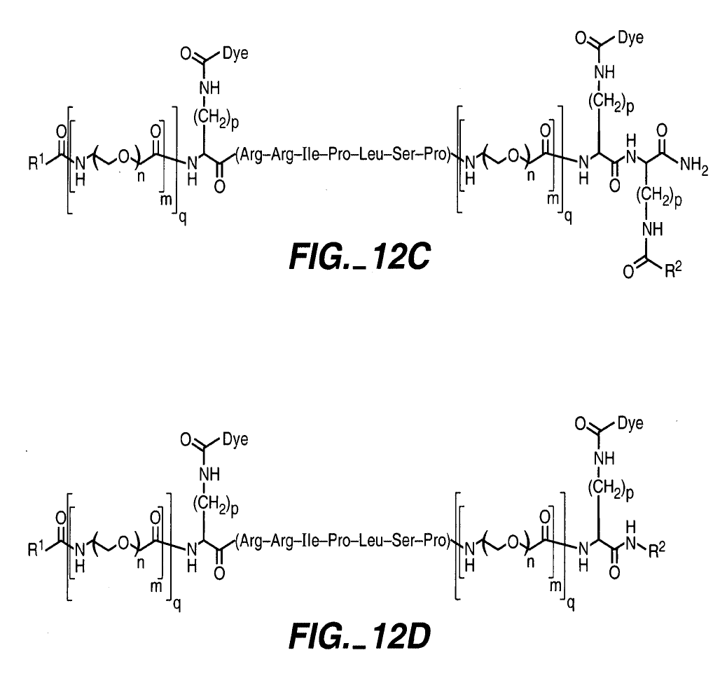

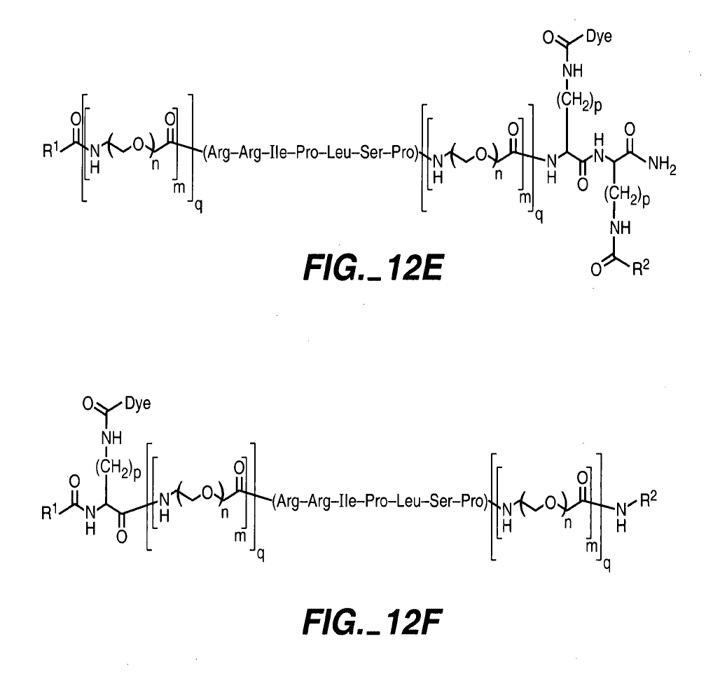

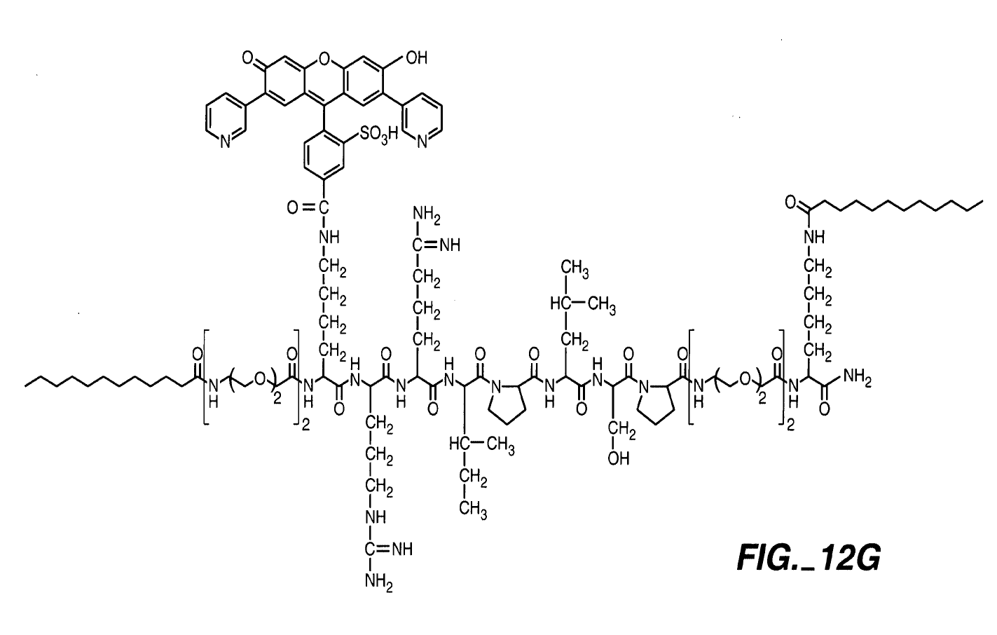

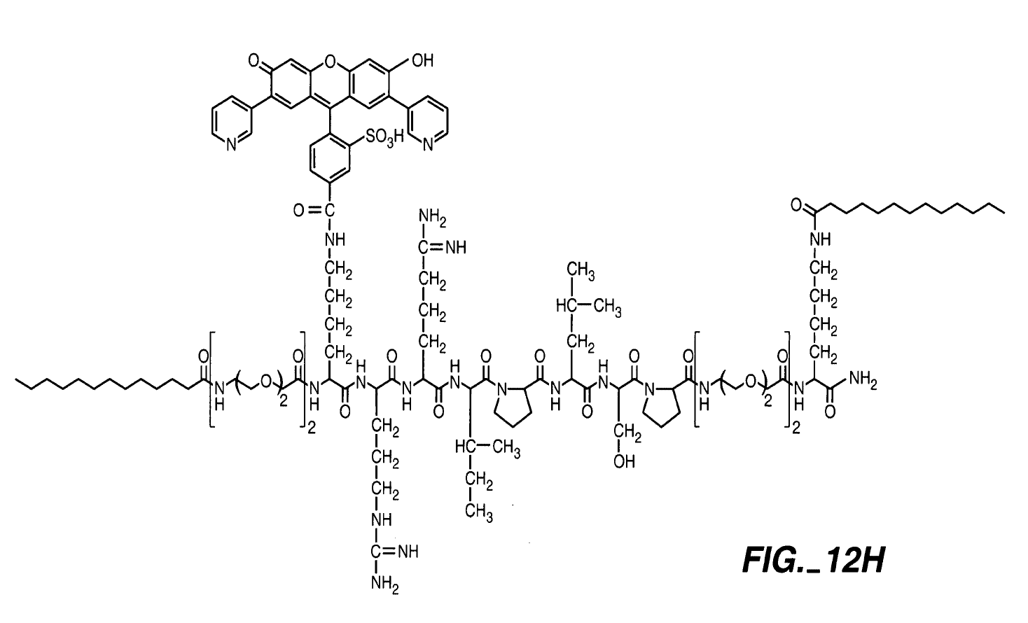

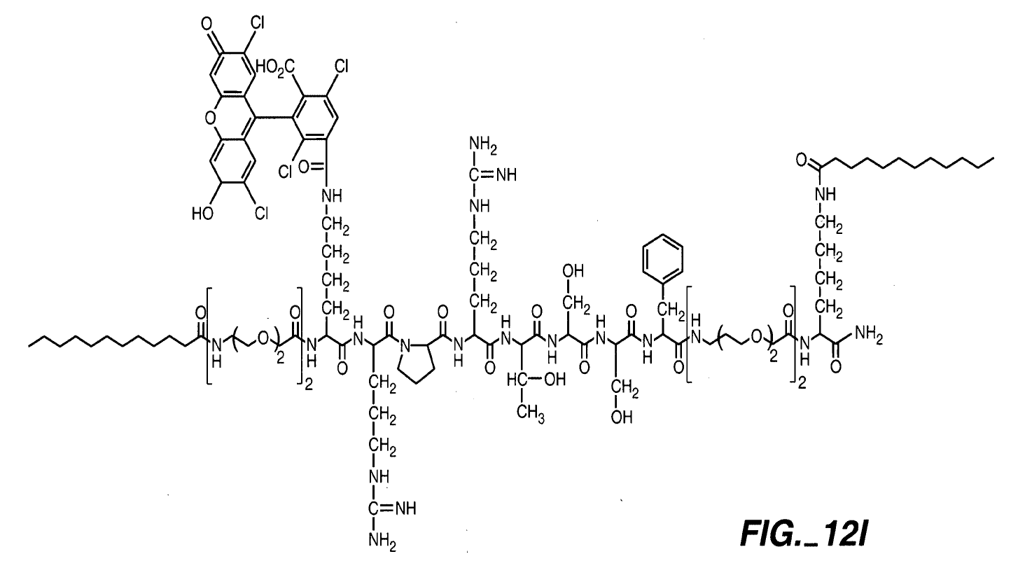

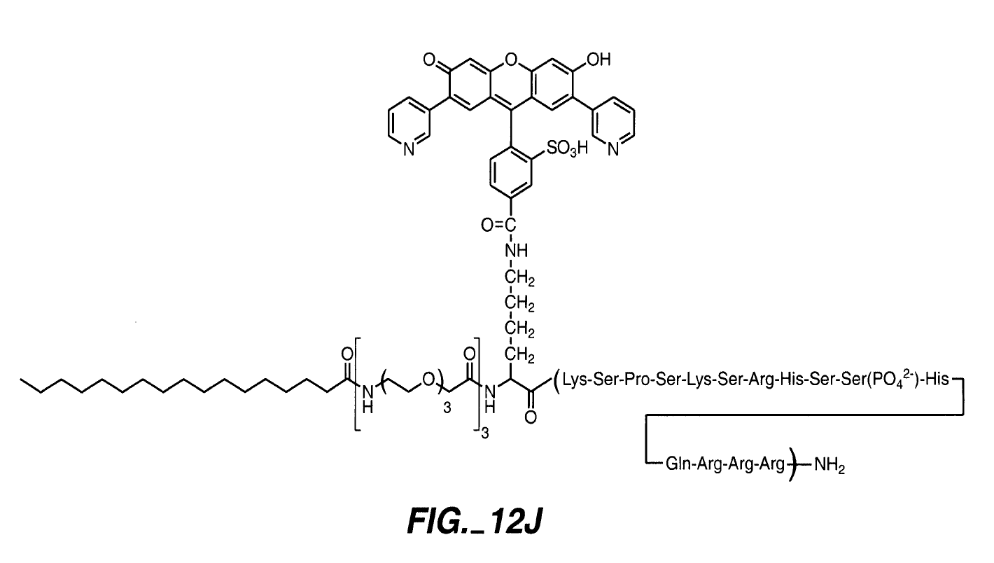

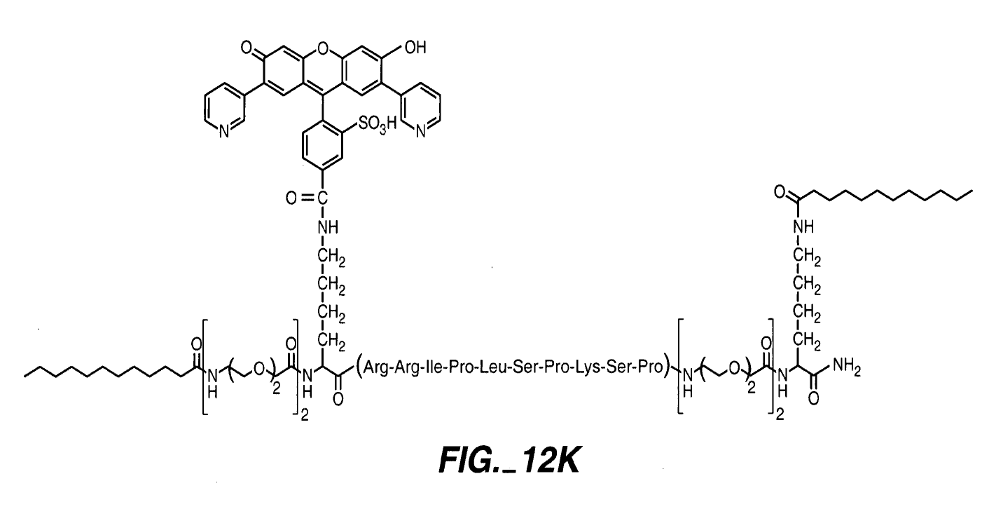

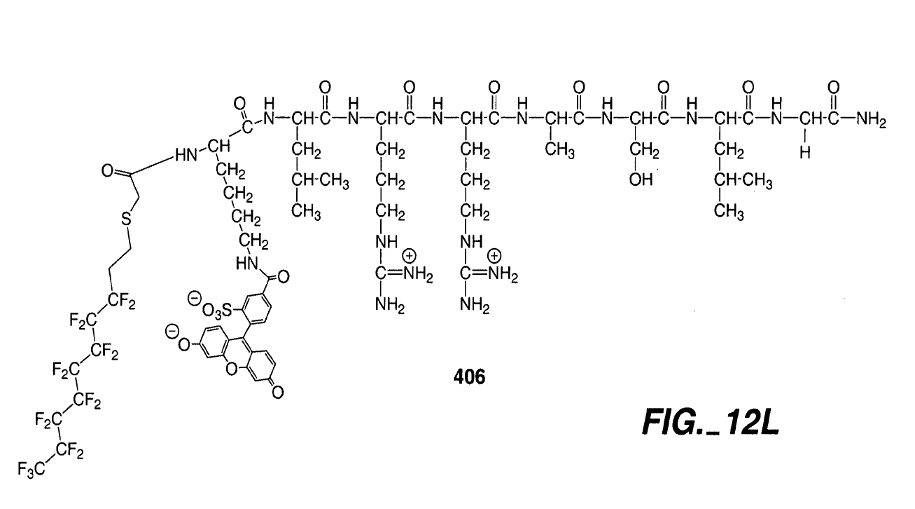

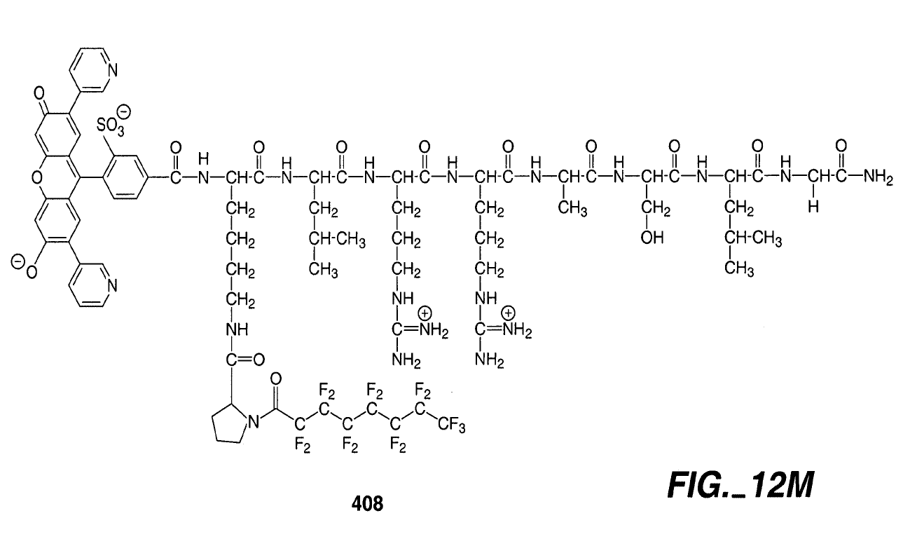

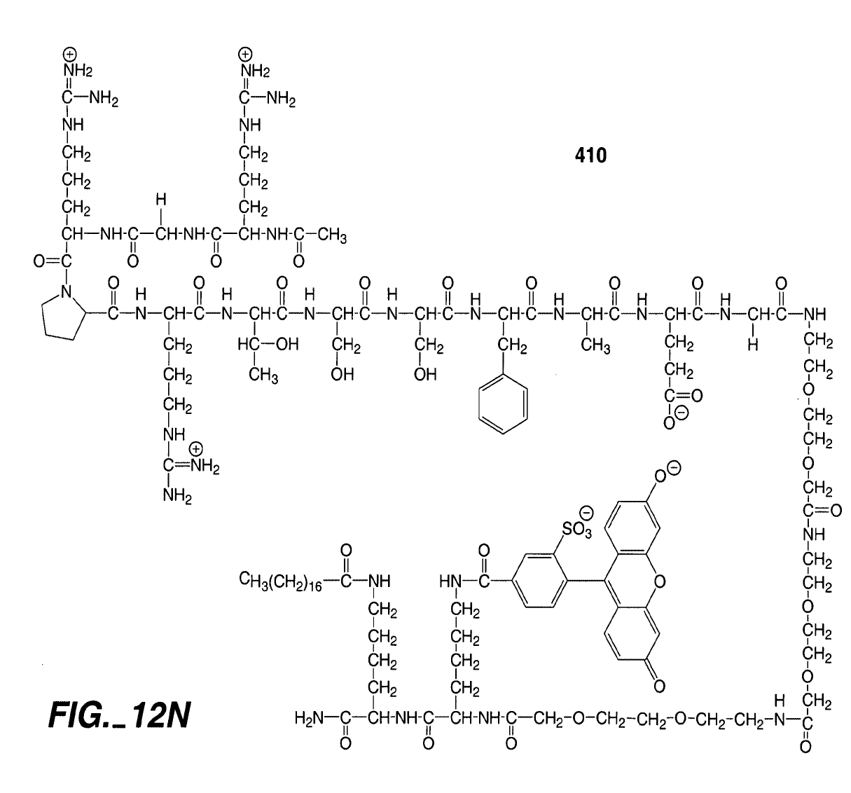

The peptide moiety, hydrophobic moiety, and fluorescent moiety can be connected or associated in any way that permits them to perform their respective functions. In some embodiments, the hydrophobic moiety and the peptide moiety are covalently linked to each other through the fluorescent moiety. In some embodiments, the hydrophobic moiety and the fluorescent moiety are covalently linked to each other through the peptide moiety. For example, the hydrophobic moiety and the fluorescent moiety can be covalently linked to opposite ends of the part of the dye-peptide signal molecule that contains the peptide moiety. In some embodiments, the hydrophobic moiety, the fluorescent moiety, and the peptide moiety are linked by a trivalent linker. Specific embodiments are illustrated in FIGS. 11A-12N.

In those embodiments in which the signal molecule comprises a trigger moiety, the hydrophobic moiety, fluorescent moiety, and trigger moiety are connected to the linker moiety in any way that permits them to perform their respective functions. In some embodiments, the hydrophobic moiety and the fluorescent moiety are each, independently of the other, directly connected to the linker moiety. In other embodiments, the hydrophobic moiety and the fluorescent moiety are each, independently of the other, indirectly connected to the linker moiety via one or more optional linkages. The optional linkages can comprise a leaving group, which upon fragmentation of the substrate compound is released from the backbone of the linker, along with the moiety that is attached to it. For example, in some embodiments, the fluorescent moiety can be attached to the backbone of the linker moiety via a linkage comprising a leaving group, while the hydrophobic moiety can be attached to the backbone of the linker moiety via a stable linkage, e.g., a linkage that does not dissociate from the backbone of the linker following the fragmentation reaction. Specific embodiments are illustrated in FIGS. 5A-5H.

Specific examples of exemplary dye-peptide signal molecules are illustrated in FIGS. 11A-12N.

Referring to FIG. 11A, the illustrated signal molecule 400 can be represented as X-L-Dye-Ser(OPO₃²⁻)LeuArgArgArgArgPheSerLys (ε-N—Ac)Gly(NH₂), wherein X is a C-16 fatty acid acyl group (palmitoyl), L is a linker (para-NHCH₂C₆H₄C(═O)NHCH₂) that links X to Dye, Dye is a fluorescent moiety (in this case, a fluorescein), e-N—Ac is an acetyl group, Ser, Leu, Arg, Phe, Ser, Lys, and Gly are standard 3-letter codes for serine, leucine, arginine, phenylalanine, lysine, and glycine, respectively, and NH₂ indicates that the carboxyl group of the C-terminal glycine is amidated.

The exemplary dye-peptide signal molecule 400 contains a phenolate anion and a carboxyl anion in the Dye moiety, and a phosphate group in the N-terminal serine residue which has two additional negative charges, for a total negative charge of −4. This is offset by the guanidinium groups in the four arginine residues, for a total of four positive charges. Thus, the net charge of the compound is about 0 at pH 8.

Molecule 400 further comprises a protein kinase recognition site in the form of a polypeptide containing an amino acid sequence that is recognized by protein kinase A. The recognition site contains an unphosphorylated serine that is capable of being phosphorylated by protein kinase A.

Referring to FIG. 11B, the illustrated dye-peptide signal molecule 402 can be represented as X-LeuArgArgArgArgPheSer(OPO₃²⁻)Lys(ε-N-Dye)Gly-NH₂, wherein X is a C-16 fatty acid acyl group (palmitoyl), Dye is a fluorescent moiety (fluorescein) that is linked to the epsilon amino group of a lysine residue, and NH₂ indicates that the carboxyl group of the C-terminal glycine is amidated. In signal molecule 402, the hydrophobic X moiety is linked directly by an amide bond to the N-terminal amino group of the polypeptide segment, without using additional linker atoms. However, it will be appreciated that a linker containing one or more linking chain atoms could also be included if desired. In addition, one or both of the hydrophobic moiety and the fluorescent moiety can be attached to internal residues within a polypeptide segment. Also, the hydrophobic moiety can be linked to a site in the phosphatase recognition site that is more N-terminal than the site where the fluorescent moiety is attached.

Prior to modification by a phosphatase, signal molecule 402 contains four positive charges that are provided by four arginine side chains, and four negative charges which are provided by two negative charges in the fluorescein Dye moiety (a phenolate anion and a carboxyl anion) and two additional negative charges in a phosphate group, for a total net charge of about 0 at pH 8. Upon hydrolysis of the phosphate group from the phosphorylated serine residue adjacent to the phenylalanine residues, the resulting modified signal molecule has a net positive charge of +2, due to loss of the two negative charges on the phosphate group. Accordingly, the modified signal molecule is expected to fluoresce more brightly than the unmodified form, due to its instability in the micelles.

One difference between exemplary signal molecules 400 and 402 is that the hydrophobic moiety and the fluorescent moiety in signal molecule 102 are located at opposite ends of a polypeptide scaffold, whereas the hydrophobic moiety and the fluorescent moiety in signal molecule 400 are relatively close together at the same end of a polypeptide scaffold. Both designs are suitable for use in the micelles and methods described herein.

FIG. 11C provides a group of dye-peptide signal molecules 404 which have different length alkyl acyl groups (X). In signal molecules 404, the hydrophobic moiety, the fluorescent moiety, and the enzyme recognition site are linked by a trivalent linker. The general structure of signal molecules 404 can be represented by X-Y(Dye)-LeuArgArgAlaSer(OR)LeuGly-NH₂, wherein X is a fatty acid acyl group of the form —CH₃(CH₂)ₙC(═O)—, with n as defined in Table 17, Y is alpha-aminomethylglycine, Dye is fluorescent moiety, such as a 4,7-dichlorofluorescein dye attached to the 2-amino group of Y by a 5-carbonyl linkage to the pendant phenyl ring of the dye, R is H or PO₃²⁻ (see Table 17), and NH₂ indicates that the carboxyl group of the C-terminal glycine is amidated. Table 17 illustrates some specific examples of signal molecules 404.

Each of signal molecules 404a, 404b, and 404c contains two positive charges from two arginine side chains, and two negative charges from the fluorescein Dye moiety (a phenolate anion and a carboxyl anion), for a total net charge of about 0 at pH 8. These molecules contain an unphosphorylated serine residue that is capable of being phosphorylated by the kinase. Upon phosphorylation, the net charge of these compounds is changed from neutral to −2.

Generally, a greater change in fluorescence provides greater assay sensitivity, provided that an adequately low signal-to-noise ratio is achieved. Therefore, it may be desirable to test multiple signal molecule variants to find a signal molecule having the most suitable fluorescence properties. Studies have been conducted on the signal molecules listed in Table 17. These signal molecules differ in the lengths of their hydrocarbon “tails” in the hydrophobic moiety (X), with chain lengths of 1, 8, 11 and 15 saturated carbon atoms. For each chain length, molecules were prepared in phosphorylated and unphosphorylated forms. For each assay, 5 μM of a'molecule selected from Table 17 was used. Fluorescence was measured in 100 mM TrisHCl buffer at pH 8.5, with excitation at 500 nm and emission at 546 nm. The results of these studies are shown in Table 17.

TABLE 17
Comparison of Fluorescence Between Phosphorylated and
Unphosphorylated Signal Molecules

| | | Fluorescence | Fluorescence | |
|---|---|---|---|---|
| | Hydrocarbon | (unphos- | (phospho- | Fluorescence |
| Molecule | Tail Length | phorylated)¹ | rylated)¹ | Ratio² |
| 404a, 404a-P | 1 | 1680 | 1930 | 1 |
| 404b, 404b-P | 8 | 575 | 1370 | 2 |
| 404c, 404c-P | 11 | 45 | 431 | 10 |
| 404d, 404d-P | 15 | 3 | 20 | 7 |

¹Fluorescence measurements in arbitrary units for unphosphorylated and phosphorylated forms of the respective molecules.
²Rounded value of Fluorescence (phosphorylated)/Fluorescence (unphosphorylated)

As can be seen, virtually no difference in fluorescence was observed between the unphosphorylated and the phosphorylated form. This suggests that an acetyl group may be too small to favor micelle formation for the unphosphorylated compound. However, significant differences in fluorescence were observed for the longer X groups. The dodecanoyl group (molecules 404c, 404c-P) appeared to provide the greatest increase upon phosphorylation (an increase of about 900%), but the tetradecanoyl group (molecules 404d, 404d-P) is also very effective, showing an increase of about 600%. The fluorescence observed for the nonanoyl group (molecules 404b, 404b-P) indicates that molecule 404b might also be useful, but is less preferred than the longer chain compounds. The results demonstrate that the presence of a hydrophobic moiety capable of integrating a compound into a micelle is effective to cause quenching of the fluorescence of the unphosphorylated compound. Without limiting the present teachings to any particular theory, the observed quenching may be due to predominance of the self-quenching micellar form, whereas the equilibrium between micellar and free forms of the phosphorylated molecules is shifted in favor of the free form, so that less signal from the phosphorylated molecule is self-quenched.

Table 17 also shows that the amplitude of the fluorescent signals of both forms of each of the molecules decreased with increasing length of the hydrophobic moiety. A possible explanation is that longer hydrophobic chains may cause an increasing proportion of the phosphorylated form to form micelles, so that some of the fluorescent signal of the phosphorylated form is suppressed due to self-quenching. However, if the equilibrium constant between free and micellar forms of the phosphorylated is greater than the corresponding equilibrium constant for the unphosphorylated form, then enzyme-catalyzed phosphorylation can generate an observable increase in fluorescence.

In some embodiments, the micellar form for the unphosphorylated dye-peptide molecule can be promoted or encouraged by including a charge balance moiety. The charge-balance moiety acts to balance the overall charge of the micelle. For example, if the dye-peptide molecule comprises one or more charged chemical groups, the presence of these groups can interfere with and/or destabilize micelle formation, thereby generating a detectable fluorescent signal in the absence of the specified enzyme. Micelle formation can be promoted or encouraged by including a charge-balance molecule designed to counter the charge of the dye-peptide molecule via the inclusion of chemical groups that have the opposite charge of the chemical groups comprising the dye-peptide molecule. Thus, by including the charge-balance moiety, micelles can be formed in the presence of destabilizing chemical groups.

The charge-balance moiety can be designed to balance the overall charge of the micelle such that net charge of the micelle is about neutral. The overall charge of the micelle depends in part on a number of factors including its chemical composition and pH of the solution comprising the micelle. For example in some embodiments, the substrate molecule comprises a florescent moiety and a substrate moiety, both of which comprise one or more charged chemical groups that can destabilize or prevent micelle formation. By including a charge-balance molecule that is capable of countering the charge of the substrate molecule, micelles with a net charge between ⁻1 to ⁺1 can be formed at a pH on the range of 6 to 8. Thus, the charge of the charge-balance molecule, depends in part, on the presence of the other charged groups comprising the micelle.

The charge-balance molecule can be designed to have a net negative or net positive charge by including an appropriate number of negatively and positively charged groups in the charge-balance moiety. For example, to establish a net positive charge (i.e., net charge ⁺2), the charge-balance moiety can be designed to contain positively charged groups, or a greater number of positively charged groups than negatively charged groups. To establish a net negative charge (i.e., net charge ⁻2), the charge-balance moiety can be designed to contain negatively charged groups, or a greater number of negatively charged groups than positively charged groups.

The overall charge of the charge-balance molecule also depends in part upon other factors such as the molar ratio of the substrate molecule:charge-balance molecule, the pH of the assay medium, and concentration of salt in the assay medium.

The ratio of charge-balance molecule to substrate molecule can be any ratio capable of balancing the overall charge of the micelle. In some embodiments, the molar ratio between the charge-balance molecule and substrate molecule is 0.5 to 1. In other embodiments, the molar ratio between the charge-balance molecule and substrate molecule is 1 to 1. In other embodiments the molar ratio between the charge-balance molecule and substrate molecule is 1 to 2, or 1 to 5, or 1 to 10. In some embodiments, the molar ratio between the substrate molecule and charge-balance molecule and is 0.5 to 1. In other embodiments, the molar ratio between the substrate molecule and charge-balance molecule is 1 to 1. In other embodiments the molar ratio between the substrate molecule and charge-balance molecule is 1 to 2, or 1 to 5, or 1 to 10.

As another specific example, if the net charge of the substrate molecule is ⁺2, the ⁺2 charge can be balanced by adding an equal molar ratio of a charge-balance molecule with a net charge of ⁻2. In other embodiments, if the net charge of the substrate molecule is ⁺2, the charge can be balanced by adding a charge-balance molecule with a net charge of ⁻1 at a 1:2 molar ratio of substrate molecule to charge-balance molecule.

Another factor effecting the charge of the charge-balance moiety is the pH of the assay medium and the pKas' of the groups comprising the charge-balance moiety. For example, in some embodiments, if the charge-balance moiety is designed to carry a positive charge at pH 7.6, then amino acids with side chains having pKas' above 7.6 can be chosen i.e. lysine (pKa 10.5) and arginine (pKa 12.5) carry a positive charge at pH 7.6. In some embodiments, if the charge-balance moiety is designed to carry a negative charge at pH 7.6, then amino acids with side chains having pKas' below 7.6 can be chosen i.e. aspartic acid (pKa 3.9) and glutamic acid (pKa 4.3) carry a negative charge at pH 7.6. The pKa values of the common amino acids at different pHs are shown in Table 18.

TABLE 18¹

| Amino Acid (IUPAC) | α-COOH pKa | α-NH₃⁺ pKa | Side chain pKa |
|---|---|---|---|
| Alanine (A) | 2.4 | 9.7 | |
| Cysteine (C) | 1.7 | 10.8 | 8.3 |
| Aspartic acid (D) | 2.1 | 9.8 | 3.9 |
| Glutamic acid (E) | 2.2 | 9.7 | 4.3 |
| Phenylalanine (F) | 1.8 | 9.1 | |
| Glycine (G) | 2.3 | 9.6 | |
| Histidine (H) | 1.8 | 9.2 | 6.0 |
| Isoleucine (I) | 2.4 | 9.7 | |
| Lysine (K) | 2.2 | 9.0 | 10.5 |
| Leucine (L) | 2.4 | 9.6 | |
| Methionine (M) | 2.3 | 9.2 | |
| Asparagine (N) | 2.0 | 8.8 | |
| Proline (P) | 2.1 | 10.6 | |
| Glutamine (Q) | 2.2 | 9.1 | |
| Arginine (R) | 2.2 | 9.0 | 12.5 |
| Serine (S) | 2.2 | 9.2 | ~13 |
| Threonine (T) | 2.6 | 10.4 | ~13 |
| Valine (V) | 2.3 | 9.6 | |
| Tryptophan (W) | 2.4 | 9.4 | |
| Tyrosine Y | 2.2 | 9.1 | 10.1 |

¹Garerett, R. H. and Grisham M. Biochemistry 2nd edition (1999) Saunders College Publishing. The pKa values depend on temperature, ionic strength, and the microenvironment of the ionizable group.

The charge-balance moiety comprises any group capable of carrying a charge. Suitable examples include amino acids, amino acid analogs, and derivatives, and quarternary compounds such as ammonium and amine compounds.

In some embodiments, the charge-balance moiety can comprise positively charged amino acids such as arginine and lysine. Lysine and arginine contain side chains that carry a single positive charge at physiological pH. The imidazole side chain of histidine has a pKa of about 6, so it carries a full positive charge at a pH of about 6 or less. The charge-balance moiety can comprise negatively charged amino acids such as aspartic acid and glutamic acid. Aspartic acid and glutamic acid contain carboxyl side chains having a single negative charge. Cysteine has a pKa of about 8, so it carries a full negative charge at a pH above 8. The charge-balance moiety can comprise a phosphorylated amino acid. For example, a phosphoserine residue carries two negative charges on a phosphate group.

In some embodiments, the charge-balance moiety can comprise uncharged amino acids such as alanine, asparagine, cysteine, glutamine, glycine, isoleucine, leucine, methionine, phenylalanine, proline, tryptophan, and valine (physiological pH 6 to 8).

In some embodiments, the charge-balance moiety can comprise uncharged amino acids analogs. Suitable examples include 2-amino-4-fluorobenzoic acid, 2-amino-3-methoxybenzoic acid, 3,4-diaminobenzoic acid, 4-aminomethyl-L-phenylalanine, 4-bromo-L-phenylalanine, 4-cyano-L-proline, 3,4,-dihydroxy-L-phenylalanine, ethyl-L-tyrosine, 7-azaatryptophan, 4-aminohippuric acid, 2 amino-3-guanidinopropionic acid, L-citrulline, and derivatives.

In some embodiments, the charge-balance moiety can comprise positively charged amino acids analogs such as N-ω,ω-dimethyl-L-arginine, a-methyl-DL-ornithine, N-ω-nitro-L-arginine, and derivatives.

In some embodiments, the charge-balance moiety can comprise negatively charged amino acids analogs such as 2-aminoadipic acid, N-a-(4-aminobenzoyl)-L-glutamic acid, iminodiacetic acid, a-methyl-L-aspartic acid, a-methyl-DL-glutamic acid, y-methylene-DL-glutamic acid, and derivatives.

In some embodiments, the charge balance moiety also comprises a modification moiety capable of being modified by a modification agent. For example, the modification agent can be a cleaving agent, such as a lipase, a phospholipase, a protease or a nuclease. The use of modification agents that do not cleave the signal and charge balance molecules may result in the formation of new aggregates or micelles comprising the modified signal and charge balance molecules, the fluorescence of which could remain quenched. In some embodiments, the modification moiety of the signal molecule and the modification moiety of the charge balance molecule are cleaved by different cleaving enzymes

In some embodiments, the charge balance molecule comprises a modification moiety and the signal molecule either does not comprise the optional modification moiety or comprises a modification moiety that is modified by a different modification agent than the modification moiety of the charge balance molecule.

FIGS. 11D-11G illustrate exemplary embodiments wherein the hydrophobic, fluorescent, substrate, and charge-balance moieties are included in a single molecule. In the exemplary embodiments depicted in FIGS. 11D-11G, hydrophobic moiety R is connected to the remainder of the substrate molecule via a peptide linkage. In some embodiments, the hydrophobic moiety R is linked to the remainder of the substrate molecule via an optional linker. R can comprise any of the hydrophobic moieties described above. In the exemplary embodiments depicted in FIGS. 11D-11G, the fluorescent moiety Dye is connected to the remainder of the substrate molecule via a ((CH₂)ₚ—NH—CO—) linkage, wherein p can be any integer form 1 to 6. FIG. 11D illustrates an exemplary embodiment wherein the charge of the substrate moiety X is balanced by an opposite charge on the charge-balance moiety Y₁. The charge of the fluorescent moiety Dye is balanced by an opposite charge on a second charge-balance moiety Y₂.

By way of illustration FIGS. 11H-11O illustrate exemplary embodiments of compositions comprising two distinct molecules, a substrate molecule (i.e. FIGS. 5H, J, L, N) and a charge-balance molecule (i.e. FIGS. 5I, K, M, O). In the exemplary embodiments depicted in FIGS. 11H-11O, hydrophobic moiety R can comprise any of the hydrophobic moieties described above. In the exemplary embodiments depicted in FIGS. 11H, K, L, and O the substrate molecule and charge-balance molecule comprise the fluorescent moiety Dye.

FIGS. 11P-11Q illustrate exemplary embodiments of a substrate molecule (FIG. 11P) and a charge-balance molecule (FIG. 11Q). FIG. 11P illustrates an exemplary substrate molecule that can be used to detect a protein kinase that recognizes a peptide consensus sequence for the tyrosine kinase Lyn, i.e. C₁₆Lys(Dye 2)OOOGluGluIleTyrGlyGluPheNH₂, wherein OOO represents the optional O-spacers, and Dye2 is 5-carboxy-2′,7′-dipyridyl-sulfonefluorescein. In the exemplary embodiment illustrated in FIG. 11P, hydrophobic moiety is a C₁₆ carbon chain and the fluorescent moiety, 5-carboxy-2′,7′-dipyridyl-sulfonefluorescein is linked to the hydrophobic moiety and an optional linker via the amino acid lysine. As will be appreciated by a person of skill in the art, the illustrated lysine is merely an exemplary linker. In FIG. 11P the substrate moiety comprises the peptide sequence Glu-Glu-Ile-Tyr-Gly-Glu-Phe.

FIG. 11Q illustrates an exemplary charge-balance molecule (i.e. C₁₆ArgArgOOOArgArgIleTyrGlyArgPheNH₂) that can be used balance the charge of the substrate molecule illustrated in FIG. 11Q. The substrate molecule illustrated in FIG. 11P comprises a fluorescent moiety containing a sulfonate anion with a charge of ⁻2. The substrate molecule illustrated in FIG. 11P further comprises a substrate moiety comprising three glutamate residues, each with a ⁻1 charge. Thus, the total negative charge of the substrate molecule illustrated in FIG. 11P is ⁻5 at physiological pH. The charge-balance molecule illustrated in FIG. 11Q comprises guanidinium groups in the five arginine residues, each having a ⁺1 charge. The total positive charge of the charge-balance molecule illustrated in FIG. 11Q is ⁺5 at pH 7.6. Thus, the net charge of the compound comprising the substrate molecule illustrated in FIG. 11P and the charge-balance molecule illustrated in FIG. 11Q is approximately zero at pH 7.6. Upon phosphorylation of the tyrosine residue by tyrosine kinase Lyn, the net charge of the micelle comprising the substrate molecule and charge-balance molecule is changed from approximately zero to ⁻2, thereby promoting the dissociation of the fluorescent moiety from the micelle, thereby reducing or eliminating the quenching effect and producing a detectable increase in fluorescence.

The various substrate and/or charge-balance molecules can comprise additional moieties. In some embodiments, a substrate molecule can comprise a charge-balance moiety and vice-versa. In some embodiments, the compositions can comprise a quenching moiety.

The sensitivity of assay can be increased by including two hydrophobic moieties in a dye-peptide signal molecule. For example, a comparison of the rates of reaction for a kinase substrate comprising two hydrophobic moieties versus a kinase substrate comprising a single hydrophobic moiety demonstrated that the kinase substrate with two hydrophobic moieties had a lower apparent Km of ATP than the kinase substrate with one hydrophobic moiety. In addition to exhibiting lower apparent Km′ of ATP, protein kinase substrates with two hydrophobic moieties also provided improved signal to noise ratios. See, e.g., Examples.

FIG. 12A illustrates an exemplary embodiment of a kinase substrate comprising two hydrophobic moieties, illustrated as R¹—C(O)— and R²—C(O)—, respectively, that are attached to opposite ends of the protein kinase recognition moiety. In the illustrated hydrophobic moieties, R¹ and R² can comprise any of the hydrophobic groups described above. For example, in some embodiments, R¹ and R² can comprise saturated or unsaturated alkyl chains, which may be the same or different.

In the exemplary embodiment illustrated in FIG. 12A, the first hydrophobic moiety R₁—C(O)— is linked to the remainder of the substrate via an optional linker 10. The presence or absence of optional linker 10 is denoted by the value for q, which may be 0 or 1. In the embodiment illustrated in FIG. 12A, optional linker 10 is provided by one or more (bis)ethylene glycol group(s), also referred to herein as an “O-spacer”. In the illustrated linker, the value of m can range broadly, but is typically an integer from 0 to 6. As used herein, each “O-spacer” corresponds to the bracketed illustrated structure. Thus, when m is an integer greater than one, such as, for example, three, the substrate is referred to herein as comprising three O-spacers (which can be abbreviated as “O—O—O”). As illustrated, the O-spacer comprises n oxyethylene units. As will be appreciated by a person skilled in the art, the number of oxyethylene units comprising an O-spacer can be selectively varied. For example, one, two, three or more oxyethylene units may be used to form an O-spacer. In some embodiments, n is an integer from 1 to 10. In other embodiments, n is 1, 2, 3, 4, 5 or 6.

Although exemplified with oxyethylene groups, an O-spacer need not be composed of oxyethylene units. Virtually any combination of the same or different oxyethylene units that permits the substrate to function as described herein may be used. In a specific example, an O-spacer may comprise from 1 to about 5 of the same or different lower oxyethylene units (e.g., —(CH₂)ₓCH₂)—, where x is an integer ranging from 0 to 6).

Although optional linker 10 of FIG. 12A is exemplified with an O-spacer, the chemical composition of optional linker 10 is not critical for success. The length and chemical composition of the linker can be selectively varied. In some embodiments, the linker can be selected to have specified properties. For example, the linker can be hydrophobic in character, hydrophilic in character, long or short, rigid, semirigid or flexible, depending upon the particular application. The linker can be optionally substituted with one or more substituents or one or more linking groups for the attachment of additional substituents, which may be the same or different, thereby providing a “polyvalent” linking moiety capable of conjugating or linking additional molecules or substances to the signal molecule. In certain embodiments, however, the linker does not comprise such additional substituents or linking groups.

A wide variety of linkers comprised of stable bonds that are suitable for use in the substrates described herein are known in the art and are discussed above.

In the exemplary kinase substrate of FIG. 12A, the linkage linking the first hydrophobic moiety to the illustrated linker 10 (as well as the linkages linking the other moieties and optional linkers to one another) is a peptide bond. Skilled artisans will appreciate that while using peptide bonds may be convenient, the various moieties comprising the substrates can be linked to one another via any linkage that is stable to the conditions under which the substrates will be used. In some embodiments, the linkages are formed from pairs of complementary reactive groups capable of forming covalent linkages with one another. “Complementary” nucleophilic and electrophilic groups (or precursors thereof that can be suitable activated) useful for effecting linkages stable to biological and other assay conditions are well known. Examples of suitable complementary nucleophilic and electrophilic groups, as well as the resultant linkages formed therefrom, are provided in Table 3, discussed above.

In the exemplary embodiment illustrated in FIG. 12A, the fluorescent moiety (Dye-C(O)— is linked to the first hydrophobic moiety and the N-terminal end of the protein recognition moiety via a multivalent (trivalent) linker, which in the specific embodiment illustrated in FIG. 12A is provided by the amino acid lysine. As will be appreciated by a person of skill in the art, the illustrated lysine is merely an exemplary trivalent linker. Any molecule having three or more “reactive” groups suitable for attaching other molecule and moieties thereto, or that can be appropriately activated to attach other molecules and moieties thereto could be used to provide a trivalent or higher order multivalent linker. Additional examples of multivalent linkers are discussed below.

The second hydrophobic moiety, represented by R²—C(O)—, is linked the C-terminal end of the protein kinase recognition moiety. As illustrated, the linkage, which is effected through the use of a multivalent lysine residue, is spaced away from the C-terminus of the protein recognition sequence via optional linker 12. Optional linker 12 is similar in concept and function to optional linker 10. Although it is illustrated as being composed of an O-spacer, like optional linker 10, it need not be. Optional linker 12 can comprise any of the various atoms and groups discussed above in connection with optional linker 10. When as illustrated in FIG. 12A, both optional linkers are present (each q=1) and composed of O-spacers. The number of O-spacers comprising each linker can be selectively varied resulting in O-linkers of different lengths.

Optional linkers 10 and 12 may both be present, they may both be absent, or, alternatively, one of linkers 10 and 12 may be present and the other absent. For example, an optional linker 10 can be used to connect the first hydrophobic moiety to the N-terminal end of the protein kinase recognition moiety, while the second hydrophobic moiety can be linked to the C-terminal end of the protein kinase recognition moiety with the aid of optional linker 12.

Although the various hydrophobic, fluorescent, protein kinase recognition and optional linker moieties comprising the exemplary kinase substrate of FIG. 12A are linked in a specified configuration, other configurations are possible. Additional exemplary embodiments of kinase substrates are illustrated in FIGS. 12B-F. In FIGS. 12B-12F, each illustrated R¹, R², Dye, n, m and q is, independently of any others that may be illustrated, as defined for FIG. 12A. Each illustrated p is, independently of the others, an integer ranging from about 1 to about 6. Exemplary kinase substrates are illustrated in FIGS. 12G-12I.

In some embodiments, the substrate compounds described in FIGS. 12A-I, and variation thereof, are not cleavable by phospholipases.

Greater assay sensitivity can also be obtained by providing dye-peptide signal molecules with two or more recognition sequences in combination with one or two hydrophobic moieties. For example, an improved signal to background ratio was observed for a kinase substrate comprising two protein kinase recognition sequences and two hydrophobic moieties versus a kinase substrate comprising a single recognition sequence and two hydrophobic moieties (see, e.g., Examples). An improved signal to background ratio was also observed for a kinase substrate comprising two recognition sequences and a single hydrophobic moiety (see, e.g., Examples).

Exemplary kinase substrates comprising two protein kinase recognition sequences are illustrated in FIGS. 12J-12K. FIG. 12 J illustrates an exemplary kinase substrate, C₁₆—OOOK(Dye2)LSPSLSRHSS(PO₄²⁻)HQRRR—NH₂, comprising two protein kinase recognition sequences, i.e., SRHSS(PO₄²⁻) and SPSLS for GSK. FIG. 12K illustrates an exemplary kinase substrate, C₁₁—OOK(dye2)RRIPLSPLSPOOKC₁₁—NH₂, comprising two protein kinase recognition sequences, i.e., -PLSP- and -PLSP- for p38βII.

Skilled artisans will appreciate that while the kinase substrates illustrated in FIGS. 12J-12-K are exemplified with different combinations of hydrophobic moieties, fluorescent moieties, protein kinase recognition sequences, phosphorylatable moieties, and optional linkers, any one or more of these features of the illustrated kinase substrates could be varied. As a specific example, while the substrates are exemplified with optional O-spacers (described above), in embodiments employing one or more linkers, any linker could be used, as described above. Moreover, while the various moieties are illustrated as being linked with amide linkages, virtually any type of chemical linkage(s) that are stable to the assay conditions and that permit the various moieties to perform their respective functions could be used. Additionally, the various illustrated features can be readily “mixed and matched” to provide other specific embodiments of exemplary kinase substrates.

Additional embodiments of exemplary dye-peptide signal molecules 406 and 408 that can be modified by a protein kinase A are illustrated in FIGS. 12L and 12M. These exemplary signal molecules comprise hydrophobic moieties comprising substituted (perfluorinated) hydrocarbons. Another exemplary embodiment of a peptide-dye signal molecule 410 modifiable by a protein kinase A is illustrated in FIG. 12N. Signal molecule 410 can be represented as N—Ac-ArgGlyArgProArgThrSerSerPheAlaGluGly-OOOLys(ε-N-Dye)Lys(ε-N—X)—NH₂, wherein X is an octadecanoyl group that is linked to the epsilon amino group of a lysine residue, Dye is a fluorescent moiety (5-carboxy-sulfofluorescein) that is linked to the epsilon amino group of a lysine residue, O is a linker provided from a 2-aminoethoxy-2-ethoxyacetyl group (“O-Linker”), and NH₂ indicates that the carboxyl group of the C-terminal glycine is amidated. In signal molecule 410, the hydrophobic X group is linked to the epsilon amino group of a lysine residue without any further linker atoms. However, it will be appreciated that a linker containing one or more linking chain atoms could also be included if desired. Furthermore, the fluorescent dye is linked directly by an amide bond to the epsilon amino group of a lysine residue, without using additional linker atoms. However, it will be appreciated that a linker containing one or more linking chain atoms could also be included if desired.

Additional specific examples of exemplary peptide-dye signal molecules including linkers are provided in Table 19, below:

TABLE 19

| | | RFUs at 10 uL | Conc | Fold |
|---|---|---|---|---|
| Kinase | Peptide | (initial→final) | (uM) | increase |
| PKA | C13-K(dye2)-LRRASLG-NH₂ | 1000→5000 | 8 | 5x |
| PKA | C13-OOOK(dye2)-LRRASLG-NH₂ | 1000→5000 | 8 | 5x |
| PKC | C16-OOOK(dye2)-RREGSFR-NH₂ | 650→3000 | 4 | 4.5x |
| PKC | C17-OOOK(tet)-RQGSFRA-NH₂ | 700→4900 | 6 | 7x |
| Src, lyn, fyn | C16-OOOK(dye2)RIGEGTYGVVRR-NH₂ | 1000→6500 | 8 | 6.5x |
| Akt | C15-OOOK(dye2)RPRTSSF-NH₂ | 1500→7500 | 8 | 4x |
| MAPK | C17-OOOK(dye2)PRTPGGR-NH₂ | 1100→5700 | 16 | 5x |
| MAPKAP2 | C16-OOOK(dye2)RLNRTLSV-NH₂ | 800→3200 | 8 | 4x |

In Table 19, each “O” represents a linker provided by a 2-aminoethoxy-2-ethoxyacetyl group; “dye 2” is a fluorescent moiety provided by 5-carboxy-2′,7′-dipyridyl-sulfonefluorescein; “tet” is a fluorescent moiety provided by 2′,7′,4,7-tetrachloro-5-carboxy fluorescein (2′,7′-dichloro-5-carboxy-4,7-dichlorofluorescein); and NH₂ indicates that the carboxy group of the C-terminal amino acid residue is amidated.

Table 19 also provides the specific protein kinase that can be used to modify each of these signal molecules, as well as the fluorescence observed with a micelle comprising the signal molecules upon treatment with the specified protein kinase.

In the specific embodiments described above for which protein kinase recognition sequences are provided, it will appreciated that these sequences are for purposes of illustration only, and that virtually any protein kinase sequence, such as the various exemplary sequences provided in Table 17, supra, may be used. Skilled artisans will be readily able to select a protein kinase recognition sequence suitable for a particular application.

Dye-peptide signal molecules can be readily formed by routine synthetic methods known in the art. Exemplary methods suitable for synthesizing dye-peptide signal molecules are taught in the Examples.

6.2.4 The Ligand Molecule

In addition to the signal molecule, optional charge balance molecules, optional quenching molecules, and other components (discussed in more detail, below), the micelle also comprises a ligand molecule. The ligand molecule comprises a binding moiety (or putative binding moiety) and one or more hydrophobic moieties that integrate the ligand molecule into the micelle. When integrated into the micelle, the binding moiety is positioned such that it is available to, or capable of, binding another molecule, such as a receptor, which in some embodiments is immobilized on a substrate.

The binding moiety of the ligand may comprise any type of molecule of interest. For instance, the binding moiety may comprise a small organic molecule, a drug, a hapten, a vitamin, a toxin, a hormone, an enzyme, a substrate, a transition state analog, a protein, a transporter, a receptor, a G-protein coupled receptor, a receptor ligand, a cytokine, a growth factor, an antigen, an antibody, a biotin, a streptavidin, an aptamer, an amino acid, a peptide, a protein, a mono- or polysaccharide, a mono- or polynucleotide, a single or double stranded DNA, an mRNA, a cDNA, a gene, a virus, a microbe, a cell, or any other conjugatable entity, or any derivative or fragment thereof.

As will be appreciated by skilled artisans, while the binding molecule may comprise an enzyme or a substrate for an enzyme, it is desirable that the binding interaction between the binding moiety and its binding partner or putative binding partner be more than transient. Thus, in most embodiments, the binding moiety and binding partner pair will not be an enzyme-substrate pair where the enzyme only transiently binds the substrate and releases it after modifying it. However, it will be understood that enzyme-substrate pairs which require a cofactor for activity and that bind in the absence of the cofactor can be used as binding moieties and binding partners as described herein by carrying out a binding assay in the absence of the cofactor, or at a cofactor concentration less than that required for enzymatic activity. Moreover, enzymes and/or substrates may be used in the presence of such cofactors in a variety of contexts where the enzyme-substrate activity does not interfere with the assay, such as, for example, in the identification of enzyme inhibitors.

As evidenced from the above non-limiting list of exemplary binding moieties, while the molecule is referred to a “ligand molecule,” this nomenclature is for convenience only and is not intended to be limiting. Specifically, “ligand molecules” are not limited to classical ligands. Indeed, even classical receptors may comprise “ligand molecules” as that expression is used herein. The expression “ligand” is merely used for convenience to identify one member of a pair of binding molecules or putative pair of binding molecules.

The ligand molecule can be formed in situ by contacting a binding moiety which comprises a suitable conjugating moiety with a pre-formed micelle that comprises a “complementary” conjugating moiety. The conjugating moiety can be any moiety capable of conjugating or linking the binding moiety to the micelle. In some embodiments, the conjugating moiety is one member of a pair of specific binding molecules, such as, for example, biotin/avidin (or streptavidin), and the complementary conjugating moiety is the other member of the pair. In another embodiment, the conjugating moiety and complementary conjugating moieties comprise groups capable of forming covalent linkages with each other, such as, for example the R^{x} and F^{x} groups described above.

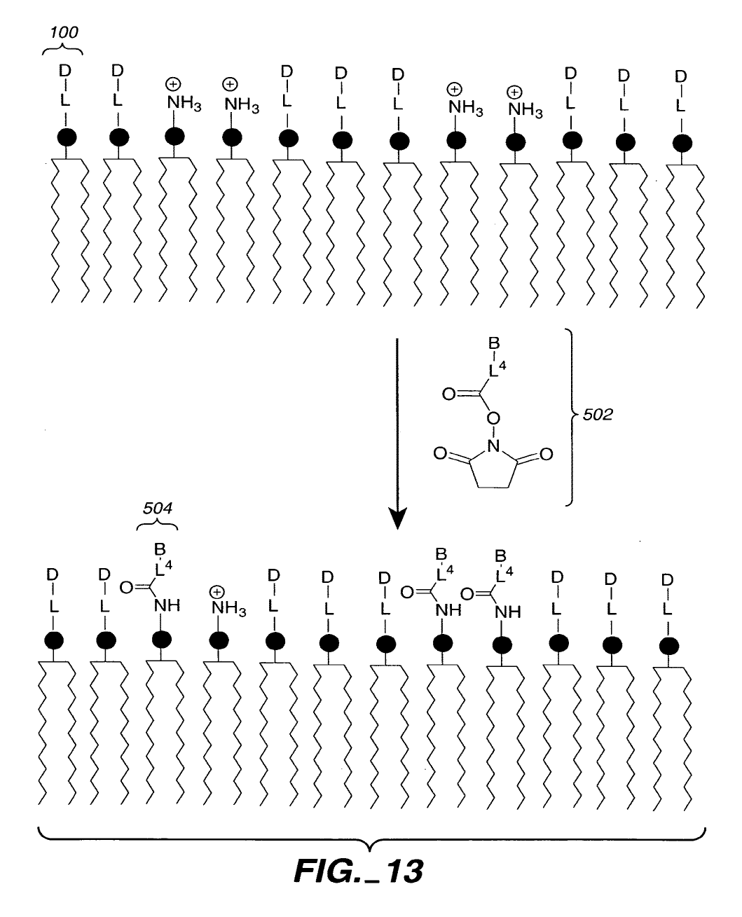

An exemplary embodiment of the formation of a ligand molecule in situ is illustrated in FIG. 13. In FIG. 13, a portion of an exemplary liposome micelle comprising phospholipids is illustrated. Each phospholipid is represented as two zigzagged lines connected to a circle. The zigzagged lines represent the hydrophobic tails of the phospholipid; the circle the polar head group (or a portion thereof). For some of the phospholipids, a group of the polar head group (in this case NH₃⁺) is illustrated. The micelle comprises glycerophospholipid signal molecules (represented by 100). In signal molecule 100, “D” represents the fluorescent moiety and “L” represents an optional linker, as previously discussed in connection with FIG. 1A.

Molecule 502, which comprises binding moiety “B” having an NHS-ester functional group linked thereto via optional linker L⁴, is contacted with the micelle. Optional linker L⁴ is similar in concept and composition to linker “L,” described above in connection with FIG. 1A. Following contact, binding moiety “B” is conjugated to the micelle via an amide linkage (ligand molecule 504). Depending upon the structures of the phospholipids comprising the micelle and/or compound 502, other linkages could be formed.

In another embodiment, the micelle is formed with pre-formed ligand molecules that comprise one or more hydrophobic moieties. Formation of micelles with preformed ligand molecules permits the molar ratio of the binding moiety in the micelle to be precisely controlled. In some embodiments, the ligand molecule naturally or endogenously comprises both the binding moiety and hydrophobic moiety(ies). For example, the hydrophobic moiety(ies) can comprise the transmembrane domain(s) of an integral membrane protein. In a specific embodiment, the ligand molecule is an integral membrane protein involved in a signal transduction cascade. For example, the ligand molecule can be a receptor for hormones, growth factors, neurotransmitters, viral proteins or other signaling molecules. In another specific embodiment, the ligand molecule can be a component of a G-protein coupled signal transduction cascade.

In another embodiment, the binding moiety is conjugated to one or more exogenous hydrophobic moieties. For example, the binding moiety can comprise a molecule that either comprises, or can be modified to comprise, a group or moiety that can be coupled to one or more hydrophobic moieties. As a specific example, the ligand molecule comprises a binding moiety, such as a protein, a drug or other molecule, linked to a fatty acid optionally by way of a linker. The optional linker can comprise virtually any combination of atoms or groups, as discussed previously in connection with the linker “L” of FIG. 1A. In some embodiments, it may be desirable to utilize a linker that is hydrophilic in character and that is long enough to permit the binding moiety to interact with and bind other molecules. Non-limiting examples of suitable hydrophilic linkers comprise, but are not limited to, linkers comprising peptides, polyalkylene glycols, such as the “O” linkers described above.

The ligand molecule can optionally comprise a modification site that can be modified by the same modification agent used to modify the modification moiety of the signal molecule, or by a different modification agent. Use of a modification moiety modifiable by a different modification agent than that used to modify the signal molecule permits selective release of the binding moiety from the micelle.

In some embodiments, the ligand molecule can correspond in structure to any of the previously-described dye-peptide signal compounds, such as the dye-peptide signal molecules of FIGS. 11-12, with the exception that the fluorescent moiety is replaced with a binding moiety.

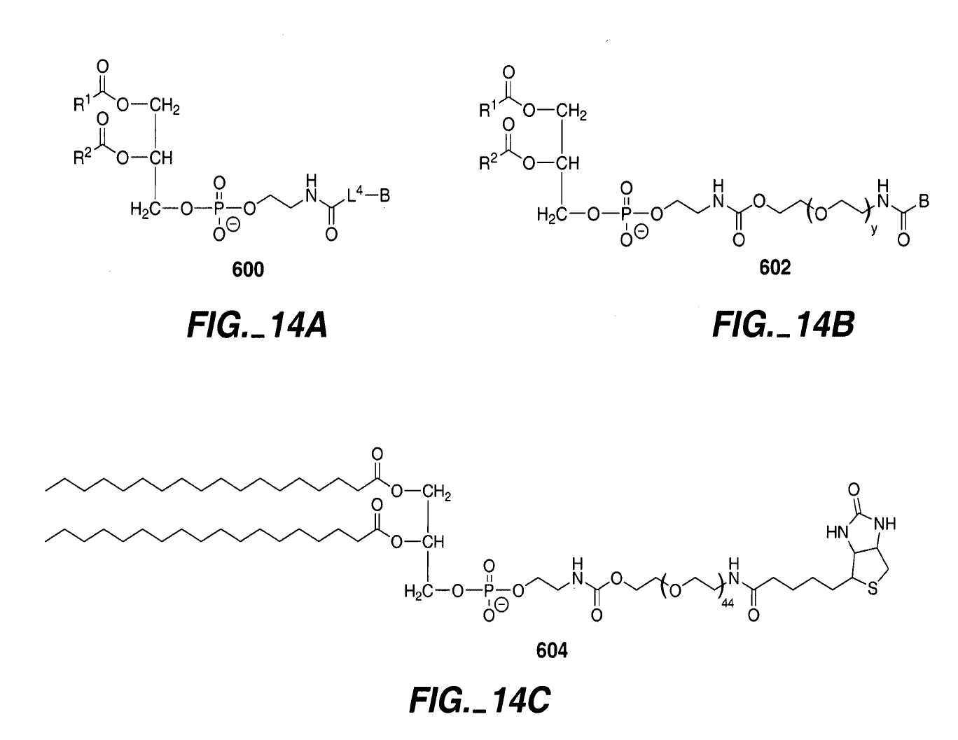

In some embodiments, the ligand molecule is an analog or a derivative of a phospholipid in which the binding moiety is attached to the phosphate moiety or the polar head group, optionally, by way of a linker. In some embodiments, the ligand molecule is an analog of the glycerophospholipid signal molecule of FIG. 1A in which the fluorescent moiety is replaced with a binding moiety. An embodiment of a ligand molecule 600 of this type is illustrated in FIG. 14A. In FIG. 14A, R¹ and R² are as defined for FIG. 1A, L⁴ is an optional linker and B represents a binding moiety. The optional linker L⁴ is similar in concept and composition to linker “L” of FIG. 1A.

A specific embodiment of ligand molecule 600 which comprises a linker L⁴ comprising polyethylene glycol groups is illustrated in FIG. 14B. In FIG. 14B, R¹ and R² and B are as defined for FIG. 14A and y is an integer that can range from 0 to one hundred, or even more, depending upon the length of the polyethylene glycol desired. Typically, y is an integer from 1 to 50. A specific embodiment of the ligand molecule 602 of FIG. 14B in which R¹ and R² are each a C17 n-alkanyl, binding moiety B comprises a biotin and y is 44 is illustrated in FIG. 14C.

Although the glycerophospholipid ligand molecules of FIGS. 14A, 14B and 14C are derivatives of phosphatidyl ethanolamine, derivatives of other glycerophospholipids, such as derivatives of phosphatidylcholine, phosphatidyl serine and phosphatidyl inositol, as well as derivatives of other lipids and/or phospholipids, such as derivatives of sphingolipids, lysophospholipids, tri-, di- or monoacylglycerols could also be used.

The phospholipid ligand molecules of FIGS. 14A, 14B and 14C comprise modification moieties that can be cleaved by phospholipases A1, A2, C and D. The linkages comprising the various different cleavage sites can be selected to yield phospholipid ligand compounds that can be cleaved by a specific phospholipase, or not cleaved by a particular phospholipase or phospholipases, as discussed above in connection with phospholipid signal molecules. In certain embodiments, the phospholipid ligand molecules and phospholipid signal molecules comprising the micelle can be designed to be cleaved by different phospholipases, permitting selective release of the fluorescent and binding moieties, as desired.

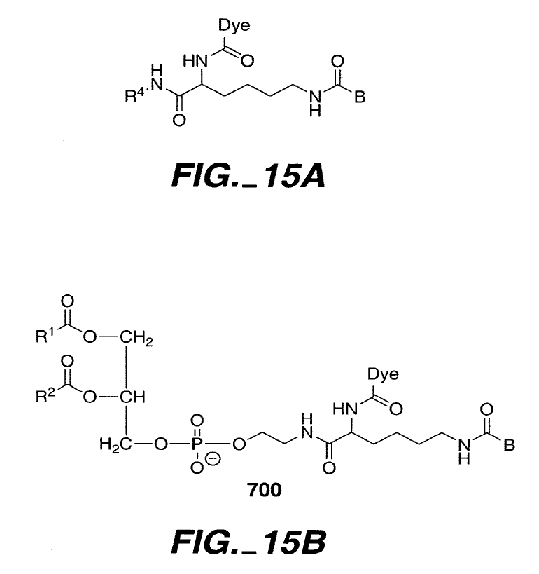

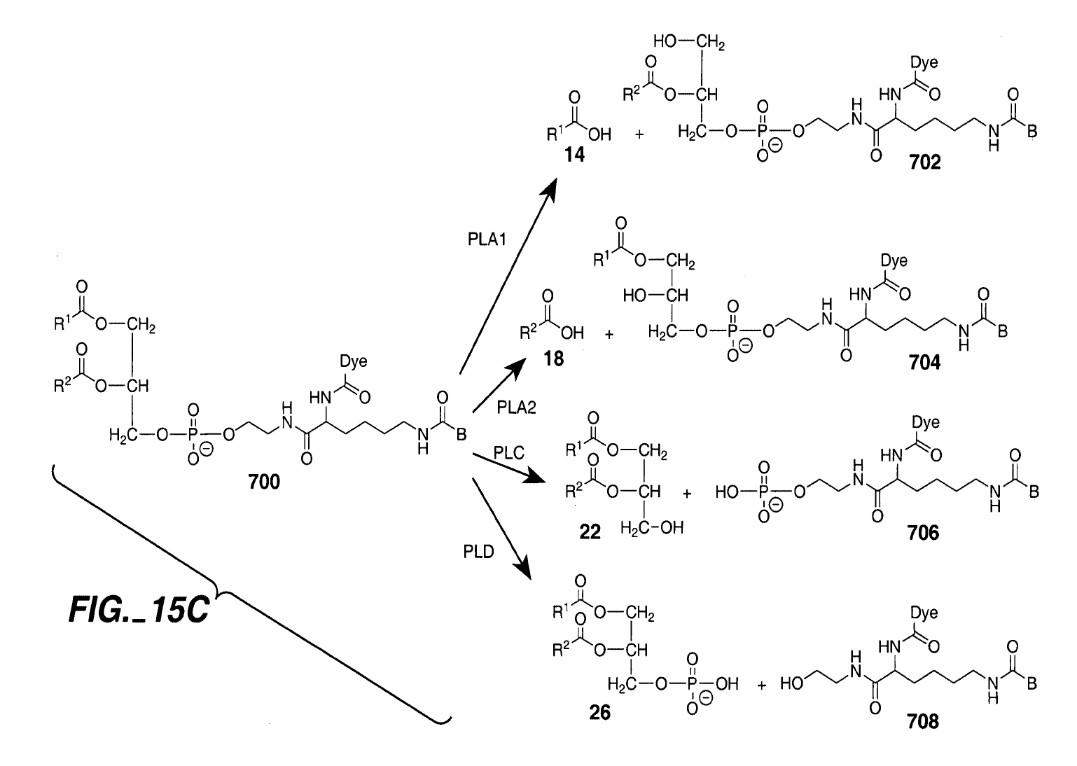

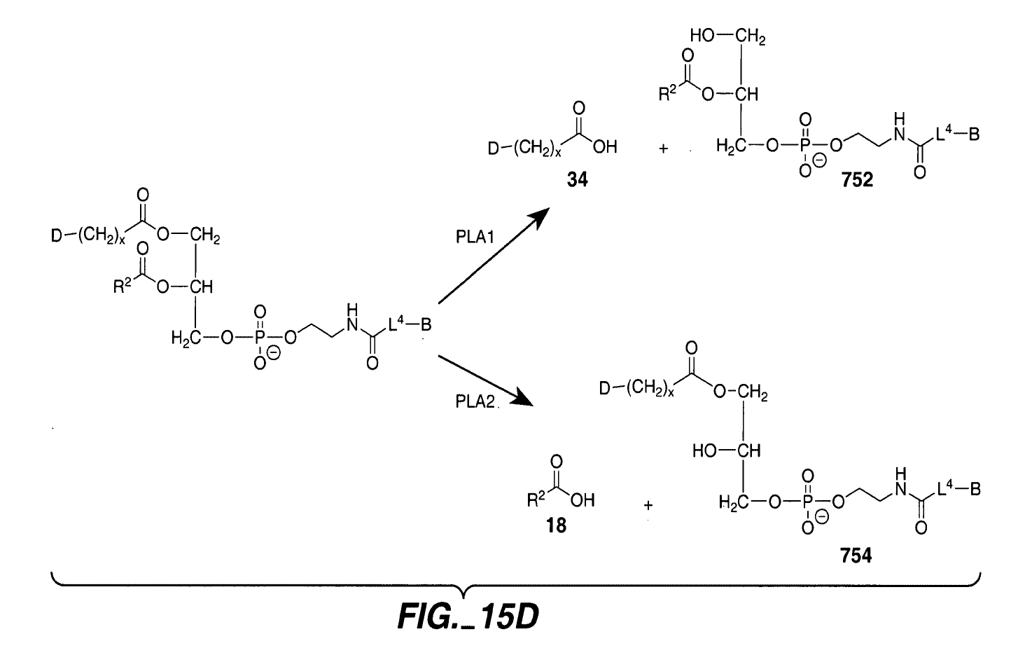

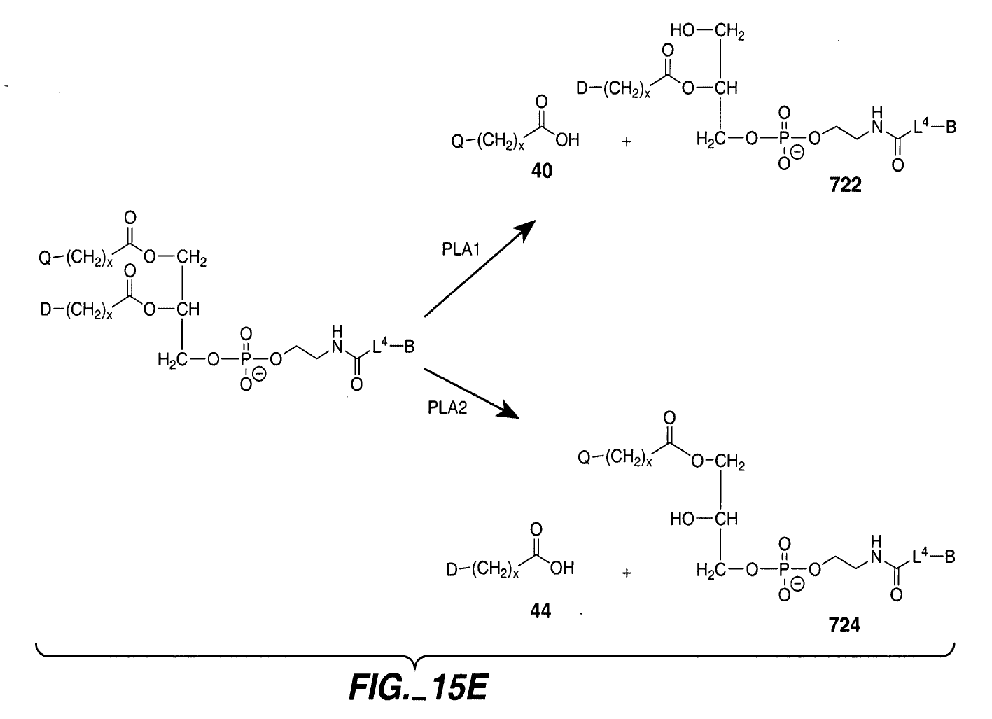

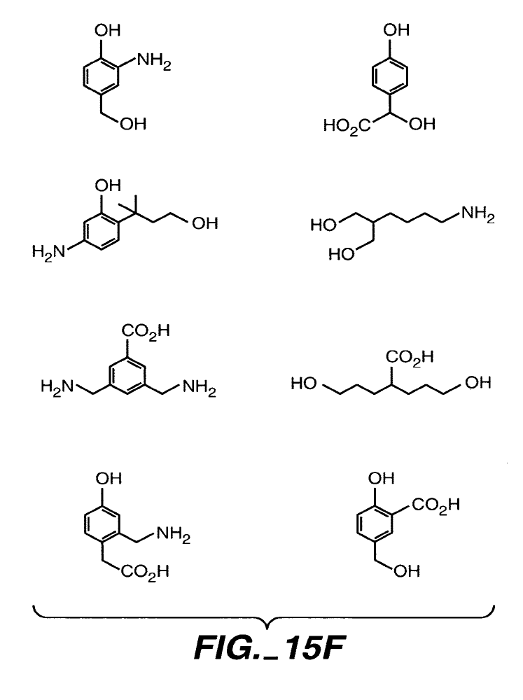

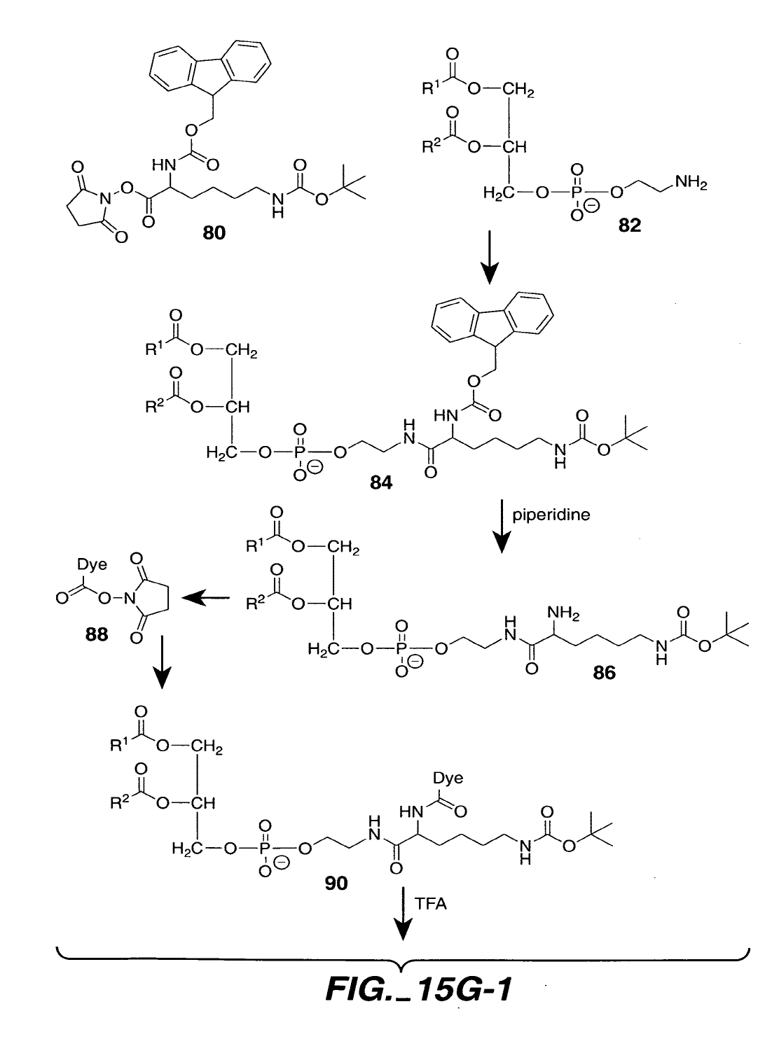

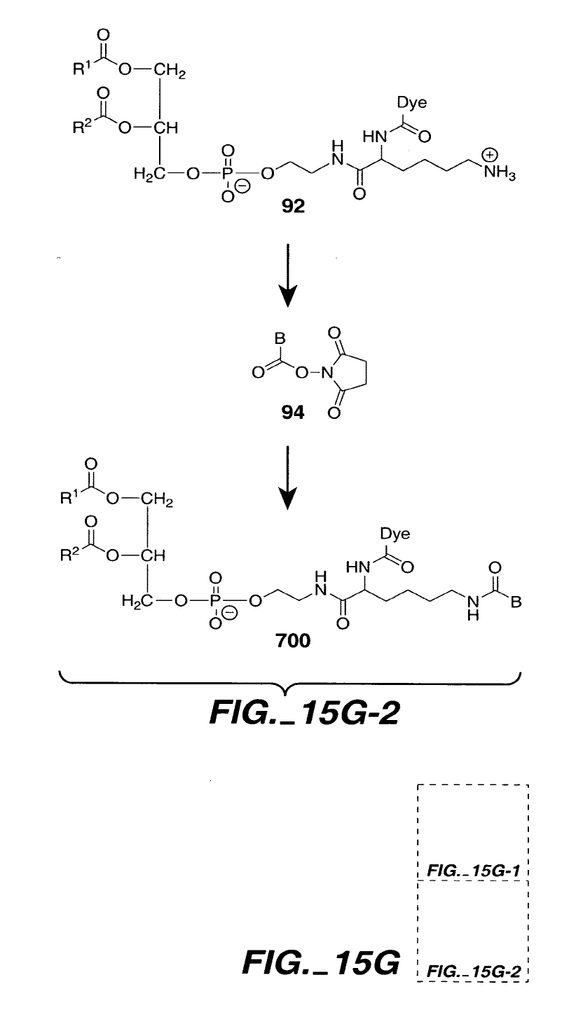

The ligand molecule can comprise additional features or moieties, such as a fluorescent moiety and/or a quenching moiety. Thus, in some embodiments, the ligand molecule can have dual roles (or more roles) within the micelle. Exemplary embodiments of ligand molecules including a fluorescent moiety (and optionally a quenching moiety) that can function as both the ligand molecule and the signal molecule in a ligand-containing micelle are illustrated in FIG. 15. Additional exemplary embodiments of suitable dual role ligand/signal molecules are described in copending U.S. application Ser. No. ______, and PCT application Ser. No. ______, entitled “Fluorogenic Homogeneous Binding Assay Methods and Compositions”, filed on Nov. 24, 2004, the disclosure of which is incorporated herein by reference.

FIG. 15A illustrates an exemplary embodiment of a dual role ligand/signal molecule in which the phospholipid hydrophobic moiety, binding moiety and fluorescent moiety are linked via a trivalent linker. In the illustrated molecule, the trivalent linker is provided by the α-amino acid lysine. The binding moiety (B—C(O)—) is linked to the side chain (epsilon) amino group, the fluorescent moiety (Dye-C(O)—) is linked to the alpha amino group and the hydrophobic moiety (R⁴—NH—), is linked to the alpha carboxyl. The binding, fluorescent and phospholipid hydrophobic moieties could be linked to the lysine linker in other arrangements from that illustrated.

As will be appreciated by skilled artisans, in FIG. 15A, the illustrated lysine is merely an exemplary trivalent linker. Any molecule having three “reactive” groups suitable for attaching other molecules and moieties thereto, or that can be appropriately activated to attach other molecules and moieties thereto, could be used. For example, the “backbone” of the linker to which the reactive (or activatable) linking groups are attached could be a linear, branched or cyclic saturated or unsaturated alkyl, a mono or polycyclic aryl or an arylalkyl. Moreover, while the previous examples are hydrocarbons, the linker backbone need not be limited to carbon and hydrogen atoms. Indeed, the linker backbone can comprise single, double, triple or aromatic carbon-carbon bonds, carbon-nitrogen bonds, nitrogen-nitrogen bonds, carbon-oxygen bonds, carbon-sulfur bonds and combinations thereof, and therefore can comprise functionalities such as carbonyls, ethers, thioethers, carboxamides, sulfonamides, ureas, urethanes, hydrazines, etc. Any type of linker backbone that permits the dual role ligand/signal molecule to function as described herein may be used.

The functional groups on the trivalent linker can be any member of a pair of complementary reactive groups capable of forming covalent linkages, as discussed above. In some embodiments, each reactive group comprising the trifunctional linker is an electrophilic group or a nucleophilic group that is capable of reacting with a complementary nucleophilic group or electrophilic group to form a covalent linkage stable to biological assay conditions, such as one of the nucleophilic or electropholic groups listed in Table 3, above.

The reactive groups on the trivalent linker may all be the same, or some or all of them may be different. In some embodiments, reactive groups are selected that have different chemical reactivities to facilitate the selective attachment of the binding, fluorescent and hydrophobic moieties, to the linker.

In some embodiments, the trifunctional linker is an amino acid, which may be an alpha amino acid, a beta amino acid, a gamma amino or other type of amino acid, that comprises a side chain having a suitable reactive functional group. Specific examples of suitable amino acids comprise, but are not limited to, lysine, glutamate, cysteine, serine, homoserine and 1,3-diaminobutyric acid. These amino acids may be in either the D- or L-configuration, or may constitute racemic or other mixtures thereof. Additional examples of suitable trivalent linkers are provided in FIG. 15F.

In the exemplary dual role signal/ligand molecule of FIG. 15A, R⁴ can be provided by a moiety that comprises a hydrophobic moiety and a modification moiety, as described herein. For example, R⁴—NH— could comprise a fatty acid linked to a peptide segment that comprises a cleavage site, such as a protease site, or a site modifiable by a protein kinase or phosphatase, as described above in connection with dye-peptide signal molecules. Alternatively, R⁴—NH— can be provided by a phospholipid, such as a glycerophospholipid or a sphingolipid. In such embodiments, the phospholipid can be covalently linked to the remainder of the ligand/signal molecule via its polar head group, although other linkages are possible. As a specific example, the R⁴—NH— group of the molecule illustrated in FIG. 15A can be provided by the glycerophospholipid phosphatidyl ethanolamine, as illustrated in FIG. 15B. In FIG. 15B, R¹ and R² can be any of the previously-described hydrophobic groups, and in a specific embodiment correspond to the alkyl moieties of the fatty acid chains of a naturally occurring phospholipid. Moreover, although the exemplary phospholipid dual role ligand/signal molecules of FIGS. 15A and 15B comprise a lysine trivalent linker, any trivalent linker could be used.

The cleavage products of dual role ligand/signal molecule 700 following treatment with phospholipases A1, A2, C and D are illustrated in FIG. 15C. Treatment of a micelle including dual role ligand/signal molecule 700 in which the fluorescent moieties are quenched yields an increase in fluorescence, as discussed above in connection with FIG. 2A.

Another embodiment of a dual role phospholipid ligand/signal molecule is illustrated in FIG. 15D. The dual phospholipid ligand/signal molecule 750 of FIG. 15D is a derivative of, and similar in concept to, signal molecule 200 of FIG. 1B. In FIG. 15D, “D,” x and R² are as defined for FIG. 1B, “L⁴” is an optional linker as described for FIG. 14A and “B” is a binding moiety. Although phospholipid ligand/signal molecule 750 comprises an ethanolamin-2-yl head group, other head groups could be used, as could other hydrophobic moieties, as described above in connection with FIG. 1B. Cleavage of phospholipid ligand/signal molecule 750 with PLA1 yields lysophospholipid derivative 752 and fluorescent moiety 34. Cleavage by PLA2 yields fatty acid 18 and lysophospholipid derivative 754.

Another embodiment of a dual role phospholipid ligand/signal molecule is illustrated in FIG. 15E. Exemplary phospholipid ligand/signal molecule 720 of FIG. 15E is a derivative of, and similar in concept to signal molecule 300 of FIG. 1C. In FIG. 15E, “Q,” “D” and x are as previously defined for FIG. 1C. “L⁴” represents an optional linker as previously discussed in connection with FIG. 14A and “B” represents a binding moiety. The cleavage products generated by treatment with phospholipases PLA1, PLA2, PLC and PLD are also shown. Micelles treated with PLC and/or PLD can be further treated with PLA1 and/or PLA2 to unquench the fluorescence moiety of fluorescent moiety “D,” leading to an observed increase in fluorescence. In some embodiments, when micelles including dual role ligand/signal molecule 720 are used and either PLC and/or PLD is used as the modification agent, the remainder of the micelle can be composed of lipids or phospholipids that are not cleaved by the PLC and/or PLD.

Phospholipid ligand molecules (as well as dual role phospholipid ligand/signal molecules) can be prepared in a manner analogous to phospholipid signal molecules. In one method, the phospholipid ligand molecule is prepared in a manner analogous to Scheme (I), supra.

Dual role ligand/signal molecule 700 can be synthesized as illustrated in FIG. 15G. Referring to FIG. 15G, protected lysine NHS-ester 80 is reacted with phospholipid 82 to yield protected compound 84. Removal of the FMOC group protecting the alpha amino group of compound 84 (for example with 30% piperidine in DMF) yields compound 86, which can be condensed with NHS-ester 88 to yield compound 90. Removal of the t-BOC group protecting the side chain (epsilon) amino group of compound 90 (for example by treatment with 1% TFA in methylene chloride for 10 minutes) yields compound 92, which can be condensed with NHS-ester 94 to yield ligand/signal molecule 700.

The various illustrated NHS-esters may be preformed, isolated and purified, or, alternatively, they may be formed in situ by reacting the corresponding carboxylic acid with the amine in the presence of some combination of: (1) a carbodiimide reagent, e.g. dicyclohexylcarbodiimide, diisopropylcarbodiimide, or a uronium reagent, e.g. TSTU (O—(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N,N′-tetramethyluronium hexafluorophosphate); (2) an activator, such as 1-hydroxybenzotriazole (HOBt) or 1-hydroxyazabenotriazole (HOAt); and (3) N-hydroxysuccinimide to give the NHS ester of the carboxylic acid.

Other activating and coupling reagents that could be used comprise TBTU (2-(1H-benzotriazo-1-yl)-1-1,3,3-tetramethyluronium hexafluorophosphate), TFFH(N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and arylsulfonyl halides, e.g. triisopropylbenzenesulfonyl chloride.

As will be appreciated by skilled artisans, activated esters and protecting groups other than those illustrated may also be employed. Suitable groups and chemistries comprise those conventionally employed in the solution phase and solid phase synthesis of peptides, such as the various groups and chemistries described, for example, in Lloyd-Williams et al., CHEMICAL APPROACHES TO THE SYNTHESIS OF PEPTIDES AND PROTEINS, CRC Press, 1997 and Atherton & Sheppard, SOLID PHASE PEPTIDE SYNTHESIS: A PRACTICAL APPROACH, IRL Press, 1989.

Suitably protected trivalent linkers, such as protected trivalent linker 88 of FIG. 15G, can be prepared using standard techniques. Methods for preparing protected amino acids that comprise orthogonal or non-orthogonal protection strategies are taught in the above references. Many suitably protected amino acids can also be purchased commercially. Protection strategies and chemistries for trivalent linkers including functional groups other than those found in amino acids are taught in standard texts, such as, for example, in Greene & Wuts, PROTECTIVE GROUPS IN ORGANIC SYNTHESIS, Second Edition, John Wiley & Sons, Inc., 1991.

Methods of synthesizing and/or obtaining fluorescent dyes corresponding in structure to compound 88 and phospholipids corresponding in structure to compound 82 are described above.

If the ligand molecule is a membrane protein, the ligand molecule can be first solubilized in a detergent solution and then reconstituted into micelle using various methods known in the art. See, for example, Schoch et al., J. RECEPT. RES. 4: 189-200 (1984); Sigel et al., NEUROSCI. LETT. 61: 165-170 (1985); Fujioka et al., BIOCHEM. BIOPHYS, RES. COMM. 156: 54-60 (1988); Lundahl and Yang, J. CHROMATOGR. 544: 283-304 (1991); Dunn et al., BIOCHEMISTRY 28: 2545-2551 (1989); Gomathi and Sharma, FEBS LETT. 330: 146-150 (1993); Gioannini et al., BIOCHEM. BIOPHYS, RES. COMM. 194: 901-908 (1993); and Balen et al., BIOCHEMISTRY 33: 1539-1544 (1994). Suitable detergents that can be used to solubilize membrane protein ligand molecules comprise, but are not limited to, deoxycholate, CHAPS, and Triton X-100. Before reconstitution, detergents can be depleted by using size-exclusion chromatography, dialysis, absorption (e.g. absorption of Triton X-100 using Bio-Beads SM-2), or other means. The use of specific phospholipids during the reconstitution procedure may help the recovery of a functionally active ligand molecule. For instance, a vesicle having a phosphatidylethanolamine:phosphatidylcholine ratio of 1:2 may be used to improve the functional reconstitution of a membrane protein ligand molecule.

6.2.5 Quenching Molecule

Although not required, in some embodiments, the ligand-containing micelle comprises a quenching molecule that functions to aid the quenching effect of the fluorescent moiety of the signal molecule(s) in the micelle. The quenching molecule comprises a quenching moiety and at least one hydrophobic moiety. The hydrophobic moiety integrates the quenching molecule into the micelle. The quenching moiety is selected such that it is capable of quenching the fluorescence of fluorescent moiety on the signal molecule comprising the micelle. If the micelle comprises a plurality of signal molecules having different fluorescent moieties, a quenching moiety capable of quenching the fluorescence of all or a subset of the fluorescent moieties may be selected. Any of the hydrophobic and quenching moieties previously described can be used to construct a quenching molecule.

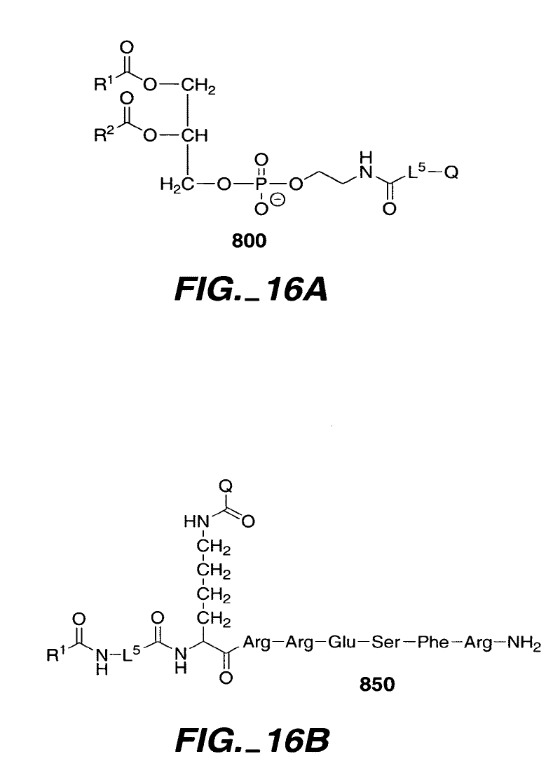

In some embodiments, the quenching molecule comprises a quenching moiety, such as, for example, one of the previously discussed quenching moieties, covalently coupled to a fatty acid or a phospholipid, optionally by way of a linker. A specific embodiment of a phospholipid quenching molecule 800 is illustrated in FIG. 16A. In FIG. 15A, R¹ and R² are hydrophobic moieties as defined for FIG. 1A, “Q” is a quenching moiety and “L⁵” is an optional linker, such as one of the linkers “L” described in connection with FIG. 1A. The length and chemical composition of optional linker “L⁵” can be selected to position quenching moiety “Q” in proximity to the fluorescent moiety of a signal molecule in the same micelle. A specific embodiment of a quenching molecule 850 that can be modified with a protein kinase C is provided in FIG. 16B. The selection of quenching moiety Q will depend, in part, on the identity of the fluorescent moiety whose fluorescence is to be quenched.

6.2.6 The Charge Balance Molecule

Although not required, in some embodiments, the ligand containing micelle comprises a charge balance molecule that functions to balance the overall charge of the micelle. The charge balance molecule comprises a charge balance moiety and at least one hydrophobic moiety. The hydrophobic moiety integrates the charge balance molecule into the micelle. The charge balance moiety is selected such that it is capable of balancing the overall charge of the micelle. Any of the hydrophobic moieties previously described can be used to construct a charge balance molecule. Examples of suitable charge balance moieties for inclusion in a charge balance molecule are described above.

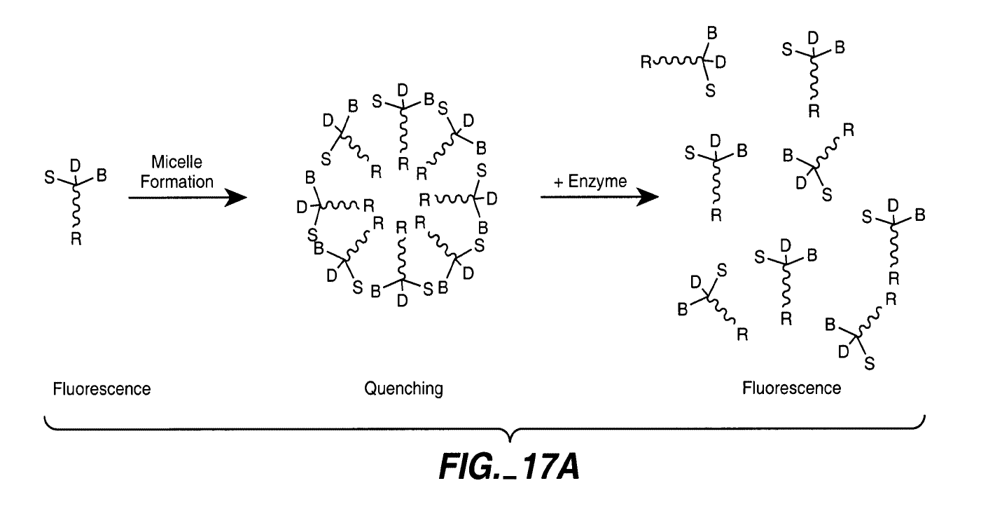

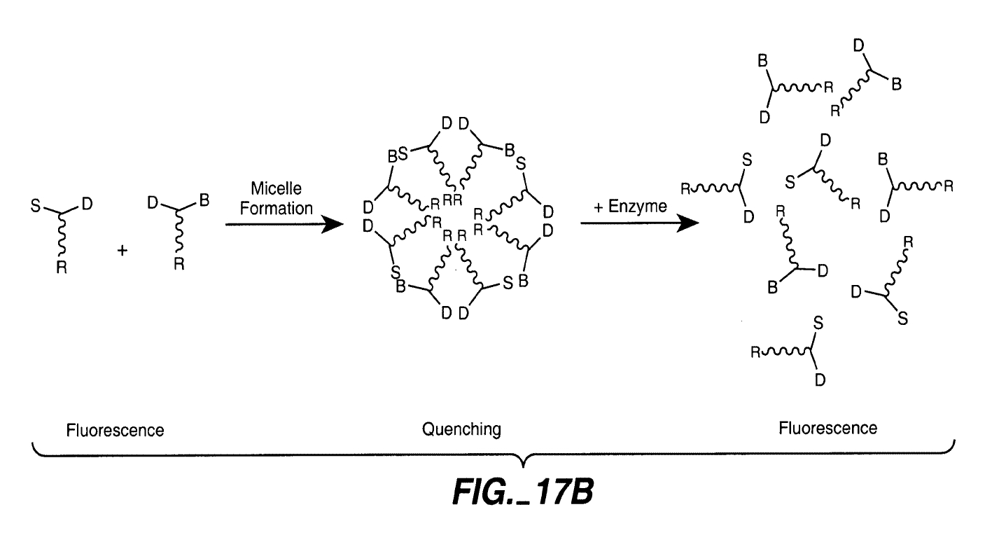

FIG. 17A illustrates an exemplary embodiment wherein the hydrophobic, fluorescent, substrate, and charge-balance moieties are included in a single molecule. In FIG. 17A, the signal molecule comprises a hydrophobic moiety R, a fluorescent moiety D, a substrate moiety S, and a charge-balance moiety B. The fluorescence of the fluorescent moiety is quenched when the signal molecule is incorporated into the micelle. The charge-balance moiety acts to balance the overall charge of the micelle such that micelle formation is promoted or encouraged. The hydrophobic moiety acts to integrate the signal molecule into a micelle when included in an aqueous solvent at or above the critical micelle concentration, thereby quenching the fluorescence of the fluorescent moieties. The addition of an enzyme that modifies the signal molecule and promotes the dissociation of the fluorescent moieties from the micelle, thereby reducing or eliminating the quenching effect caused by the interactions between the fluorescent moieties and the micelle.

FIG. 17B illustrates an exemplary embodiment wherein the hydrophobic, fluorescent, substrate, and charge-balance moieties are included in two different, distinct molecules. The signal molecule comprises a hydrophobic moiety R, a fluorescent moiety D, and a substrate moiety S. The charge-balance molecule comprises a hydrophobic moiety R, a fluorescent moiety D, and a charge-balance moiety B. The fluorescence of the fluorescent moieties is quenched when the signal molecule and charge-balance molecule are incorporated into the micelle. The charge-balance moiety acts to balance the overall charge of the micelle such that micelle formation is promoted or encouraged. The hydrophobic moieties act to integrate the signal molecule and the charge-balance molecule of the composition into a micelle when included in an aqueous solvent at or above the critical micelle concentration, thereby quenching the fluorescence of the fluorescent moieties. The addition of an enzyme that modifies the signal molecule and promotes the dissociation of the fluorescent moieties from the micelle, thereby reducing or eliminating the quenching effect caused by the interactions between the fluorescent moieties and the micelle.

### 6.3 Ligand-Containing Micelles

The ligand molecule, signal molecule, and optional charge balance molecule and quenching molecule are incorporated into a micelle such that the fluorescence of the fluorescent moiety of the signal molecule is quenched in the micelle. Depending upon the mechanism by which the quenching effect is achieved (e.g., whether by self-quenching or with the aid of a quenching moiety or quenching molecule). The signal molecule can comprise a primary component or constituent of the micelle or, alternatively, the signal molecule can comprise a minor component or constituent of the micelle. If a dual role ligand/signal molecule is used, the ligand/signal molecule can constitute the only component of the micelle, or it may be one of several components or constituents.

The form of the micelle is not critical to success. The micelle can range in form from a “detergent-like” micelle which does not enclose a part of the aqueous solvent to a “vesicle-like” micelle which encloses a part of the aqueous solvent. Such vesicle-like micelles can be small or large in size, and can be unilamellar or multilamellar. The micelle can also take on any type of three-dimensional shape or structure, including, for example, spherical, oblate, discoidal and cubic.

The micelles can be formed in situ during the course of an assay, or they can be preformed and added to an assay in micellar form. Micelles formed in situ can be prepared by mixing the ligand molecule, signal molecule and any optional quenching molecules or other components comprising the micelle in the assay buffer at concentrations at or above their critical micelle concentrations. The assay buffer can be optionally agitated to promote micelle formation.

The ligand molecule, signal molecule and optional quenching molecule should be included in the micelle at molar ratios that permit them to perform their respective functions. For example, the ligand molecule should be included in a molar ratio that provides a sufficient number of binding moieties such that binding between the ligand and another molecule is likely to occur. The signal molecule and optional quenching molecule should be included in molar ratios that yield an acceptable dynamic range of fluorescence signal under the assay conditions. For example, the signal molecule and optional quenching molecule can be included in the micelles at molar ratios sufficient to provide quenching of the fluorescent moieties in the micelle and a detectable increase in fluorescence over this quenched background when the micelle is treated with the specified modification agent. Embodiments in which the quenching effect is achieved by self-quenching of the fluorescent moieties without the aid of quenching moieties and/or quenching molecules may require a higher molar ratio of signal molecule than embodiments employing quenching moieties and/or quenching molecules.

For any particular micellar form and desired ligand molecule, signal molecule and optional quenching molecule, suitable molar ratios of ligand molecule, signal molecule and optional quenching molecule can be determined empirically. For example, the appropriate amount of signal molecule and optional quenching molecule can be determined by preparing several batches of micelles comprising varying molar ratios of signal molecule and optional quenching molecule and comparing the increase in fluorescence observed upon treatment with the specified modification agent. Once a suitable signal is achieved, the molar ratio of the ligand molecule can be optionally varied and the micelles tested for suitable signal dynamic range in a control binding experiment with a known binding partner for the ligand. As will be appreciated, other methods could also be used to empirically determine optimal molar ratios of ligand, signal and optional quenching molecules for particular applications.

In preferred embodiments, the micelle is a liposome. A liposome is a self-closed vesicle where one or several lipid membranes encapsulate part of the solvent. The composition and form of these lipid vesicles are analogous to that of cell membranes with hydrophilic polar groups directed inward and outward toward the aqueous media and hydrophobic fatty acids intercalated within the bilayer. Liposomes are formed when thin lipid films or lipid cakes are hydrated and stacks of liquid crystalline bilayers become fluid and swell. Liposomes may be unilamellar and/or multilamellar. Unilamellar liposome vesicles are typically classified as small (SUVs) (less than 50 nm in diameter), large (LUVs) (50-250 nm in diameter) or giant (approx. 1 micron in diameter). Small (SMV) and large, multilamellar liposome vesicles (LMV) can also be formed. Multilamellar liposomes are classically described as having concentric bilayers, an “onion morphology.” A type of multilamellar liposome termed oligolamellar liposomes are typically described as multilamellar liposomes which have increased aqueous space between bilayers or which have liposomes nested within bilayers in a nonconcentric fashion. Once these complexes have formed, reducing the size of the complex requires energy input in the form of sonic energy (sonication) or mechanical energy (extrusion).

Liposomes are typically comprised of phospholipids having hydrophobic tails or other bulky hydrophobic moieties that disfavor the formation of detergent-like micelles. Liposomes can be formed from any single type of phospholipids or mixture of phospholipids. A liposome preparation can comprise one or more of phosphatidic acid, phosphatidylethanolamine, phosphatidylcholine, phosphatidylinositols, phosphatidylglycerol, sphingomylelin, cardiolipin, lecithin, phosphatidylserine, cephalin, cerebrosides, dicetylphosphate, steroids, terpenes, stearylamine, dodecylamine, hexadecylamine, acetylpalmitate, glycerol ricinoleate, hexadecyl stearate, isopropyl myristate, dioctadecylammonium bromide, amphoteric polymers, triethanolamine lauryl sulfate and cationic lipids, 1-alkyl-2-acyl-phosphoglycerides, and 1-alkyl-1-enyl-2-acyl-phosphoglycerides. Other lipids useful in forming liposomes include cationic lipids, examples of which include dioctadecyl dimethyl ammonium bromide/chloride (DODAB/C) and dioleoyloxy-3-(trimethylammonio)propane (DOTAP). See, for example, Lasic, LIPOSOMES IN GENE DELIVERY, CRC Press, New York, pp. 81-86 (1997). Cholesterols may also be used.

A wide variety of suitable lipids are commercially available (such as from Avanti Polar Lipids, Inc. Alabaster, Ala.). Liposome kits are commercially available (e.g. from Boehringer-Mannheim, ProMega, and Life Technologies (Gibco)). Non-limiting examples of suitable lipids include 1,2-dimyristoyl-sn-glycero-3-phosphate (Monosodium Salt) (DMPA•Na) (Avanti catalog no. 830845), 1,2-dimyristoyl-sn-glycero-3-phosphate (Monosodium Salt) (DOPS•Na) (Avanti catalog no. 830035), and 1,2-dioleoyl-3-trimethylammonium-propane (Chloride Salt) (DTOAP•Cl) (Avanti catalog no. 890890).

Liposomes can also comprise synthetic lipid compounds such as D-erythro (C-18) derivatives including sphingosine, ceramide derivatives, and sphinganine; glycosylated (C18) sphingosine and phospholipid derivatives; D-erythro (C17) derivatives; D-erythro (C20) derivatives; and L-threo (C18) derivatives, all of which are commercially available (Avanti).

Liposomes can comprise or be formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally comprises an ester moiety (ester of a fatty acid), the ester linkage can be replaced with an ether linkage at position 1 and/or 2.

In certain embodiments, ligand-containing liposomes which have been found to be useful include, in addition to the ligand and signal molecules, lipids such as phosphatidylcholine (PC) and phosphatidylethanolamine (PE). Preferably, phosphatidylcholine ranges from about 50 to 95 mol percent of the total lipid content of the liposome, and phosphatidylethanolamine ranges from 2 to 20 mol percent. More preferably, phosphatidylcholine ranges from about 60 to 90 mol percent, and phosphatidylethanolamine ranges from about 4 to 12 mol percent.

Ligand-containing liposomes may comprise cholesterol. Cholesterol can intercalate within the liposome bilayer by occupying the regions created by the bulky phospholipid head groups. This increases the packing density and structural stability of the bilayer (New, R.R.C. (ed): LIPOSOMES: A PRACTICAL APPROACH, Oxford University Press, New York, pp 19-21 (1990)). Cholesterol also affect the fluidity and permeability of the membrane. The concentration of cholesterol in liposomes can range, for example, from about 5 to about 60 mol percent.

The composition of the ligand-containing liposomes can be selected based an a variety of factors including cost, transition temperature of the lipids, stability during storage, and stability of the liposomes under the reaction conditions, and the presence of the enzyme activities being used to modify the fluorescently-labeled molecule.

Properties of liposomes can vary depending on the composition (cationic, anionic, neutral lipid species). However, the same preparation method may be used for all lipid vesicles regardless of composition. The general elements of the procedure involve preparation of the lipid for hydration, hydration with agitation, and sizing to a homogeneous distribution of vesicles.

Ligand-containing liposomes can be prepared using conventional methods, such as described in Lasic, LIPOSOMES IN GENE DELIVERY, CRC Press, New York, pp. 67-112 (1997); ANN. REV. BIOPHYS. BIOENG. 9:467-508 (1980); U.S. Pat. Nos. 4,229,360, 4,235,871, 4,241,046, 6,458,381 and 6,534,018. When preparing liposomes with mixed lipid composition, the lipids can first be dissolved and mixed in an organic solvent to assure a homogeneous mixture of lipids. Usually this process is carried out using chloroform or chloroform:methanol mixtures. Typically lipid solutions can be prepared at 10-20 mg lipid/ml organic solvent, although higher concentrations may be used if the lipid solubility and mixing are acceptable. Once the lipids are thoroughly mixed in the organic solvent, the solvent is removed to yield a lipid film. For small volumes of organic solvent (<1 ml), the solvent may be evaporated using a dry nitrogen or argon stream in a fume hood. For larger volumes, the organic solvent can be removed by rotary evaporation yielding a thin lipid film on the sides of a round bottom flask. The lipid film is thoroughly dried to remove residual organic solvent by placing the vial or flask on a vacuum pump overnight. If the use of chloroform is objectionable, tertiary butanol, cyclohexane or other alternatives can be used to dissolve the lipid(s). The lipid solution is transferred to containers and frozen by placing the containers on a block of dry ice or swirling the container in a dry ice-acetone or alcohol (ethanol or methanol) bath. Care should be taken when using the bath procedure that the container can withstand sudden temperature changes without cracking. After freezing completely, the frozen lipid cake is placed on a vacuum pump and lyophilized until dry (1-3 days depending on volume). The thickness of the lipid cake preferably is no more than the diameter of the container being used for lyophilization. Dry lipid films or cakes can be removed from the vacuum pump, the container close tightly and taped, and stored frozen until ready to hydrate.

Hydration of the dry lipid film/cake is accomplished simply by adding an aqueous medium to the container of dry lipid and agitating. The temperature of the hydrating medium should be above the gel-liquid crystal transition temperature (Tc) of the lipid that has the highest Tc. After addition of the hydrating medium, the lipid suspension is maintained above the Tc during the hydration period. For high transition lipids, this is easily accomplished by transferring the lipid suspension to a round bottom flask and placing the flask on a rotary evaporation system without a vacuum. Spinning the round bottom flask in the warm water bath maintained at a temperature above the Tc of the lipid suspension allows the lipid to hydrate in its fluid phase with adequate agitation. Hydration time may differ slightly among lipid species and structure. A hydration time of 1 hour with vigorous shaking, mixing, or stirring is recommended. It is also believed that allowing the vesicle suspension to stand overnight (aging) prior to downsizing may make the sizing process easier and improves the homogeneity of the size distribution. The hydration medium is generally determined by the application of the lipid vesicles. Suitable hydration media comprise distilled water, buffer solutions, saline, and nonelectrolytes such as sugar solutions, for example. During hydration some lipids form complexes unique to their structure. Highly charged lipids have been observed to form a viscous gel when hydrated with low ionic strength solutions. The gel formation can be alleviated by addition of salt or by downsizing the lipid suspension. The product of hydration usually is a large, multilamellar vesicle (LMV) analogous in structure to an onion, with each lipid bilayer separated by a water layer. LMV can be directly used in the present composition and methods. LMV can also be further downsized by a variety of techniques, including sonication or extrusion.

Disruption of LMV suspensions using sonic energy (sonication) typically produces small, unilamellar vesicles (SUV) with diameters in the range of 15-50 nm. Instrumentation for preparation of sonicated particles includes bath, probe tip and cup-horn sonicators. Sonication of an LMV dispersion is accomplished by placing a test tube containing the suspension in a bath sonicator (or placing the tip of the sonicator in the test tube) and sonicating for 5-10 minutes above the Tc of the lipid. The lipid suspension should begin to clarify to yield a slightly hazy transparent solution. The haze is due to light scattering induced by residual large particles remaining in the suspension. These particles can be removed by centrifugation to yield a clear suspension of SUV. Mean size and distribution is influenced by composition and concentration, temperature, sonication time and power, volume, and sonicator tuning.

An alternative method for sizing is extrusion. Lipid extrusion is a technique in which a lipid suspension is forced through a polycarbonate filter with a defined pore size to yield particles having a diameter near the pore size of the filter used. Prior to extrusion through the final pore size, LMV suspensions can be disrupted either by several freeze-thaw cycles or by prefiltering the suspension through a larger pore size (typically 0.2-1.0 μm). This method helps prevent the membranes from fouling and improves the homogeneity of the size distribution of the final suspension. As with all procedures for downsizing LMV dispersions, the extrusion preferably is done at a temperature above the Tc of the lipid. Extrusion through filters with 100 nm pores typically yields large, unilamellar vesicles (LUV) with a mean diameter of 120-140 nm. Mean particle size also depends on lipid composition and is reproducible from batch to batch.

Preparations of ligand-containing liposomes can comprise stabilizing agents, such as, for example, antioxidants, such as α-tocopherol and chelators. Other agents, including ascorbic acid, cysteine, monothioglycerol, sodium bisulfite, sodium metabisulfite, gentisic acid, and inositol, may also be used. Ligand-containing liposomes can be lyophilized for storage and/or for inclusion in kits.

The micelles can comprise more than one type of ligand molecule, signal molecule and/or optional quenching and charge balance molecules. For example, a micelle can comprise two different types of ligand molecules and a single type of signal molecule. An observed increase in the fluorescence signal in a binding assay carried out with this type of micelle indicates that one or both of the ligand molecules bound the molecule(s) in the sample.

In embodiments which utilize a dual role ligand/signal molecule, the fluorescent moieties on the different ligand/signal molecules can be selected such that their fluorescence signals are spectrally resolvable. In this manner, the different binding moieties comprising the micelle can be correlated to different colored signals. An increase in fluorescence signals at a specified wavelength can indicate not only that the micelle bound the molecule(s) in the sample, but also which binding moiety bound.

Micelles that are vesicle-like, such as liposomes, can optionally encapsulate agents within their interior. In some embodiments, the liposome can encapsulate a fluorescent dye (or combination of dyes) which can be used as a tracer to assess the integrity of the liposomes during preparation, storage and/or subsequent use.

The encapsulated dyes could also be used to identify the structure of the ligand molecule comprising the liposome. For example, a signal molecule can be selected such that the integrity of the micelle is maintained following modification and release of the fluorescent moiety. Subsequent disruption of the micelle, for example by treatment with detergent or a phospholipase that disrupts the liposome integrity, releases the encapsulated dye(s). If an assay is carried out with a plurality of liposomes, each of which encapsulates a different, spectrally resolvable fluorescent dye (or combination of dyes), the release of the encapsulated dye(s) can be used to reveal which ligand molecule(s) bound the sample molecule.

In another embodiment, an encapsulated agent can be selected that quenches the fluorescence of the signal molecules. As discussed above in connection with quenching moieties and quenching molecules, such quenching agents can be “dark,” or alternatively, they may themselves be fluorescent.

In those embodiments in which fluorescent dyes or quenching agents are encapsulated within the micelle, conventional methods can be used for loading, such as reverse phase methods and sonication (e.g. Lasic, LIPOSOMES IN GENE DELIVERY, CRC Press, New York, p. 93 (1997); and U.S. Pat. No. 4,888,288).

### 6.4 Methods

The ligand-containing micelles can be used in a variety of different assays to detect and/or screen for binding interactions between the ligand and other molecules. In some embodiments, a composition comprising a ligand-containing micelle is contacted with a sample comprising a binding molecule or putative binding molecule. In preferred embodiments, the binding molecule or putative binding molecule is immobilized on a substrate so as to facilitate removal of unbound micelles. Following contact and removal of unbound micelles, the sample is treated with a modification agent that modifies the micelle to unquench the fluorescence of the fluorescent moieties of the signal molecules, producing an increase in fluorescence of the sample. In this embodiment, the increase in fluorescence correlates with the presence of a binding interaction between the ligand molecule of the micelle and the binding molecule or putative binding molecule of the sample.

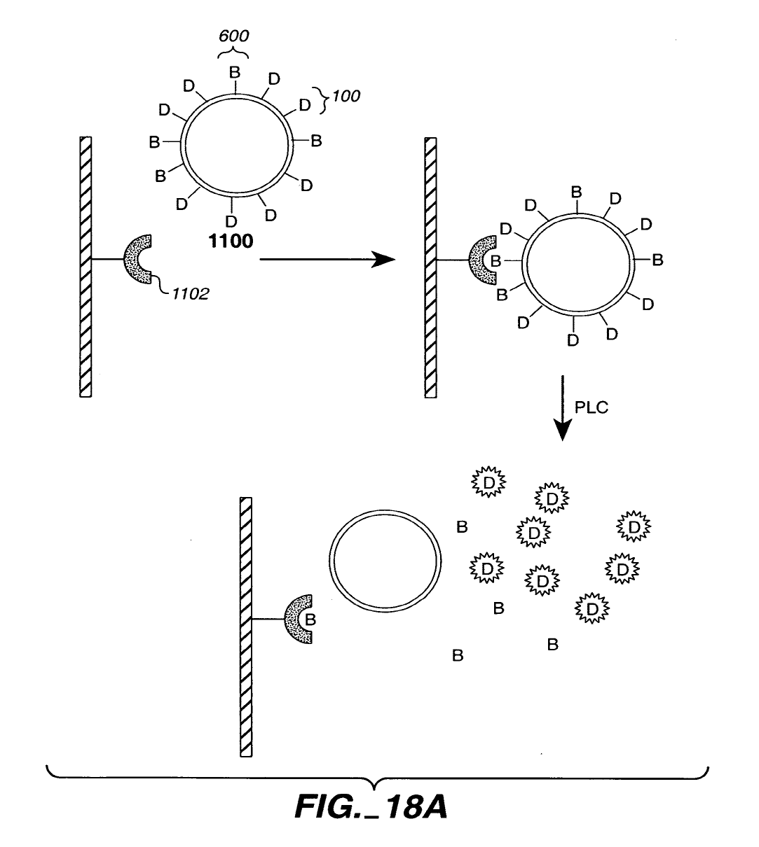

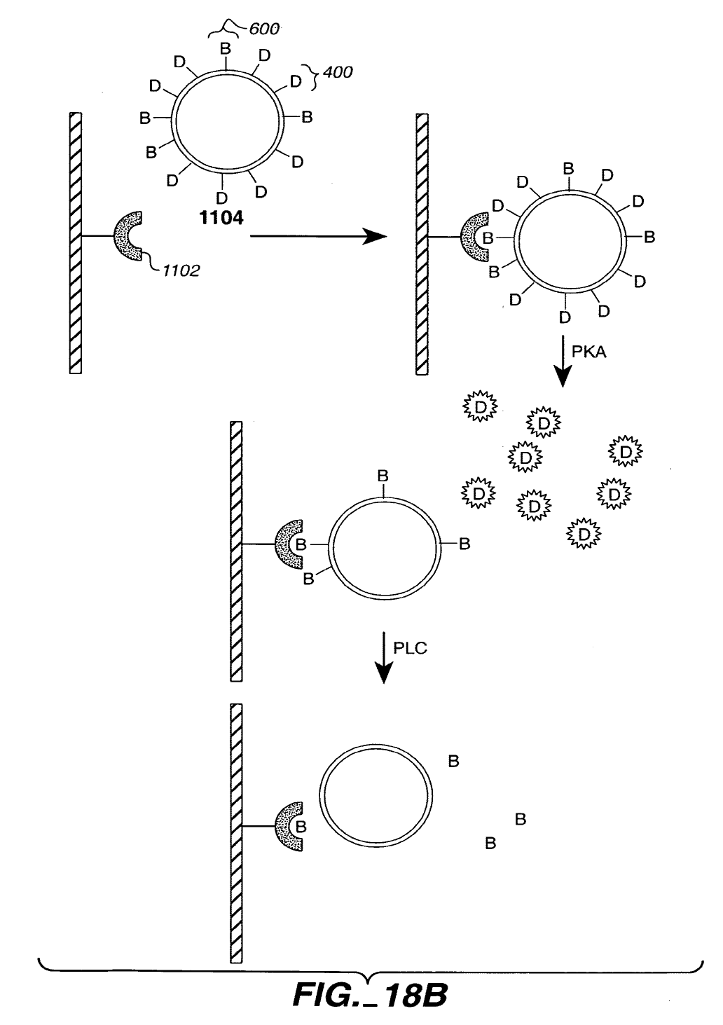

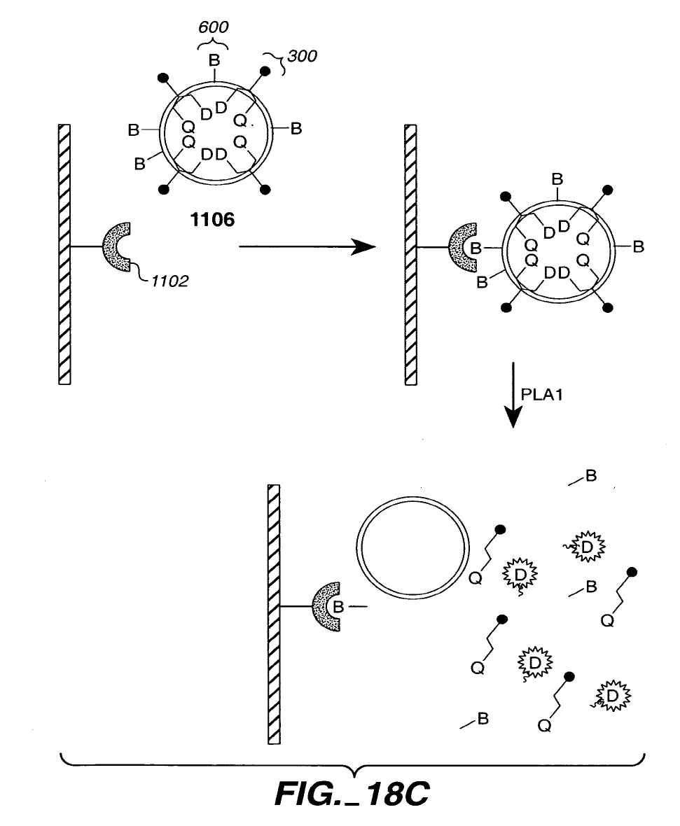

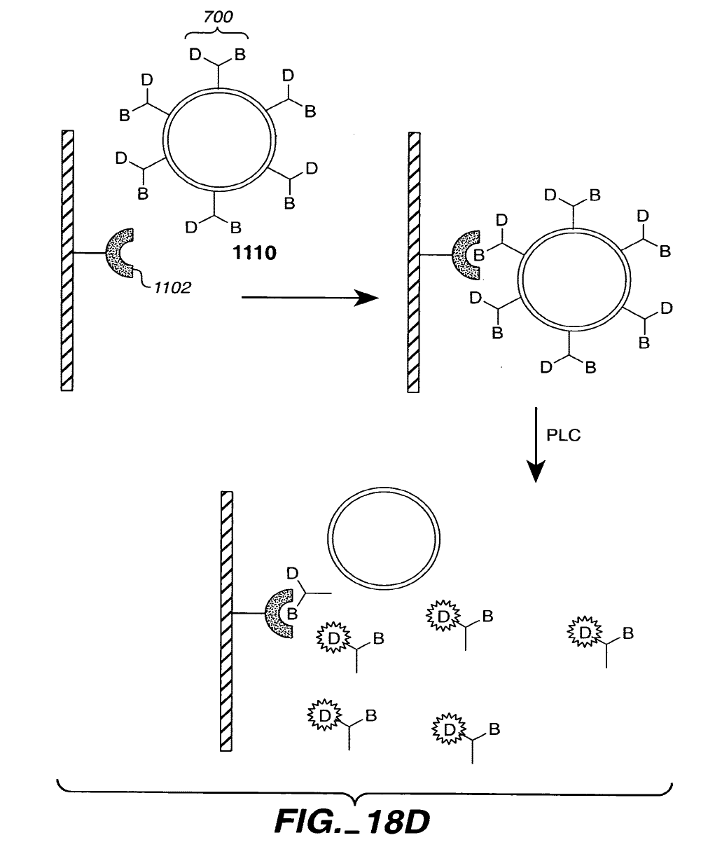

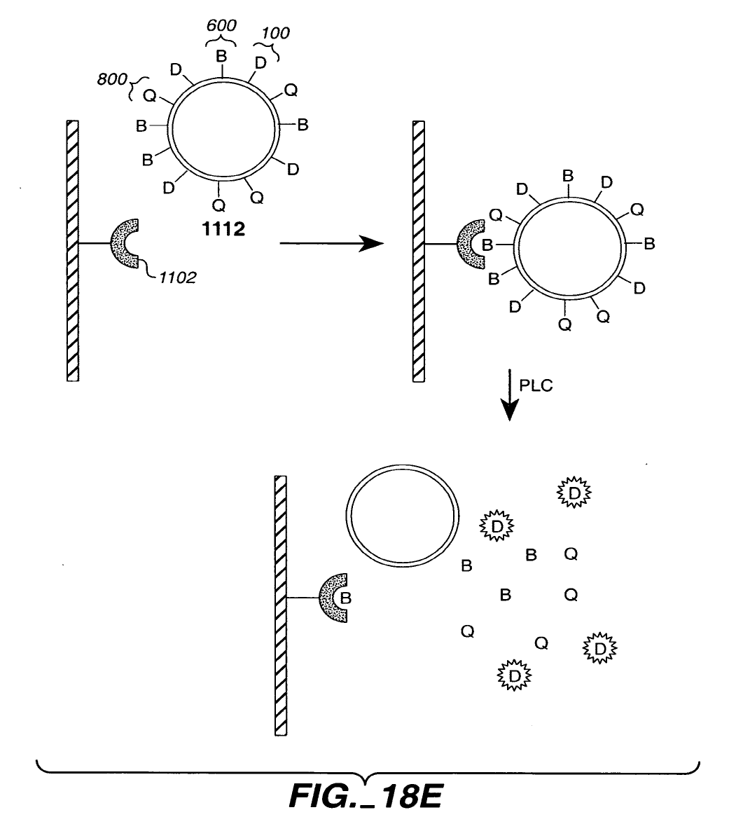

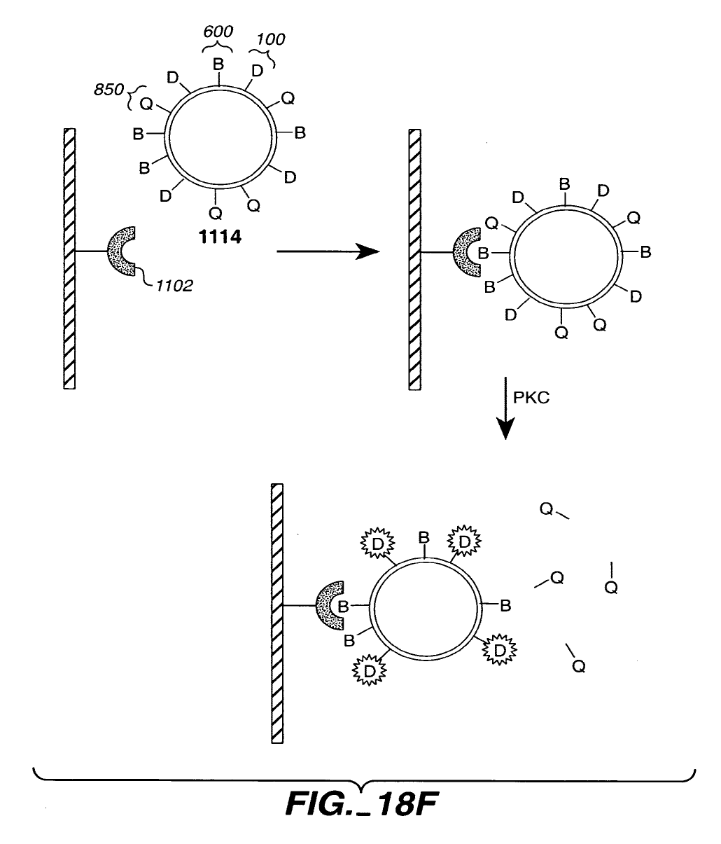

As discussed above, the quenching effect of the signal molecules in the micelle can be caused by a variety of different mechanisms, or a combination of mechanisms. For example, the quenching may be caused by intermolecular “self-quenching” between fluorescent moieties of the same type present on different signal molecules. An exemplary embodiment of a binding assay in which the fluorescent moieties are self-quenched is illustrated in FIG. 18A. In FIG. 18A, the ligand-containing micelle comprises phospholipid signal molecules and phospholipid ligand molecules. For purposes of illustration, the signal molecule can correspond to the signal molecule 100 of FIG. 1A and the ligand molecule can correspond to the ligand molecule 600 of FIG. 6A. The liposome 1100 is contacted with a sample comprising an immobilized putative binding partner 1102. Following removal of unbound micelles, such as by washing, the sample is treated with phospholipase C, which cleaves the fluorescent moieties from the signal molecules comprising the micelle, unquenching their fluorescence, thereby resulting in an increase in fluorescence of the sample. The sample could also be treated with PLA1, PLA2 or PLD.

While the exemplary assay of FIG. 18A is illustrated with a single type of liposome, the assay could be carried out with a plurality of liposomes, each of which comprises a different ligand molecule. In some embodiments, each liposome also comprises a fluorescent moiety having a fluorescence spectrum that can be resolved from the others such that the particular ligand molecules can be correlated with a particular fluorescence spectrum or “color.” An increase in fluorescence at the specified wavelength(s) indicates which of the ligand molecules bound the sample.

Moreover, while the exemplary embodiment of FIG. 18A employs a ligand molecule which comprises a modification site that is modified by the same modification agent as the signal molecule (in this case a phospholipase cleavage site), the liposome could comprise a ligand molecule that does not comprise a modification site or that is modifiable by a different modification agent than the signal molecule. For example, the signal molecule could correspond in structure to the signal molecule 400 of FIG. 11A and the ligand molecule could correspond in the structure to the ligand molecule 600 of FIG. 14A. As illustrated in FIG. 18B, treatment with a protein kinase A releases fluorescent moiety “D,” causing an increase in fluorescence. However, the binding moiety “B” is not released. Optional subsequent treatment with a phospholipase (e.g., PLC) releases the binding moiety “B,” which can be removed and analyzed, if desired.

An exemplary embodiment in which the quenching is caused by a signal molecule comprising a quenching moiety is illustrated in FIG. 18C. In FIG. 18C, the micelle 1106 (in this case a liposome) comprises a phospholipid signal molecule corresponding in structure to the phospholipid signal molecule 300 of FIG. 1C, where the fluorescence of fluorescent moiety “D” is quenched intramolecularly (and/or intermolecularly) by quenching moiety “Q.” The ligand molecule corresponds in structure to the phospholipid molecule 600 of FIG. 14A, although other ligand molecules could be used. Following contact and removal of unbound micelles, the sample is treated with PLA1 or PLA2, which release the fluorescent moiety “D” and the quenching moiety “Q” from their close (quenching) proximity. While not intending to be bound by any theory of operation, it is believed that one or both of the resultant fatty acid and lysophospholipid cleavage products comprising the fluorescent or quenching moieties leave the liposome and enter the assay medium. Regardless of the mechanism of action, treatment with PLA1 or PLA2 results in an increase in fluorescence of the sample.

Another exemplary embodiment, in which the ligand molecule comprises a fluorescent moiety, is illustrated in FIG. 18D. In FIG. 18D, the ligand molecule corresponds to the dual role ligand/signal molecule 700 of FIG. 15B. As in the embodiment illustrated in FIG. 18C, following binding and removal of unbound micelles, treatment of the micelle with a phospholipase such as PLA1, PLA2, PLC or PLD, leads to unquenching of the fluorescent moiety and an increase in fluorescence of the sample. In instances where the binding moiety of dual role ligand/signal molecule 700 is net hydrophobic in character, the cleavage product could potentially form micelles, causing quenching of their fluorescent moieties and quenching of its fluorescence signal of the assay. To avoid such quenching of the assay signal, in many embodiments it may be preferable to utilize ligand/signal molecules 700 in which both the binding and fluorescent moieties are net hydrophilic in character.

In still another exemplary embodiment, illustrated in FIG. 18E, the micelle comprises a signal molecule, a ligand molecule and a quenching molecule. The signal molecule corresponds in structure to the signal molecule 100 of FIG. 1A, the ligand molecule corresponds in structure to the ligand molecule 600 of FIG. 14A and the quenching molecule corresponds in structure to quenching molecule 800 of FIG. 16, although signal, ligand and quenching molecules having different structures could be used. As illustrated, following binding and removal of unbound micelles, treatment with PLC releases fluorescent moiety “D” and quenching moiety “Q” from their close (quenching) proximity, resulting in an increase in fluorescence of the sample. Other phospholipases, such as PLA1, PLA2 or PLD could also be used with similar results.

An alternative embodiment in which treatment by the modification moiety cleaves the quenching moiety of the quenching molecule but not the ligand or signal molecule is illustrated in FIG. 18F. In this exemplary embodiment, ligand and signal molecules are selected that either do not comprise modification moieties, or that comprise modification moieties that are not modified by the modification moiety that modifies the quenching molecule. As a specific example, the ligand molecule could correspond in structure to ligand molecule 600 of FIG. 14A, the signal molecule could correspond in structure to signal molecule 100 of FIG. 1A, and the quenching molecule could correspond in structure to quenching molecule 850 of FIG. 16B. Following binding and removal of unbound micelles, treatment with protein kinase C releases quenching molecule 850 from the micelle, unquenching the fluorescence of signal molecule 100, resulting in an increase in the fluorescence of the sample.

As illustrated in FIG. 18F, the fluorescence of the micelle becomes unquenched while the micelle is bound to the immobilized binding partner, making micelles of this type ideally suited to applications in which pluralities of compounds are assessed for their ability to bind the binding moiety, as discussed previously. If desired, the fluorescent moiety could be released into the assay medium by treatment with a phospholipase.

Although the exemplary embodiments of FIGS. 18B-18F are illustrated with a single type of micelle, skilled artisans will appreciate that the assays could be carried out with a plurality of micelles, as discussed above for the exemplary assay of FIG. 18A.

Regardless of how the assay is carried out, the ligand-binding molecule preferably is immobilized on a solid substrate. Suitable solid substrates include, but are not limited to, beads, microtiter plates, glasses, silica, ceramics, nylon, quartz wafers, gels, metals, nitrocellulose, gold and paper. The substrates can be flexible or rigid. Preferably, the substrate is non-reactive with the ligand molecule or any component in the ligand-conjugated micelle.

Methods for coupling molecules to a solid support are well known in the art and have been widely used in the making of affinity columns, ELISA assay plates, support-bound peptide and drug candidate libraries and polynucleotide arrays. See, for example, Sigel et al., FEBS LETT. 147: 45-48 (1982). Any of the various chemistries and methodologies can be used to immobilize binding molecules or putative binding molecules. The ligand-binding molecule can be stably attached to a solid substrate by covalent and/or non-covalent interactions. For instance, the ligand-binding molecule can be covalently deposited to the surface of a solid support via cross-linking agents, such as glutaraldehyde, borohydride, or other bifunctional agents. The ligand-binding molecule may also be covalently linked to the substrate via an alkylamino-linker group or a polymer linker. The polymer linkers may improve the accessibility of the ligand-binding molecule to the ligand. Preferred coupling methods should not substantially affect the binding specificity and/or affinity between the ligand and the ligand-binding molecule.

The binding assay taught herein typically comprises the use of a buffer, such as a buffer described in the “Biological Buffers” section of the 2003 Sigma-Aldrich Catalog. Exemplary buffers include sodium phosphate, sodium acetate, PBS, MES, MOPS, HEPES, Tris (Trizma), bicine, TAPS, CAPS, and the like. The buffer is present in an amount sufficient to generate and maintain a desired pH and/or ionic strength. The pH of the binding buffer can be selected according to the pH dependency of the binding activity. For example, the pH can be from 2 to 12, from 4 to 11, or from 6 to 10. The buffer may also contain any necessary cofactors or agents required for binding and/or for the modification agent (e.g. Ca²⁺ ion). The identities and concentration of such cofactors and/or agents will depend upon the particular assay system and will be apparent to those of skill in the art. The concentration of the ligand-containing micelles in the binding assay may vary substantially. For example, the assay buffer can comprise from about 1 pM to 1 mM ligand-containing micelles. In some embodiments, the assay buffer comprises from about 1 pM to 1 μM ligand-containing micelles. If a plurality of different types of ligand-containing micelles is used, each may comprise in the assay buffer in the above concentration ranges.

The binding assay typically does not require the presence of detergents or other components. In general, it is desirable to avoid high concentrations of components in the reaction mixture that can adversely affect the fluorescence properties of the reaction product, or that can interfere with the analysis of modulators, such as described herein below.

Following binding, the unbound micelles are removed, typically by washing the sample with one or more volumes of buffer. As for the binding assay buffer, the washing buffer should comprise any cofactors and/or agents required for the binding interaction.

After removal of unbound micelles, the sample is treated with the appropriate modification agent(s). The modification agent can be added directly to the sample if it includes any cofactors and/or agents required for activity, or, alternatively, it can be added in a buffer system including such cofactors and/or agents. The amount of modification agent added is not critical and may depend upon a variety of factors, including, for example, the amount or quantity of bound micelles in the sample. An appropriate amount of modification agent to add to a particular application can be readily determined empirically.

In the methods described herein, the fluorescence signal can be monitored using conventional methods and instruments. In certain embodiments, a multiwavelength fluorescence detector can be utilized. The detector can be used to excite the fluorescent labels at one wavelength and detect emissions at multiple wavelengths, or excite at multiple wavelengths and detect at one emission wavelength. Alternatively, the sample can be excited using “zero-order” excitation in which the full spectrum of light (e.g., from xenon lamp) illuminates the cuvette. Each fluorescent moiety can absorb at its characteristic wavelength of light and then emit maximum fluorescence. The multiple emission signals can be monitored independently. Preferably, a suitable detector can be programmed to detect more than one excitation emission wavelength substantially simultaneously, such as that commercially available under the trade designation HP1100 (G1321A), from Hewlett Packard, Wilmington, Del. Thus, the signal molecule can be detected at programmed emission wavelengths at various intervals during a reaction.

Detection of fluorescent signal can be performed in any appropriate way. Advantageously, the micelles and methods can be used in a continuous monitoring phase, in real time, to allow the user to rapidly determine whether there is a binding event between the ligand and the ligand-binding molecule. The fluorescent signal can be measured from at least two different time points, usually before and after the modification by the specified agent.

Alternatively, the fluorescent signal can be measured in an end-point embodiment in which a signal is measured after a certain amount of time, and the signal is compared against a control signal (e.g., before start of the modification), threshold signal, or standard curve.

The teachings described herein contemplate not only detecting binding interactions, but also methods involving: (1) screening for, identifying and/or quantifying binding compounds in a sample, (2) determining dissociation constants with respect to selected binding partners, (3) detecting, screening for, identifying and/or characterizing inhibitors, activators, and/or modulators of binding interactions, and (4) determining binding specificities and/or binding consensus sequences or binding consensus structures for selected molecules.

For example, in screening for binding activity, a sample that contains, or may contain, a known or candidate binding compound is mixed with a binding substrate. Following removal of unbound micelles, the fluorescence is measured to determine whether an increase in fluorescence has occurred. Screening may be performed on numerous samples simultaneously in a multi-well or multi-reaction plate or device to increase the rate of throughput. The dissociation constant (Kd) of the interaction may be determined by standard methods.

In some embodiments, the assay mixture may contain two or more different candidate compounds. This may be useful, for example, to screen multiple candidates simultaneously to determine if at least one of the candidate compounds binds the binding moiety.

In other embodiments, the assay mixture may contain two or more different binding substrates. This may be useful, for example, to screen multiple binding moieties simultaneously to determine if at least one of the binding moieties binds a compound of interest in the sample.

In assays employing different binding substrates, each different substrate may be tested separately in different assay mixtures, or two or more substrates may be present simultaneously in a reaction mixture. In embodiments in which the different substrates are present simultaneously in the reaction mixture, the substrates can contain the same fluorescent moiety, in which case the observed fluorescent signal is the sum of the signals from binding with both substrates. Alternatively, the different substrates can contain different, fluorescently distinguishable fluorescent moieties that allow separate monitoring and/or detection of binding with each different substrate simultaneously in the same mixture. The fluorescent moieties can be selected such that all or a subset of them are excitable by the same excitation source, or they may be excitable by different excitation sources. They can also be selected to have additional properties, such as, for example, the ability to quench one another when in close proximity thereto, by, for example, orbital overlap, collisional quenching, FRET or another mechanism (or combination of mechanisms).

In some embodiments, assays carried out with a plurality of different binding substrates may utilize pre-formed micelles, each composed of a different binding substrate.

Detecting, screening for, identifying and/or characterizing inhibitors, activators, and/or modulators of binding interactions can be performed by forming assay mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant) and the like to characterize the concentration dependence of the effect that such substances have on binding activity.

6.4.1 Kits

Also provided are kits for making the ligand-containing micelles and/or for carrying out the various methods described herein. In some embodiments, the kit comprises a ligand molecule, a signal molecule and a modification agent. The kit may optionally comprise a quenching molecule and/or additional components for making the ligand-containing micelles. In some embodiments, the ligand molecule, signal molecule and optional quenching molecule and/or other components are packaged in a form such that they can be used to make ligand-containing micelles. In some embodiments, the ligand molecule, signal molecule and optional quenching molecule and other components are provided in a kit in the form of pre-formed lyophilized micelles that can be reconstituted for use, or in the form of pre-formed micelles in solution.

In other embodiments, the kit may optionally comprise a charge balance molecule and/or additional components for making the ligand-containing micelles. In some embodiments, the ligand molecule, signal molecule and optional charge balance molecule and/or other components are packaged in a form such that they can be used to make ligand-containing micelles. In some embodiments, the ligand molecule, signal molecule and optional charge balance molecule and other components are provided in a kit in the form of pre-formed lyophilized micelles that can be reconstituted for use, or in the form of pre-formed micelles in solution.

The kit may also comprise a binding assay buffer, or a component thereof. The buffer may be provided in a container in dry or liquid form. The choice of a particular buffer may depend on various factors, such as the pH optimum for the binding reaction, and the solubility and fluorescence properties of the fluorescent moiety of the amphiphilic molecule. In some embodiments, the buffer is provided as a stock solution having a pre-selected pH and buffer concentration. Upon mixture with the sample, the buffer produces a final pH that is suitable for the binding or modulator assays, as discussed above. In addition, the kit may comprise other components that are beneficial to the activity of the modification agent, such as salts (e.g., KCl, NaCl, or NaOAc, CaCl₂, MgCl₂, MnCl₂, ZnCl₂) and/or other components that may be useful for a particular assay. These other components can be provided separately from each other, such as in separate containers, or mixed together in dry or liquid form.

Unless defined otherwise, all technical and scientific terms used herein have the same meaning as commonly understood by one of ordinary skill in the art to which compositions and methods belong. Unless mentioned otherwise the techniques employed or contemplated herein are standard methodologies well known to one of ordinary skill in the art. The materials, methods and examples are illustrative only and not limiting.

All numerical ranges in this specification are intended to be inclusive of their upper and lower limits.

All patent applications, patents, and literature references cited in this specification are hereby incorporated by reference in their entirety. In case of conflict or inconsistency, the present description, including definitions, will control.

### 7. EXAMPLES

### 7.1 Preparation of 1,2-dioleoyl-sn-glycero-3-phosphoethanolamine-N-(Oregon green 488, 5-isomer)

### Compound 57; FIG. 3B

Referring to FIG. 3B, Oregon green 488 carboxylic acid, succinimidyl ester, 5-isomer (compound 55; 25 mg, 49 μmol, Molecular Probes Product Number O-6147) was dissolved in dry DMF (1 ml) and added to 1,2-dioleoyl-sn-glycero-3-phosphoethanolamine (“DOPE”; compound 53; Avanti Polar Lipids Product Number 850725, 24 mg, 33 μmol) dissolved in dichloromethane (1 ml) with added triethylamine (46 μl, 330 μmol). After 15 min the solvent was evaporated and the residue was dissolved in aqueous triethylammonium acetated buffer (“TEAA,” 20 ml, 2 M). The crude product was purified by reverse phase C18 HPLC eluting with a mixture of methanol and 100 mM TEAA (90:10 to 95:5). Pure fractions were combined, concentrated and desalted with a short plug of C18 reverse phase media to afford a yellow solid (17 mg, 13 μmol, 39%).

### 7.2 Preparation of 100 nm Monodisperse Ligand-Containing Liposomes

Large unilamellar vesicles (LUV) of diameter 100 nm containing signal molecule 57 and a biotin-containing ligand molecule 604 (FIG. 14C) were prepared by the extrusion method (see Subroto Chatterjee and Dipak K. Banerjee in Methods in Molecular Biology: Liposome Methods and Protocols, Ed. by S. Basu and M. Basu, Humana Press, 2002, vol. 199, chapter 1). For example, DOPC (12 mg, 15 μmol, Avanti Polar Lipids Product Number 850375), cholesterol (1 mg, 2 μmol, Avanti Polar Lipids Product Number 700000), biotin-PEG₂₀₀₀DSPE (compound 604, 3 mg, 1 μmol, Northern Lipids, Inc. Product Number AL-044) and compound 57 (3 mg, pH 7.2) were dissolved in chloroform (5 ml) in a 25 ml recovery flask. The solvent was thoroughly evaporated under high vacuum to leave a thin film. Aqueous PBS buffer (2 ml, pH 7.2) was added and the suspension was subjected to five cycles of freezing (−78° C., dry ice acetone bath) and thawing (40° C.) to hydrate the lipids. The resulting LUVs were extruded ten times through 2 stacked 100 nm polycarbonate membranes (Nuclepore tarck-etch membrane, Whatman Product Number 110605) using a Lipex™ Extruder (Northern Lipids, Inc., British Columbia, Canada, Product Number T.001). The LUVs were purified by Sephadex™ GM-25 gel filtration (PD-10 column, Amersham Biosciences Product Number 17-0851-01) eluting with PBS. The vesicle size and dispersity was determined by dynamic light scattering using a Nicomp 370 particle size analyzer (Lee Miller, Fine Particle Technology, Menlo Park, Calif.).

### 7.3 Liposome Biosensor on a Biacore CM5 Chip

A solution of 1-[3-(dimethylamino)propyl]-3-ethylcarbodiimide hydrochloride (200 mM, Aldrich part #16, 146-2) and N-hydroxysuccinimide (20 mM, Aldrich part #13,067-2) in running buffer (100 mM HEPES/150 mM NaCl, pH 7.4) was flowed over a CM5 chip (Biacore part #99-1000BR) in a Biacore 3000 instrument for 7 min (flow rate 5 μl/min). A solution of streptavidin (0.5 nM) in running buffer was flowed over the activated CM5 chip for 7 min (10 μl/min) to immobilize the protein. A solution of ethanolamine (1 M) in sodium borate buffer (pH 8.5, 100 mM) was flowed over the CM5 chip for 7 min (10 μl/min) to deactivate it. The Biacore 3000 response was 21,425 RU which indicated efficient immobilization of streptavidin to the CM5 chip.

A solution of the biotinylated liposomes from Section 6.2 (1 nM) in running buffer were bound to the immobilized streptavidin by flowing the solution over the CM5 chip for 5 min (10 μl/min). Running buffer was flowed over the CM5 chip for 10 min (10 μl/min) to wash away unbound liposomes. The Biacore 3000 response increased to 21,600 RU (Δ=175 RU) indicating efficient binding of biotinylated liposomes. A solution of phospholipase C (1 μM, Sigma part #P7147) in running buffer was flowed over the CM5 chip for 5 min (10 μl/min) to cleave the liposomes. The Biacore 3000 response decreased to 21,425 RU (Δ=0 RU) indicating efficient cleavage of the liposomes.

### 7.4 Liposome Biosensor Using Magnetic Bead Assay

Streptavidin coated magnetic beads (1 mg, Dynabeads M-280, part #112.05) were placed in a 1.5 ml vial and washed twice with PBS buffer. A solution of biotinylated liposomes from Section 6.2 (1 nM, 0.5 ml) in PBS was added to the beads and left overnight at 0° C. The liposome solution was removed and the beads were washed three times with PBS. The streptavidin beads were split into two portions. One portion was treated with PBS (0.5 ml) and the other was treated with a solution of phospholipase C (0.5 ml, 1 μM, Sigma part #P7147) in PBS. After 6 hr the supernatants were removed and the fluorescent intensities were measured using a PerkinElmer LS50. The phospholipase C treated solution had a fluorescent intensity 300 fold greater than the PBS treated solution.

### 7.5 Preparation of Compound 7, FIG. 6B

A prophetic example for the synthesis of compound 7 is illustrated in FIGS. 6A-6B. Referring to FIG. 6A, bromo 2,3,4,6-tetra-O-acetyl-α-D-galactopyranoside (4.0 g, 24 mmol, Toronto Research Chemicals catalogue #B687000) and 4-hydroxy-3-nitrobenzaldehyde (10 g, 24 mmol, Aldrich catalogue #14, 432-0) can be dissolved in acetonitrile (200 ml). Silver (I) oxide (25 g, 108 mmol) can be added and the suspension stirred at room temperature for 3 hours. The reaction mixture can be filtered with suction through a pad of celite, the filtrate collected and the solvent evaporated. The crude product can be purified by silica gel chromatography eluting with a 98:2 mixture of dichloromethane (DCM) and methanol (MeOH). A pale yellow foam (1, 10 g, 20 mmol, 83%) can be obtained after collecting the fractions and evaporating the solvent.

Compound 1 (3.4 g, 6.8 mmol) can be dissolved in DCM (150 ml). The solution can be sparged with argon for 10 min and then 10% Pd/C (0.5 g) can be added. The flask can be charged with hydrogen and shaken with a Parr apparatus. After 3 hr the reaction mixture can be filtered with suction through a pad of celite The filtrate can be concentrated and the crude product can be purified by silica gel chromatography eluting with a 98:2 mixture of DCM and MeOH. A colorless foam (2, 2.5 g, 5.3 mmol, 78%) can be obtained after collecting the fractions and evaporating the solvent.

Compound 2 (2.9 g, 6.2 mmol) can be dissolved in dry dimethylformamide (DMF, 20 ml). Imidazole (0.63 g, 9.3 mmol) and tert-butyldimethylsilyl chloride (1.4 g, 9.3 mmol) can be added. After 30 min most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless oil (3, 4.5 g, 7.7 mmol, 67%) can be obtained after collecting the fractions and evaporating the solvent.

Compound 3 (4.5 g, 7.7 mmol) and myristic acid (1.8 g, 7.7 mmol) can be dissolved in DMF (20 ml). N,N-diisopropylethylamine (DIPEA, 0.99 g, 7.7 mmol) can be added followed by N-[(dimethylamino)-1H-1,2,3-triazolo[4,5-b]pyridin-1-ylmethylene]-N-methylmethanaminium hexafluorophosphate N-oxide (HATU, 2.9 g, 7.7 mmol). After 30 min most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless solid (4, 4.8 g, 6 mmol, 78%) can be obtained after collecting the fractions and evaporating the solvent.

Compound 4 (2.4 g, 3 mmol) can be dissolved in a solution of HCl in MeOH (60 mM, 16.7 ml, 1 mmol HCl). After 30 min the acid can be neutralized with NaHCO₃ (84 mg, 1 mmol) in water (3 ml). Most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless solid (compound 5, 1.6 g, 2.4 mmol, 79%) can be obtained after collecting the fractions and evaporating the solvent.

Compound 5 (16 mg, 23 μmol) can be dissolved in warm acetonitrile (2 ml). N,N′-disuccinimidyl carbonate (DSC, 6 mg, 23 μmol) and DIPEA (6 mg, 8 μl, 46 μmol) can then be added. After 1 h 5-(aminomethyl)fluorescein hydrochloride (9 mg, 23 μmol) can be added. The crude product 6 can be used in the next step.

Ammonium hydroxide solution (15 M, 1 ml) can be added to the above crude product 6 and left to sit overnight. The reaction mixture can be diluted with water (18 ml) and purified by reverse phase HPLC eluting with a 2:3 mixture of triethylammonium acetate buffer (100 mM) and methanol. Fractions can be combined and most of the solvent evaporated. The product can be desalted on a short plug of C18 reverse phase media. The product should be obtained as an orange solid (7, 5 mg, 5 μmol, 21%).

### 7.5 Preparation of Compound 4, FIG. 6C

Referring to FIG. 6C, 4-Hydroxymandelic acid (Aldrich catalogue #16, 832-7) can be coupled with 1-tetradecylamine under standard peptide coupling conditions to yield amide 1. The phenolic hydroxyl group can be selectively glycosylated under Koenig-Knorr conditions to give β-glycoside 2. The benzylic hydroxyl group of compound 2 can be reacted with N,N′-disuccinimidyl carbonate (DSC) or other phosgene synthetic equivalent to give the mixed carbonate. 5-Aminomethyl fluorescein (Molecular Probes catalogue #A-1353) can be coupled with the mixed carbonate under basic conditions to give carbamate 3. The four acetate protecting groups on the sugar can be hydrolysed with catalytic sodium methoxide in methanol to give compound 4.

### 7.6 Preparation of compound 5, FIG. 6D

Referring to FIG. 6D, 5-Formylsalicylic acid (Aldrich catalogue #F1, 760-1) can be coupled with 1-tetradecylamine under peptide coupling conditions to give amide 1. The phenolic hydroxyl group can be glycosylated under Koenig-Knorr conditions to give β-glycoside 2. The benzaldehyde group can be reduced under catalytic hydrogenation conditions to give compound 3. The benzylic hydroxyl group of compound 3 can be reacted with N,N′-disuccinimidyl carbonate (DSC) or other phosgene synthetic equivalent to give the mixed carbonate. 5-Aminomethyl fluorescein (Molecular Probes catalogue #A-1353) can be coupled with the mixed carbonate under basic conditions to give carbamate 4. The four acetate protecting groups on the sugar can be hydrolysed with catalytic sodium methoxide in methanol to give compound 5.

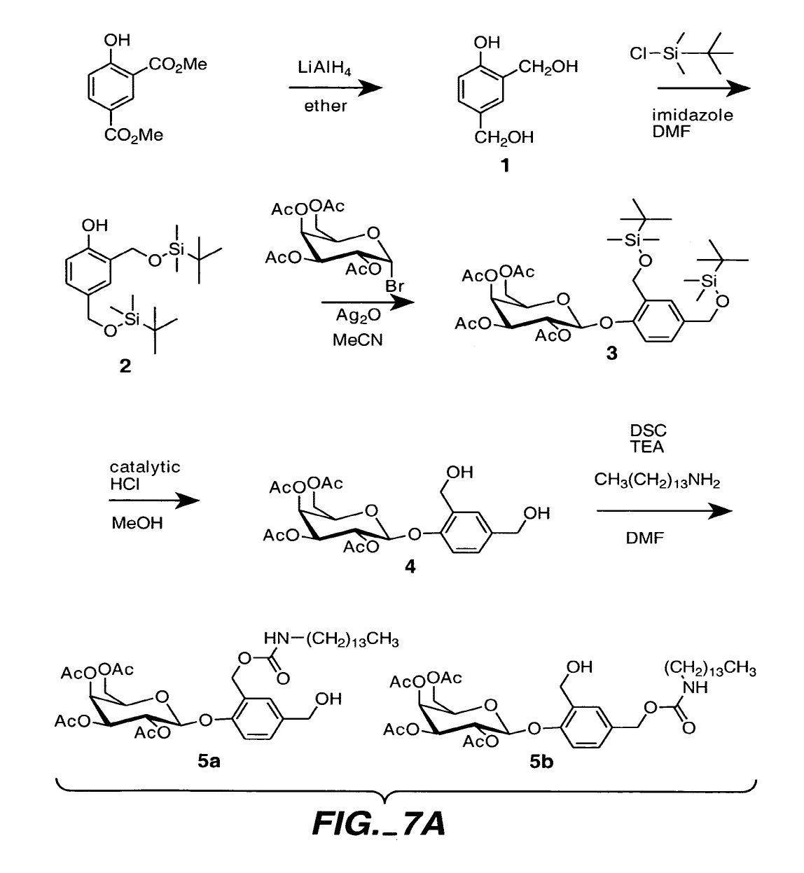

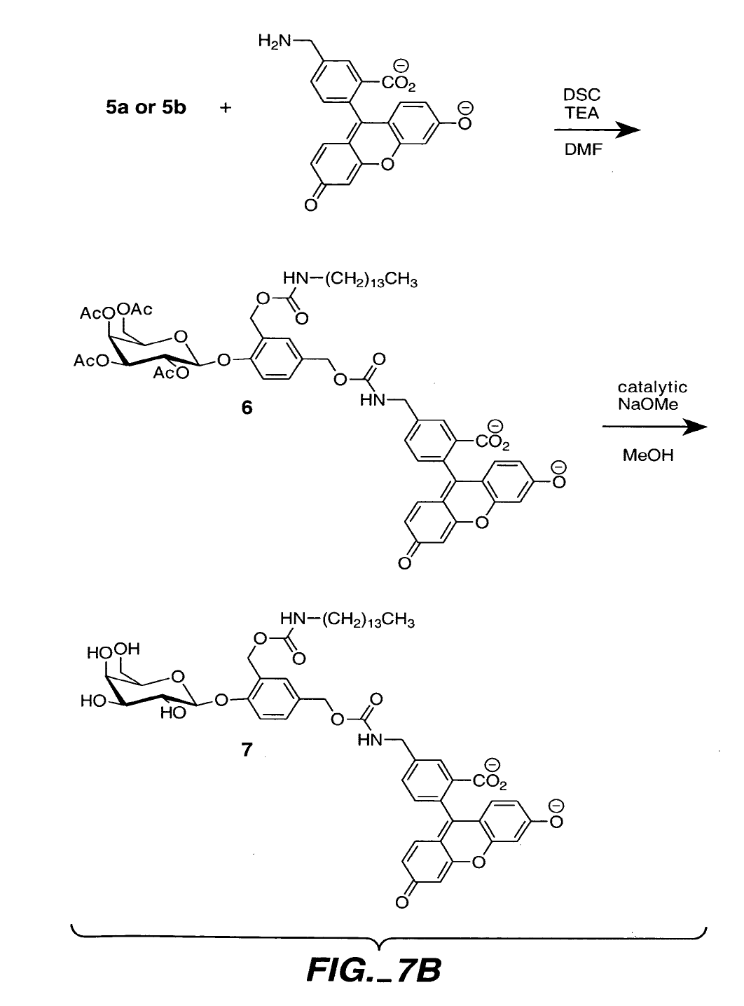

### 7.7. Preparation of compound 7, FIG. 7

Referring to FIG. 7A, dimethyl 4-hydroxyisophthalate (Aldrich catalogue #541095) can be reduced with lithium aluminum hydride to give the triol 1. The benzylic alcohols can be selectively protected with tert-butyldimethylsilyl chloride to give compound 2. The phenol can be glycosylated under Koenig-Knorr conditions to give β-glycoside 3. The silyl protecting groups can be hydrolysed with catalytic hydrochloric acid in methanol to give diol 4. One equivalent of N,N′-disuccinimidyl carbonate (DSC) or other phosgene synthetic equivalent can be added to compound 4 to give a mixture of two regioisomeric monocarbonates. 1-Tetradecylamine can be added to the mixture of monocarbonates to give a mixture of regioisomeric monocarbamates 5a,b. The regioisomers may be separated by chromatography if desired. One equivalent of N,N′-disuccinimidyl carbonate (DSC) or other phosgene synthetic equivalent can be added to compound 5 to give a mixed carbonate. 5-Aminomethyl fluorescein (Molecular Probes catalogue #A-1353) can be coupled with the mixed carbonate under basic conditions to give carbamate 6. The four acetate protecting groups on the sugar can be hydrolysed with catalytic sodium methoxide in methanol to give compound 7.

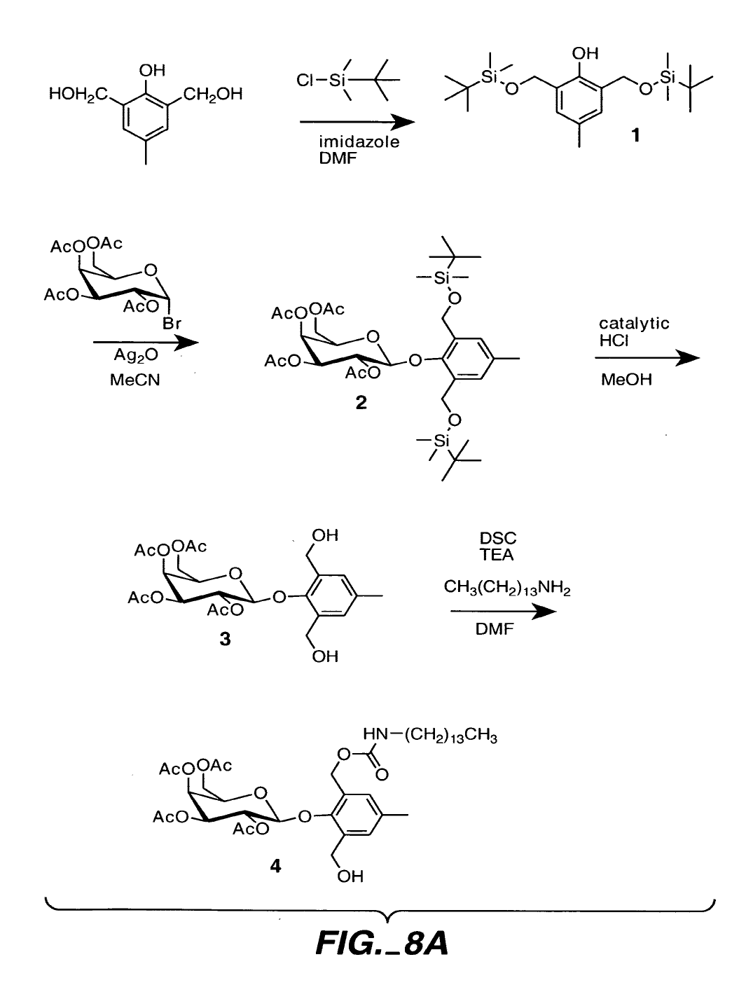

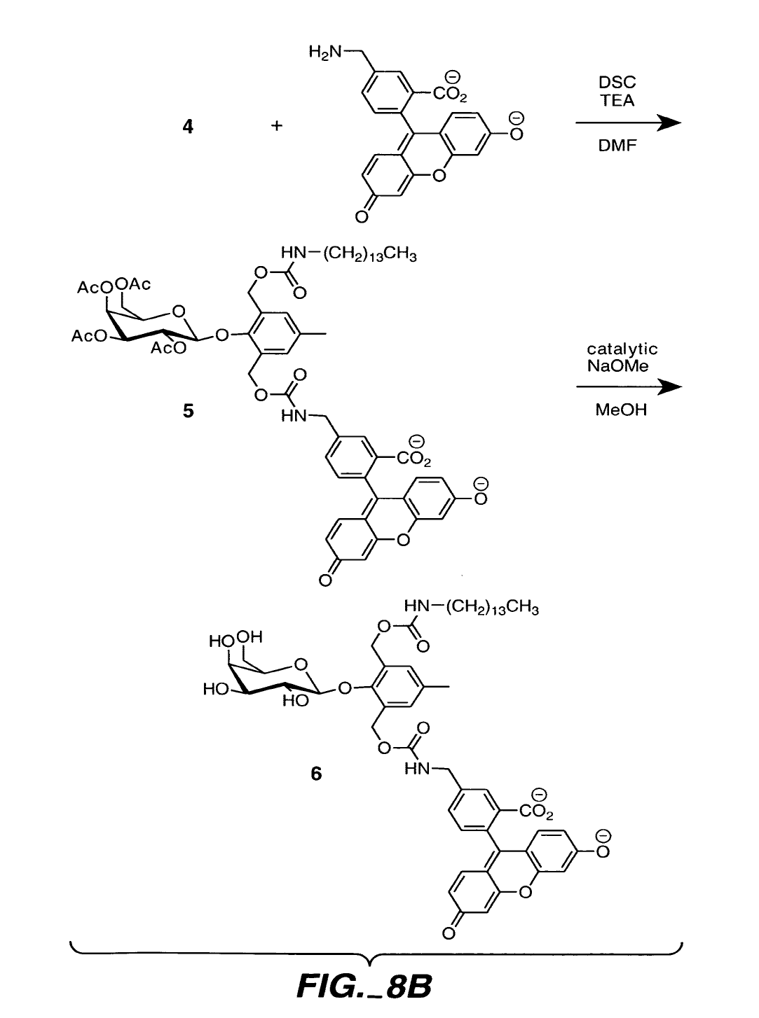

### 7.8 Preparation of compound 6, FIG. 8B

Referring to FIG. 8A, 2,6-Bis(hydroxymethyl)-p-cresol (Aldrich catalogue #22, 752-8) can be selectively protected with two equivalents of tert-butyldimethylsilyl chloride to give 1. The phenol can be glycosylated under Koenig-Knorr conditions to give β-glycoside 2. The silyl protecting groups can be hydrolysed with catalytic hydrochloric acid in methanol to give diol 3. One equivalent of N,N′-disuccinimidyl carbonate (DSC) or other phosgene synthetic equivalent can be added to compound 3 to give a mixed carbonate. 1-Tetradecylamine can be added to the mixed carbonate under basic conditions to give carbamate 4. One equivalent of N,N′-disuccinimidyl carbonate (DSC) or other phosgene synthetic equivalent can be added to compound 4 to give a mixed carbonate. 5-Aminomethyl fluorescein (Molecular Probes catalogue #A-1353) can be coupled with the mixed carbonate under basic conditions to give carbamate 5. The four acetate protecting groups on the sugar can be hydrolysed with catalytic sodium methoxide in methanol to give compound 6.

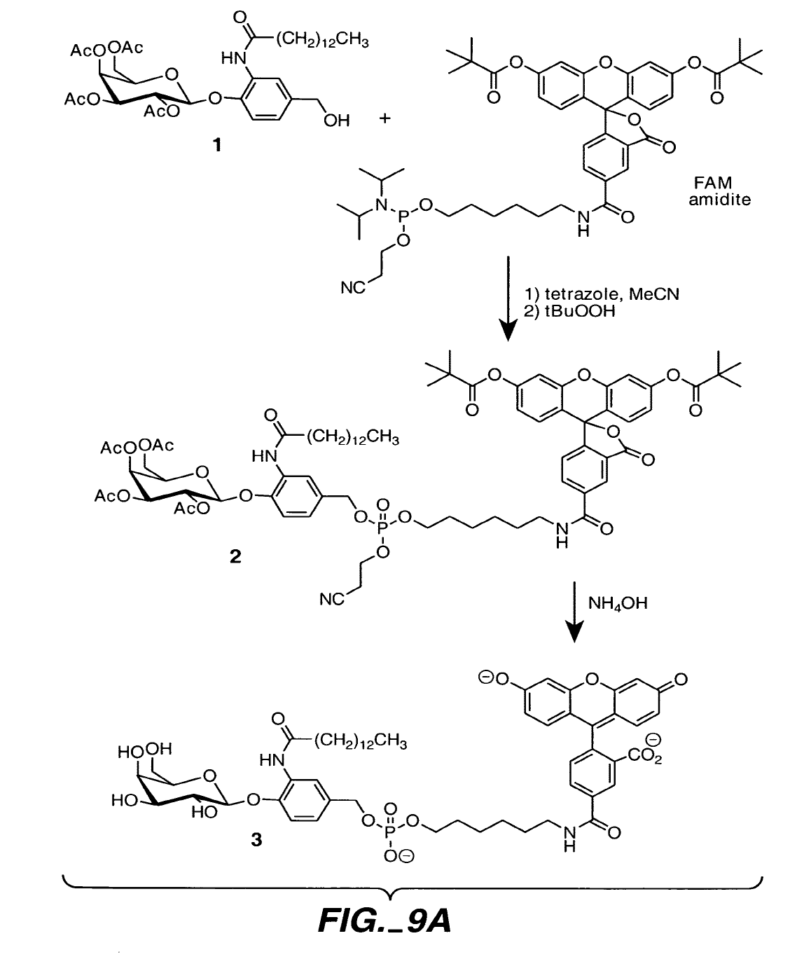

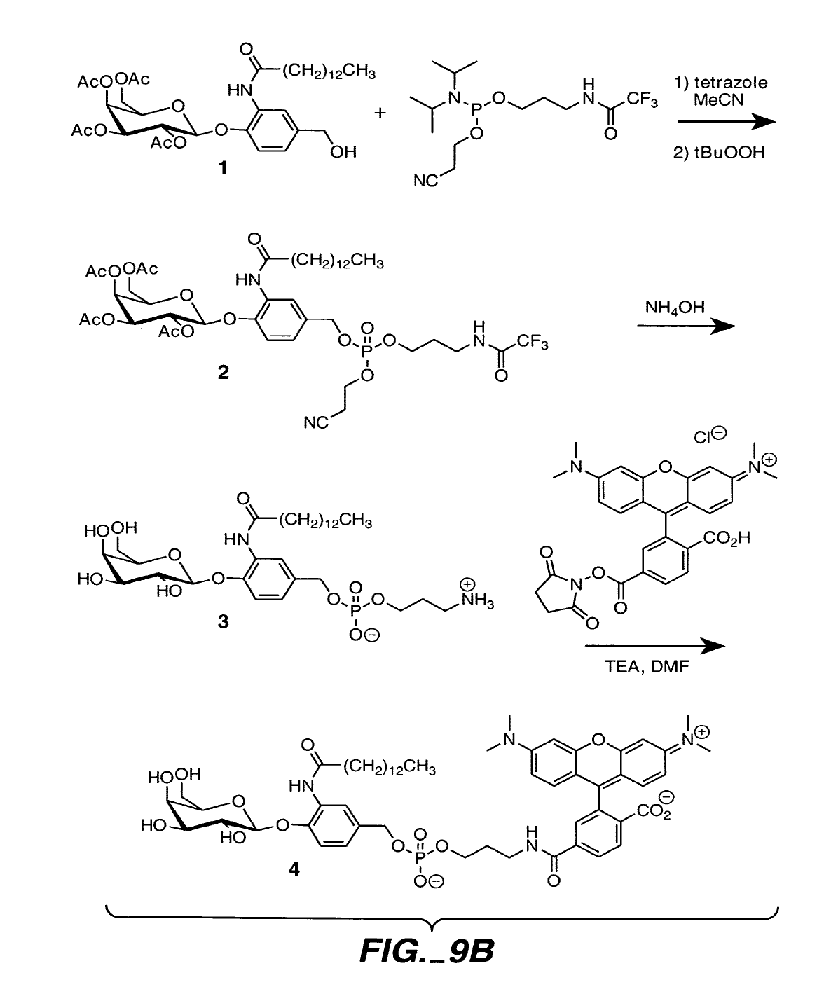

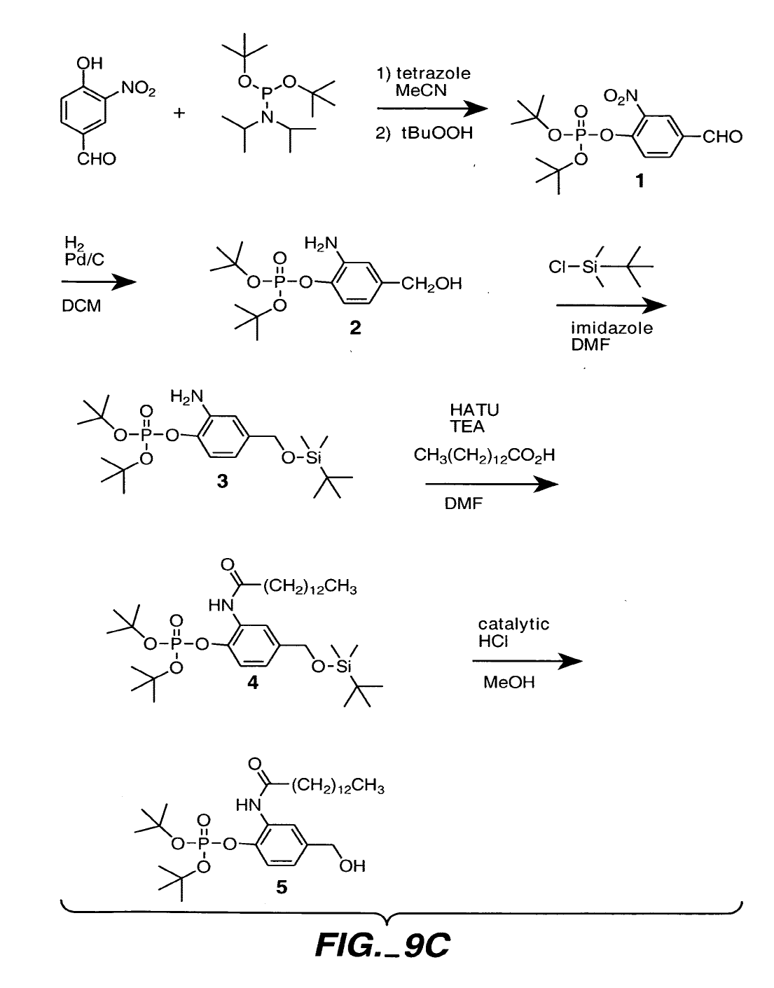

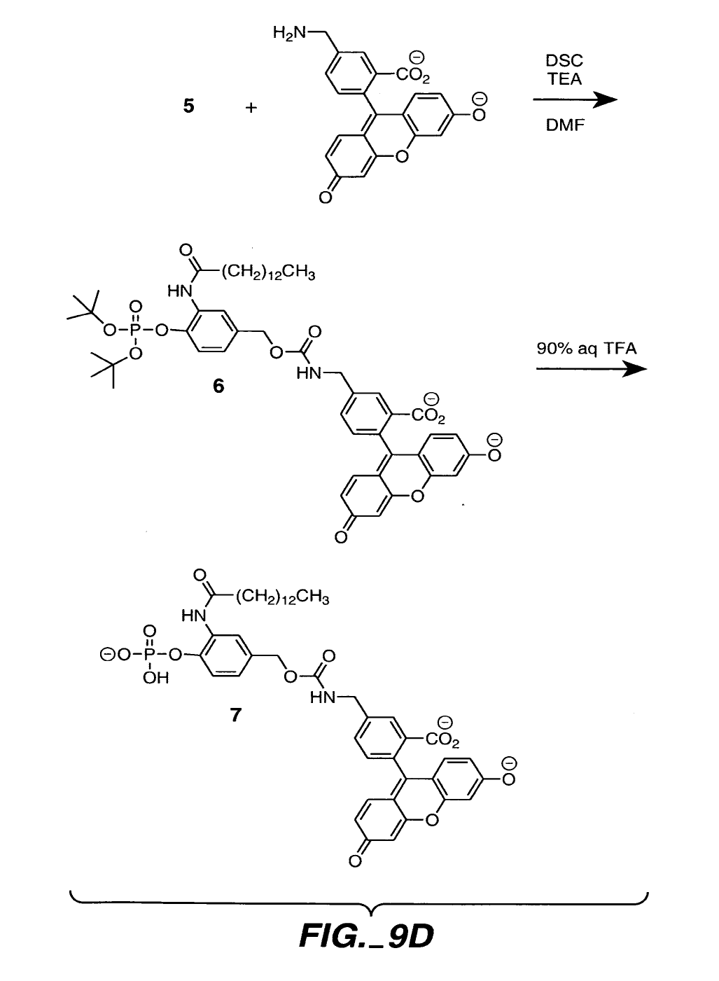

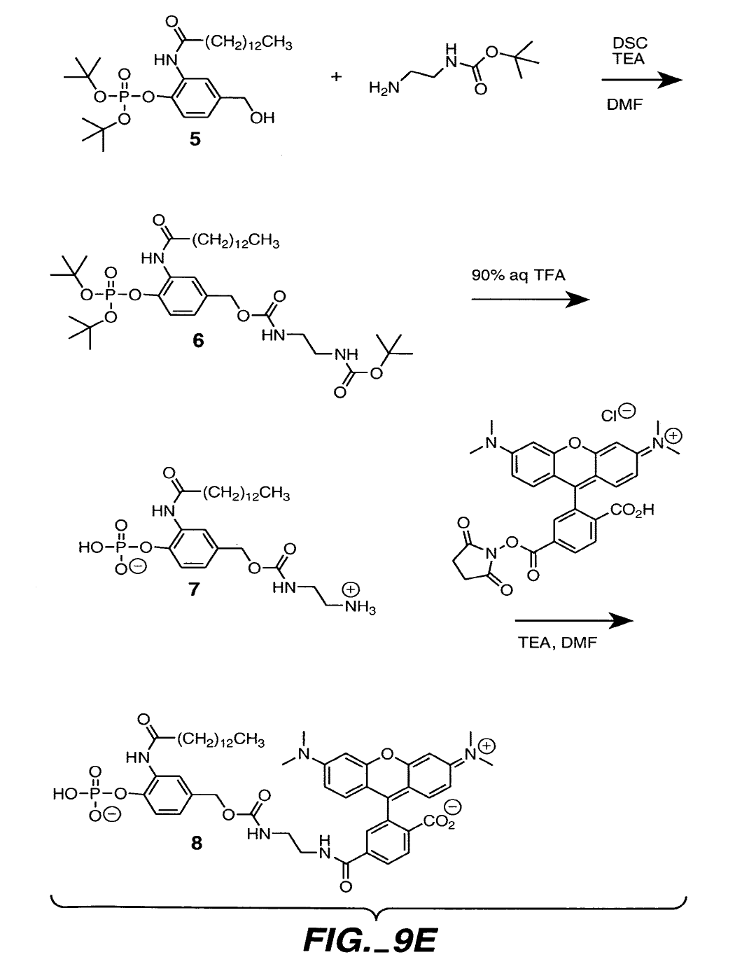

### 7.9 Preparation of compound 3, FIG. 9A

Referring to FIG. 9A, the benzylic alcohol of compound 1 can be reacted with FAM® phosphoramidite (Applied Biosystems catalogue #401527) under standard tetrazole coupling conditions. The phosphite can be oxidized with tert-butylhydroperoxide to give the phosphate 2. Concentrated ammonium hydroxide can be used to cleave the cyanoethyl, four acetyl, and two pivaloyl protecting groups to give compound 3.

### 7.10 Preparation of Compound 4, FIG. 9B

Referring to FIG. 9B, Compound 1 can be reacted with TFA aminolink phosphoramidite (Applied Biosystems catalogue #402872) under standard tetrazole conditions. The phosphite can be oxidized with tert-butylhydroperoxide to give phosphate 2. Concentrated ammonium hydroxide can be used to cleave the trifluoroacetyl, cyanoethyl, and four acetyl protecting groups to give 3. Carboxytetramethylrhodamine succinimidyl ester (Molecular Probes catalogue #C2211) can be coupled to the primary amine under basic conditions to give 4.

### 7.11 Preparation of compound 7, FIG. 9C

Referring to FIG. 9C, 4-Hydroxy-3-nitrobenzaldehyde (Aldrich catalogue #14, 432-0) can be reacted with di-tert-butyl-N,N-diisopropylphosphoramidite (Novabiochem catalogue #01-60-0031) to give a phosphite that can be subsequently oxidized to the phosphate with tert-butylhydroperoxide. The benzaldehyde and nitro groups of compound 1 can be reduced under catalytic hydrogenation conditions to give the aminoalcohol 2. The hydroxyl group can be protected as its tert-butyldimethylsilyl ether. Myristic acid can be coupled with the aniline under standard peptide coupling conditions to give 4. The silyl ether protecting group can be hydrolyzed with catalytic hydrochloric acid in methanol to give 5. The benzyl alcohol can be reacted with DSC or other phosgene synthetic equivalent to give the mixed carbonate. 5-Aminomethyl fluorescein (Molecular Probes catalogue #A-1353) can be added under basic conditions to give the carbamate 6. The two tert-butyl protecting groups on the phosphate can be hydrolysed with 90% aqueous trifluoroacetic acid to give 7.

### 7.12 Preparation of compound 8, FIG. 9E

Referring to FIG. 9E, the benzyl alcohol of compound 5 can be reacted with DSC or other phosgene synthetic equivalent to give the mixed carbonate. N-Boc-ethylenediamine (Fluka catalogue #15369) can be added under basic conditions to give the carbamate 6. The two tert-butyl and boc protecting groups can be hydrolysed with 90% aqueous trifluoroacetic acid to give 7. Carboxytetramethylrhodamine succinimidyl ester (Molecular Probes catalogue #C2211) can be coupled to the primary amine under basic conditions to give 8.

### 7.13 Preparation of compound 13, FIG. 10B

Referring to FIGS. 10A-10B, compound 1 can be reacted with methyl 3,3-dimethylacrylate in methanesulfonic acid to give compound 2. Reduction of 2 with lithium aluminum hydride can give the diol 3. The phenol and alkyl alcohol can be protected with tert-butyldimethylsilyl chloride and imidazole to give 4. The aniline group can be reacted with myristic acid under standard peptide coupling conditions to give amide 5. Selective hydrolysis of the phenolic silyl ether can be performed under basic conditions to give 6. Phosphorylation of 6 with tetrabenzyl pyrophosphate and potassium tert-butoxide can give 7. The alkyl silyl ether can be hydrolysed with catalytic acid in methanol to give 8. Oxidation of the alcohol with Jones reagent in acetone can give 9. Coupling of mono BOC protected ethylenediamine with 9 can be performed under standard peptide coupling conditions. Catalytic hydrogenation of 10 can cleave the benzyl protecting groups on the phosphate. Trifluoracetic acid treatment of 11 can cleave the BOC protecting group to give 12. Tetramethylrhodamine succinimidyl ester can be coupled with 12 under basic conditions to give the final product 13.

### 7.14 Preparation and Use of Substrate Molecules and Charge-Balance Molecules

Resins and reagents for peptide synthesis, Fmoc amino acids, 5-carboxyfluorescein succinimidyl ester were obtained from Applied Biosystems (Foster City, Calif.). Fmoc-Lys(Mtt)-OH, Fmoc-Ser(OPO(OBzl(OH)—OH and Fmoc-Dpr(ivDde) were obtained from Novabiochem. All other chemicals and buffers were obtained from Sigma/Aldrich.

Peptide synthesis was performed on an Applied Biosystems Model 433A Peptide Synthesizer. HPLC was performed on an Agilent 1100 series HPLC. UV-Vis measurements were performed on a Cary 3E UV-Vis spectrophotometer. MALDI Mass spectral data were obtained on an Applied Biosystems Voyager using cyano-4-hydroxycinnamic acid as matrix material.

An exemplary substrate molecule useful for detecting protein tyrosine kinase Lyn, C₁₆Lys(Dye 2)OOOGluGluIleTyrGlyGluPheNH₂ was prepared as follows. The peptide OOOK(ivDde)GluGluIleTyrGlyGluPhe(Mtt) was constructed via solid phase peptide synthesis using standard FastMoc™ chemistry on 125 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was treated with additional 1 mL portions of 5% TFA until the washes were colorless. The resin was washed with DCM (1 mL). Dodecanoic acid (20 mg), HBTU/HOBT solution (0.1 mL) and diisopropylethylamine (0.04 mL) were added to the resin and the mixture was agitated gently for 20 min. The resin was washed with DMF (5×1 mL) and treated with 10% hydrazine in DMF for ten minutes. 5-Carboxy-2′,7′-dipyridylsulfonefluorescein (10 mg), HBTU/HOBT solution (0.1 mL) and diisopropylethylamine (0.04 mL) were added to the resin and the mixture agitated for 45 minutes. The resin was washed with 8×1 mL DMF, 1×1 mL acetonitrile. The peptide was cleaved from the resin with 1 mL cleavage solution (950 μL TFA, 50 μL water). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 um) using a 30% to 70% gradient over 10 min of 0.1% TFA in acetonitrile vs. 0.1% TFA in water. The dye-labeled peptide was analyzed by MALDI mass spectrometry, which resulted in the expected M/z=2234. The peptide solution was evaporated to dryness, redissolved in water, and quantitated. The extinction coefficient of 5-Carboxy-2′,7′-dipyridylsulfonefluorescein was assumed to be 80,000 cm⁻¹M⁻¹·B.

An exemplary charge-balance molecule C₁₆ArgArgOOOArgArgIleTyrGlyArg PheNH₂ useful for balancing the charge of substrate molecule C₁₆Lys(Dye 2)OOOGluGluIleTyrGlyGluPheNH₂, was prepared as follows. The peptide ArgArgOOOArgArgIleTyrGlyArgPheNH₂ (Mtt) was constructed via solid phase peptide synthesis using standard FastMoc™ chemistry on 125 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was treated with additional 1 mL portions of 5% TFA until the washes were colorless. The resin was washed with DCM (1 mL). Dodecanoic acid (20 mg), HBTU/HOBT solution (0.1 mL) and diisopropylethylamine (0.04 mL) were added to the resin and the mixture was agitated gently for 20 min. The resin was washed with DMF (5×1 mL) and treated with 10% hydrazine in DMF for ten minutes. 5-Carboxy-2′,7′-dipyridylsulfonefluorescein (10 mg), HBTU/HOBT solution (0.1 mL) and diisopropylethylamine (0.04 mL) were added to the resin and the mixture agitated for 45 minutes. The resin was washed with 8×1 mL DMF, 1×1 mL acetonitrile. The peptide was cleaved from the resin with 1 mL cleavage solution (950 μL TFA, 50 μL water). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 um) using a 30% to 70% gradient over 10 min of 0.1% TFA in acetonitrile vs. 0.1% TFA in water. The peptide was analyzed by MALDI mass spectrometry, which resulted in the expected M/z=1952. The peptide solution was evaporated to dryness, redissolved in water, and quantitated.

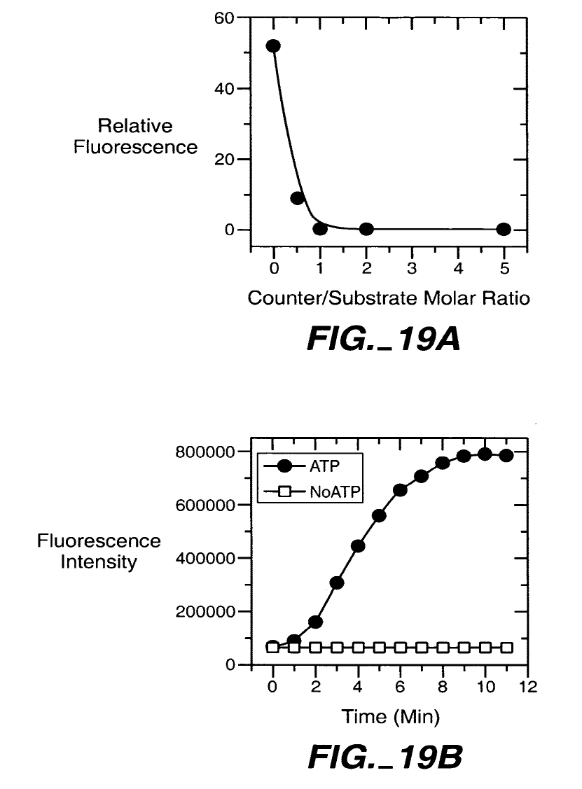

A reaction solution was prepared containing 10 μM substrate molecule C₁₆Lys(Dye 2)OOOGluGluIleTyrGlyGluPheNH₂ and 25 mM Tris (pH 7.6), 5 mM MgCl and 5 mM DTT. Varying concentrations of the charge-balance molecule C₁₆ArgArgOOOArgArgIleTyrGlyArg PheNH₂ were added (final concentration 0, 5 μM, 10 μM, 20 μM, 50 μM) and the fluorescence was determined. The results are shown in FIG. 19A.

Kinase assays were performed using Corning 384-well, black, non-binding surface (NBS), microwell plates. Fluorescence was read in real time using a Molecular Dynamics Gemini XS plate reader, with excitation and emission set at 500 and 550 respectively. The plate was read every minute for one hour at ambient temperature.

Concentrations of the substrate molecule C₁₆Lys(Dye 2)OOOGluGluIleTyrGlyGluPheNH₂ and charge-balance molecule C₁₆ArgArgOOOArgArgIleTyrGlyArg PheNH₂ were determined by dilution of the purified peptides into dimethylformamide (200 μL) with 1 M NaOH (5 μL) and measuring the absorbance of 5-carboxy-2′,7′-dipyridyl-sulfonefluorescein (Dye2) at its absorbance maximum (548 nm). The extinction coefficient of Dye2 was assumed to be 80,000 cm⁻¹M⁻¹.

A reaction solution was prepared containing the substrate molecule C₁₆Lys(Dye 2)OOOGluGluIleTyrGlyGluPheNH₂ (2 mM), and charge-balance molecule C₁₆ArgArgOOOArgArgIleTyrGlyArg PheNH₂ (2 μM), 20 mM Tris buffer, pH 7.6, MgCl₂ (5 mM), DTT (5 mM) and Lyn (5 nM). The solution was pipetted into wells of a 384-well plate (10 mL per well). ATP (0 or 100 μM) was added to initiate the kinase reaction. The plate was read at 500 nm excitation, 550 nm emission, each minute for 1 hour. The results are shown in FIG. 19B.

### 7.15 Detection of Kinase Activity Using a Substrate Compound with Two Hydrophobic Moieties

The substrate compounds were prepared as described in Example 7.14.

Kinase assays were performed using Corning 384-well, black, non-binding surface (NBS), microwell plates. Fluorescence was read in real time using a Molecular Dynamics Gemini XS plate reader, with excitation and emission set at 500 and 550 respectively. The plate was read every minute for one hour at ambient temperature

Concentrations of dye-labeled peptides were determined by dilution of the purified peptides into dimethylformamide (200 μL) with 1 M NaOH (5 μL) and measuring the absorbance of either 5-carboxy-2′,7′-dipyridyl-sulfonefluorescein (i.e. dye2) at its absorbance maximum (548 nm) or 2′,7′,4,7-tetrachloro-5-carboxy fluorescein (i.e. 2′,7′-dichloro-5-carboxy-4,7-dichlorofluorescein or “tet”) at its absorbance maximum (541 nm). The extinction coefficient of both dyes was assumed to be 80,000 cm⁻¹M⁻¹.

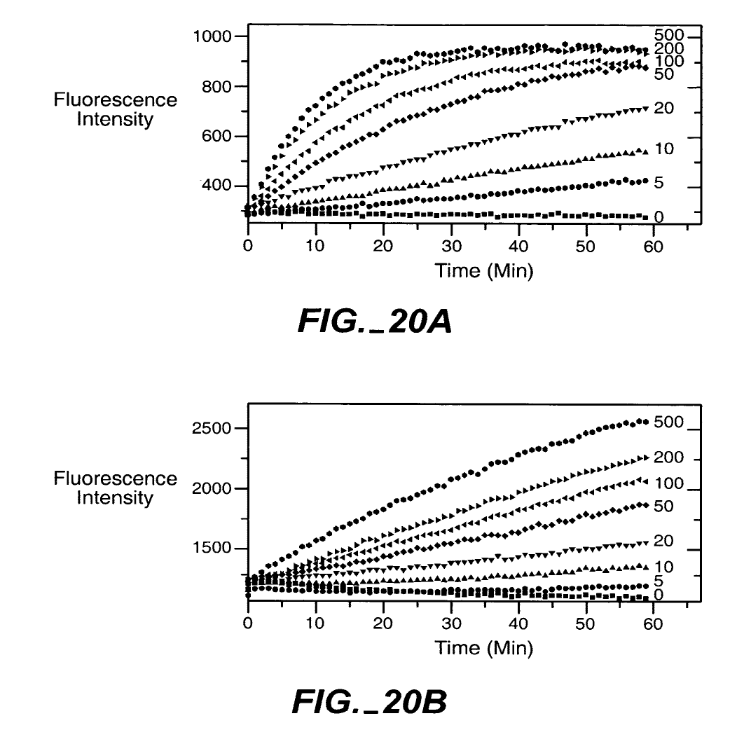

A reaction solution was prepared containing compound 1 (2 mM) 20 mM Tris buffer, pH 7.4, MgCl2 (5 mM), DTT (5 mM) and p38bII (14 nM). The solution was pipetted into wells of a 384-well plate (10 mL per well). Varying concentrations of ATP (final conc 0, 5, 10, 20, 50, 100, 200, 500 mM) were added to the wells to initiate the kinase reaction. The plate was read at 500 nm excitation, 550 nm emission, each minute for 1 hour. The results are shown in FIGS. 20A-20B. The rates of the reaction were fitted to the Michaelis-Menton equation and the apparent Km of ATP calculated to be 90 μM for C₁₂OOK(dye 2)RRIPLSPOOK(C₁₂)NH₂ (FIG. 20A). The same experiment using C₁₆OOOK(dye2)RRIPLSPNH₂ (FIG. 20B) provided an apparent Km of ATP of >200 μM. Thus, the compound with two shorter hydrocarbons, gave a lower Km of ATP than the same sequence with a single hydrocarbon.

### 7.16 Detection of Kinase Activity Using a Substrate Compound with Two Recognition Sequences

The substrate compounds were prepared as described in Example 7.14. The kinase assay was done as described in Example 7.15.

Concentrations of dye-labeled peptides were determined by dilution of the purified peptides into dimethylformamide (200 μL) with 1 M NaOH (5 μL) and measuring the absorbance of either 5-carboxy-2′,7′-dipyridyl-sulfonefluorescein (i.e. dye2) at its absorbance maximum (548 nm) or 2′,7′,4,7-tetachloro-5-carboxy fluorescein (i.e. 2′,7′-dichloro-5-carboxy-4,7-dichlorofluorescein or “tet”) at its absorbance maximum (541 nm). The extinction coefficient of both dyes was assumed to be 80,000 cm⁻¹M⁻¹.

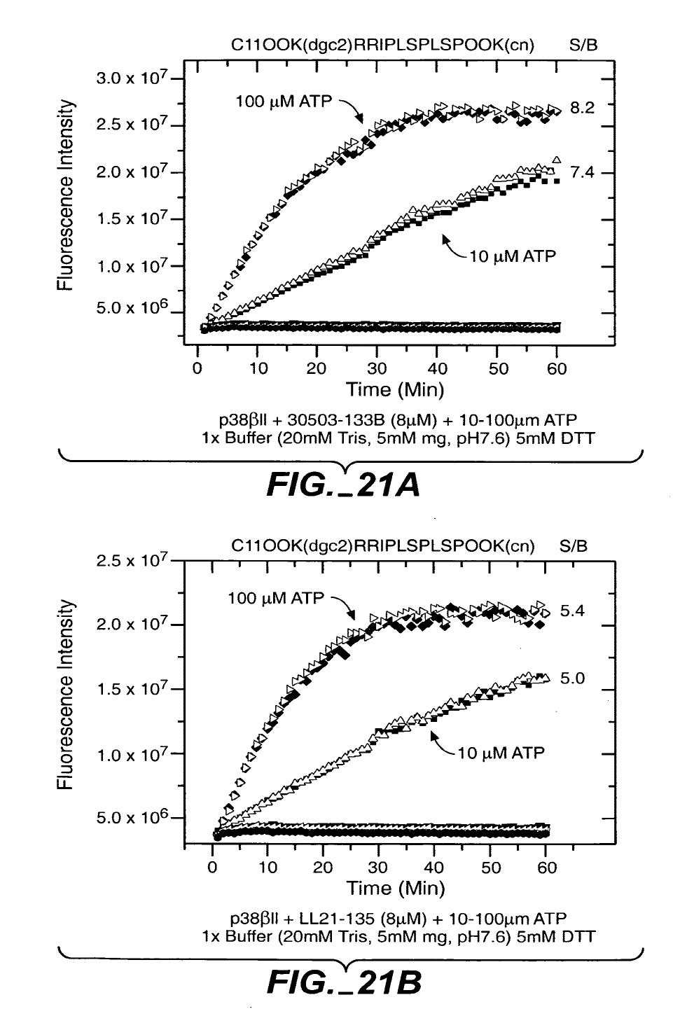

A reaction solution was prepared containing compound 1 (2 mM) 20 mM Tris buffer, pH 7.4, MgCl2 (5 mM), DTT (5 mM) and p38bII (14 nM). The solution was pipetted into wells of a 384-well plate (10 mL per well). Varying concentrations of ATP (final conc 10 and 100 μM) were added to the wells to initiate the kinase reaction. The plate was read at 500 nm excitation, 550 nm emission, each minute for 1 hour. The results are shown in FIGS. 21A and 21B. The signal to background ratio for the kinase substrate with two recognition sequences (FIG. 21B) was improved as compared to the signal to background ratio for the kinase substrate with one recognition sequence. Thus, kinase substrates with two recognition sequence provide improved signal to background ratios than the same substrate with one sequence moiety (FIG. 21A).

While the foregoing has presented specific embodiments, it is to be understood that these embodiments have been presented by way of example only. It is expected that others will perceive and practice variations which, though differing from the foregoing, do not depart form the spirit and scope of the teachings as described and claimed herein.

1. A micelle comprising:
(i) a ligand molecule comprising one or more hydrophobic moieties capable of integrating the ligand molecule in the micelle and a binding moiety; and
(ii) a signal molecule comprising one or more hydrophobic moieties capable of integrating the signal molecule in the micelle, one or more fluorescent moieties and a modification moiety modifiable by a modification agent, wherein the fluorescence of the fluorescent moieties are quenched within the micelle.

2. The micelle of claim 1 in which the modification moiety of the signal molecule comprises one or more enzyme recognition moiety(ies) including a cleavage site capable of being cleaved by a cleaving enzyme.

3. The micelle of claim 2 in which the cleaving enzyme is a phospholipase.

4. The micelle of claim 1 in which the modification moiety of the signal molecule comprises one enzyme recognition moiety including a protein kinase recognition sequence comprising one or more residues capable of being phosphorylated or dephosphorylated.

5. The micelle of claim 1 in which the modification moiety of the signal molecule comprises an enzyme recognition moiety including two or more protein kinase recognition sequences, wherein each enzyme recognition sequence, independently of the other, comprises one or more residues capable of being phosphorylated or dephosphorylated.

6. The micelle of claim 5 in which the first protein kinase recognition sequence is linked directly to the second protein kinase recognition sequence and the second protein kinase recognition sequence is linked to the fluorescent moiety via an optional linker and the hydrophobic moiety is linked to the fluorescent moiety via an optional linker.

7. The micelle of claim 5 in which the first protein kinase recognition sequence is linked to the second protein kinase recognition sequence through one or more optional linkers.

8. The micelle of claim 5 in which at least one unphosphorylated reside is tyrosine, serine or threonine.

9. The micelle of claim 5 in which each of the protein kinase recognition sequences, independently of the other, comprises N amino acid residues, wherein N represents the total number of amino acid residues comprising the recognition sequence, and is an integer from 1 to 10.

10. The micelle of claim 5 in which one of the protein kinase recognition sequences independently of the other, comprises N-u amino acid residues, wherein u represents the number of amino acid residues that can be omitted from the kinase recognition sequence, and is an integer from 1 to 9.

11. The micelle of claim 5 in which each protein kinase recognition sequence, independently from the other, is recognized by the same protein kinase.

12. The micelle of claim 5 in which each protein kinase recognition sequence, independently from the other, is recognized by a different protein kinase.

13. The micelle of claim 5 in which the signal molecule comprises two hydrophobic moieties, wherein one hydrophobic moiety is linked to the protein kinase recognition sequence through the fluorescent moiety, optionally via a linker, and the second hydrophobic moiety is linked to the protein kinase recognition sequence optionally via a linker.

14. The micelle claim 1 that further comprises a charge-balance molecule comprising a hydrophobic moiety capable of integrating the charge-balance molecule into the micelle and a charge-balance moiety capable of balancing the overall charge of the micelle, such that the net charge of the micelle ranges from ⁻1 to ⁺1 at physiological pH.

15. The micelle of claim 1 in which the signal molecule further comprises a charge-balance moiety capable of balancing the overall charge of the micelle, such that the net charge of the micelle ranges from ⁻1 to ⁺1 at physiological pH.

16. The micelle of claim 4 in which the signal molecule comprises a modification moiety comprising an enzyme recognition moiety recognized by a phosphatase, sulfatase or peptidase.

17. The micelle of claim 4 in which the signal molecule comprises a modification moiety comprising an enzyme recognition moiety comprising a peptide sequence selected from the group consisting of:

| | |
|---|---|
| -R-R-X-S/T-Z- | (SEQ ID NO: 1) |
| -L-R-R-A-S-L-G- | (SEQ ID NO: 2) |
| -R-X-X-S/T-F-F- | (SEQ ID NO: 3) |
| -R-Q-G-S-F-R-A- | (SEQ ID NO: 4) |
| -S/T-P-X-R/K- | (SEQ ID NO: 5) |
| -P-X-S/T-P- | (SEQ ID NO: 6) |
| -R-R-I-P-L-S-P- | (SEQ ID NO: 7) |
| -K-K-K-K-R-F-S-F-K- | (SEQ ID NO: 8) |
| -X-R-X-X-S-X-R-X- | (SEQ ID NO: 9) |
| -L-R-R-L-S-D-S-N-F- | (SEQ ID NO:10) |
| -K-K-L-N-R-T-L-T-V-A- | (SEQ ID NO: 11) |
| -E-E-I-Y-E/G-X-F- | (SEQ ID NO: 12) |
| -E-E-I-Y-G-E-F-R- | (SEQ ID NO: 13) |
| -E-I-Y-E-X-I/V- | (SEQ ID NO: 14) |
| -I-Y-M-F-F-F- | (SEQ ID NO: 15) |
| -Y-M-M-M- | (SEQ ID NO: 16) |
| -E-E-E-Y-F- | (SEQ ID NO: 17) |
| -R-I-G-E-G-T-Y-G-V-V-R-R- | (SEQ ID NO: 18) |
| -R-P-R-T-S-S-F- | (SEQ ID NO: 19) |
| -P-R-T-P-G-G-R- | (SEQ ID NO: 20) |
| -R-L-N-R-T-L-S-V- | (SEQ ID NO: 21) |
| -D-R-R-L-S-S-L-R- | (SEQ ID NO: 22) |
| -E-A-I-Y-A-A-P-F-A-R-R-R- | (SEQ ID NO: 23) |
| -K-V-E-K-I-G-E-G-T-Y-G-V-V-Y-K | (SEQ ID NO: 24) |
| -E-E-E-I-Y-G-E-F- | (SEQ ID NO: 25) |
| -R-H-S-S-P-H-Q-S(PO₄²⁻)-E-D-E-E- | (SEQ ID NO: 26) |
| -R-R-K-D-L-H-D-D-E-E-D-E-A-M-S-I- | (SEQ ID NO: 27) |
| T-A | |
| -S(PO₄²⁻)-X-X-S/T- | (SEQ ID NO: 28) |
| -S-X-X-E/D- | (SEQ ID NO: 29) |
| -R-R-R-D-D-D-S-D-D-D- | (SEQ ID NO: 30) |
| -K-G-P-W-L-E-E-E-E-E-A-Y-G-W-L-D- | (SEQ ID NO: 31) |
| F-; | |
| and, | |

analogs and conservative mutants thereof, wherein X represents any residue, Z represents a hydrophobic residue, and S(PO₄²⁻) represents a phosphorylated residue.

18. The micelle of claim 14 in which the charge-balance moiety comprises amino acids having charged side chain groups.

19. The micelle of claim 14 in which the signal molecule comprises a modification moiety comprising an enzyme recognition moiety comprising the peptide sequence -E-E-I-Y-G-E-F- (SEQ ID NO:32) and the charge-balance moiety comprises the peptide sequence -R-R-E-I-Y-G-R-F- (SEQ ID NO:33).

20-53. (canceled)
